SEQUENCE LISTING (5 sequences)

SEQ ID NO: 1 - DNA, 1535 bp, Streptomyces lydicus
gcattcacggagagtttgatcctggctcaggacgaacgctggcggcgtgcttaacacatgcaagtcgaacgatgaacctccttcgggaggggattagtggcgaacgggtgagtaacacgtgggcaatctgcccttcactctgggacaagccctggaaacggggtctaataccggatacgacacggggtcgcatgacctccgtgtggaaagctccggcggtgaaggatgagcccgcggcctatcagcttgttggtggggtgatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgagagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaaaccctggagacagggtcccccttgtggtcggtgtacaggtggtgcatggctgtcgtcagctcgtgtcgtgagatgttgggttaagtcccgcaacgagcgcaacccttgttctgtgttgccagcatgcccttcggggtgatggggactcacaggagactgccggggtcaactcggaggaaggtggggacgacgtcaagtcatcatgccccttatgtcttgggctgcacacgtgctacaatggccggtacaatgagctgcgataccgcgaggtggagcgaatctcaaaaagccggtctcagttcggattggggtctgcaactcgaccccatgaagtcggagttgctagtaatcgcagatcagcattgctgcggtgaatacgttcccgggccttgtacacaccgcccgtcacgtcacgaaagtcggtaacacccgaagccggtggcccaaccccttgtgggagggaatcgtcgaaggtgggactggcgattgggacgaagtcgtaacaaggtagccgtaccggaaggtgcggctggatcacctcctttcta

SEQ ID NO: 2 - DNA, 1531 bp, Streptomyces sp. 3211.1
acattcatggagagtttgatcctggctcaggacgaacgctggcggcgtgcttaacacatgcaagtcgaacgatgaagcccttcggggtggattagtggcgaacgggtgagtaacacgtgggcaatctgcccttcactctgggacaagccctggaaacggggtctaataccggataatactcctgcctgcatgggcgggggttgaaagctccggcggtgaaggatgagcccgcggcctatcagcttgttggtggggtaatggcccaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccgaggcttaacctcgggtctgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatataccggaaagcattagagatagtgccccccttgtggtcggtatacaggtggtgcatggctgtcgtcagctcgtgtcgtgagatgttgggttaagtcccgcaacgagcgcaacccttgtcctgtgttgccagcatgcccttcggggtgatggggactcacaggagaccgccggggtcaactcggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcccaacccgtaagggagggagctgtcgaaggtgggactggcgattgggacgaagtcgtaacaaggtagccgtaccggaaggtgcggctggatcacctcctttcta

SEQ ID NO: 3 - DNA, 1245 bp, Brevibacillus laterosporus
atcagtcggcgtgctatacatgcagtcgagcgagggtcttcggaccctagcggcggacgggtgagtaacacgtaggcaacctgcctgtgagactgggataacatagggaaacttatgctaataccggatagggttttgcttcgcctgaagcgaaacggaaagatggcgcaagctatcacttacagatgggcctgcggcgcattagctagttggtgaggtaacggctcaccaaggcgacgatgcgtagccgacctgagagggtgaccggccacactgggactgagacacggcccagactcctacgggaggcagcagtagggaattttccacaatggacgaaagtctgatggagcaacgccgcgtgaacgatgaaggctttcgggtcgtaaagttctgttgttagggaagaaacagtgctatttaaataagatagcaccttgacggtacctaacgagaaagccacggctaactacgtgccagcagccgcggtaatacgtaggtggcaagcgttgtccggaattattgggcgtaaagcgcgcgcaggtggctatgtaagtctgatgttaaagcccgaggctcaacctcggttcgcattggaaactgtgtagcttgagtgcaggagaggaaagtggtattccacgtgtagcggtgaaatgcgtagagatgtggaggaacaccagtggcgaaggcgactttctggcctgtaactgacactgaggcgcgaaagcgtggggagcaaacaggattagataccctggtagtccacgccgtaacgatgagtgctaggtgttaggggtttcaatacccttagtgccgcagctaacgcaataagcactccgcctgggagtacgctcgcaagagtgaaactcaaaggaattgacgggggcccgcacaagcggtggagcatgtggtttaattcgaagcacgcgaagaaccttaccaggtcttgacatcccactgaccgctctagagataagagcttcccttcggggcagtgtgacagtggtgcatgttgtcgtcagctcgtgtcgtggagaatgtggtagtcccgcaacgagcgccacccttatctttagtgccagcattcagttggcactctaagaagagactgcgtcgaacaagacggaggaaggcgggatgacgtcatcattcatgccgtagacttgggctaccacgtgctacagtgtgtacacgagtcaatctccgggagagaagtccaatcctaaaagccagtctagattcgag

SEQ ID NO: 4 - DNA, 1243 bp, Brevibacillus laterosporus
gggcaacacctacccttcggcggctggctccttacggttacctcaccgcacttcgcggtgttgcaaactcccgtggtgtgacgggcggtgtgtacaaggcccgggaacgtattcaccgcggcatgctgatccgcgattactagcgattccgacttcatgtaggcgagttgcagcctacaatccgaactgagattggttttaagagattagcatcttctcgcgaagtagcatcccgttgtaccaaccattgtagcacgtgtgtagcccaggtcataaggggcatgatgatttgacgtcatccccgccttcctccgtcttgtcgacggcagtctctctagagtgcccaactgaatgctggcaactaaagataagggttgcgctcgttgcgggacttaacccaacatctcacgacacgagctgacgacaaccatgcaccacctgtcaccactgccccgaagggaagctctatctctagagcggtcagtgggatgtcaagacctggtaaggttcttcgcgttgcttcgaattaaaccacatgctccaccgcttgtgcgggcccccgtcaattcctttgagtttcactcttgcgagcgtactccccaggcggagtgcttattgcgttagctgcggcactaagggtattgaaacccctaacacctagcactcatcgtttacggcgtggactaccagggtatctaatcctgtttgctccccacgctttcgcgcctcagtgtcagttacaggccagaaagtcgccttcgccactggtgttcctccacatctctacgcatttcaccgctacacgtggaaataccactttcctctcctgcactcaagctacacagtttccaatgcgaaccgaggttgagcctcgggctttaacatcagacttacatagccacctgcgcgcgctttacgcccaataattccggacaacgcttgccacctacgtattacgcggctgctggcacgtagttagcgtggctttctcgttaggtaccgtcaaggtgctatcttatttaaatagcactgtttcttccctaacaacagaacttacgacccgaagcttcatcgtcacgcgcgtgctcatcagacttcgtcatgtgaaatcctactgctgcctccgtagagtctggcgtgttctcagttcaagtgtggaccgttcacctctcaggttcgctagcatcgtgccttgagagccgttaactaccaactaggcctaatgggctgctgcgaagtccat

SEQ ID NO: 5 - DNA, 1243 bp, Brevibacillus laterosporus
atggacttcgcagcagcccattaggcctagttggtagttaacggctctcaaggcacgatgctagcgaacctgagaggtgaacggtccacacttgaactgagaacacgccagactctacggaggcagcagtaggatttcacatgacgaagtctgatgagcacgcgcgtgacgatgaagcttcgggtcgtaagttctgttgttagggaagaaacagtgctatttaaataagatagcaccttgacggtacctaacgagaaagccacgctaactacgtgccagcagccgcgtaatacgtaggtggcaagcgttgtccggaattattgggcgtaaagcgcgcgcaggtggctatgtaagtctgatgttaaagcccgaggctcaacctcggttcgcattggaaactgtgtagcttgagtgcaggagaggaaagtggtatttccacgtgtagcggtgaaatgcgtagagatgtggaggaacaccagtggcgaaggcgactttctggcctgtaactgacactgaggcgcgaaagcgtggggagcaaacaggattagataccctggtagtccacgccgtaaacgatgagtgctaggtgttaggggtttcaatacccttagtgccgcagctaacgcaataagcactccgcctggggagtacgctcgcaagagtgaaactcaaaggaattgacgggggcccgcacaagcggtggagcatgtggtttaattcgaagcaacgcgaagaaccttaccaggtcttgacatcccactgaccgctctagagatagagcttcccttcggggcagtggtgacaggtggtgcatggttgtcgtcagctcgtgtcgtgagatgttgggttaagtcccgcaacgagcgcaacccttatctttagttgccagcattcagttgggcactctagagagactgccgtcgacaagacggaggaaggcggggatgacgtcaaatcatcatgccccttatgacctgggctacacacgtgctacaatggttggtacaacgggatgctacttcgcgagaagatgctaatctcttaaaaccaatctcagttcggattgtaggctgcaactcgcctacatgaagtcggaatcgctagtaatcgcggatcagcatgccgcggtgaatacgttcccgggccttgtacacaccgcccgtcacaccacgggagtttgcaacaccgcgaagtgcggtgaggtaaccgtaaggagccagccgccgaagggtaggtgttgccc

(19) United States
(12) Patent Application Publication
(10) Pub. No.: US 2021/0251237 A1
(43) Pub. Date: Aug. 19, 2021

(54) PLATFORM FOR DEVELOPING SOIL-BORNE PLANT PATHOGEN INHIBITING MICROBIAL CONSORTIA

(71) Applicant: REGENTS OF THE UNIVERSITY OF MINNESOTA, Minneapolis, MN (US)

(72) Inventor: Linda L. Kinkel, Minneapolis, MN (US)

(21) Appl. No.: 17/262,541

(22) PCT Filed: Jul. 25, 2019
(86) PCT No.: PCT/US2019/043539

§ 371 Date: Jan. 22, 2021

Related U.S. Application Data
(60) Provisional application No. 62/858,446, filed on Jun. 7, 2019.
(60) Provisional application No. 62/713,394, filed on Aug. 1, 2018.
(60) Provisional application No. 62/703,060, filed on Jul. 25, 2018.

(51) Int. Cl. A01N 63/22; A01N 63/28

(52) U.S. Cl. CPC A01N 63/22; A01N 63/28

(57) ABSTRACT

The disclosure relates to a systemic platform for developing soil-borne plant pathogen inhibiting microbial consortia. The platform utilizes multivariate computer modelling and multidimensional ecological function balancing (MEFB) nodal analysis to develop microbial consortia, consortia, and inoculants. The disclosure further relates to a prescriptive biocontrol system that will enable farmers to have site-specific agricultural biologics developed for their specific site and crop of interest.

### CROSS-REFERENCE TO RELATED APPLICATIONS

This application claims the benefit of priority to U.S. Provisional Application No. 62/858,446, filed on Jun. 7, 2019; U.S. Provisional Application No. 62/713,394, filed on Aug. 1, 2018; and U.S. Provisional Application No. 62/703,060, filed on Jul. 25, 2018, the contents of each of which is incorporated herein by reference in their entirety for all purposes.

### GOVERNMENT FUNDING

This invention was made with government support under MCB-9977907 awarded by National Science Foundation and under 2006-35319-17445 awarded by the National Institute of Food and Agriculture, USDA. The government has certain rights in the invention.

### FIELD

The disclosure relates to a systemic platform for developing soil-borne plant pathogen inhibiting microbial consortia. The platform utilizes multivariate computer modelling and multidimensional ecological function balancing (MEFB) nodal analysis to develop microbial consortia, and inoculants. The disclosure further relates to a prescriptive biocontrol system that will enable farmers to have site-specific agricultural biologics developed for their specific site and crop of interest.

### STATEMENT REGARDING SEQUENCE LISTING

The sequence listing associated with this application is provided in text format in lieu of a paper copy, and is hereby incorporated by reference into the specification. The name of the text file containing the sequence listing is BICL_001_00US_ST25.txt. The text file is 10 kb, was created on Jul. 25, 2019, and is being submitted electronically via EFS-Web.

### BACKGROUND

Soil borne pathogens represent a significant challenge to crop production globally. Virtually all crops, including annual and perennial field, fruit, vegetable, and horticultural species in temperate, tropical, and greenhouse production systems suffer significant losses in plant establishment, yields, and plant or crop quality due to soil borne pathogens. While both plant resistance and fungicide use can offer some protection against losses due to soil borne pathogens, many crops lack resistance to diverse soil borne pathogens, and fungicides are costly, can vary in effectiveness, and have negative environmental impacts.

Biological control of soil borne pathogens has been limited by, for example, a limited base of microbes upon which development is focused and/or a focus on single-strain inoculants. In particular, single strain inoculants can fail to provide a level of disease suppression sufficient to satisfy market demands. The capacity for a single microbial strain to provide protection against any possible soil borne pathogen on diverse crops in a wide range of physical and environmental conditions, and in the presence of complex and highly-variable naturally occurring soil microbial communities, is low.

Therefore, there is a need for effective compositions and methods for the bioremediation of pathogen-infested soils. Further, compositions and methods that can be customized to be effective towards inhibiting plant pathogens in the context of different types of pathogens, crops, locations and soils would be particularly valuable.

### SUMMARY

The disclosure provides methods of creating a soil-borne plant pathogen inhibiting microbial consortia, comprising: (a) accessing or creating a soil-borne plant pathogen suppressive microbial library; (b) utilizing microbes from the library of step a) to access or create one or more ecological function balancing nodal microbial libraries, selected from the group consisting of: a mutual inhibitory activity microbial library, a carbon nutrient utilization complementarity microbial library, an antimicrobial signaling capacity and responsiveness microbial library, a plant growth promotion ability microbial library, and an antimicrobial resistance to clinical antimicrobials library; (c) performing a multi-dimensional ecological function balancing (MEFB) nodal analysis utilizing said one or more nodal microbial libraries; and (d) selecting at least two microbes from the soil-borne plant pathogen suppressive microbial library based on the MEFB nodal analysis, thereby producing a soil-borne plant pathogen inhibiting microbial consortia having a targeted ecological function in at least one dimension.

The disclosure provides methods of creating a soil-borne plant pathogen inhibiting microbial consortia, comprising: (a) accessing or creating a soil-borne plant pathogen suppressive microbial library; (b) utilizing microbes from the library of step a) to access or create one or more ecological function balancing nodal microbial libraries, selected from the group consisting of: a mutual inhibitory activity microbial library, a carbon nutrient utilization complementarity microbial library, an antimicrobial signaling capacity and responsiveness microbial library, a plant growth promotion ability microbial library, and an antimicrobial resistance to clinical antimicrobials library; (c) performing a multi-dimensional ecological function balancing (MEFB) nodal analysis utilizing said one or more nodal microbial libraries; (d) assembling a library of microbial consortia, each microbial consortia comprising at least two microbes from the soil-borne plant pathogen suppressive microbial library, selected based on the MEFB nodal analysis; (e) screening microbial consortia from the library of microbial consortia in the presence of a plurality of soil-borne plant pathogens to produce a soil-borne plant pathogen suppressive profile for each screened microbial consortia; (f) optionally ranking microbial consortia from the library of screened microbial consortia based upon at least one dimension of the soil-borne plant pathogen suppressive profile of each microbial consortia; and (g) selecting a soil-borne plant pathogen inhibiting microbial consortia having the desired soil-borne plant pathogen suppressive profile from the library.

The disclosure provides methods for creating a soil-borne plant pathogen inhibiting microbial consortia having a targeted and complementary soil-borne plant pathogen suppressive profile, comprising: a) screening a population of microbial isolates in the presence of a plurality of soil-borne plant pathogens, including a target soil-borne pathogen, to create a soil-borne plant pathogen suppressive profile for each individual microbial isolate in said population; b) assembling a library of microbial consortia, each microbial consortia comprising a combination of microbial isolates from those screened in step a), wherein each microbial consortia in said library has a predicted soil-borne plant pathogen suppressive profile that is distinct from any individual microbial isolate soil-borne plant pathogen suppressive profile from step a) in at least one dimension of the soil-borne plant pathogen suppressive profile; wherein at least one microbial isolate in each of the assembled microbial consortia suppresses the growth of the target soil-borne pathogen; c) optionally ranking microbial consortia from the library of microbial consortia based upon at least one dimension of the predicted soil-borne plant pathogen suppressive profile of each microbial consortia; and d) selecting a soil-borne plant pathogen inhibiting microbial consortia from the library of microbial consortia, said selected consortia having the desired targeted and complementary soil-borne plant pathogen suppressive profile.

The disclosure provides methods for creating a soil-borne plant pathogen inhibiting microbial consortia having a targeted and complementary soil-borne plant pathogen suppressive profile, comprising: a) screening a population of microbial isolates in the presence of a plurality of soil-borne plant pathogens, including a target soil-borne pathogen, to create a soil-borne plant pathogen suppressive profile for each individual microbial isolate in said population; b) assembling a library of microbial consortia, each microbial consortia comprising a combination of microbial isolates from those screened in step a), wherein each microbial consortia in said library has a predicted soil-borne plant pathogen suppressive profile that is distinct from any individual microbial isolate soil-borne plant pathogen suppressive profile from step a) in at least one dimension of the soil-borne plant pathogen suppressive profile; wherein at least one microbial isolate in each of the assembled microbial consortia suppresses the growth of the target soil-borne pathogen; c) screening microbial consortia from the library of microbial consortia in the presence of a plurality of soil-borne plant pathogens, including the target soil-borne pathogen, to produce a soil-borne plant pathogen suppressive profile for each screened microbial consortia; d) optionally ranking microbial consortia from the library of screened microbial consortia based upon at least one dimension of the soil-borne plant pathogen suppressive profile of each microbial consortia; and e) selecting a soil-borne plant pathogen inhibiting microbial consortia having the desired targeted and complementary soil-borne plant pathogen suppressive profile from the library.

The disclosure provides methods for creating a soil-borne plant pathogen inhibiting microbial consortia having a designed level of mutual inhibitory activity, comprising: a) assembling a library of microbial consortia, each consortia comprising a combination of at least two microbial isolates; b) screening microbial consortia of the assembled library for relative degree of mutual inhibitory activity displayed by each microbial isolate towards every other microbial isolate within its microbial consortia; c) developing an n-dimensional mutual inhibitory activity matrix for microbial consortia based on the mutual inhibitory activities screened in step (b); and d) selecting a soil-borne plant pathogen inhibiting microbial consortia having the designed level of mutual inhibitory activity from the library based upon the n-dimensional mutual inhibitory activity matrix.

### BRIEF DESCRIPTION OF THE FIGURES

FIG. 1 depicts non-metric multidimensional scaling (NMDS) analysis of nutrient use profiles in Biolog SF-P2 Microplate™ for Streptomyces isolates from non-amended soil and soil with carbon amendments. This figure illustrates the selective impacts of nutrients on the soil Streptomyces.

FIG. 2 depicts the niche width (left panel; mean number of nutrients utilized by each isolate) and niche overlap (right panel; mean percent of nutrient use shared between all possible pairwise combinations of isolates) among Streptomyces isolates from non-amended soil and soil with carbon amendments. The data were analyzed using Fisher's LSD test; p-value of <0.05; error bars represent standard error.

FIG. 3 depicts the frequency distribution of inhibitory phenotypes amongst Streptomyces isolates from non-amended soil and soil with carbon amendments (left panel), and the percent of the Streptomyces isolates that are inhibitory against Streptomyces from non-amended soils and soil with carbon amendments (right panel).

FIG. 4 depicts the relationship between niche overlap and intensity of inhibition among Streptomyces isolates from soil with carbon amendments against isolates from non-amended soil (left panel), and among Streptomyces from non-amended soil against isolates from soil with carbon amendments (right panel). This illustrates the significant capacities of soil carbon amendments to select for enhanced inhibitory phenotypes against indigenous resource competitors (left-hand panel), and the lack of capacity of those competitors to reciprocally antagonize the enhanced inhibitors (right-hand panel).

FIG. 5 depicts the percent scab control (reduction in scab severity in inoculated plots versus non-inoculated plots in the same field trial) in relation to disease on non-inoculated plots. Each data point represents results from an individual inoculated trial. The present figure was constructed utilizing methods from: “Example 10: Various Protocols and Field Trials Utilizing Microbes Developed via the Platform—C, E, & F.”

FIG. 6 depicts a photograph of a potato with potato scab disease.

FIG. 7A-B. Inoculant included in this figure: GS1. FIG. 7A depicts the severity of disease on 3-week-old greenhouse-grown soybean plants. The present figure was constructed utilizing methods from Example 9(F)(iii). FIG. 7B shows biological control of Phytophthora on alfalfa in greenhouse trials. Briefly, soils were inoculated with a liquid spore suspension of a single Streptomyces isolate at planting. Alfalfa seeds were planted into field soil that was naturally-infested with the pathogen Phytophthora medicaginis. After 28 days, plants were harvested for disease and biomass assessments. Disease was significantly reduced and plant biomass was significantly increased on inoculated vs. non-inoculated plants.

FIG. 8 depicts the reduction in sudden death root symptoms from 3-35% on inoculated plants harvested at six weeks in greenhouse trials. The present data was constructed utilizing methods from Example 10(G). Microbial inoculants included in each combinations: A: GS1, SS2, SS3; B: GS1, SS3, PS5; C: GS1, SS6, PS5; D: GS1, SS2, PS7; E: GS1, SS6, PS7

FIG. 9 depicts the increases seen in marketable yields of potato following use of microbial inoculants in field trials at Becker, Rosemount, and St. Paul research farms. There were two trials each at Becker and Rosemount, and seven inoculant trials at Saint Paul. The present data was constructed utilizing methods from Examples 10(A) and 10(D). Inoculant isolates: Becker, Bar 1: GS1, PS3; Becker, Bar 2: GS1; Rosemount, Bar 1: GS1, PS3; Rosemount, Bar 2: PS3, SS4; STP, Bar 1: GS1, SS2, SS3; STP, Bar 2: GS1, SS2, PS7; STP, Bar 3: GS1, PS3, PS7; STP, Bar 4: GS1, SS2, PS1; STP, Bar 5: GS1, PS3, PS1; STP, Bar 6: PS3, SS2, PS1; STP, Bar 7: GS1, SS5, PS5.

FIG. 10 depicts the biomass (greenhouse trials) and seed yield increases on soybean observed with the use of microbial inoculants in greenhouse and in field trials at two locations. Greenhouse biomass: mean value from treatment with 7 different isolate combinations; GS1, SS2, SS3; GS1, SS3, SS6; GS1, SS3, PS5; GS1, SS6, PS5; GS1, SS2, PS7; GS1, SS7, SS8; GS1, SS6, PS7. 2014 seed yield represents a mean among replicates for a single treatment: PS3. 2015 seed yield represents a mean generated from 2 treatments: GS2, SS2, PS3, PS4; GS1, SS2, SS3, PS4, PS2.

FIG. 11 depicts the increases seen in seed yields with the use of microbial inoculants in field plots at four locations in Minnesota. The present data was constructed utilizing methods from Example 10(F): Various Protocols and Field Trials Utilizing Microbes Developed via the Platform—H.” For each location, Bar 1: GS1, SS2, SS3, PS2, PS4; Bar 2: GS1, SS2, SS3; Bar 3: GS1, SS2, PS4, PS3.

FIG. 12A-B depicts potato scab disease on tubers in two experimental field plots following amendments with rice, microbial inoculants, carbon (nutrient'), or the combination of the microbial inoculants and the carbon nutrient. Bars highlighted with different letters are significantly different (ANOVA), with probability values for the analysis indicated in the upper right-hand corner of the figure. Inoculum was prepared by growing individual strains on nutrient medium, harvesting spores, and mixing spores with rice to produce a granular inoculant. The experiment was a randomized complete block design. Potatoes were inoculated at planting, with and without carbon amendment, including both a non-inoculated and a rice-only control. Potatoes were grown using standard production conditions, and disease was assessed at harvest for all treatments. FIG. 12A depicts the percent of scab disease in non-fumigated plots (left panel), or plots fumigated with chloropicrin. FIG. 12B depicts the percent scab disease as calculated from the combined fumigated and non-fumigated datasets.

FIG. 13 depicts an exemplary protocol for determining the effects of soil carbon amendment on Streptomyces soil isolates relative to non-amended control soil.

FIG. 14 illustrates a flowchart of the Soil-borne Plant Pathogen Inhibiting Microbial Consortia (SPPIMC) development platform.

FIG. 15 illustrates the SPPIMC development platform with more granularity.

FIG. 16 shows the mean individual tuber weights and total tuber yields per plot among treatments. The inoculant included 2 of the 3 strains (GS1, PS3) included in the optimized 3-strain LLK3-2017 inoculant combination.

FIG. 17A-B. FIG. 17A shows an example of pathogen inhibition assays. FIG. 17B shows an example of complementarity in pathogen inhibition profiles among a collection of Streptomyces.

FIG. 18 illustrates antibiotic inhibition of Streptomyces isolate (S-87) by antibiotics (C=chloramphenicol; S=streptomycin; R=rifampin/rifampicin; T=tetracycline).

FIG. 19A-B. FIG. 19A shows pairwise inhibition assays between Streptomyces isolates. FIG. 19B shows inhibitory interaction networks among three different sets of microbial isolates.

FIG. 20 shows variation in nutrient use preferences across 95 nutrients for a collection of bacterial isolates. Darker shading indicates higher growth under a specific media.

FIG. 21 shows signaling interactions between bacterial pairs. Individual bacterial isolates (isolates 15 and 18 on the left, isolates 12 and 15 on the right) are dotted onto nutrient medium at the same time, and grown for 3 days. Subsequently, a second layer of agar medium is overlaid onto the plates, and a pathogen is introduced to the plate (either by spread-plating an inoculum suspension, or by introducing a disk of a fresh fungal culture into the center of the plate; these plates show a spread-plated target). After 3-5 days (depending upon the pathogen), the capacity of the dot isolate to inhibit the pathogen ALONE (individual dot of isolate 15) is compared to its ability to inhibit the pathogen in the presence of another isolate (isolate 15 immediately adjacent to isolate 18 or isolate 12). In panel A, isolates 18 suppresses the capacity of isolate 15 to inhibit the pathogen. In contrast, in panel B, isolate 12 enhances the ability of isolate 15 to inhibit the pathogen. Note that panels A and B target different pathogens.

FIG. 22 shows signaling interactions that alter inhibitory phenotypes among 3 different collections of microbes.

FIG. 23 Numbers of isolates inhibitory against 0-5 target populations following 9 month amendments with low vs. high doses of glucose or lignin in soil mesocosms. Amendment with high doses of carbon resulted in more inhibitory Streptomyces populations than amendment with low doses.

FIG. 24 shows a plating strategy for studying signaling interactions among simple (n=2) to more complex (n=4-8) mixtures of bacteria.

FIG. 25 shows variation in growth potential at different temperatures among microbial inoculants. Isolates vary significantly in their maximum growth potential as well as the reductions in potential at cool temperatures. Data based upon incubation of individual isolates in Biolog SF-P2 Microplate™ plates at the specified temperatures over 5 days. Optical density measurements (y-axis) were summed over all nutrients on which the individual isolate exhibited growth. This figure illustrates the relative value of individual isolates will vary with temperature: GS1 and SS2 have a significant growth advantage over PS4 and PS2 at warmer temperatures, but this advantage disappears when considering growth in cooler temperature settings (e.g. early spring soil).

FIG. 26 shows biological control of potato scab using Streptomyces inoculants. Briefly, soils were inoculated in the field at planting with isolate PonSSII. Potatoes were grown using standard production methods, and harvested in early September. Potato scab severity was significantly reduced on the inoculated (left-hand side) vs. the non-inoculated tubers.

FIG. 27 shows the variation in growth promotion by different microbial inoculants on different plant hosts.

FIG. 28 illustrates the decreasing disease intensity (mean number of lesions per tuber, y-axis) with increasing numbers of Streptomyces isolates in the inoculant (inoculum combinations, x-axis).

FIG. 29 depicts the benefits of incorporation of a signaling inoculant into the mixture for enhancing disease suppression. Specifically, potato scab disease intensities (either mean lesion number—top panel, or percent surface infected—bottom panel) in field trials are significantly smaller when a signaler is inoculated with a collection of antagonists vs. when there no added signaler. Each bar represents the mean disease intensity over 5 different inoculants mixtures PLUS the signaler (Isolate 1231.5), or with no added signaler.

FIG. 30 shows the change in Streptomyces density in the presence or absence of carbon amendments.

FIG. 31 shows Herr's assay used to quantify the numbers and kill zones of Streptomyces inhibitors of 5 different plant pathogens.

FIG. 32 depicts the differences in the mean proportion of Streptomyces that are inhibitory against the collection of 5 plant pathogens (left-hand panel) and the mean kill zone of inhibitory Streptomyces (right-hand panel) from soils varying in plant species diversity (1, 4, 8, or 16 plant species).

FIG. 33 depicts mean percent niche overlap among Streptomyces from soils associated with plants growing in monoculture or in 16-species polycultures. Niche overlap is significantly smaller among populations in polycultures than in monocultures.

FIG. 34 depicts carbon compound richness for soils associated with two different plant hosts (the C4 grass Andropogon gerardii or the legume Lespedeza capitata). Data are the number of carbon peaks based upon pyrolysis gas chromatographic mass spectrometry data.

### DETAILED DESCRIPTION

### Definitions

While the following terms are believed to be well understood by one of ordinary skill in the art, the following definitions are set forth to facilitate explanation of the presently disclosed subject matter.

The term “a” or “an” may refer to one or more of that entity, i.e. can refer to plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

Reference throughout this specification to “one embodiment”, “an embodiment”, “one aspect”, or “an aspect” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As used herein, in particular embodiments, the terms “about” or “approximately” when preceding a numerical value indicates the value plus or minus a range of 10% unless otherwise stated or otherwise evident by the context, and except where such a range would exceed 100% of a possible value, or fall below 0% of a possible value, such as less than 0 CFU/ml of a bacteria, or more than 100% of a inhibition of growth.

As used herein the terms “microorganism” or “microbe” should be taken broadly. These terms are used interchangeably and include, but are not limited to, the two prokaryotic domains, Bacteria and Archaea, eukaryotic fungi and protozoa, as well as viruses.

The term “microbial community” means a group of microbes comprising two or more species or strains. Unlike microbial consortia, a microbial community does not have to be carrying out a common function, or does not have to be participating in, or leading to, or correlating with, a recognizable parameter, such as a phenotypic trait of interest (e.g. antimicrobial activity or production of compounds beneficial to plant growth).

As used herein, “isolate,” “isolated,” “isolated microbe,” and like terms, are intended to mean that the one or more microorganisms has been separated from at least one of the materials with which it is associated in a particular environment (for example soil, water, plant tissue).

As used herein, “soil” refers to any plant growth medium including any agriculturally acceptable growing media. Growing media may include, for example, soil, sand, compost, peat, soilless growing media containing organic and/or inorganic ingredients, artificial plant-growth substrates, polymer-based growth matrices, hydroponic nutrient and growth solutions, and combinations or mixtures thereof.

Microbes of the present disclosure may include spores and/or vegetative cells. In some embodiments, microbes of the present disclosure include microbes in a viable but non-culturable (VBNC) state, or a quiescent state. See Liao and Zhao (US Publication US2015267163A1). In some embodiments, microbes of the present disclosure include microbes in a biofilm. See Merritt et al. (U.S. Pat. No. 7,427,408).

Thus, an “isolated microbe” does not exist in its naturally occurring environment; rather, it is through the various techniques described herein that the microbe has been removed from its natural setting and placed into a non-naturally occurring state of existence. Thus, the isolated strain or isolated microbe may exist as, for example, a biologically pure culture, or as spores (or other forms of the strain) in association with an acceptable carrier.

As used herein, “spore” or “spores” refer to structures produced by bacteria and fungi that are adapted for survival and dispersal. Spores are generally characterized as dormant structures; however, spores are capable of differentiation through the process of germination. Germination is the differentiation of spores into vegetative cells that are capable of metabolic activity, growth, and reproduction. The germination of a single spore results in a single fungal or bacterial vegetative cell. Fungal spores are units of asexual reproduction, and in some cases are necessary structures in fungal life cycles. Bacterial spores are structures for surviving conditions that may ordinarily be nonconductive to the survival or growth of vegetative cells.

As used herein, “microbial composition” refers to a composition comprising one or more microbes of the present disclosure, wherein a microbial composition, in some embodiments, is administered to the soil, field, or plants described herein.

As used herein, “carrier”, “acceptable carrier”, or “agricultural carrier” refers to a diluent, adjuvant, excipient, or vehicle with which the compound is administered.

In some embodiments, carriers may be granular in structure, such as soil, sand, soil particles, or sand particles. In further embodiments, the carriers may be dry, as opposed to a moist or wet carrier. In some embodiments, carriers can be in solid or liquid form.

The terms “multi strain inoculate composition”, “consortium”, “bioconsortia,” “microbial consortia,” and “synthetic consortia” interchangeably refer to a composition comprising two or more microbes identified by methods, systems, and/or apparatuses of the present disclosure. In some embodiments, the microbes in the consortium do not exist together in a naturally occurring environment. In some embodiments, the microbes are present in the consortium at ratios or amounts that are not naturally occurring. In some embodiments, the consortium comprises two or more species, or two or more strains of a species, of microbes.

In certain embodiments of the disclosure, the isolated microbes exist as isolated and biologically pure cultures (e.g., microbial isolate(s)). It will be appreciated by one of skill in the art, that an isolated and biologically pure culture of a particular microbe, denotes that said culture is substantially free (within scientific reason) of other living organisms and contains only the individual microbe in question. The culture can contain varying concentrations of said microbe. The present disclosure notes that isolated and biologically pure microbes often “necessarily differ from less pure or impure materials.” See, e.g. In re Bergstrom, 427 F.2d 1394, (CCPA 1970)(discussing purified prostaglandins), see also, In re Bergy, 596 F.2d 952 (CCPA 1979)(discussing purified microbes), see also, Parke-Davis & Co. v. H.K. Mulford & Co., 189 F. 95 (S.D.N.Y. 1911) (Learned Hand discussing purified adrenaline), aff'd in part, rev'd in part, 196 F. 496 (2d Cir. 1912), each of which are incorporated herein by reference. Furthermore, in some embodiments, the disclosure provides for certain quantitative measures of the concentration, or purity limitations, that must be found within an isolated and biologically pure microbial culture. The presence of these purity values, in certain embodiments, is a further attribute that distinguishes the presently disclosed microbes from those microbes existing in a natural state. See, e.g., Merck & Co. v. Olin Mathieson Chemical Corp., 253 F.2d 156 (4th Cir. 1958) (discussing purity limitations for vitamin B12 produced by microbes), incorporated herein by reference.

As used herein, “individual isolates” should be taken to mean a composition, or culture, comprising a predominance of a single genera, species, or strain, of microorganism, following separation from one or more other microorganisms. The phrase should not be taken to indicate the extent to which the microorganism has been isolated or purified. However, “individual isolates” can comprise substantially only one genus, species, or strain, of microorganism.

The term “growth medium” as used herein, is any medium which is suitable to support growth of a microbe. By way of example, the media may be natural or artificial. It should be appreciated that the media may be used alone or in combination with one or more other media. It may also be used with or without the addition of exogenous nutrients.

The medium may be amended or enriched with additional compounds or components, for example, a component which may assist in the interaction and/or selection of specific groups of microorganisms. For example, antibiotics (such as penicillin) or sterilants (for example, quaternary ammonium salts and oxidizing agents) could be present and/or the physical conditions (such as salinity, nutrients (for example organic and inorganic minerals (such as phosphorus, nitrogenous salts, ammonia, potassium and micronutrients such as cobalt and magnesium), pH, and/or temperature), methionine, prebiotics, ionophores, and beta glucans could be amended.

As used herein, “improved” should be taken broadly to encompass improvement of a characteristic of interest, as compared to a control group, or as compared to a known average quantity associated with the characteristic in question. In the present disclosure, “improved” does not necessarily demand that the data be statistically significant (i.e. p <0.05); rather, any quantifiable difference demonstrating that one value (e.g. the average treatment value) is different from another (e.g. the average control value) can rise to the level of “improved.”

As used herein, “inhibiting and suppressing” and like terms should not be construed to require complete inhibition or suppression, although this may be desired in some embodiments.

The term “marker” or “unique marker” as used herein is an indicator of unique microorganism type, microorganism strain or activity of a microorganism strain. A marker can be measured in biological samples and includes without limitation, a nucleic acid-based marker such as a ribosomal RNA gene, a peptide- or protein-based marker, and/or a metabolite or other small molecule marker.

The term “metabolite” as used herein is an intermediate or product of metabolism. A metabolite in one embodiment is a small molecule. Metabolites have various functions, including in fuel, structural, signaling, stimulatory and inhibitory effects on enzymes, as a cofactor to an enzyme, in defense, and in interactions with other organisms (such as pigments, odorants and pheromones). A primary metabolite is directly involved in normal growth, development and reproduction. A secondary metabolite is not directly involved in these processes but usually has an important ecological function. Examples of metabolites include but are not limited to antibiotics and pigments such as resins and terpenes, etc. Some antibiotics use primary metabolites as precursors, such as actinomycin which is created from the primary metabolite, tryptophan. Metabolites, as used herein, include small, hydrophilic carbohydrates; large, hydrophobic lipids and complex natural compounds.

As used herein, the term “genotype” refers to the genetic makeup of an individual cell, cell culture, tissue, organism, or group of organisms.

As used herein, the term “allele(s)” means any of one or more alternative forms of a gene, all of which alleles relate to at least one trait or characteristic. In a diploid cell, the two alleles of a given gene occupy corresponding loci on a pair of homologous chromosomes. Since the present disclosure, in embodiments, relates to QTLs, i.e. genomic regions that may comprise one or more genes or regulatory sequences, it is in some instances more accurate to refer to “haplotype” (i.e. an allele of a chromosomal segment) instead of “allele”, however, in those instances, the term “allele” should be understood to comprise the term “haplotype”. Alleles are considered identical when they express a similar phenotype. Differences in sequence are possible but not important as long as they do not influence phenotype.

As used herein, the term “locus” (loci plural) means a specific place or places or a site on a chromosome where for example a gene or genetic marker is found.

As used herein, the term “genetically linked” refers to two or more traits that are co-inherited at a high rate during breeding such that they are difficult to separate through crossing.

A “recombination” or “recombination event” as used herein refers to a chromosomal crossing over or independent assortment. The term “recombinant” refers to an organism having a new genetic makeup arising as a result of a recombination event.

As used herein, the term “molecular marker” or “genetic marker” refers to an indicator that is used in methods for visualizing differences in characteristics of nucleic acid sequences. Examples of such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Markers further include polynucleotide sequences encoding 16S or 18S rRNA, and internal transcribed spacer (ITS) sequences, which are sequences found between small-subunit and large-subunit rRNA genes that have proven to be especially useful in elucidating relationships or distinctions among when compared against one another. Mapping of molecular markers in the vicinity of an allele is a procedure which can be performed by the average person skilled in molecular-biological techniques.

The primary structure of major rRNA subunit 16S comprise a particular combination of conserved, variable, and hypervariable regions that evolve at different rates and enable the resolution of both very ancient lineages such as domains, and more modern lineages such as genera. The secondary structure of the 16S subunit include approximately 50 helices which result in base pairing of about 67% of the residues. These highly conserved secondary structural features are of great functional importance and can be used to ensure positional homology in multiple sequence alignments and phylogenetic analysis. Over the previous few decades, the 16S rRNA gene has become the most sequenced taxonomic marker and is the cornerstone for the current systematic classification of bacteria and archaea (Yarza et al. 2014. Nature Rev. Micro. 12:635-45).

A sequence identity of 94.5% or lower for two 16S rRNA genes is strong evidence for distinct genera, 86.5% or lower is strong evidence for distinct families, 82% or lower is strong evidence for distinct orders, 78.5% is strong evidence for distinct classes, and 75% or lower is strong evidence for distinct phyla. The comparative analysis of 16S rRNA gene sequences enables the establishment of taxonomic thresholds that are useful not only for the classification of cultured microorganisms but also for the classification of the many environmental sequences. Yarza et al. 2014. Nature Rev. Micro. 12:635-45).

As used herein, the term “trait” refers to a characteristic or phenotype. A trait may be inherited in a dominant or recessive manner, or in a partial or incomplete-dominant manner. A trait may be monogenic (i.e. determined by a single locus) or polygenic (i.e. determined by more than one locus) or may also result from the interaction of one or more genes with the environment.

As used herein, the term “phenotype” refers to the observable characteristics of an individual cell, cell culture, organism (e.g., a bacterium), or group of organisms which results from the interaction between that individual's genetic makeup (i.e., genotype) and the environment.

As used herein, the term “chimeric” or “recombinant” when describing a nucleic acid sequence or a protein sequence refers to a nucleic acid, or a protein sequence, that links at least two heterologous polynucleotides, or two heterologous polypeptides, into a single macromolecule, or that re-arranges one or more elements of at least one natural nucleic acid or protein sequence. For example, the term “recombinant” can refer to an artificial combination of two otherwise separated segments of sequence, e.g., by chemical synthesis or by the manipulation of isolated segments of nucleic acids by genetic engineering techniques.

As used herein, a “synthetic nucleotide sequence” or “synthetic polynucleotide sequence” is a nucleotide sequence that is not known to occur in nature or that is not naturally occurring. Generally, such a synthetic nucleotide sequence will comprise at least one nucleotide difference when compared to any other naturally occurring nucleotide sequence.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homologue” or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

In some embodiments, the cell or organism has at least one heterologous trait. As used herein, the term “heterologous trait” refers to a phenotype imparted to a transformed host cell or transgenic organism by an exogenous DNA segment, heterologous polynucleotide or heterologous nucleic acid. These results can be achieved by providing expression of heterologous products or increased expression of endogenous products in organisms using the methods and compositions of the present disclosure.

As used herein “shelf-stable” refers to a functional attribute and new utility acquired by the microbes formulated according to the disclosure, which enable said microbes to exist in a useful/active state outside of their natural environment in a plant or soil (i.e. a markedly different characteristic). Thus, shelf-stable is a functional attribute created by the formulations/compositions of the disclosure and denoting that the microbe formulated into a shelf-stable composition can exist under ambient conditions for a period of time that can be determined depending upon the particular formulation utilized, but in general means that the microbes can be formulated to exist in a composition that is stable under ambient conditions for at least a few days and generally at least one week. Accordingly, a “shelf-stable soil treatment” is a composition comprising one or more microbes of the disclosure, said microbes formulated in a composition, such that the composition is stable under ambient conditions for at least one week.

### Soil-Borne Plant Pathogen Inhibiting Microbial Consortia Development Platform

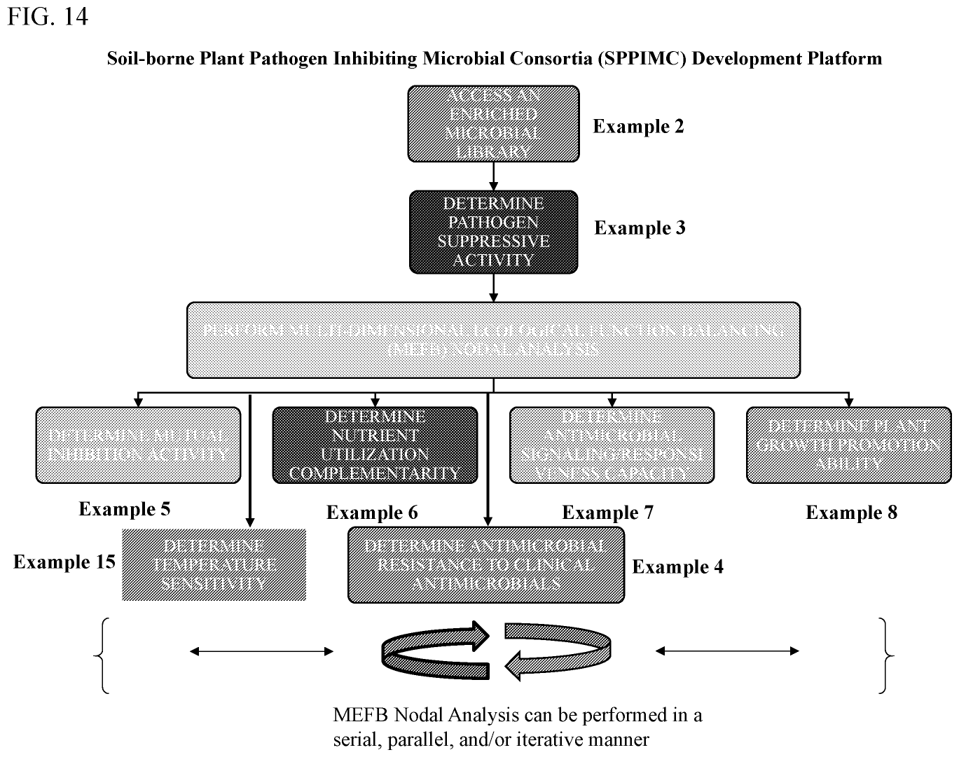

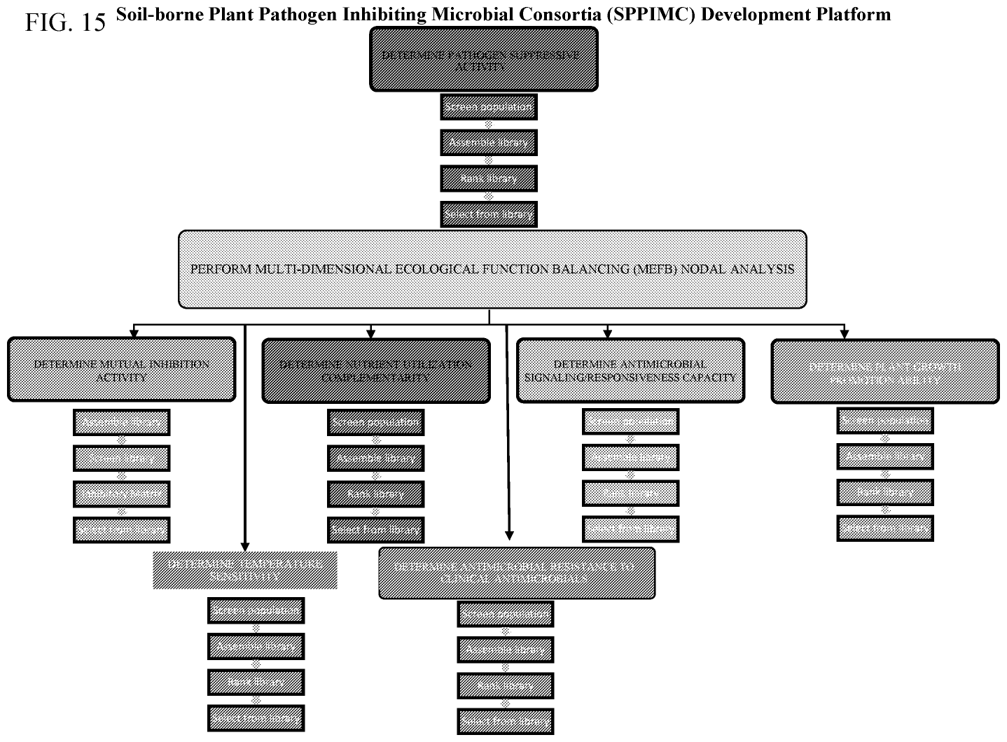

The disclosure provides a soil-borne plant pathogen inhibiting microbial consortia (SPPIMC) development platform. The platform is drawn to creating and utilizing an enriched microbial library of microbes. See FIGS. 14 and 15. In some embodiments, the enriched microbial library comprises a soil-borne plant pathogen suppressive microbial library.

In some embodiments, the platform comprises methods of creating a soil-borne plant pathogen inhibiting microbial consortia. In some embodiments, the method of creating a soil-borne plant pathogen inhibiting microbial consortia comprises the following steps: (1) accessing a soil-borne plant pathogen suppressive microbial library; (2) utilizing microbes from the library of step a) to create one or more ecological function balancing nodal microbial libraries, selected from the group consisting of: a mutual inhibitory activity microbial library, a carbon nutrient utilization complementarity microbial library, an antimicrobial signaling capacity and responsiveness microbial library, a plant growth promotion ability microbial library, and, in some embodiments, a resistance to clinical antimicrobial library; (3) performing a multi-dimensional ecological function balancing (MEFB) nodal analysis utilizing said one or more nodal microbial libraries; and (4) selecting at least two microbes from the soil-borne plant pathogen suppressive microbial library based on the MEFB nodal analysis, thereby producing a soil-borne plant pathogen inhibiting microbial consortia having a targeted ecological function in at least one dimension. Each node in this workflow is discussed in detail below.

### Enriched Microbial Libraries

In some embodiments, the present disclosure teaches enriched microbial libraries for use in the SPPIMC development platform. In some embodiments, the enriched microbial libraries refer to actual physical microbial strain collections that are collected and stored for use in the SPPIMC development platform. In some embodiments, the microbial strains in the enriched microbial libraries of the present disclosure have been collected for future analysis/use. In some embodiments, the microbial strains within the enriched microbial libraries have already undergone one or more nodes of the SPPIMC analysis (e.g., the strain has already been tested for mutual inhibitory activity, carbon nutrient utilization complementarity, antimicrobial signaling capacity and responsiveness, plant growth promotion, resistance to clinical antimicrobials, and soil-borne pathogen suppressive activity). Indeed, the microbial strain of the enriched microbial libraries may, in some embodiments, be part of one or more microbial consortia developed under the presently disclosed methods. That is, in some embodiments, the present disclosure teaches re-using microbial isolates.

In some embodiments, the enriched microbial libraries can refer to a collection of data associated with specific microbial strains. That is, in some embodiments, the step of “accessing an enriched microbial library” refers to accessing stored information related to a previously collected and analyzed microbial strain. For example, in some embodiments, the SPPIMC development platform can be used to develop new microbial consortia based on data from previously stored assays (i.e., data regarding one or more microbial strains' inhibitory activity, carbon nutrient utilization complementarity, antimicrobial signaling capacity and responsiveness, plant growth promotion, resistance to clinical antimicrobials, and soil-borne pathogen suppressive activity).

In some embodiments, the data associated with microbial strains of the enriched microbial libraries is stored within each SPPIMC node. At its simplest, the enriched microbial library can comprise identifying information about a collected or studied microbial isolate. For example, in some embodiments, the enriched microbial library can comprise genetic sequence information about a microbial isolate (e.g., 16s rRNA sequence), deposit information about a microbial isolate or related consortia, collection locus information about a microbial isolate (e.g., GPS coordinates, or soil conditions and carbon amendments). Other examples of data stored within the libraries of the present disclosure are discussed below.

For example, in some embodiments, data related to a microbial isolate's inhibitory activity is stored within an n-dimensional mutual inhibitory activity matrix or the like. In some embodiments, data related to a microbial isolate's carbon nutrient utilization complementarity is stored within a carbon nutrient utilization profile or the like. In some embodiments, data related to a microbial isolate's antimicrobial signaling capacity and responsiveness is stored within a signaling capacity and responsiveness profile or the like. In some embodiments, data related to a microbial isolate's plant growth promotion is stored within an n-dimensional plant growth promotion matrix or the like. In some embodiments, data related to a microbial isolate's resistance to clinical antimicrobials is stored within an antibiotic resistance utilization profile, or the like. In some embodiments, data related to a microbial isolate's soil-borne pathogen suppressive activity is stored within an n-dimensional soil-borne plant pathogen suppressive profile, or the like.

In some embodiments, each node within the SPPIMC development platform can be represented as a subset of the enriched microbial library. Thus, in some embodiments, physical collections of microbial isolates present in the enriched microbial library can be categorized or subdivided into one or more ecological function balancing nodal microbial libraries, each containing a collection of microbial isolates tested under that particular node (e.g., the mutual inhibition activity nodal library may contain microbial isolates for which mutual inhibition against one other microbial isolate has been tested.

Analogously, in some embodiments, collections of SPPIMC data stored as part of the enriched microbial library can be categorized/stored in individual one or more ecological function balancing nodal microbial libraries. For example, information regarding the nutrient utilization of previously screened microbial isolates may be stored within the nutrient utilization complementarity nodal microbial library (e.g., as a grouping/database of carbon nutrient utilization profiles).

Finally, in some embodiments, each node within the SPPIMC development platform can refer to a series of analysis steps or instructions. For example, the “determine pathogen suppressive activity” node of the SPPIMC development platform, can refer to the steps of assessing pathogen suppressive activity for one or more microbial isolates (e.g., screening a population of microbial isolates against one or more soil-borne pathogens to evaluate each microbial isolate's ability to suppress pathogen growth).

In summary, the nodes of the soil-borne plant pathogen inhibition microbial consortia (SPPIMC) development platform of the present disclosure can comprise, depending on the context in which they are presented/discussed: i) physical libraries (collections) of microbial isolates, ii) information about microbial isolates and consortia, and/or iii) instructions/analysis steps for gathering information about a particular microbial isolate or consortia. Additional details about the nature and implementation of the SPPIMC development platform are provided below.

### Collecting Microbes for the Enriched Microbial Library

In some embodiments, the present disclosure teaches an enriched microbial library for use in the SPPIMC development platform. In some embodiments, the library is developed by collecting and isolating various microbes from various loci, including soils, plant tissue, or other biological sources. In some embodiments, the soil is collected from forests, grasslands, deserts, tundra, freshwater sediment near land, saltwater sediment near land, brackish water sediment near land, agricultural soils, prairie soils, wetland soils, savannah soils, and peat soils. In some embodiments, the forest soil is collected from a temperate forest, a tropical rain forest, or boreal or taiga forests. In some embodiments, the grassland soil is collected from tropical grassland or temperate grassland. In some embodiments, the tropical grassland soil is collected from savannas of sub-Saharan Africa or northern Australia. In some embodiments, the temperate grassland soil is collected from Eurasian steppes, North American prairies, and Argentine pampas. In some embodiments, the desert soil is collected from hot and dry deserts, semiarid deserts, coastal deserts, or cold deserts. In some embodiments, the tundra soil is collected from arctic tundra, Antarctic tundra, or alpine tundra.

In some embodiments, the soil is obtained from one or more continents. In some embodiments, the soil is obtained from one or more habitats. In some embodiments, the one or more habitats are chosen so as to capture a wide range of ecological conditions. In some embodiments, the one or more habitats are chosen to target locations where: i) inhibitory phenotypes are likely to be enriched; and/or ii) long-term agricultural management has been imposed. The identification of locations where inhibitory phenotypes are likely to be enriched is further discussed in Kinkel et al, 2012, the contents of which are incorporated herein by reference in its entirety for all purposes. In some embodiments, the soil comprises Streptomyces, Bacillus, Fusarium, and Pseudomonas isolates.

In some embodiments, any one of the microbes disclosed herein is not naturally found in association within the same soil sample. In some embodiments, any one of the microbes disclosed herein is not naturally found in association within the same geographical region. In some embodiments, any one of the microbes disclosed herein is not naturally found in association within the same crop. In some embodiments, two or more microbes in a microbial consortia are obtained from different geographic locations. In some embodiments, the geographic location may be determined based upon the predominant soil type in a region, the predominant climate in a region, the predominant plant community present in a region, the predominant plant community present in a region, the distance between regions, and the average rainfall in a region, among others. In some embodiments, at least one microbe in a microbial consortia disclosed herein is native to, or was acquired from, a geographic region at least about 1 m, 10 m, 100 m, 1 km, 10 km, 100 km, 1,000 km, or 10,000 km from the location of one or more of the other microbes in the consortia.

Microbial Consortia with Pathogen Suppressive Activity (“Determine Pathogen Suppressive Activity”)

As discussed above, in some embodiments, the pathogen suppressive activity node of the SPPIMC development platform represents a subsection of the enriched microbial library. Therefore, in some embodiments, the pathogen suppressive activity node is a collection of microbial isolates or consortia capable of suppressing the activity of one or more soil-borne pathogens.

In some embodiments, the present disclosure teaches methods for determining a microbial isolate's pathogen suppressive activity. In some embodiments, the step of creating soil-borne plant pathogen suppressive microbial library comprises the step of (a) screening a population of microbial isolates in the presence of a plurality of individual soil-borne plant pathogens, to create a soil-borne plant pathogen suppressive profile for each individual microbial isolate in said population. As used herein, the “soil-borne plant pathogen suppressive profile” provides information on one or more features related to the ability of the microbe to suppress one or more of a plurality of plant pathogens. For instance, the one or more features may be the number of pathogens that are suppressed by the microbe, the strength of this suppressive activity and the specificity of this suppressive activity. Thus, as discussed above, the pathogen suppressive library can, in some embodiments, comprise information about each microbial isolate's ability to suppress the growth of at least one soil-borne pathogen.

In some embodiments, the present disclosure teaches methods for developing a microbial consortia with suppressive activity. In some embodiments, the microbial consortia can be developed solely based on the pathogen suppressive profiles of individual microbial isolates. Thus, in some embodiments, the disclosure provides methods for creating a soil-borne plant pathogen inhibiting microbial consortia having a targeted and complementary soil-borne plant pathogen suppressive profile, comprising: (a) screening a population of microbial isolates in the presence of a plurality of soil-borne plant pathogens (for example, a target soil-borne pathogen) to create a soil-borne plant pathogen suppressive profile for each individual microbial isolate in said population (or, alternatively, relying on previously gathered soil-borne plant pathogen suppressive profiles); (b) assembling a library of microbial consortia, each microbial consortium comprising a combination of microbial isolates from those screened (or saved in the previously gathered pathogen suppressive profile) in step a); (c) optionally ranking microbial consortia from the library of screened microbial consortia based upon at least one dimension of the soil-borne plant pathogen suppressive profile of each microbial consortia; and (d) selecting a soil-borne plant pathogen inhibiting microbial consortia having the desired targeted and complementary soil-borne plant pathogen suppressive profile from the library.

In some embodiments, the present disclosure teaches methods of generating and empirically testing microbial consortia for their pathogen suppressive activities. Thus, in some embodiments, determining pathogen suppressive activity comprises the steps of: (a) screening a population of microbial isolates in the presence of a plurality of individual soil-borne plant pathogens, to create a soil-borne plant pathogen suppressive profile for each individual microbial isolate in said population (or, alternatively, relying on previously gathered soil-borne plant pathogen suppressive profiles); (b) assembling a library of microbial consortia, each microbial consortia comprising a combination of microbial isolates from those screened in step a), wherein each microbial consortia in said library has a soil-borne plant pathogen suppressive profile that is distinct from any individual microbial isolate soil-borne plant pathogen suppressive profile from step a) in at least one dimension of the soil-borne plant pathogen suppressive profile; (c) screening microbial consortia from the library of microbial consortia in the presence of a plurality of soil-borne plant pathogens, to produce a soil-borne plant pathogen suppressive profile for each screened microbial consortia; (d) optionally ranking microbial consortia from the library of screened microbial consortia based upon at least one dimension of the soil-borne plant pathogen suppressive profile of each microbial consortia; and (e) selecting a soil-borne plant pathogen inhibiting microbial consortia having the desired soil-borne plant pathogen suppressive profile from the library.

In some embodiments, the present disclosure teaches an iterative approach for creating soil-borne plant pathogen inhibiting consortia. Thus, in some embodiments, the method comprises repeating steps (a) screening through (c) screening, or (a) through (d) (optional ranking) one or more times. In some embodiments, the method comprises repeating steps (b) through (c), or (b) through (d) one or more times. Without wishing to be bound by any one theory, the present inventors hypothesize that the iterative production and testing of microbial consortia can result in improved pathogen suppression due to the discovery of unappreciated synergies.

In some embodiments, each microbial consortia in said library has a soil-borne plant pathogen suppressive profile that is distinct from any individual microbial isolate soil-borne plant pathogen suppressive profile from step a) in at least one dimension of the soil-borne plant pathogen suppressive profile.

As used herein, a “dimension of the soil-borne plant pathogen suppressive profile” is any feature or characteristic related to, associated with, contributing to, or caused by the soil-borne plant pathogen suppressive profile, such as, for example, the number of pathogens that are suppressed by a microbial isolate. Therefore, in some embodiments, the at least one dimension is selected from the group consisting of: (i) strength of suppressive activity against any one or more member of the plurality of soil-borne plant pathogens, (ii) specificity against any one or more member of the plurality of soil-borne plant pathogens, and (iii) breadth of activity against any one or more member of the plurality of soil-borne plant pathogens. In some embodiments, at least one microbial isolate in each of the assembled microbial consortia suppresses the growth of the target soil-borne pathogen.

In some embodiments, the microbes are screened on solid or liquid media to determine the pathogen suppressive activity of each of the microbes against one or more plant pathogens. In some embodiments, the pathogen suppressive activity of the microbes is determined using any one of the methods for pathogen suppressive activity disclosed herein, for example, any method disclosed the Example section of the present disclosure.

In some embodiments, the pathogen suppressive activity of the microbes is determined using any one of the methods that is commonly used in the art for this purpose, such as, for example, as described in Davelos, A. L., Kinkel, L. L., Samac, D. A. (2004). Spatial variation in the frequency and intensity of antibiotic interactions among Streptomycetes from Prairie Soil. Applied and Environmental Microbiology 70: 1051-1058, which is incorporated herein by reference in its entirety for all purposes.

In some embodiments, the consortia comprises two or more microbes such that each microbe in the consortia possesses the capability to suppress at least one plant pathogen that is not suppressed by the other microbes in the consortia. In some embodiments, the two or more microbes in the consortia possess complementary soil-borne plant pathogen suppressive profiles. That is, in some embodiments, the two or more microbes in the consortia do not share the capability to suppress any one particular plant pathogen. In some embodiments, the two or more microbes in the consortia share the capability to suppress at least one plant pathogen.

As used herein, a plant pathogen is a pathogenic organism that infects a plant. As used herein, a soil-borne plant pathogen is a plant pathogen found in the soil. In some embodiments, the soil-borne plant pathogen is predominantly found in the soil, as compared to other habitats. That is, in some embodiments, soil-borne pathogens may be found on plant tissue, including above-ground tissue. In some embodiments, the soil-borne plant pathogen is only found in the soil. In some embodiments, the target soil-borne plant pathogen is a species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii. In some embodiments, soil-borne plant pathogens include bacteria, such as species of Erwinia, Rhizomonas, some Streptomyces (such as Streptomyces scabies), Pseudomonas, and Xanthomonas.

### Multi-dimensional Ecological Function Balancing (MEFB) Nodal Analysis

The MEFB nodal analysis comprises four nodes that can be performed in serial, parallel, and/or iterative manner. The four nodes include (1) Mutual Inhibition Activity Determination, (2) Nutrient Utilization Complementarity Determination, (3) Antimicrobial Signaling/Responsiveness Capacity Determination, and (4) Plant Growth Promotion Ability Determination. In some embodiments, the MEFB nodal analysis further comprises a fifth node of Resistance to Antimicrobial Determination. In some embodiments, the MEFB nodal analysis further comprises a sixth node of Temperature Sensitivity Determination. In some embodiments, the MEFB nodal analysis comprises conducting all nodes. In some embodiments the MEFB nodal analysis comprises conducting any 1, 2, 3, 4, 5, or 6 of the nodes. See FIGS. 14 and 15. In some embodiments, the SPPIMC platform comprises the determination of pathogen suppressive activity. In some embodiments, the MEFB analysis comprises the mutual inhibition activity node and the antimicrobial signaling/responsiveness activity node.

In some embodiments, the nodes of the MEFB analysis are selected based on several factors, including the type of soil, number of microbes in the soil, type of plant pathogen in the soil, type of plant/crop being exposed to the plant pathogen, location of field, climate, and planting and growing season of the plants. In some embodiments, the nodes of the MEFB analysis are selected based on the type of soil that is to be amended by the microbial consortia selected from the MEFB analysis. In some embodiments, the MEFB analysis comprises the nutrient utilization node when the soil is a high nutrient soil. In some embodiments, microbial consortia in which individual microbes have complementary nutrient preferences or have the greatest differences in nutrient preferences, are better for applications in high nutrient soils. In some embodiments, the MEFB analysis comprises the nutrient utilization node when the soil is a low nutrient soil. In some embodiments, isolates that have similar nutrient preferences are chosen to be in a multi-strain inoculant composition exposed to low-nutrient soil.

In some embodiments, the MEFB analysis comprises the node for determination of antimicrobial resistance to antimicrobials when the soil is known to be rich in a wide range of anti-microbial-producing microbes. In some embodiments, the MEFB comprises the node for determination of temperature sensitivity, depending on the climate of the location at which the soil is to be amended with the microbial consortia selected from the MEFB analysis, and the growing/planting season of the plant. In some embodiments, the MEFB comprises the temperature sensitivity determination node, depending on the biome from which the soil is to be amended with the microbial consortia selected from the MEFB analysis. In some embodiments, the biome is tropical, temperate, boreal, Mediterranean, desert, tundra, coastal or mountain ranges. In some embodiments, the MEFB analysis comprises the node for determining the plant growth promotion ability of the microbe or microbial consortia. In some embodiments, the plant growth promotion ability is evaluated based on the type of plant that is being grown.

A. Microbial Consortia with Mutual Inhibition Activity (“Determine Mutual Inhibition Activity”)

As discussed above, in some embodiments, the mutual inhibition activity node of the SPPIMC development platform represents a subsection of the enriched microbial library. Therefore, in some embodiments, the mutual inhibition activity node is a collection of microbial isolates with information related to their ability to inhibit (or not inhibit) at least one other microbial isolate.

In some embodiments, the present disclosure teaches methods for determining a microbial isolate's ability to inhibit one or more other microbial isolates. The disclosure provides methods for creating a mutual inhibitory activity library, comprising the following steps: i) assembling a library of test microbial consortia, each test consortia comprising a combination of at least two microbial isolates from the soil-borne plant pathogen suppressive microbial library; ii) screening test microbial consortia of the assembled library for the relative degree of mutual inhibitory activity displayed by each microbial isolate towards every other individual microbial isolate in the test microbial consortia; iii) developing an n-dimensional mutual inhibitory activity matrix for test microbial consortia based on the mutual inhibitory activities screened in step (ii); and optionally, select one or more test consortia for further development, analysis, or use. As used herein, the “mutual inhibitory activity matrix” provides information on one or more features related to the ability of each microbe to suppress every other microbe in the test consortia. For instance, the one or more features may be the number of microbes that are inhibited by the microbe, the strength of this inhibitory activity and the specificity of this inhibitory activity. Thus in some embodiments, the mutual inhibitory activity microbial library comprises information about each microbial isolate's ability to inhibit the growth of at least one other microbial isolate. In some embodiments, the mutual inhibitory activity microbial library comprises information about each microbial isolate's ability to inhibit the growth of every other microbial isolate in the library.

In some embodiments, the present disclosure teaches methods of developing a microbial consortia having a designed level of mutual inhibitory activity. In some embodiments, the microbial consortia can be developed solely based on the n-dimensional mutual inhibitory activity matrix of individual microbial isolates. Thus, in some embodiments, the disclosure provides methods for creating a soil-borne plant pathogen inhibiting microbial consortia having a designed level of mutual inhibitory activity comprising the following steps: (a) assembling a library of microbial consortia, each consortia comprising a combination of at least two microbial isolates; (b) screening microbial consortia of the assembled library for the relative degree of mutual inhibitory activity displayed by each microbial isolate towards every other individual microbial isolate in the microbial consortia; and (c) developing an n-dimensional mutual inhibitory activity matrix for microbial consortia based on the mutual inhibitory activities screened in step (b); and (d) optionally selecting a soil-borne plant pathogen inhibiting microbial consortia having the designed level of mutual inhibitory activity from the library based upon the n-dimensional mutual inhibitory activity matrix for further development/analysis, or use.

Additionally, the disclosure provides methods for creating a soil-borne plant pathogen inhibiting microbial consortia having a designed level of mutual inhibitory activity comprising the following steps: (a) screening a population of microbial isolates for the relative degree of mutual inhibitory activity displayed by each microbial isolate towards at least one other individual microbial isolate in the screened population, to create an n-dimensional mutual inhibitory activity matrix based on the mutual inhibitory activities for the screened population (or, alternatively, relying on a previously gathered mutual inhibitory activity matrix); (b) assembling a library of microbial consortia, each microbial consortia comprising a combination of microbial isolates from those screened in step a), wherein the individual microbial isolates of each microbial consortia in said library are expected to be able to grow together based on the n-dimensional mutual inhibitory activity matrix of step a) and/or, based on a previously gathered mutual inhibitory activity matrix.

In some embodiments, the present disclosure teaches methods for developing and empirically testing microbial consortia for their mutual inhibition activities Thus, in some embodiments, the disclosure provides methods for creating a soil-borne plant pathogen inhibiting microbial consortia having a designed level of mutual inhibitory activity comprising the following steps: (a) screening a population of microbial isolates for the relative degree of mutual inhibitory activity displayed by each microbial isolate towards at least one other individual microbial isolate in the screened population, to create an n-dimensional mutual inhibitory activity matrix based on the mutual inhibitory activities for the screened population; (b) assembling a library of microbial consortia, each microbial consortia comprising a combination of microbial isolates from those screened in step a), wherein the individual microbial isolates of each microbial consortia in said library are expected to be able to grow together based on the n-dimensional mutual inhibitory activity matrix of step a) (c) optionally screening consortia from the library of microbial consortia by growing said consortia in a growth medium and monitoring the continued presence of each microbial isolate within each consortia; and (d) selecting a soil-borne plant pathogen inhibiting microbial consortia having the level of mutual inhibitory activity from the library of step b).

In some embodiments, the present disclosure teaches an iterative approach for creating a soil-borne plant pathogen inhibiting microbial consortia having a designed level of mutual inhibitory activity. Thus, in some embodiments, the method comprises repeating steps (a) screening through (c) screening, or (a) through (d) one or more times. In some embodiments, the method comprises repeating steps (b) through (c), or (b) through (d) one or more times. Without wishing to be bound by any one theory, the present inventors hypothesize that the iterative production and testing of microbial consortia can result in improved level of mutual inhibitory activity due to the discovery of unappreciated synergies/interactions within the microbial isolates forming each consortia.

As used herein, a “dimension of the mutual inhibitory matrix” is any feature or characteristic related to, associated with, contributing to, or cause by the mutual inhibition between two or more microbial isolates. In some embodiments, the n-dimensional mutual inhibitory activity matrix described above comprises a dimension selected from the group consisting of: (i) strength of inhibitory activity against one or more member microbial isolates, (ii) specificity against one or more member of a plurality of microbial isolates, and (iii) breadth of activity against any one or more member of the plurality of microbial isolates. In some embodiments, microbial consortia of the present disclosure are designed so that their component microbial isolates have minimal mutual inhibition activities against each other. In some embodiments, microbial consortia of the present disclosure are designed to exhibit inhibition activities against at least one microbial isolate that is not part of the microbial consortia.

In some embodiments, the screening of the microbial consortia for the relative degree of mutual inhibitory activity is conducted in pairs, such that each microbial isolate in the consortia is individually tested with one other microbial isolate in the microbial consortia. In some embodiments, pairwise screening of inhibitory activities can be conducted in parallel, such that multiple inhibition activities are tested at a time. Embodiments for parallel screening are discussed below.

In some embodiments, the screening of the microbial consortia for the relative degree of mutual inhibitory activity is conducted in groups of three or more, such that a first microbial isolate is screened for mutual inhibitory activity toward another microbial isolate, when said first microbial isolate is grown adjacent or in contact with a third microbial isolate; wherein the first, second and third microbial isolates are all part of the screened microbial consortia. In some embodiments, the screening of the microbial consortia for the relative degree of mutual inhibitory activity comprises testing the relative degree of inhibitory activity against each microbial isolate in the consortia, caused by the combination of all other remaining microbial isolates in the microbial consortia.

In some embodiments, the method further comprises the step of screening a population of microbial isolates in the presence of a plurality of soil-borne plant pathogens to create a soil-borne plant pathogen suppressive profile for each individual microbial isolate in said population, wherein the microbial consortia of step (a) comprise soil-borne plant pathogen suppressive profile. In some embodiments, the library of microbial consortia refers to the actual physical microbial strain collection that is generated using the methods disclosed herein. In some embodiments, the library of microbial consortia refers to a virtual database or collection of microbes that are determined to be part of the library.

In some embodiments, the mutual inhibitory activity of the microbes is determined using any one of the methods for mutual inhibitory activity disclosed herein, for example, in the Examples. In some embodiments, the mutual inhibitory activity of two microbes is determined by a method comprising growing the two microbes together on solid media. In some embodiments, the method comprises overlaying a first microbe on a plate comprising a colony of a second microbe. In some embodiments, an inhibition zone is formed around the colony of the second microbe. In some embodiments, the mutual inhibitory activity is quantified by determining the size of the inhibition zone. As used herein, “inhibition zone” represents an average of two 90° length measurements from the edge of the first microbial colony to the edge of the cleared zone where the overlaid second microbe did not grow.

Inhibition activity assays, can in some embodiments, be conducted in a pairwise manner, such that one microbial isolate overlay is tested against one previously dotted microbial isolate colony. In some embodiments, the present disclosure also teaches parallel pairwise inhibition assays. For example, in some embodiments, multiple microbial isolates are dotted onto a single petri dish, and then tested against an overlay microbial isolates. In this embodiment, the inhibition zones created around each of the initially dotted microbial isolates would be measured to provide information about the inhibition activity of each of the dotted microbial isolates against the overlaid isolate. In some embodiments, the initially dotted microbial isolates are grown at a sufficient distance to avoid signaling between them. In other embodiments, the initially dotted microbial isolates are grown adjacent to each other, to permit signaling between the isolates, and identify inhibition activities resulting from the signaling/synergy of the combination of microbial isolates dotted together.

In some embodiments, the mutual inhibitory activity of the microbes is determined using any one of the methods that is commonly used in the art for this purpose, such as, for example, as described in Kinkel, L. L., Schlatter, D. S., Xiao, K., and Baines, A. D. (2014). Sympatric inhibition and niche differentiation suggest alternative coevolutionary trajectories among Streptomycetes. ISME 8: 249-256. doi:10.1038/ismej.2013.175, which is incorporated herein by reference in its entirety for all purposes.

B. Microbial Consortia with Nutrient Utilization Complementarity (“Determine Nutrient Utilization Complementarity”)

As discussed above, in some embodiments, the nutrient utilization complementary node of the SPPIMC development platform represents a subsection of the enriched microbial library. Therefore, in some embodiments, the pathogen suppressive activity node is a collection of microbial isolates or consortia for which nutrient utilization information is known.

In some embodiments, the present disclosure teaches methods for determining a microbial isolate's nutrient utilization profile. As used herein, the “nutrient utilization profile” provides information on one or more features related to the ability of each microbe to utilize a range of nutrients. For instance, the one or more features may be the number of nutrients that are utilized by the microbe, the strength of this utilization activity and the specificity of this utilization activity. Thus, in some embodiments, the step of creating a carbon nutrient utilization complementarity microbial library comprises the step of: i) screening a population of microbial isolates for carbon nutrient utilization by growing said microbial isolates in a plurality of different nutrient media that comprise a distinct single carbon source to create a carbon nutrient utilization profile for each individual microbial isolate in said population. Thus in some embodiments, the carbon nutrient utilization complementarity microbial library comprises information about each microbial isolate's ability to grow on at least one carbon source.

In some embodiments, the present disclosure teaches methods for developing/creating microbial consortia with complementary nutrient utilization profiles. In some embodiments, the microbial consortia can be created based on the nutrient utilization profiles of individual microbial isolates. Thus, in some embodiments, the disclosure provides methods for creating a soil-borne plant pathogen inhibiting microbial consortia having an optimal and designed level of carbon nutrient utilization complementarity, comprising the following steps: (a) screening a population of microbial isolates for carbon nutrient utilization by growing said microbial isolates in a plurality of different nutrient media that comprise a distinct single carbon source to create a carbon nutrient utilization profile for each individual microbial isolate in said population (or, alternatively, relying on previously gathered nutrient utilization profiles); (b) assembling a library of microbial consortia, each microbial consortia comprising a combination of microbial isolates from those screened in step a) and/or those included in the previously gathered nutrient utilization profiles; (c) optionally ranking microbial consortia from the library based upon at least one dimension of the combined carbon nutrient utilization profile of each microbial consortia in said library; and (d) optionally selecting a soil-borne plant pathogen inhibiting microbial consortia having an optimal and designed level of carbon nutrient utilization complementarity from the library.

In some embodiments, the present disclosure teaches an iterative approach for creating microbial consortia with complementary nutrient utilization profiles. Thus, in some embodiments, the method comprises repeating steps a) through c) one or more times. In some embodiments, the method comprises repeating steps b) through c) one or more times. In some embodiments, the library of microbial consortia refers to the actual physical microbial strain collection that is generated using the methods disclosed herein. In some embodiments, the library of microbial consortia refers to a virtual database or collection of microbes that are determined to be part of the library.

In some embodiments, each microbial consortia in the library of the present invention has a carbon nutrient utilization profile that is distinct from any individual member microbial isolate carbon nutrient utilization profile in at least one dimension of the carbon nutrient utilization profile. As used herein, a “dimension of the carbon nutrient utilization profile” is any feature or characteristic related to, associated with, contributing to, or caused by the carbon nutrient utilization profile, such as, for example, the number of nutrients that are utilized by a microbial isolate. In some embodiments, the at least one dimension is selected from the group consisting of: (i) binary ability to grow in any one distinct single carbon source found in said plurality of different nutrient media; (ii) strength of ability to grow in any one distinct single carbon source found in said plurality of different nutrient media; (iii) binary ability to grow in at least two distinct single carbon sources found in said plurality of different nutrient media, and (iv) strength of ability to grow in at least two distinct single carbon sources found in said plurality of different nutrient media.

In some embodiments, the consortia comprises at least two microbes that do not have the same nutrient utilization profile. In some embodiments, the consortia comprises at least two microbes that do not compete for nutrients with each other. In some embodiments, no two microbes of the consortia have the same nutrient utilization profile. In some embodiments, no two microbes in the consortia compete for nutrients with each other. In some embodiments, at least one microbe in the consortia exhibits a nutrient utilization overlap with at least one other microbe in the consortia.

Persons having skill in the art will recognize that the carbon sources of the present disclosure can be any carbon source capable of sustaining or contributing to the energy needs of a microbial isolate. In some embodiments, the nutrient sources are complex nutrient sources. In some embodiments, the nutrient sources are simple nutrient sources. In some embodiments, the nutrients sources comprise one or more of the following: water, α-cyclodextrin, β-cyclodextrin, dextrin, glycogen, inulin, mannan, TWEEN 40, TWEEN 80, N-acetyl-D-glucosamine, N-acetyl-β-D-mannosamine, amygdalin, L-arabinose, D-arabitol, arbutin, D-cellobiose, D-fructose, L-fucose, D-galactose, D-galacturonic acid, gentiobiose, D-gluconic acid, α-D-glucose, m-inositol, α-D-lactose, lactulose, maltose, maltotriose, D-mannitol, D-mannose, D-melezitose, D-melibiose, α-methyl-D-galactoside, β-metyl-D-galactoside, 3-methyl-D-glucose, α-methyl-D-glucoside, β-methyl-D-glucoside, α-methyl-D-mannoside, palatinose, D-psicose, D-raffinose, L-rhamnose, D-ribose, salicin, sedoheptulosan, D-sorbitol, stachyose, sucrose, D-tagatose, D-trehalose, turanose, xylitol, D-xylose, acetic acid, α-hydroxybutyric acid, β-hydroxybutryic acid, γ-hydroxybutyric acid, p-hydroxy-phenylacetic acid, α-ketoglutaric acid, α-ketovaleric acid, lactamide, D-lactic acid methyl ester, L-lactic acid, D-malic acid, L-malic acid, pyruvic acid methyl ester, succinic acid mono-methyl ester, propionic acid, pyruvic acid, succinamic acid, succinic acid, N-acetyl-L-glutamic acid, L-alaninamide, D-alanine, L-alanine, L-alanyl-glycine, L-asparagine, L-glutamic acid, Glycyl-L-glutamic acid, L-pyroglutamic acid, L-serine, Putrescine, 2,3-butanediol, glycerol, adenosine, 2′-deoxy adenosine, inosine, thymidine, uridine, adenosine-5′-monophosphate, thymidine-5′-monophosphate, uridine-5′-monophosphate, D-fructose-6-phosphate, α-D-glucose-1-phosphate, D-glucose-6-phosphate, and D-L-α-glycerol phosphate.

In some embodiments, the nutrient utilization profile of microbes is determined using any of the methods for nutrient utilization disclosed herein, including those disclosed in the Example section of the specification. In some embodiments, nutrient use profiles are generated by attempting to grow microbial isolates in media containing a single carbon source substrate (e.g., glucose or fructose), and measuring the optical density of the microbial isolate after being cultured for a sufficient period of time. In some embodiments, nutrient use profiles can be generated by growing the microbial isolates on 95 different nutrient substrates in Biolog SF-P2 Microplate™ plates (available at the World Wide Web at bilog.com/Certificates%20of%20Analysis%20/%20Safety%20Data%20Sheets/ms-1511-1514-sfn2-sfp2-specialty-microplates-2/) for three to seven days.

In some embodiments, the present disclosure teaches at least two metrics for measuring nutrient usage. In some embodiments, the present disclosure teaches niche width, which is defined as the number of substrates a microbial isolate can grow on. In some embodiments, the present disclosure teaches niche overlap between two strains, defined by the formula: Niche overlap (microbial Isolate “Y” against microbial isolate “X”)=Σ₁₋₉₅ (min [Xᵢ, Yᵢ]/Xᵢ)/(niche width [isolate X]), wherein X and Y each denote a different isolate, and Xi and Yi represent the growth (OD600) of the X and Y isolates in a particular media, respectively.

In some embodiments, the present disclosure teaches that, isolates with complementary nutrient preferences or those with the greatest differences in nutrient preferences, are better for applications in high nutrient soils. In some embodiments, nutrient complementarity is less critical in low nutrient soils. Therefore, in some embodiments, isolates that have different nutrient preferences are chosen to be in a multi-strain inoculant composition exposed to high-nutrient soil. In some embodiments, isolates that have similar nutrient preferences are chosen to be in a multi-strain inoculant composition exposed to low-nutrient soil.

Without being bound by theory, it is believed that nutrient differentiation is much more crucial to successful coexistence in high nutrient sites. In high nutrient soils, microbes are able to coexist at least in part by tending to utilize different nutrients (showing nutrient utilization complementarity and higher niche differentiation), which reduces competitive interactions. Without being bound by theory, it is believed that niche differentiation in this setting allows the microbes to avoid the metabolic costs of antibiotic production, and thus optimize on fitness. On the other hand, in low nutrient soils, microbes have low nice differentiation and higher niche overlap—i.e., they tend to utilize the same nutrients, which can result in resource competitive interactions manifested by the production of antibiotics targeted at the competing microbes. However, the metabolic cost of antibiotic production is high. Therefore, niche differentiation offers an alternative means for mediating interactions with competitors. Furthermore, it is believed that microbes with high niche differentiation are more efficient at utilizing their respective nutrients, as compared with microbes with low nutrient differentiation.

In low-nutrient sites, there is much more niche overlap, which may be a consequence of at least 2 different things: (1) It is important in a low nutrient site that an isolate be able to consume any nutrients that are available =high niche width; and (2) isolates may be simultaneously using inhibition to establish spatial differentiation, which is likely to be much more important in a low-nutrient than a high-nutrient soil. For example, if 2 isolates are capable of utilizing one nutrient each, they have 100% niche differentiation; but they are not very good at colonizing because of their limited nutrient utilization capability. Therefore, in some embodiments, isolates in a multi-strain inoculant composition are selected such that niche width and growth efficiency of the individual isolates is maximized. In some embodiments, isolates in a multi-strain inoculant composition are selected such that the niche overlap of the individual isolates is minimized.

C. Microbial Consortia with Antimicrobial Signaling/Responsiveness Capacity (“Determine Antimicrobial Signaling/Responsiveness Capacity”)

As discussed above, in some embodiments, the antimicrobial signaling/responsiveness capacity node of the SPPIMC development platform represents a subsection of the enriched microbial library. Therefore, in some embodiments, the antimicrobial signaling/responsiveness capacity node is a collection of microbial isolates with known information regarding their ability to signal (or be signaled by) at least one other microbial isolate.

In some embodiments, the present disclosure teaches methods for determining a microbial isolate's antimicrobial signaling and/or responsiveness capacity. Thus, in some embodiments, the present disclosure teaches a method for creating an antimicrobial signaling capacity and responsiveness profile for a microbial isolate, said method comprising the steps of: (i) screening a population of microbial isolates for the ability of each microbial isolate to signal and modulate the production of antimicrobial compounds in other microbial isolates from the population of microbial isolates; and/or (ii) screening a population of microbial isolates for the ability of each microbial isolate to be signaled and have their production of antimicrobial compounds modulated by other microbial isolates from the population of microbial isolates; thereby creating an antimicrobial signaling capacity and responsiveness profile for each screened microbial isolate. As used herein, the “antimicrobial signaling capacity and responsiveness profile” provides information on one or more features related to the ability of each microbe to signal and modulate the production of antimicrobial compounds in other microbes. For instance, the one or more features may be the number of microbes that are signaled by the microbe, the strength of this signaling activity and the specificity of this signaling activity. Thus in some embodiments, the antimicrobial signaling capacity and responsiveness microbial library comprises information about each microbial isolate's ability to signal or be signaled by at least one other microbial isolate.

In some embodiments, the present disclosure teaches methods of developing microbial consortia with microbial isolates exhibiting signaling or responsiveness synergies (e.g., gaining additional anti-pathogenic features triggered by the signaling between two or more microbial isolates). In some embodiments, the microbial consortia can be developed/created based on the antimicrobial signaling capacity and responsiveness profile of individual microbial isolates. Thus, in some embodiments, the disclosure provides methods for creating a soil-borne plant pathogen inhibiting microbial consortia having an optimal and designed level of antimicrobial signaling capacity and responsiveness, comprising the following steps: (a) creating an antimicrobial signaling capacity and responsiveness profile for each individual microbial isolate of a microbial population (or alternatively, relying on a previously gathered antimicrobial signaling capacity and responsiveness profile); (b) assembling a library of microbial consortia, each consortium comprising a plurality of microbial isolates from those screened in step a); (c) optionally ranking microbial consortia from the library of microbial consortia based upon at least one dimension of the antimicrobial signaling capacity and responsiveness profile of each microbial consortia in said library; and (d) selecting a soil-borne plant pathogen inhibiting microbial consortia having an optimal and designed level of antimicrobial signaling capacity and responsiveness from the library.

In some embodiments, the present disclosure teaches methods for developing and empirically testing a soil-borne plant pathogen inhibiting microbial consortia having an optimal and designed level of antimicrobial signaling capacity and responsiveness profiles. Thus, in some embodiments, the method for creating a soil-borne plant pathogen inhibiting microbial consortia having an optimal and designed level of antimicrobial signaling capacity and responsiveness further comprises a step of screening microbial consortia from the library of microbial consortia in the presence of a soil-borne pathogen targeted by the antimicrobial compound(s) produced as a consequence of the antimicrobial signaling capacity or responsiveness of at least one microbial isolate in the microbial consortia, identified in step (a).

Thus, the disclosure further provides methods for creating a soil-borne plant pathogen inhibiting microbial consortia having an optimal and designed level of antimicrobial signaling capacity and responsiveness, comprising: (a) creating an antimicrobial signaling capacity and responsiveness profile for each individual microbial isolate of a microbial population; (b) assembling a library of microbial consortia, each consortia comprising a plurality of microbial isolates from those screened in step a); (c) screening microbial consortia from the library of microbial consortia in the presence of a soil-borne pathogen targeted by the antimicrobial compound(s) produced as a consequence of the antimicrobial signaling capacity or responsiveness of at least one microbial isolate in the microbial consortia, identified in step (a); (d) ranking microbial consortia from the library of screened microbial consortia based upon at least one dimension of the antimicrobial signaling capacity and responsiveness profile of each screened microbial consortia; and (e) selecting a soil-borne plant pathogen inhibiting microbial consortia having an optimal and designed level of antimicrobial signaling capacity and responsiveness from the library.

In some embodiments, the present disclosure teaches an iterative approach for creating a soil-borne plant pathogen inhibiting microbial consortia having an optimal and designed level of antimicrobial signaling capacity and responsiveness. Thus, in some embodiments, the method comprises repeating steps (a) through (c) one or more times. In some embodiments, the method comprises repeating steps (b) through (c) one or more times. In some embodiments, the method comprises repeating steps (a) through (d) one or more times. In some embodiments, the method comprises repeating steps (b) through (d) one or more times.

In some embodiments, the present disclosure teaches a method for creating a soil-borne plant pathogen inhibiting microbial consortia having an optimal and designed level of antimicrobial signaling capacity and responsiveness, comprising: (a) assembling a library of microbial consortia, each consortia comprising a plurality of microbial isolates wherein at least one of said microbial isolates exhibits antimicrobial signaling capacity towards at least one other microbial isolate in the microbial consortia (e.g., as measured and described above); (b) screening microbial consortia from the library of microbial consortia in the presence of a soil-borne pathogen targeted by the antimicrobial compound(s) produced as a consequence of the antimicrobial signaling capacity or responsiveness of at least one microbial isolate in the microbial consortia; (c) optionally ranking microbial consortia from the library of screened microbial consortia based upon at least one dimension of the antimicrobial signaling capacity and responsiveness profile of each screened microbial consortia; and (d) selecting a soil-borne plant pathogen inhibiting microbial consortia having an optimal and designed level of antimicrobial signaling capacity and responsiveness from the library.

In some embodiments, the method comprises repeating steps (a) through (c) one or more times. In some embodiments, the method comprises repeating steps (a) through (b) one or more times.

In some embodiments, each microbial consortia in said library has an antimicrobial signaling capacity and responsiveness profile that is distinct from the antimicrobial signaling capacity and responsiveness profile of any of individual microbial isolate within the consortia. In some embodiments, the profiles are distinct in at least one dimension of the antimicrobial signaling capacity and responsiveness profile. As used herein, a “dimension of the antimicrobial signaling capacity and responsiveness profile” is any feature or characteristic related to, associated with, contributing to, or caused by the antimicrobial signaling capacity and responsiveness profile. In some embodiments, the at least one dimension of the antimicrobial signaling capacity and responsiveness profile is selected from (i) binary ability to signal and modulate the production of antimicrobial compounds in other microbial isolates, and (ii) strength of ability to signal and modulate the production of antimicrobial compounds in other microbial isolates. In some embodiments, the at least one dimension of the antimicrobial signaling capacity and responsiveness profile is selected from (i) binary ability to be signaled and have their production of antimicrobial compounds modulated by other microbial isolates, and (ii) strength of ability to be signaled and have their production of antimicrobial compounds modulated by other microbial isolates.

In some embodiments, the screening of a population of microbial isolates in step a) comprises: utilizing genomic information, transcriptomic information, and/or growth culture information.

D. Microbial Consortia with Plant Growth Promotion Ability (“Determine Plant Growth Promotion Ability”)

As discussed above, in some embodiments, the determine plant growth promotion ability node of the SPPIMC development platform represents a subsection of the enriched microbial library. Therefore, in some embodiments, the plant growth promotion ability node is a collection of microbial isolates with known information related to their ability to promote plant growth.

In some embodiments, the present disclosure teaches methods for determining a microbial isolate's ability to promote the growth of a plant. The present disclosure thus teaches methods comprising screening and evaluating a population of microbial isolates for their plant growth ability, said method comprising the steps of a) applying each microbial isolate in the population to a test plant, b) cultivating the test plant in growth chamber, greenhouse, or field conditions, and c) comparing the growth of the test plant against that of a control plant that did not receive the microbial isolate.

In some embodiments, the present disclosure teaches methods for developing microbial consortia having an optimal and designed level of plant growth promoting ability. In some embodiments, the microbial consortia can be developed solely based on the plant growth promoting ability profile of individual microbial isolates. As used herein, the “plant growth promoting ability profile” provides information on one or more features related to the ability of each microbe to promote the growth of a plant. For instance, the one or more features may be the increase in yield of the plant, decrease in disease symptoms, types of plants for which growth is promoted and the like. Thus, in some embodiments, the disclosure provides methods for creating a soil-borne plant pathogen inhibiting microbial consortia having an optimal and designed level of plant growth promoting ability, comprising the following steps: (a) creating plant growth promoting ability profile for each individual microbial isolate of a microbial population by screening and evaluating each microbial isolate's ability to promote growth of one or more plants; (b) assembling a library of microbial consortia, each consortium comprising a plurality of microbial isolates from those screened in step a); (c) optionally ranking microbial consortia from the library of microbial consortia based upon at least one dimension of the plant growth promoting ability of each microbial consortia in said library; and (d) selecting a soil-borne plant pathogen inhibiting microbial consortia having an optimal and designed level of plant growth promoting ability from the library.

Thus, in some embodiments, the present disclosure teaches methods for developing microbial consortia having an optimal and designed level of plant growth promoting ability, comprising the following steps: (a) assembling a library of microbial consortia, each consortium comprising a plurality of microbial isolates, wherein microbial isolates are selected based on each isolate's plant growth promoting ability profile; (c) optionally ranking microbial consortia from the library of microbial consortia based upon at least one dimension of the plant growth promoting ability of each microbial consortia in said library; and (d) selecting a soil-borne plant pathogen inhibiting microbial consortia having an optimal and designed level of plant growth promoting ability from the library.

In some embodiments, the present disclosure teaches methods for developing and empirically testing microbial consortia having an optimal and designed level of plant growth promoting ability. Thus, in some embodiments, the present disclosure teaches a method for creating a soil-borne plant pathogen inhibiting microbial consortia having an optimal and designed level of plant growth promoting ability, comprising the following steps: (a) creating plant growth promoting ability profile for each individual microbial isolate of a microbial population by screening and evaluating each microbial isolate's ability to promote growth of one or more plants; (b) assembling a library of microbial consortia, each consortium comprising a plurality of microbial isolates from those screened in step a); (c) screening microbial consortia from the library of microbial consortia by: i) applying microbial consortia from the library to a test plant, ii) cultivating the test plant in growth chamber, greenhouse, or field conditions, and iii) comparing the growth of the test plant against that of a control plant that did not receive the microbial consortia; thereby producing a plant growth promoting ability profile for each screened microbial consortia; (d) optionally ranking microbial consortia from the library of screened microbial consortia based upon at least one dimension of the plant growth promoting ability profile of each screened microbial consortia; and (d) selecting a soil-borne plant pathogen inhibiting microbial consortia having an optimal and designed level of plant growth promoting ability from the library.

In some embodiments, the present disclosure teaches an iterative approach for creating a soil-borne plant pathogen inhibiting microbial consortia having an optimal and designed level of plant growth promoting ability. Thus, in some embodiments, the method of comprises repeating steps a) through c) one or more times. In some embodiments, the method comprises repeating steps a) through d) one or more times. In some embodiments, the method comprises repeating steps b) through c) one or more times. In some embodiments, the method comprises repeating steps b) through d) one or more times.

As used herein, a “dimension of the plant growth promoting ability” is any feature or characteristic related to, associated with, contributing to, or caused by the plant growth promoting ability of the microbial consortia. In some embodiments, the at least dimension of the plant growth promoting ability of each microbial is selected from the group consisting of: i. binary ability to promote growth of a particular plant; ii. the degree of growth promotion for the particular plant; iii. the mechanism by which the consortia promotes the growth of the particular plant and resistance of the plant to one or more plant pathogens.

In some embodiments, the at least dimension of the plant growth promoting ability of each microbial consortia comprises one or more of the following: increased growth rate; increased growth rate in saline-limiting soils, drought, nutrient-limited, or other environmentally stressful habitats; a decrease in the damage in plants exposed to severe insect damage or disease epidemic; an increase in certain metabolites and other compounds that include fiber content, oil content, and the like; increased crop yield; an increase in the displaying of desirable colors, tastes, or smells. In some embodiments, the at least dimension of the plant growth promoting ability of each microbial consortia comprises one or more of the following: increased growth rate; increased germination rate; earlier germination rate; earlier maturation time,; increased size (including but not limited to weight, height, leaf size, stem size, branching patter, or the size of any part of the plant); increased biomass produced; increased root and/or leaf/shoot growth that leads to an increased yield (herbage, grain, fiber, and/or oil); and increased seed yield.

E. Microbial Consortia with Antibiotic Resistance Ability (“Determine Antimicrobial Resistance to Clinical Antimicrobials”)

As discussed above, in some embodiments, the antibiotic resistance ability node of the SPPIMC development platform represents a subsection of the enriched microbial library. Therefore, in some embodiments, the antibiotic resistance ability node is a collection of microbial isolates or consortia with known resistance to at least one antibiotic (see FIG. 14).

In some embodiments, the present disclosure teaches methods for determining a microbial isolate's ability to resist one or more antibiotics. In some embodiments, the present disclosure provides methods for creating an antibiotic resistance library, comprising i) screening a population of microbial isolates for resistance to a plurality of antibiotics to create an n-dimensional antibiotic resistance profile. As used herein, the “antibiotic resistance profile” provides information on one or more features related to the ability of each microbe to grow in the presence of an antibiotic. For instance, the one or more features may be the number of antibiotics that the microbe is resistant to, the strength of the resistance and the specificity of the resistance. In some embodiments, the antibiotic resistance microbial library comprises information about each microbial isolate's resistance against one or more antibiotics.

In some embodiments, the present disclosure teaches methods of developing a microbial consortia having an optimal and designed level of antibiotic resistance. In some embodiments, the microbial consortia can be developed solely based on the n-dimensional antibiotic resistance profile of individual microbial isolates. Thus, in some embodiments, the disclosure provides methods for creating a soil-borne plant pathogen inhibiting microbial consortia having an optimal and designed level of antibiotic resistance, said method comprising the following steps (a) creating antibiotic resistance profile for each individual microbial isolate of a microbial population (or, alternatively, relying on a previously created antibiotic resistance profile); (b) assembling a library of microbial consortia, each consortium comprising a plurality of microbial isolates from those screened in step a); (c) optionally ranking microbial consortia from the library of microbial consortia based upon at least one dimension of the antibiotic resistance profile of each microbial consortia in said library; and (d) selecting a soil-borne plant pathogen inhibiting microbial consortia having an optimal and designed level of antibiotic resistance from the library.

In some embodiments, the present disclosure teaches methods for developing and empirically testing microbial consortia for their antibiotic resistance. Thus, in some embodiments, the disclosure provides methods for creating a soil-borne plant pathogen inhibiting microbial consortia having an optimal and designed level of antibiotic resistance comprising the following steps: (a) screening a population of microbial isolates for resistance to a plurality of antibiotics to create an n-dimensional antibiotic resistance profile (or, alternatively, relying on a previously created antibiotic resistance profile); (b) assembling a library of microbial consortia, each microbial consortia comprising a combination of microbial isolates from those screened in step a), wherein each microbial consortia in said library is expected to share the antibiotic resistance profile of the individual microbial isolates within the consortia; (c) screening consortia from the library of microbial consortia by growing said microbial consortia in a growth medium comprising an antibiotic for which all of the microbial isolates in the microbial consortia are individually resistant to, and monitoring the continued presence of each microbial isolate within each consortia; and (d) selecting a soil-borne plant pathogen inhibiting microbial consortia having the optimal and designed level of antibiotic resistance.

In some embodiments, the present disclosure teaches an iterative approach for creating a soil-borne plant pathogen inhibiting microbial consortia having an optimal and designed level of antibiotic resistance. Thus, in some embodiments, the method comprises repeating steps (a) through (c), or (a) through (d) one or more times. In some embodiments, the method comprises repeating steps (b) through (c), or (b) through (d) one or more times. Without wishing to be bound by any one theory, the present inventors hypothesize that the iterative production and testing of microbial consortia can result in improved level of antibiotic resistances due to the discovery of unappreciated synergies/interactions within the microbial isolates forming each consortia.

As used herein, a “dimension of antibiotic resistance profile” is any feature or characteristic related to, associated with, contributing to, or caused by the antibiotic resistance profile of the microbial consortia. In some embodiments, the at least one dimension of the antibiotic resistance profile comprises one or more of the following: (i) binary ability to resist the presence of an antibiotic; (ii) strength of the resistance to the antibiotic; (iii) range of antibiotics against which resistance is shown. In some embodiments, the antibiotic resistance of the microbial consortia is measured using any one of the methods disclosed herein, in the Examples. In some embodiments, the antibiotic resistance of the microbial consortia is measured using any one of the methods known in the art for this purpose, such as, for example, as described in Vaz-Jauri, P., Bakker, M. G., Salomon, C. E., and Kinkel, L. L. (2013), Subinhibitory antibiotic concentrations mediate nutrient use and competition among soil Streptomyces. PLoS One 8:12 e81064, and Lee, Chang-Ro, Cho, Ill Hwan, Jeong, Byeong Chul, and Lee, Sang Hee. 2013. Strategies to minimize antibiotic resitsance. International Journal of Environmental Research and Public Health 10: 4274-4305.

The antibiotic used in the methods described herein is not limited, and may be any antibiotic that is known in the art. In some embodiments, the antibiotic is selected from the group consisting of tetracycline, chloramphenicol, vancomycin, erythromycin, novobiocin, streptomycin, azithromycin, kanamycin, and rifampin.

F. Microbial Consortia with Temperature Sensitivity (“Determine Temperature Sensitivity” node)

In some embodiments, the temperature sensitivity node of the SPPIMC development platform represents a subsection of the enriched microbial library. Therefore, in some embodiments, the temperature sensitivity ability node is a collection of microbial isolates or consortia with unknown resistance to difference temperatures tested (see FIG. 14).

In some embodiments, the present disclosure teaches methods for determining a microbial isolate's ability to grow at one or more temperatures. In some embodiments, the present disclosure provides methods for creating a temperature sensitivity library, comprising i) screening a population of microbial isolates for ability to grow at different temperatures to create an n-dimensional temperature sensitivity profile. As used herein, the “temperature sensitivity profile” provides information on one or more features related to the ability of each microbe to grow at different temperatures. For instance, the one or more features may be the range of temperatures that the microbe has the ability to grow on, the strength of the ability and the specificity of the ability. In some embodiments, the temperature sensitivity microbial library comprises information about each microbial isolate's ability to grow at one or more temperatures. In some embodiments, the temperature tested is in the range of about 5° C. to about 45° C., for example about 8° C., about 10° C., about 12° C., about 15° C., about 20° C., about 25° C., about 30° C., about 35° C., or about 40° C.

In some embodiments, the present disclosure teaches methods for determining a microbial isolate's ability to grow at a wide range of temperatures. In some embodiments, the present disclosure teaches methods for determining a microbial isolate's ability to grow at a desired temperature. In some embodiments, the microbial consortia can be developed solely based on the n-dimensional temperature sensitivity profile of individual microbial isolates. Thus, in some embodiments, the disclosure provides methods for creating a soil-borne plant pathogen inhibiting microbial consortia having an optimal and designed ability to grow at a certain temperature, said method comprising the following steps (a) creating temperature sensitivity profile for each individual microbial isolate of a microbial population (or, alternatively, relying on a previously created temperature sensitivity profile); (b) assembling a library of microbial consortia, each consortium comprising a plurality of microbial isolates from those screened in step a); (c) optionally ranking microbial consortia from the library of microbial consortia based upon at least one dimension of the temperature sensitivity profile of each microbial consortia in said library; and (d) selecting a soil-borne plant pathogen inhibiting microbial consortia having an optimal and designed level of temperature sensitivity from the library.

In some embodiments, the present disclosure teaches methods for developing and empirically testing microbial consortia for their temperature sensitivity. Thus, in some embodiments, the disclosure provides methods for creating a soil-borne plant pathogen inhibiting microbial consortia having an optimal and designed level of an ability to grow at a certain temperature comprising the following steps: (a) screening a population of microbial isolates for ability to grow at various different temperatures to create an n-dimensional temperature sensitivity profile (or, alternatively, relying on a previously created temperature sensitivity profile); (b) assembling a library of microbial consortia, each microbial consortia comprising a combination of microbial isolates from those screened in step a), wherein each microbial consortia in said library is expected to share the temperature sensitivity profile of the individual microbial isolates within the consortia; (c) screening consortia from the library of microbial consortia by growing said microbial consortia in a growth medium at different temperatures, and monitoring the continued presence of each microbial isolate within each consortia; and (d) selecting a soil-borne plant pathogen inhibiting microbial consortia having the optimal and designed ability to grow at a certain temperature.

In some embodiments, the present disclosure teaches an iterative approach for creating a soil-borne plant pathogen inhibiting microbial consortia having an optimal and designed ability to grow at a certain temperature. Thus, in some embodiments, the method comprises repeating steps (a) through (c), or (a) through (d) one or more times. In some embodiments, the method comprises repeating steps (b) through (c), or (b) through (d) one or more times. Without wishing to be bound by any one theory, the present inventors hypothesize that the iterative production and testing of microbial consortia can result in improved abilities to grow at certain temperatures due to the discovery of unappreciated synergies/interactions within the microbial isolates forming each consortia.

As used herein, a “dimension of temperature sensitivity profile” is any feature or characteristic related to, associated with, contributing to, or caused by the temperature sensitivity profile of the microbial consortia. In some embodiments, the at least one dimension of the temperature sensitivity profile comprises one or more of the following: (i) binary ability to grow at a particular temperature; (ii) strength of the ability to grow at that temperature; (iii) range of temperatures at which the microbe can grow. In some embodiments, the temperature sensitivity of the microbial consortia is measured using any one of the methods disclosed herein, in the Examples. In some embodiments, the temperature sensitivity of the microbial consortia is measured using any one of the methods known in the art for this purpose.

### Assembling Microbial Consortia According to SPPIMC

In some embodiments, the primary goal/intended purpose of the SPPIMC will be to develop microbial consortia with targeted and complementary pathogen suppressive profiles. As used herein, the term “soil-borne plant pathogen suppressive profile” refers to information about a microbial isolate or a microbial consortia's ability to suppress one or more soil-borne pathogens. In some embodiments, the soil-borne plant pathogen suppressive profile is a database, a list, a network, or any other storage format. As discussed in further detail below, the soil-borne plant pathogen suppressive profile may, in some embodiments, comprise more than one dimension. In some embodiments, dimensions of the soil-borne plant pathogen suppressive profile comprise, for each microbial isolate and/or microbial consortia, the number of pathogens suppressed, a list of pathogens suppressed (represented e.g., by genus species, sequence information, or location of stored culture), the degree of suppression of a pathogen, and the mechanism of suppression. In some embodiments, the soil-borne plant pathogen suppressive profile will include more complex data, including the breadth of activity against pathogens, and the specificity against pathogens. In some embodiments, the SPPIMC platform of the present disclosure teaches the selection of microbial isolates or microbial consortia that have soil-borne plant pathogen suppressive profiles that effectively target the desired or “target” pathogens, while reducing the suppressive activity on non-target pathogens (i.e., targeted profiles, as claimed). In some embodiments, a microbial isolate is selected for inclusion in a microbial consortia because its soil-borne plant pathogen suppressive profile is complementary to the soil-borne plant pathogen suppressive profile of another microbial isolate (i.e. complementary profile). That is, in some embodiments, a microbial isolate is selected because it targets different pathogens than a first microbial isolate, or because it targets the same pathogen via a mechanism that allows for an additive or synergistic suppression due to the combination of both isolates. In other embodiments, a microbial isolate is selected because of its ability to suppress the growth of a pathogen that is only weakly suppressed by another isolate. In some embodiments, the soil-borne plant pathogen suppressive profile of a microbial consortia can be predicted based on the soil-borne plant pathogen suppressive profile of its member microbial isolates (i.e. predicted soil-borne plant pathogen suppressive profile). That is, in some embodiments, the predicted soil-borne plant pathogen suppressive profile of a consortia can be represented by the merging of data from the soil-borne plant pathogen suppressive profiles of each of its member microbial isolates.

In some embodiments, the present disclosure teaches a dynamic approach to MEFB analysis. In some embodiments, the present disclosure teaches creating one or more ecological function balancing nodal microbial libraries, and assembling one or more microbial consortia based on the MEFB analysis. In other embodiments, the present disclosure teaches accessing one or more libraries, and assembling one or more microbial consortia based on the MEFB analysis. As used herein, the term “accessing one or more microbial libraries” means accessing one of the ecological function libraries of the present disclosure (e.g., a mutual inhibitory activity microbial library, a carbon nutrient utilization complementarity microbial library, an antimicrobial signaling capacity and responsiveness microbial library, a plant growth promotion ability microbial library, and and/or an antimicrobial resistance to clinical antimicrobials library). As discussed in further detail below, the aforementioned libraries will, in some embodiments, contain information about each microbial isolate/consortia's features, as determined in each MEFB node.

In some embodiments, the present disclosure teaches assembling a microbial consortia based on the results of the MEFB analysis. Selection of microbial isolates under the MEFB analysis is, in some embodiments, influenced by the design parameters of the intended application.

For example, in some embodiments, the MEFB analysis involves the consideration of the mutual inhibitory activity microbial library. That is, some embodiments, the present disclosure teaches the assembling of a microbial consortia based on the n-dimensional mutual inhibitory activity matrix of a library/population of microbial isolates (e.g., microbial isolates from the soil-borne pathogen suppressive microbial library). In some embodiments, the term “n-dimensional mutual inhibitory activity matrix” refers to information about a microbial isolate or a microbial consortia's ability to inhibit the growth of one other microbial isolate. In some embodiments, the n-dimensional mutual inhibitory activity matrix is a database, a list, a network, or any other storage format. As discussed in further detail below, the n-dimensional mutual inhibitory activity matrix may, in some embodiments, comprise more than one dimension. In some embodiments, the dimensions of the n-dimensional mutual inhibitory activity matrix represent various aspects of the mutual inhibition activities of a microbial isolate or microbial consortia. For example, a dimension of the n-dimensional mutual inhibitory activity matrix can, in some embodiments, comprise for each microbial isolate, a list of microbial isolates inhibited, the degree of inhibition of any microbial isolate, the mechanisms of inhibition, and/or any requirements to trigger that inhibition (e.g., environmental conditions). As used herein, the term inhibition activities, or mutual inhibition activities refers, in some embodiments, to a microbial isolate's ability to suppress/inhibit the growth of another microbial isolate.

In some embodiments the present disclosure teaches assembling microbial consortia with a designed level of mutual inhibitory activity. In some embodiments, the present disclosure teaches selecting microbial isolates that do not strongly inhibit the growth of other microbial isolates within the microbial consortia. In some embodiments, the present disclosure teaches selecting microbial isolates that would also not inhibit other microbial isolates not within the microbial consortia, but which are still considered desirable. In some embodiments, it may be desirable to select a microbial isolate that inhibits the growth of a microbe not within the consortia, but known to inhabit the locus in which the consortia will be applied.

For example, in some embodiments, the MEFB analysis involves the consideration of the carbon nutrient utilization complementarity microbial library. That is, some embodiments, the present disclosure teaches the assembling of a microbial consortia based on a carbon nutrient utilization profile of a library/population of microbial isolates (e.g., microbial isolates from the soil-borne pathogen suppressive microbial library). In some embodiments, the term “a carbon nutrient utilization profile” refers to information about a microbial isolate or a microbial consortia's ability to carbon usage preferences (e.g., the types of carbon substrates on which the organism can survive). In some embodiments, the carbon nutrient utilization profile is a database, a list, a network, or any other storage format. As discussed in further detail below, the carbon nutrient utilization profile may, in some embodiments, comprise more than one dimension. In some embodiments, the dimensions of the carbon nutrient utilization profile represent various aspects of the carbon use preferences of a microbial isolate or microbial consortia. For example, a dimension of the n-dimensional mutual inhibitory activity matrix can, in some embodiments, comprise for each microbial isolate, a list of microbial carbon nutrients capable of supporting life, the degree of growth under each carbon source, the type of metabolism used to process the nutrient growth, and the effect of the carbon source on a microbe's metabolic processes.

In some embodiments the present disclosure teaches assembling microbial consortia with an optimal and designed level of carbon nutrient utilization complementarity. In some embodiments, the term optimal refers to the ability of two microorganisms to subsist in an environment with a particular carbon source profile (i.e. nutrient profile). In some embodiments, optimal, refers to the combination of microbial isolates that achieve the desirable ratio of growth of all the microbial isolates in a consortia. That is, in some embodiments, it may be necessary to have one microbial isolate inhabit the site of application at a higher concentration than other isolates. In some embodiments, the present disclosure teaches that, isolates with complementary nutrient preferences or those with the greatest differences in nutrient preferences, are better for applications in high nutrient soils. In some embodiments, nutrient complementarity is less critical in low nutrient soils. Therefore, in some embodiments, isolates that have different nutrient preferences are chosen to be in a multi-strain inoculant composition exposed to high-nutrient soil. In some embodiments, isolates that have similar nutrient preferences are chosen to be in a multi-strain inoculant composition exposed to low-nutrient soil.

In some embodiments, the present disclosure teaches assembling microbial consortia with a predicted carbon nutrient utilization profile. In some embodiments, the predicted carbon nutrient utilization profile is the carbon nutrient utilization profile of a microbial consortia that is predicted from the carbon nutrient utilization profile of each of the member microbial isolates. That is, in some embodiments, the predicted carbon nutrient utilization profile is a combination of the carbon nutrient utilization profiles of each of the member microbial isolates.

For example, in some embodiments, the MEFB analysis involves the consideration of the an antimicrobial signaling capacity and responsiveness microbial library. That is, some embodiments, the present disclosure teaches the assembling of a microbial consortia based on the antimicrobial signaling capacity and responsiveness profile of a library/population of microbial isolates (e.g., microbial isolates from the soil-borne pathogen suppressive microbial library). In some embodiments, the term “antimicrobial signaling capacity and responsiveness profile ” refers to information about a microbial isolate or a microbial consortia's ability to signal (or be signaled by) at least one other microbial isolate. In some embodiments, the term “signal,” in this context refers to one microbial isolate's ability to trigger the production of one or more anti-pathogenic activities in another microbial isolate (e.g., increasing the production of an antibiotic in a microbe by the presence of a second microbe). In some embodiments, the antimicrobial signaling capacity and responsiveness profile is a database, a list, a network, or any other storage format. As discussed in further detail below, the antimicrobial signaling capacity and responsiveness profile may, in some embodiments, comprise more than one dimension (i.e., the antimicrobial signaling capacity and responsiveness profile may be an n-dimensional antimicrobial signaling capacity and responsiveness profile). In some embodiments, the dimensions of the n-dimensional antimicrobial signaling capacity and responsiveness profile represent various aspects of the signaling/receiving activities of a microbial isolate or microbial consortia. For example, a dimension of the n-dimensional antimicrobial signaling capacity and responsiveness profile can, in some embodiments, comprise for each microbial isolate, a binary ability to signal and modulate the production of antimicrobial compounds in other microbial isolates, strength of ability to signal and modulate the production of antimicrobial compounds in other microbial isolates. For example, a dimension of the n-dimensional antimicrobial signaling capacity and responsiveness profile can, in some embodiments, comprise for each microbial isolate, a binary ability to be signaled and have their production of antimicrobial compounds modulated by other microbial isolates, and strength of ability to be signaled and have their production of antimicrobial compounds modulated by other microbial isolates.

In some embodiments the present disclosure teaches assembling microbial consortia with an optimal and designed level of antimicrobial signaling capacity and responsiveness. In some embodiments, optimal antimicrobial signaling capacity and responsiveness profiles are ones in which a desirable signaling event occurs so as to provide the microbial consortia greater pathogen suppressive activities than any of its individual microbial isolates alone. In some embodiments, optimal antimicrobial signaling capacity and responsiveness profiles exhibit synergistic properties, as measured by the Colby formula disclosed herein. Signaling/responsiveness capacity can be an important part of a microbial consortia. In some embodiments, the present disclosure teaches microbial consortia in which at least one microbial isolate exhibits a signaling activity. In some embodiments, the signaling activity can be towards one other microbial isolate in the consortia. In other embodiments, the signaling activity affects two or more microbial isolates in the consortia. In some embodiments the signaling is mediated by only one microbial isolate. In other embodiments, the signaling activity requires two or more microbial isolates. In some embodiments, the microbial isolates selected for a microbial consortia can also signal microbes outside of the microbial consortia (e.g., other microbes present at the locus of application).

In some embodiments, the present disclosure teaches assembling microbial consortia with a predicted antimicrobial signaling capacity and responsiveness profile. In some embodiments, the predicted antimicrobial signaling capacity and responsiveness profile is the antimicrobial signaling capacity and responsiveness profile of a microbial consortia that is predicted from the antimicrobial signaling capacity and responsiveness profile of each of the member microbial isolates. That is, in some embodiments, the predicted antimicrobial signaling capacity and responsiveness profile is a combination of the antimicrobial signaling capacity and responsiveness profiles of each of the member microbial isolates. In some embodiments the predicted antimicrobial signaling capacity and responsiveness profiles are accurate. In some embodiments, empirical testing of the microbial consortia may reveal unappreciated antimicrobial signaling capacity and responsiveness activities that were not apparent from pairwise comparisons, or which may occur only in the presence of particular environmental factors. Thus, in some embodiments, the present disclosure teaches SPPIMC methods with iterative testing of microbial consortia.

For example, in some embodiments, the MEFB analysis involves the consideration of the a plant growth promotion ability microbial library. That is, some embodiments, the present disclosure teaches the assembling of a microbial consortia based on the plant growth promoting ability profile of a library/population of microbial isolates (e.g., microbial isolates from the soil-borne pathogen suppressive microbial library). In some embodiments, the term “plant growth promoting ability profile” refers to information about a microbial isolate or a microbial consortia's ability to promote the growth of at least one plant. In some embodiments, the term “promote,” in this context refers to one microbial isolate or microbial consortia's ability to enhance the growth of one plant. In some embodiments, the microbial isolate or microbial consortia enhance growth by, for example, increasing growth rate, increasing plant health, or marketable product. In some embodiments, the microbial isolate or microbial consortia enhance growth by providing the plant with a nutrient (e.g., nitrogen). In some embodiments, the microbial isolate or microbial consortia enhance growth by providing reducing pathogen pressure. In some embodiments, the plant growth promoting ability profile is a database, a list, a network, or any other storage format. As discussed in further detail below, the plant growth promoting ability profile may, in some embodiments, comprise more than one dimension (i.e., the plant growth promoting ability profile may be an n-dimensional plant growth promoting ability profile). In some embodiments, the dimensions of the n-dimensional plant growth promoting ability profile represent various aspects of the plant growth promotion activities of a microbial isolate or microbial consortia. For example, a dimension of the n-dimensional plant growth promoting ability profile can, in some embodiments, comprise for each microbial isolate, information about the binary ability to promote growth of a particular plant, the degree of growth promotion for the particular plant; and the mechanism by which the microbial isolate or consortia promotes the growth of the particular plant.

In some embodiments the present disclosure teaches assembling microbial consortia with an optimal and designed level of plant growth promoting ability. In some embodiments, optimal plant growth promoting ability profiles are ones which have the most positive effect on plant growth and development. As used herein, the term growth refers to not only size of the plant, but also the development of marketable product from the plant. That is, in some embodiments, an optimal plant growth promoting ability may not correlate with the largest plant, but may instead be associate with greater return on investment for the farmer due to e.g., more fruit, or higher quality fruit. In some embodiments, optimal plant growth promoting ability profiles exhibit additive properties, where at the plant growth effects of two microbial isolates contribute to the overall growth and development of the plant. In some embodiments, optimal plant growth promoting ability profiles exhibit synergistic properties, as measured by the Colby formula disclosed herein. Person's having skill in the art will recognize upon reading this disclosure the desirable features of a microbial consortia assembled under this node. In some embodiments, microbial isolates are selected based on their different effects on plant growth. In some embodiments, microbial isolates are selected based on similar effects on plant growth, where those effects are additive or synergistic, for example due to having different mechanisms, or due to a plant's capacity to accept greater enhancement from additional isolates. In some embodiments, microbial consortia are constructed to promote the growth of a plant by inhibiting the growth of another plant, either for spacing of similar plants in a monoculture, or to control weeds or other undesirable plants.

In some embodiments, the present disclosure teaches assembling microbial consortia with a predicted plant growth promoting ability profile. In some embodiments, the predicted plant growth promoting ability profile of a microbial consortia is the plant growth promoting ability profile of a microbial consortia that is predicted from the plant growth promoting ability profile of each of the member microbial isolates. That is, in some embodiments, the predicted plant growth promoting ability profile is a combination of the plant growth promoting ability profile of each of the member microbial isolates. In some embodiments the predicted plant growth promoting ability profile are accurate. In some embodiments, empirical testing of the microbial consortia may reveal unappreciated plant growth promoting abilities that were not apparent from the plant growth promoting ability profiles of individual microbial isolates. Thus, in some embodiments, the present disclosure teaches SPPIMC methods with iterative testing of microbial consortia. For example, in some embodiments, the MEFB analysis involves the consideration of the an antimicrobial resistance to clinical antimicrobials library. That is, some embodiments, the present disclosure teaches the assembling of a microbial consortia based on the n-dimensional antibiotic resistance profile of a library/population of microbial isolates (e.g., microbial isolates from the soil-borne pathogen suppressive microbial library). In some embodiments, the term “n-dimensional antibiotic resistance profile” refers to information about a microbial isolate or a microbial consortia's ability to grow in the presence of one or more antibiotics. In some embodiments, the present disclosure teaches selecting microbial isolates and microbial consortia capable of resisting antibiotics known or suspected to be present in the application locus. In some embodiments, the n-dimensional antibiotic resistance profile is a database, a list, a network, or any other storage format. As discussed in further detail below, the n-dimensional antibiotic resistance profile may, in some embodiments, comprise more than one dimension. In some embodiments, the dimensions of the n-dimensional antibiotic resistance profile represent various aspects of the antibiotic resistance properties of a microbial isolate or microbial consortia. For example, a dimension of the n-dimensional antibiotic resistance profile can, in some embodiments, comprise for each microbial isolate, information about the binary ability to resist the presence of an antibiotic, strength of the resistance to the antibiotic and/or range of antibiotics against which resistance is shown.

In some embodiments the present disclosure teaches assembling microbial consortia with an optimal and designed level of antibiotic resistance. In some embodiments, optimal antibiotic resistance profiles are those which result in the highest resistance to antibiotics that are (or are expected) to be present in the application locus. In some embodiments, optimal antibiotic resistance profiles exhibit additive or synergistic properties, where a combination of microbial isolates cause the microbial consortia as a whole to be more resistant to an antibiotic than any one microbial isolate, or than all microbial isolates in the consortia individually.

In some embodiments, the present disclosure teaches assembling microbial consortia with a predicted antibiotic resistance profile. In some embodiments, the predicted antibiotic resistance profile of a microbial consortia is the antibiotic resistance profile of a microbial consortia that is predicted from the antibiotic resistance profile of each of the member microbial isolates. That is, in some embodiments, the predicted antibiotic resistance profile is a combination of the antibiotic resistance profile of each of the member microbial isolates. In some embodiments the predicted antibiotic resistance profiles are accurate. In some embodiments, empirical testing of the microbial consortia may reveal unappreciated antibiotic resistances that were not apparent from the antibiotic resistance profiles of individual microbial isolates. Thus, in some embodiments, the present disclosure teaches SPPIMC methods with iterative testing of microbial consortia.

For example, in some embodiments, the MEFB analysis involves the consideration of the a temperature sensitivity ability library (temperature sensitivity ability node). That is, some embodiments, the present disclosure teaches the assembling of a microbial consortia based on the temperature sensitivity profile of a library/population of microbial isolates (e.g., microbial isolates from the soil-borne pathogen suppressive microbial library). In some embodiments, the term “temperature sensitivity profile” refers to information about a microbial isolate or a microbial consortia' s ability to grow at different temperatures. In some embodiments, the temperature sensitivity profile is a database, a list, a network, or any other storage format. As discussed in further detail below, the temperature sensitivity profile may, in some embodiments, comprise more than one dimension (i.e., the temperature sensitivity profile may be an n-dimensional plant temperature sensitivity profile). In some embodiments, the dimensions of the n-dimensional temperature sensitivity profile represent various aspects of the microbial isolate's response to temperatures. For example, a dimension of the n-dimensional temperature sensitivity profile can, in some embodiments, comprise for each microbial isolate, information about the binary ability of a microbe to grow at a temperature, the degree of growth at a temperature; and its ability to produce antibiotic compounds at various temperatures.

In some embodiments the present disclosure teaches assembling microbial consortia with an optimal and designed level temperature sensitivity. In some embodiments, optimal temperature sensitivity profiles are ones which permit the microbial isolate/microbial consortia to grow have the desired effect at the locus and season of intended application. Persons having skill in the art will recognize the temperature differentials between different fields located at different latitudes, or in different climates or seasons. In some embodiments, the purpose of the temperature sensitivity profile node is to ensure that the resulting microbial isolate is tailored to yet another environmental factor at the application locus.

### Isolated Microbes

The disclosure provides microbes with plant pathogen suppressive activity. In some embodiments, the microbes comprise polynucleotide sequences that share at least 70%, 75%, 80%, 81%, 82%, 83%, 84%, 85%, 86%, 87%, 88%, 89%, 90%, 91%, 92%, 93%, 94%, 95%, 95.1%, 95.2%, 95.3%, 95.4%, 95.5%, 95.6%, 95.7%, 95.8%, 95.9%, 96%, 96.1%, 96.2%, 96.3%, 96.4%, 96.5%, 96.6%, 96.7%, 96.8%, 96.9%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, or 100% sequence identity with the 16S rRNA sequence, 18S rRNA sequence, 23S rRNA sequence, the internal transcribed spacer (ITS1) sequence and/or ITS2 sequence of any one of the microbes listed in this specification.

In some embodiments, the microbes comprise 16S rRNA sequences that share at least 70%, 75%, 80%, 81%, 82%, 83%, 84%, 85%, 86%, 87%, 88%, 89%, 90%, 91%, 92%, 93%, 94%, 95%, 95.1%, 95.2%, 95.3%, 95.4%, 95.5%, 95.6%, 95.7%, 95.8%, 95.9%, 96%, 96.1%, 96.2%, 96.3%, 96.4%, 96.5%, 96.6%, 96.7%, 96.8%, 96.9%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, or 100% sequence identity with any one of SEQ ID NOs: 1-5.

In some embodiments, the microbes are fungi or bacteria. In some embodiments, the microbe is a species of Mycorrhizae, Bacillus, Pseudomonas, Streptomyces, Trichoderma, Tallaromyces, Gliocladium, non-pathogenic Fusarium, nitrogen fixing bacteria or yeast.

In some embodiments, the microbes belong to the genus of Streptomycetes. In some embodiments, the microbes are isolated species of Streptomyces. In some embodiments, the isolated species of Streptomyces are selected from Streptomyces abietis, Streptomyces abikoensis, Streptomyces aburaviensis, Streptomyces achromogenes, Streptomyces acidiscabies, Streptomyces actinomycinicus, Streptomyces acrimycini, Streptomyces actuosus, Streptomyces aculeolatus, Streptomyces adustus, Streptomyces abyssalis, Streptomyces afghaniensis, Streptomyces aidingensis, Streptomyces africanus, Streptomyces alanosinicus, Streptomyces albaduncus, Streptomyces albiaxialis, Streptomyces albidochromogenes, Streptomyces albiflavescens, Streptomyces albiflaviniger, Streptomyces albidoflavus, Streptomyces albofaciens, Streptomyces alboflavus, Streptomyces albogriseolus, Streptomyces albolongus, Streptomyces alboniger, Streptomyces albospinus, Streptomyces albulus, Streptomyces albus, Streptomyces aldersoniae, Streptomyces alfalfa, Streptomyces alkaliphilus, Streptomyces alkalithermotolerans, Streptomyces almquistii, Streptomyces alni, Streptomyces althioticus, Streptomyces amakusaensis, Streptomyces ambofaciens, Streptomyces amphotericinicus, Streptomyces amritsarensis, Streptomyces anandii, Streptomyces andamanensis, Streptomyces angustmyceticus, Streptomyces anthocyanicus, Streptomyces antibioticus, Streptomyces antimycoticus, Streptomyces anulatus, Streptomyces aomiensis, Streptomyces araujoniae, Streptomyces ardus, Streptomyces aridus, Streptomyces arenae, Streptomyces armeniacus, Streptomyces artemisiae, Streptomyces arcticus, Streptomyces ascomycinicus, Streptomyces asenjonii, Streptomyces asiaticus, Streptomyces asterosporus, Streptomyces atacamensis, Streptomyces atratus, Streptomyces atriruber, Streptomyces atroolivaceus, Streptomyces atrovirens, Streptomyces aurantiacus, Streptomyces aurantiogriseus, Streptomyces auratus, Streptomyces aureocirculatus, Streptomyces aureofaciens, Streptomyces aureorectus, Streptomyces aureoverticillatus, Streptomyces aureus, Streptomyces avellaneus, Streptomyces avermitilis, Streptomyces avicenniae, Streptomyces avidinii, Streptomyces axinellae, Streptomyces azureus, Streptomyces bacillaris, Streptomyces badius, Streptomyces bambergiensis, Streptomyces bambusae, Streptomyces bangladeshensis, Streptomyces baliensis, Streptomyces barkulensis, Streptomyces beijiangensis, Streptomyces bellus, Streptomyces bikiniensis, Streptomyces blastmyceticus, Streptomyces bluensis, Streptomyces bobili, Streptomyces bohaiensis, Streptomyces boninensis, Streptomyces bottropensis, Streptomyces brasiliensis, Streptomyces brevispora, Streptomyces bullii, Streptomyces bungoensis, Streptomyces burgazadensis, Streptomyces bryophytorum, Streptomyces cacaoi, Streptomyces caelestis, Streptomyces caeruleatus, Streptomyces caldifontis, Streptomyces calidiresistens, Streptomyces calvus, Streptomyces camponoticapitis, Streptomyces canalis, Streptomyces canaries, Streptomyces canchipurensis, Streptomyces candidus, Streptomyces cangkringensis, Streptomyces caniferus, Streptomyces canus, Streptomyces capparidis, Streptomyces capillispiralis, Streptomyces capoamus, Streptomyces carpaticus, Streptomyces carpinensis, Streptomyces castelarensis, Streptomyces catbensis, Streptomyces catenulae, Streptomyces cavourensis, Streptomyces cellostaticus, Streptomyces celluloflavus, Streptomyces cellulolyticus, Streptomyces cellulosae, Streptomyces capitiformicae, Streptomyces cerasinus, Streptomyces chartreusis, Streptomyces chattanoogensis, and Streptomyces cheonanensis.

In some embodiments, the isolated species of Streptomyces are selected from Streptomyces chiangmaiensis, Streptomyces chitinivorans, Streptomyces chrestomyceticus, Streptomyces chromofuscus, Streptomyces chryseus, Streptomyces chilikensis, Streptomyces chlorus, Streptomyces chumphonensis, Streptomyces cinereorectus, Streptomyces cinereoruber, Streptomyces cinereospinus, Streptomyces cinereus, Streptomyces cinerochromogenes, Streptomyces cinnabarinus, Streptomyces cinnabarigriseus, Streptomyces cinnamonensis, Streptomyces cinnamoneus, Streptomyces cirratus, Streptomyces ciscaucasicus, Streptomyces clavifer, Streptomyces clavuligerus, Streptomyces coacervatus, Streptomyces cocklensis, Streptomyces coelescens, Streptomyces coelicoflavus, Streptomyces coelicolor, Streptomyces coeruleoflavus, Streptomyces coeruleofuscus, Streptomyces coeruleoprunus, Streptomyces coeruleorubidus, Streptomyces coerulescens, Streptomyces collinus, Streptomyces colombiensis, Streptomyces corchorusii, Streptomyces costaricanus, Streptomyces cremeus, Streptomyces crystallinus, Streptomyces cuspidosporus, Streptomyces cyaneofuscatus, Streptomyces cyaneus, Streptomyces cyanoalbus, Streptomyces cyslabdanicus, Streptomyces daghestanicus, Streptomyces daliensi, Streptomyces daqingensis, Streptomyces davaonensis, Streptomyces deccanensis, Streptomyces decoyicus, Streptomyces demainii, Streptomyces deserti, Streptomyces diastaticus, Streptomyces diastatochromogenes, Streptomyces djakartensis, Streptomyces drozdowiczii, Streptomyces durhamensis, Streptomyces durmitorensis, Streptomyces echinatus, Streptomyces echinoruber, Streptomyces ederensis, Streptomyces emeiensis, Streptomyces endophyticus, Streptomyces endus, Streptomyces enissocaesilis, Streptomyces erythrogriseus, Streptomyces erringtonii, Streptomyces eurocidicus, Streptomyces europaeiscabiei, Streptomyces eurythermus, Streptomyces exfoliates, Streptomyces fabae, Streptomyces fenghuangensis, Streptomyces ferralitis, Streptomyces filamentosus, Streptomyces fildesensis, Streptomyces filipinensis, Streptomyces fimbriatus, Streptomyces finlayi, Streptomyces flaveolus, Streptomyces flaveus, Streptomyces flavofungini, Streptomyces flavotricini, Streptomyces flavovariabilis, Streptomycesflavovirens, Streptomycesflavoviridis, Streptomyces formicae, Streptomyces fractus, Streptomyces fradiae, Streptomyces fragilis, Streptomyces fukangensis, Streptomyces fulvissimus, Streptomyces fulvorobeus, Streptomyces fumanus, Streptomyces fumigatiscleroticus, Streptomyces fuscichromogenes, Streptomyces fuscigenes, Streptomyces galbus, Streptomyces galilaeus, Streptomyces gamaensis, Streptomyces gancidicus, Streptomyces gardneri, Streptomyces gelaticus, Streptomyces geldanamycininus, Streptomyces geysiriensis, Streptomyces ghanaensis, Streptomyces gilvifuscus, Streptomyces glaucescens, Streptomyces glauciniger, Streptomyces glaucosporus, Streptomyces glaucus, Streptomyces globisporus, Streptomyces globosus, Streptomyces glomeratus, Streptomyces glomeroaurantiacus, Streptomyces glycovorans, Streptomyces gobitricini, Streptomyces goshikiensis, Streptomyces gougerotii, Streptomyces graminearus, Streptomyces gramineus, Streptomyces graminifolii, Streptomyces graminilatus, Streptomyces graminisoli, Streptomyces griseiniger, Streptomyces griseoaurantiacus, Streptomyces griseocarneus, Streptomyces griseochromogenes, Streptomyces griseoflavus, Streptomyces griseofuscus, Streptomyces griseoincarnatus, Streptomyces griseoloalbus, Streptomyces griseolus, and Streptomyces griseoluteus.

In some embodiments, the isolated species of Streptomyces are selected from Streptomyces griseomycini, Streptomyces griseoplanus, Streptomyces griseorubens, Streptomyces griseoruber, Streptomyces griseorubiginosus, Streptomyces griseosporeus, Streptomyces griseostramineus, Streptomyces griseoviridis, Streptomyces griseus, Streptomyces guanduensis, Streptomyces gulbargensis, Streptomyces hainanensis, Streptomyces haliclonae, Streptomyces halophytocola, Streptomyces halstedii, Streptomyces harbinensis, Streptomyces hawaiiensis, Streptomyces hebeiensis, Streptomyces heilongjiangensis, Streptomyces heliomycini, Streptomyces helvaticus, Streptomyces herbaceous, Streptomyces herbaricolor, Streptomyces himastatinicus, Streptomyces hiroshimensis, Streptomyces hirsutus, Streptomyces hokutonensis, Streptomyces hoynatensis, Streptomyces humidus, Streptomyces humiferus, Streptomyces hundungensis, Streptomyces hyaluromycini, Streptomyces hyderabadensis, Streptomyces hygroscopicus, Streptomyces hypolithicus, Streptomyces iakyrus, Streptomyces iconiensis, Streptomyces incanus, Streptomyces indiaensis, Streptomyces indigoferus, Streptomyces indicus, Streptomyces indoligenes, Streptomyces indonesiensis, Streptomyces intermedius, Streptomyces inusitatus, Streptomyces ipomoeae, Streptomyces iranensis, Streptomyces janthinus, Streptomyces javensis, Streptomyces jeddahensis, Streptomyces jietaisiensis, Streptomyces jiujiangensis, Streptomyces kaempferi, Streptomyces kanamyceticus, Streptomyces karpasiensis, Streptomyces kasugaensis, Streptomyces katrae, Streptomyces kalpinensis, Streptomyces kebangsaanensis, Streptomyces klenkii, Streptomyces koyangensis, Streptomyces kunmingensis, Streptomyces kurssanovii, Streptomyces kronopolitis, Streptomyces krungchingensis, Streptomyces labedae, Streptomyces lacrimifluminis, Streptomyces lactacystinicus, Streptomyces lacticiproducens, Streptomyces laculatispora, Streptomyces lanatus, Streptomyces lannensis, Streptomyces lasiicapitis, Streptomyces lateritius, Streptomyces laurentii, Streptomyces lavendofoliae, Streptomyces lavendulae, Streptomyces lavenduligriseus, Streptomyces leeuwenhoekii, Streptomyces lavendulocolor, Streptomyces levis, Streptomyces libani, Streptomyces lienomycini, Streptomyces lilacinus, Streptomyces lincolnensis, Streptomyces litmocidini, Streptomyces litoralis, Streptomyces lohii, Streptomyces lomondensis, Streptomyces longisporoflavus, Streptomyces longispororuber, Streptomyces lopnurensis, Streptomyces lonarensis, Streptomyces longisporus, Streptomyces longwoodensis, Streptomyces lucensis, Streptomyces lunaelactis, Streptomyces lunalinharesii, Streptomyces luridiscabiei, Streptomyces luridus, Streptomyces lusitanus, Streptomyces lushanensis, Streptomyces luteireticuli, Streptomyces luteogriseus, Streptomyces luteosporeus, Streptomyces luteus, Streptomyces lydicus, Streptomyces macrosporus, Streptomyces malachitofuscus, Streptomyces malachitospinus, Streptomyces malaysiensis, Streptomyces mangrove, Streptomyces marinus, Streptomyces marokkonensis, Streptomyces mashuensis, Streptomyces massasporeus, Streptomyces matensis, Streptomyces mayteni, Streptomyces mauvecolor, Streptomyces megaspores, Streptomyces melanogenes, Streptomyces melanosporofaciens, Streptomyces mexicanus, Streptomyces michiganensis, Streptomyces microflavus, Streptomyces milbemycinicus, Streptomyces minutiscleroticus, Streptomyces mirabilis, Streptomyces misakiensis, Streptomyces misionensis, Streptomyces mobaraensis, and Streptomyces monomycini.

In some embodiments, the isolated species of Streptomyces are selected from Streptomyces mordarskii, Streptomyces morookaense, Streptomyces muensis, Streptomyces murines, Streptomyces mutabilis, Streptomyces mutomycini, Streptomyces naganishii, Streptomyces nanhaiensis, Streptomyces nanshensis, Streptomyces narbonensis, Streptomyces nashvillensis, Streptomyces netropsis, Streptomyces neyagawaensis, Streptomyces niger, Streptomyces nigrescens, Streptomyces nitrosporeus, Streptomyces niveiciscabiei, Streptomyces niveiscabiei, Streptomyces niveoruber, Streptomyces niveus, Streptomyces noboritoensis, Streptomyces nodosus, Streptomyces nogalater, Streptomyces nojiriensis, Streptomyces noursei, Streptomyces novaecaesareae, Streptomyces ochraceiscleroticus, Streptomyces odonnellii, Streptomyces olivaceiscleroticus, Streptomyces olivaceoviridis, Streptomyces olivaceus, Streptomyces olivicoloratus, Streptomyces olivochromogenes, Streptomyces olivomycini, Streptomyces olivoverticillatus, Streptomyces omiyaensis, Streptomyces osmaniensis, Streptomyces orinoci, Streptomyces oryzae, Streptomyces ovatisporus, Streptomyces pactum, Streptomyces palmae, Streptomyces panacagri, Streptomyces panaciradicis, Streptomyces paradoxus, Streptomyces parvulus, Streptomyces parvus, Streptomyces pathocidini, Streptomyces paucisporeus, Streptomyces peucetius, Streptomyces phaeochromogenes, Streptomyces phaeofaciens, Streptomyces phaeogriseichromatogenes, Streptomyces phaeoluteichromatogenes, Streptomyces phaeoluteigriseus, Streptomyces phaeopurpureus, Streptomyces pharetrae, Streptomyces pharmamarensis, Streptomyces phyllanthi, Streptomyces phytohabitans, Streptomyces pilosus, Streptomyces pini, Streptomyces platensis, Streptomyces plicatus, Streptomyces plumbiresistens, Streptomyces pluricolorescens, Streptomyces pluripotens, Streptomyces polyantibioticus, Streptomyces polychromogenes, Streptomyces polygonati, Streptomyces polymachus, Streptomyces poonensis, Streptomyces prasinopilosus, Streptomyces prasinosporus, Streptomyces prasinus, Streptomyces pratens, Streptomyces platensis, Streptomyces prunicolor, Streptomyces psammoticus, Streptomyces pseudoechinosporeus, Streptomyces pseudogriseolus, Streptomyces pseudovenezuelae, Streptomyces pulveraceus, Streptomyces puniceus, Streptomyces puniciscabiei, Streptomyces purpeofuscus, Streptomyces purpurascens, Streptomyces purpureus, Streptomyces purpurogeneiscleroticus, Streptomyces qinglanensis, Streptomyces racemochromogenes, Streptomyces radiopugnans, Streptomyces rameus, Streptomyces ramulosus, Streptomyces rapamycinicus, Streptomyces recifensis, Streptomyces rectiviolaceus, Streptomyces regensis, Streptomyces resistomycificus, Streptomyces reticuliscabiei, Streptomyces rhizophilus, Streptomyces rhizosphaericus, Streptomyces rhizosphaerihabitans, Streptomyces rimosus, Streptomyces rishiriensis, Streptomyces rochei, Streptomyces roietensis, Streptomyces rosealbus, Streptomyces roseiscleroticus, Streptomyces roseofulvus, Streptomyces roseolilacinus, Streptomyces roseolus, Streptomyces roseosporus, Streptomyces roseoviolaceus, Streptomyces roseoviridis, Streptomyces ruber, Streptomyces rubidus, Streptomyces rubiginosohelvolus, Streptomyces rubiginosus, Streptomyces rubrisoli, Streptomyces rubrogriseus, Streptomyces rubrus, Streptomyces rutgersensis, Streptomyces salilacus, Streptomyces samsunensis, Streptomyces sanglieri, Streptomyces sannanensis, Streptomyces sanyensis, Streptomyces sasae, Streptomyces scabiei, Streptomyces scabrisporus, and Streptomyces sclerotialus.

In some embodiments, the isolated species of Streptomyces are selected from Streptomyces scopiformis, Streptomyces scopuliridis, Streptomyces sedi, Streptomyces seoulensis, Streptomyces seranimatus, Streptomyces seymenliensis, Streptomyces shaanxiensis, Streptomyces shenzhenensis, Streptomyces showdoensis, Streptomyces silaceus, Streptomyces siamensis, Streptomyces similanensis, Streptomyces sindenensis, Streptomyces sioyaensis, Streptomyces smyrnaeus, Streptomyces sodiiphilus, Streptomyces solisilvae, Streptomyces somaliensis, Streptomyces sudanensis, Streptomyces sparsogenes, Streptomyces sparsus, Streptomyces specialis, Streptomyces spectabilis, Streptomyces speibonae, Streptomyces speleomycini, Streptomyces spinoverrucosus, Streptomyces spiralis, Streptomyces spiroverticillatus, Streptomyces spongiae, Streptomyces spongiicola, Streptomyces sporocinereus, Streptomyces sporoclivatus, Streptomyces spororaveus, Streptomyces sporoverrucosus, Streptomyces staurosporininus, Streptomyces stelliscabiei, Streptomyces stramineus, Streptomyces subrutilus, Streptomyces sulfonofaciens, Streptomyces sulphureus, Streptomyces sundarbansensis, Streptomyces synnematoformans, Streptomyces tacrolimicus, Streptomyces tanashiensis, Streptomyces tateyamensis, Streptomyces tauricus, Streptomyces tendae, Streptomyces termitum, Streptomyces thermoalcalitolerans, Streptomyces thermoautotrophicus, Streptomyces thermocarboxydovorans, Streptomyces thermocarboxydus, Streptomyces thermocoprophilus, Streptomyces thermodiastaticus, Streptomyces thermogriseus, Streptomyces thermolineatus, Streptomyces thermospinosisporus, Streptomyces thermoviolaceus, Streptomyces thermovulgaris, Streptomyces thinghirensis, Streptomyces thioluteus, Streptomyces tritici, Streptomyces torulosus, Streptomyces toxytricini, Streptomyces tremellae, Streptomyces tritolerans, Streptomyces tricolor, Streptomyces tsukubensis, Streptomyces tubercidicus, Streptomyces tuirus, Streptomyces tunisiensis, Streptomyces turgidiscabies, Streptomyces tyrosinilyticus, Streptomyces umbrinus, Streptomyces variabilis, Streptomyces variegatus, Streptomyces varsoviensis, Streptomyces verticillus, Streptomyces vastus, Streptomyces venezuelae, Streptomyces verrucosisporus, Streptomyces vietnamensis, Streptomyces vinaceus, Streptomyces vinaceusdrappus, Streptomyces violaceochromogenes, Streptomyces violaceolatus, Streptomyces violaceorectus, Streptomyces violaceoruber, Streptomyces violaceorubidus, Streptomyces violaceus, Streptomyces violaceusniger, Streptomyces violarus, Streptomyces violascens, Streptomyces violens, Streptomyces vixens, Streptomyces virginiae, Streptomyces viridis, Streptomyces viridiviolaceus, Streptomyces viridobrunneus, Streptomyces viridochromogenes, Streptomyces viridodiastaticus, Streptomyces viridosporus, Streptomyces vitaminophilus, Streptomyces wedmorensis, Streptomyces wellingtoniae, Streptomyces werraensis, Streptomyces wuyuanensis, Streptomyces xanthochromogenes, Streptomyces xanthocidicus, Streptomyces xantholiticus, Streptomyces xanthophaeus, Streptomyces xiamenensis, Streptomyces xinghaiensis, Streptomyces xishensis, Streptomyces xylanilyticus, Streptomyces yaanensis, Streptomyces yanglinensis, Streptomyces yangpuensis, Streptomyces yanii, Streptomyces yatensis, Streptomyces yeochonensis, Streptomyces yerevanensis, Streptomyces yogyakartensis, Streptomyces yokosukanensis, Streptomyces youssoufiensis, Streptomyces yunnanensi, Streptomyces zagrosensis, Streptomyces zaomyceticus, Streptomyces zhaozhouensis, Streptomyces zhihengii, Streptomyces zinciresistens, and Streptomyces ziwulingensis.

In some embodiments, the microbe may be isolated from a field, site or soil, in which agricultural monoculture has been practiced for a specified length of time. The specified length of time may be in the range of about 1 week to more than 60 years, for example, about 3 weeks, about 2 months, about 6 months, about 2 years, about five years, about ten years, about twenty years, or about thirty years, including all values and subranges that lie therebetween. In some embodiments, the microbe isolated from such an environment may have been subjected to directed selection from long-term high-nutrient conditions of agricultural monoculture. In some embodiments, the microbe isolated from an agricultural soil is GS1.

In some embodiments, the microbe is isolated from a nonagricultural field, site or soil. As used herein, the term “nonagricultural” refers to a field, site or soil that has not been subject to agricultural chemicals or plowing, tilling, or other agricultural soil disturbance for a specified length of time. The specified length of time may be in the range of about 1 week to about 50 years, for example, about 3 weeks, about 2 months, about 6 months, about 2 years, about five years, about ten years, about twenty years, or about thirty years, including all values and subranges that lie therebetween. In some embodiments, the microbe isolated from such an environment are subjected to selection under long-term low-nutrient conditions. In some embodiments, microbes isolated from a nonagricultural environment may be more likely to support plant growth and/or mediate antibiotic production. In some embodiments, the microbe isolated from a non-agricultural site is PS1.

In some embodiments, the microbes of the present disclosure that have been genetically modified. In some embodiments, the genetically modified or recombinant microbes comprise polynucleotide sequences which do not naturally occur in said microbes. In some embodiments, the microbes may comprise heterologous polynucleotides. In further embodiments, the heterologous polynucleotides may be operably linked to one or more polynucleotides native to the microbes.

In some embodiments, the heterologous polynucleotides may be reporter genes or selectable markers. In some embodiments, reporter genes may be selected from any of the family of fluorescence proteins (e.g., GFP, RFP, YFP, and the like), β-galactosidase, or luciferase. In some embodiments, selectable markers may be selected from neomycin phosphotransferase, hygromycin phosphotransferase, aminoglycoside adenyltransferase, dihydrofolate reductase, acetolactase synthase, bromoxynil nitrilase, β-glucuronidase, dihydrogolate reductase, and chloramphenicol acetyltransferase. In some embodiments, the heterologous polynucleotide may be operably linked to one or more promoter. In some embodiments the isolated microbial strains express transgenic or native molecules or compounds that have antimicrobial activity.

A. Microbial Consortia with Mutual Inhibition Activity (“Determine Mutual Inhibition Activity”)

### Microbial Consortia

The disclosure provides plant pathogen inhibiting microbial consortia, comprising two or more of the any of the microbes disclosed herein. In some embodiments, the microbial consortia comprises at least one microbe isolated from a monoculture agricultural soil. In some embodiments, the microbial consortia comprises at least one microbe isolated from non-agricultural soil. In some embodiments, the microbial consortia comprises at least one microbe isolated from a monoculture agricultural soil and at least one microbe isolated from non-agricultural soil.

In some embodiments, the disclosure provides microbial consortia comprising a combination of at least any two microbes disclosed in the present disclosure. In certain embodiments, the microbial consortia of the present disclosure comprise two microbes, or three microbes, or four microbes, or five microbes, or six microbes, or seven microbes, or eight microbes, or nine microbes, or ten or more microbes. Said microbes of the compositions are different microbial species, or different strains of a microbial species.

In some embodiments, the microbial consortia comprises at least one isolated microbial species belonging to genus Streptomyces and/or Bacillus. In some embodiments, the microbial consortia comprises any two microbial isolates selected from the group consisting of: Streptomyces GS 1 (Streptomyces lydicus), Streptomyces PS1 (Streptomyces sp. 3211.1) and Brevibacillus PS3 (Brevibacillus laterosporus). In some embodiments, the microbial consortia comprises Streptomyces GS 1 (Streptomyces lydicus), Streptomyces PS1 (Streptomyces sp. 3211.1) and Brevibacillus PS3 (Brevibacillus laterosporus).

The disclosure provides plant pathogen inhibiting microbial consortium, comprising (a) Brevibacillus laterosporus; (b) Streptomyces lydicus; and (c) Streptomyces sp. 3211.1. The disclosure provides plant pathogen inhibiting microbial consortium, comprising (a) a Brevibacillus sp. comprising a 16S nucleic acid sequence sharing at least 97% sequence identity to any one of SEQ ID NO: 3,4, or 5; (b) a Streptomyces sp. comprising a 16S nucleic acid sequence sharing at least 97% sequence identity to SEQ ID NO: 2; and (c) a Streptomyces lydicus comprising a 16S nucleic acid sequence sharing at least 97% sequence identity to SEQ ID NO: 1. The disclosure provides plant pathogen inhibiting microbial consortium, comprising (a) a Brevibacillus having deposit accession number B-67819, or a strain having all of the identifying characteristics of Brevibacillus B-67819, or a mutant thereof; (b) a Streptomyces having deposit accession number B-67820, or a strain having all of the identifying characteristics of Streptomyces B-67820, or a mutant thereof and (c) a Streptomyces having deposit accession number B-67821, or a strain having all of the identifying characteristics of Streptomyces B-67821, or a mutant thereof.

The disclosure further provides plant pathogen inhibiting microbial consortium LLK3-2017, comprising a microbial consortia having deposit accession number PTA-124320, or a consortia of strains having all of the identifying characteristics of LLK3-2017, or mutants thereof.

In some embodiments, the plant pathogen inhibiting microbial consortium comprises two microbes. In some embodiments, the plant pathogen inhibiting microbial consortium comprises two microbial species—a first microbial species and a second microbial species. In some embodiments, the first microbial species provides pathogen suppression. In some embodiments, the second microbial species has the ability to signal and modulate the production of antimicrobial compounds in the first microbial species.

In some embodiments, the plant pathogen inhibiting microbial consortium comprises three microbes. In some embodiments, the plant pathogen inhibiting microbial consortium comprises three microbial species—a first microbial species, a second microbial species and a third microbial species. In some embodiments, the first microbial species provides pathogen suppression. In some embodiments, the second microbial species has the ability to signal and modulate the production of antimicrobial compounds in the either or both of the other microbial species. In some embodiments, the third microbial isolate provides the plant growth-promoting ability.

The microbes disclosed herein may be matched to their nearest taxonomic groups by utilizing classification tools of the Ribosomal Database Project (RDP) for 16s rRNA sequences and the User-friendly Nordic ITS Ectomycorrhiza (UNITE) database for ITS rRNA sequences. Examples of matching microbes to their nearest taxa may be found in Lan et al. (2012. PLOS one. 7(3):e32491), Schloss and Westcott (2011. Appl. Environ. Microbiol. 77(10):3219-3226), and Koljalg et al. (2005. New Phytologist. 166(3):1063-1068).

The isolation, identification, and culturing of the microbes of the present disclosure can be effected using standard microbiological techniques. Examples of such techniques may be found in Gerhardt, P. (ed.) Methods for General and Molecular Microbiology. American Society for Microbiology, Washington, D.C. (1994) and Lennette, E. H. (ed.) Manual of Clinical Microbiology, Third Edition. American Society for Microbiology, Washington, D.C. (1980), each of which is incorporated by reference.

Isolation can be effected by streaking the specimen on a solid medium (e.g., nutrient agar plates) to obtain a single colony, which is characterized by the phenotypic traits described herein (e.g., Gram positive/negative, capable of forming spores aerobically/anaerobically, cellular morphology, carbon source metabolism, acid/base production, enzyme secretion, metabolic secretions, etc.) and to reduce the likelihood of working with a culture which has become contaminated.

For example, for microbes of the disclosure, biologically pure isolates can be obtained through repeated subculture of biological samples, each subculture followed by streaking onto solid media to obtain individual colonies or colony forming units. Methods of preparing, thawing, and growing lyophilized bacteria are commonly known, for example, Gherna, R. L. and C. A. Reddy. 2007. Culture Preservation, p 1019-1033. In C. A. Reddy, T. J. Beveridge, J. A. Breznak, G. A. Marzluf, T. M. Schmidt, and L. R. Snyder, eds. American Society for Microbiology, Washington, D.C., 1033 pages; herein incorporated by reference. Thus freeze dried liquid formulations and cultures stored long term at −70° C. in solutions containing glycerol are contemplated for use in providing formulations of the present disclosure.

The microbes of the present disclosure can be propagated in a liquid or solid medium under aerobic conditions, or alternatively anaerobic conditions. Medium for growing the bacterial strains of the present disclosure may include a carbon source, a nitrogen source, and inorganic salts, as well as specially required substances such as vitamins, amino acids, nucleic acids and the like. In some embodiments, the media comprises water and agar. Examples of suitable carbon sources which can be used for growing the microbes include, but are not limited to, starch, peptone, yeast extract, amino acids, sugars such as glucose, arabinose, mannose, glucosamine, maltose, and the like; salts of organic acids such as acetic acid, fumaric acid, adipic acid, propionic acid, citric acid, gluconic acid, malic acid, pyruvic acid, malonic acid and the like; alcohols such as ethanol and glycerol and the like; oil or fat such as soybean oil, rice bran oil, olive oil, corn oil, sesame oil. The amount of the carbon source added varies according to the kind of carbon source and is typically between 1 to 100 gram(s) per liter of medium. Preferably, glucose, starch, and/or peptone is contained in the medium as a major carbon source, at a concentration of 0.1-5% (W/V). Examples of suitable nitrogen sources which can be used for growing the bacterial strains of the present disclosure include, but are not limited to, amino acids, yeast extract, tryptone, beef extract, peptone, potassium nitrate, ammonium nitrate, ammonium chloride, ammonium sulfate, ammonium phosphate, ammonia or combinations thereof. The amount of nitrogen source varies according to the type of nitrogen source, typically between 0.1 to 30 gram(s) per liter of medium. The inorganic salts, potassium dihydrogen phosphate, dipotassium hydrogen phosphate, disodium hydrogen phosphate, magnesium sulfate, magnesium chloride, ferric sulfate, ferrous sulfate, ferric chloride, ferrous chloride, manganous sulfate, manganous chloride, zinc sulfate, zinc chloride, cupric sulfate, calcium chloride, sodium chloride, calcium carbonate, sodium carbonate can be used alone or in combination. The amount of inorganic acid varies according to the kind of the inorganic salt, typically between 0.001 to 10 gram(s) per liter of medium. Examples of specially required substances include, but are not limited to, vitamins, nucleic acids, yeast extract, peptone, meat extract, malt extract, dried yeast and combinations thereof. Cultivation can be effected at a temperature, which allows the growth of the microbial strains, essentially, between 20° C. and 46° C. In some embodiments, a temperature range is 30° C.-39° C. For optimal growth, in some embodiments, the medium can be adjusted to pH 6.0-7.4. It will be appreciated that commercially available media may also be used to culture the microbial strains, such as Nutrient Broth or Nutrient Agar available from Difco, Detroit, Mich. It will be appreciated that cultivation time may differ depending on the type of culture medium used and the concentration of sugar as a major carbon source.

In some embodiments, cultivation lasts between about 24 to about 96 hours. In some embodiments, cultivation lasts longer than 96 hours, such as, for example, about 4 days, about 5 days, about 1 week, about 2 weeks, about 3 weeks, about 4 weeks, about 6 weeks, or about 2 months. Microbial cells thus obtained are isolated using methods, which are well known in the art. Examples include, but are not limited to, membrane filtration and centrifugal separation. The pH may be adjusted using sodium hydroxide and the like and the culture may be dried using a freeze dryer, until the water content becomes equal to 4% or less. Microbial co-cultures may be obtained by propagating each strain as described hereinabove. In some embodiments, microbial multi-strain cultures may be obtained by propagating two or more of the strains described hereinabove. It will be appreciated that the microbial strains may be cultured together when compatible culture conditions can be employed.

### Microbial Compositions

The disclosure provides microbial compositions comprising any one or more of the microbes and/or microbial consortia disclosed herein. In some embodiments, the microbial compositions may further comprise suitable carrier and other additives. In some embodiments, the microbial compositions of the present disclosure are solid. Where solid compositions are used, it may be desired to include one or more carrier materials including, but not limited to: mineral earths such as silicas, talc, kaolin, limestone, chalk, clay, dolomite, diatomaceous earth; calcium sulfate; magnesium sulfate; magnesium oxide; zeolites, calcium carbonate; magnesium carbonate; trehalose; chitosan; shellac; and starch.

In some embodiments, the microbial compositions of the present disclosure are liquid. In further embodiments, the liquid comprises a solvent that may include water or an alcohol or a saline or carbohydrate solution, and other plant-safe solvents. In some embodiments, the microbial compositions of the present disclosure include binders such as plant-safe polymers, carboxymethylcellulose, starch, polyvinyl alcohol, and the like.

In some embodiments, the microbial compositions of the present disclosure comprise thickening agents such as silica, clay, natural extracts of seeds or seaweed, synthetic derivatives of cellulose, guar gum, locust bean gum, alginates, and methylcelluloses. In some embodiments, the microbial compositions comprise anti-settling agents such as modified starches, polyvinyl alcohol, xanthan gum, and the like.

In some embodiments, the microbial compositions of the present disclosure comprise colorants including organic chromophores classified as nitroso; nitro; azo, including monoazo, bisazo and polyazo; acridine, anthraquinone, azine, diphenylmethane, indamine, indophenol, methine, oxazine, phthalocyanine, thiazine, thiazole, triarylmethane, xanthene. In some embodiments, the microbial compositions of the present disclosure comprise trace nutrients such as salts of iron, manganese, boron, copper, cobalt, molybdenum and zinc. In some embodiments, the microbial compositions comprise dyes, both natural and artificial.

In some embodiments, the microbial compositions of the present disclosure may include combinations of fungal spores and bacterial spores, fungal spores and bacterial vegetative cells, fungal vegetative cells and bacterial spores, fungal vegetative cells and bacterial vegetative cells. In some embodiments, compositions of the present disclosure comprise bacteria only in the form of spores. In some embodiments, compositions of the present disclosure comprise bacteria only in the form of vegetative cells. In some embodiments, compositions of the present disclosure comprise bacteria in the absence of fungi. In some embodiments, compositions of the present disclosure comprise fungi in the absence of bacteria. In some embodiments, compositions of the present disclosure comprise viable but non-culturable (VBNC) bacteria and/or fungi. In some embodiments, compositions of the present disclosure comprise bacteria and/or fungi in a quiescent state. In some embodiments, compositions of the present disclosure include dormant bacteria and/or fungi. Bacterial spores may include endospores and akinetes. Fungal spores may include statismospores, ballistospores, autospores, aplanospores, zoospores, mitospores, megaspores, microspores, meiospores, chlamydospores, urediniospores, teliospores, oospores, carpospores, tetraspores, sporangiospores, zygospores, ascospores, basidiospores, ascospores, and asciospores.

In some embodiments, the microbial compositions of the present disclosure comprise a plant-safe virucide, parasiticide, bacteriocide, fungicide, or nematicide. In some embodiments, microbial compositions of the present disclosure comprise one or more oxygen scavengers, denitrifies, nitrifiers, heavy metal chelators, and/or dechlorinators; and combinations thereof.

In some embodiments, microbial compositions of the present disclosure comprise one or more preservatives. The preservatives may be in liquid or gas formulations. The preservatives may be selected from one or more of monosaccharide, disaccharide, trisaccharide, polysaccharide, acetic acid, ascorbic acid, calcium ascorbate, erythorbic acid, iso-ascorbic acid, erythrobic acid, potassium nitrate, sodium ascorbate, sodium erythorbate, sodium iso-ascorbate, sodium nitrate, sodium nitrite, nitrogen, benzoic acid, calcium sorbate, ethyl lauroyl arginate, methyl-p-hydroxy benzoate, methyl paraben, potassium acetate, potassium benzoiate, potassium bisulphite, potassium diacetate, potassium lactate, potassium metabisulphite, potassium sorbate, propyl-p-hydroxy benzoate, propyl paraben, sodium acetate, sodium benzoate, sodium bisulphite, sodium nitrite, sodium diacetate, sodium lactate, sodium metabisulphite, sodium salt of methyl-p-hydroxy benzoic acid, sodium salt of propyl-p-hydroxy benzoic acid, sodium sulphate, sodium sulfite, sodium dithionite, sulphurous acid, calcium propionate, dimethyl dicarbonate, natamycin, potassium sorbate, potassium bisulfite, potassium metabisulfite, propionic acid, sodium diacetate, sodium propionate, sodium sorbate, sorbic acid, ascorbic acid, ascorbyl palmitate, ascorbyl stearate, butylated hydro-xyanisole, butylated hydroxytoluene (BHT), butylated hydroxyl anisole (BHA), citric acid, citric acid esters of mono- and/or diglycerides, L-cysteine, L-cysteine hydrochloride, gum guaiacum, gum guaiac, lecithin, lecithin citrate, monoglyceride citrate, monoisopropyl citrate, propyl gallate, sodium metabisulphite, tartaric acid, tertiary butyl hydroquinone, stannous chloride, thiodipropionic acid, dilauryl thiodipropionate, distearyl thiodipropionate, ethoxyquin, sulfur dioxide, formic acid, or tocopherol(s).

In some embodiments, the microbial compositions are shelf stable in a refrigerator (35-40° F.) for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable in a refrigerator (35-40° F.) for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks. In some embodiments, the microbial compositions are shelf stable in a refrigerator (35-40° F.) for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 years.

In some embodiments, the microbial compositions are shelf stable at room temperature (68-72° F.) or between 50-77° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable at room temperature (68-72° F.) or between 50-77° F. fora period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks. In some embodiments, the microbial compositions are shelf stable at room temperature (68-72° F.) or between 50-77° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 years.

In some embodiments, the microbial compositions are shelf stable at -23-35° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable at −23-35° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks. In some embodiments, the microbial compositions are shelf stable at −23-35° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 years.

In some embodiments, the microbial compositions are shelf stable at 77-100° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable at 77-100° F. fora period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks. In some embodiments, the microbial compositions are shelf stable at 77-100° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 years.

In some embodiments, the microbial compositions are shelf stable at 101-213° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable at 101-213° F. fora period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks. In some embodiments, the microbial compositions are shelf stable at 101-213° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 years.

In some embodiments, the microbial compositions of the present disclosure are shelf stable at refrigeration temperatures (35-40° F.), at room temperature (68-72° F.), between 50-77° F., between -23-35° F., between 70-100° F., or between 101-213° F. for a period of about 1 to 100, about 1 to 95, about 1 to 90, about 1 to 85, about 1 to 80, about 1 to 75, about 1 to 70, about 1 to 65, about 1 to 60, about 1 to 55, about 1 to 50, about 1 to 45, about 1 to 40, about 1 to 35, about 1 to 30, about 1 to 25, about 1 to 20, about 1 to 15, about 1 to 10, about 1 to 5, about 5 to 100, about 5 to 95, about 5 to 90, about 5 to 85, about 5 to 80, about 5 to 75, about 5 to 70, about 5 to 65, about 5 to 60, about 5 to 55, about 5 to 50, about 5 to 45, about 5 to 40, about 5 to 35, about 5 to 30, about 5 to 25, about 5 to 20, about 5 to 15, about 5 to 10, about 10 to 100, about 10 to 95, about 10 to 90, about 10 to 85, about 10 to 80, about 10 to 75, about 10 to 70, about 10 to 65, about 10 to 60, about 10 to 55, about 10 to 50, about 10 to 45, about 10 to 40, about 10 to 35, about 10 to 30, about 10 to 25, about 10 to 20, about 10 to 15, about 15 to 100, about 15 to 95, about 15 to 90, about 15 to 85, about 15 to 80, about 15 to 75, about 15 to 70, about 15 to 65, about 15 to 60, about 15 to 55, about 15 to 50, about 15 to 45, about 15 to 40, about 15 to 35, about 15 to 30, about 15 to 25, about 15 to 20, about 20 to 100, about 20 to 95, about 20 to 90, about 20 to 85, about 20 to 80, about 20 to 75, about 20 to 70, about 20 to 65, about 20 to 60, about 20 to 55, about 20 to 50, about 20 to 45, about 20 to 40, about 20 to 35, about 20 to 30, about 20 to 25, about 25 to 100, about 25 to 95, about 25 to 90, about 25 to 85, about 25 to 80, about 25 to 75, about 25 to 70, about 25 to 65, about 25 to 60, about 25 to 55, about 25 to 50, about 25 to 45, about 25 to 40, about 25 to 35, about 25 to 30, about 30 to 100, about 30 to 95, about 30 to 90, about 30 to 85, about 30 to 80, about 30 to 75, about 30 to 70, about 30 to 65, about 30 to 60, about 30 to 55, about 30 to 50, about 30 to 45, about 30 to 40, about 30 to 35, about 35 to 100, about 35 to 95, about 35 to 90, about 35 to 85, about 35 to 80, about 35 to 75, about 35 to 70, about 35 to 65, about 35 to 60, about 35 to 55, about 35 to 50, about 35 to 45, about 35 to 40, about 40 to 100, about 40 to 95, about 40 to 90, about 40 to 85, about 40 to 80, about 40 to 75, about 40 to 70, about 40 to 65, about 40 to 60, about 40 to 55, about 40 to 50, about 40 to 45, about 45 to 100, about 45 to 95, about 45 to 90, about 45 to 85, about 45 to 80, about 45 to 75, about 45 to 70, about 45 to 65, about 45 to 60, about 45 to 55, about 45 to 50, about 50 to 100, about 50 to 95, about 50 to 90, about 50 to 85, about 50 to 80, about 50 to 75, about 50 to 70, about 50 to 65, about 50 to 60, about 50 to 55, about 55 to 100, about 55 to 95, about 55 to 90, about 55 to 85, about 55 to 80, about 55 to 75, about 55 to 70, about 55 to 65, about 55 to 60, about 60 to 100, about 60 to 95, about 60 to 90, about 60 to 85, about 60 to 80, about 60 to 75, about 60 to 70, about 60 to 65, about 65 to 100, about 65 to 95, about 65 to 90, about 65 to 85, about 65 to 80, about 65 to 75, about 65 to 70, about 70 to 100, about 70 to 95, about 70 to 90, about 70 to 85, about 70 to 80, about 70 to 75, about 75 to 100, about 75 to 95, about 75 to 90, about 75 to 85, about 75 to 80, about 80 to 100, about 80 to 95, about 80 to 90, about 80 to 85, about 85 to 100, about 85 to 95, about 85 to 90, about 90 to 100, about 90 to 95, or 95 to 100 weeks.

In some embodiments, the microbial compositions of the present disclosure are shelf stable at refrigeration temperatures (35-40° F.), at room temperature (68-72° F.), between 50-77° F., between -23-35° F., between 70-100° F., or between 101-213° F. for a period of 1 to 100, 1 to 95, 1 to 90, 1 to 85, 1 to 80, 1 to 75, 1 to 70, 1 to 65, 1 to 60, 1 to 55, 1 to 50, 1 to 45, 1 to 40, 1 to 35, 1 to 30, 1 to 25, 1 to 20, 1 to 15, 1 to 10, 1 to 5, 5 to 100, 5 to 95, 5 to 90, 5 to 85, 5 to 80, 5 to 75, 5 to 70, 5 to 65, 5 to 60, 5 to 55, 5 to 50, 5 to 45, 5 to 40, 5 to 35, 5 to 30, 5 to 25, 5 to 20, 5 to 15, 5 to 10, 10 to 100, 10 to 95, 10 to 90, 10 to 85, 10 to 80, 10 to 75, 10 to 70, 10 to 65, 10 to 60, 10 to 55, 10 to 50, 10 to 45, 10 to 40, 10 to 35, 10 to 30, 10 to 25, 10 to 20, 10 to 15, 15 to 100, 15 to 95, 15 to 90, 15 to 85, 15 to 80, 15 to 75, 15 to 70, 15 to 65, 15 to 60, 15 to 55, 15 to 50, 15 to 45, 15 to 40, 15 to 35, 15 to 30, 15 to 25, 15 to 20, 20 to 100, 20 to 95, 20 to 90, 20 to 85, 20 to 80, 20 to 75, 20 to 70, 20 to 65, 20 to 60, 20 to 55, 20 to 50, 20 to 45, 20 to 40, 20 to 35, 20 to 30, 20 to 25, 25 to 100, 25 to 95, 25 to 90, 25 to 85, 25 to 80, 25 to 75, 25 to 70, 25 to 65, 25 to 60, 25 to 55, 25 to 50, 25 to 45, 25 to 40, 25 to 35, 25 to 30, 30 to 100, 30 to 95, 30 to 90, 30 to 85, 30 to 80, 30 to 75, 30 to 70, 30 to 65, 30 to 60, 30 to 55, 30 to 50, 30 to 45, 30 to 40, 30 to 35, 35 to 100, 35 to 95, 35 to 90, 35 to 85, 35 to 80, 35 to 75, 35 to 70, 35 to 65, 35 to 60, 35 to 55, 35 to 50, 35 to 45, 35 to 40, 40 to 100, 40 to 95, 40 to 90, 40 to 85, 40 to 80, 40 to 75, 40 to 70, 40 to 65, 40 to 60, 40 to 55, 40 to 50, 40 to 45, 45 to 100, 45 to 95, 45 to 90, 45 to 85, 45 to 80, 45 to 75, 45 to 70, 45 to 65, 45 to 60, 45 to 55, 45 to 50, 50 to 100, 50 to 95, 50 to 90, 50 to 85, 50 to 80, 50 to 75, 50 to 70, 50 to 65, 50 to 60, 50 to 55, 55 to 100, 55 to 95, 55 to 90, 55 to 85, 55 to 80, 55 to 75, 55 to 70, 55 to 65, 55 to 60, 60 to 100, 60 to 95, 60 to 90, 60 to 85, 60 to 80, 60 to 75, 60 to 70, 60 to 65, 65 to 100, 65 to 95, 65 to 90, 65 to 85, 65 to 80, 65 to 75, 65 to 70, 70 to 100, 70 to 95, 70 to 90, 70 to 85, 70 to 80, 70 to 75, 75 to 100, 75 to 95, 75 to 90, 75 to 85, 75 to 80, 80 to 100, 80 to 95, 80 to 90, 80 to 85, 85 to 100, 85 to 95, 85 to 90, 90 to 100, 90 to 95, or 95 to 100 weeks.

In some embodiments, the microbial compositions of the present disclosure are shelf stable at refrigeration temperatures (35-40° F.), at room temperature (68-72° F.), between 50-77° F., between -23-35° F., between 70-100° F., or between 101-213° F. for a period of about 1 to 36, about 1 to 34, about 1 to 32, about 1 to 30, about 1 to 28, about 1 to 26, about 1 to 24, about 1 to 22, about 1 to 20, about 1 to 18, about 1 to 16, about 1 to 14, about 1 to 12, about 1 to 10, about 1 to 8, about 1 to 6, about 1 one 4, about 1 to 2, about 4 to 36, about 4 to 34, about 4 to 32, about 4 to 30, about 4 to 28, about 4 to 26, about 4 to 24, about 4 to 22, about 4 to 20, about 4 to 18, about 4 to 16, about 4 to 14, about 4 to 12, about 4 to 10, about 4 to 8, about 4 to 6, about 6 to 36, about 6 to 34, about 6 to 32, about 6 to 30, about 6 to 28, about 6 to 26, about 6 to 24, about 6 to 22, about 6 to 20, about 6 to 18, about 6 to 16, about 6 to 14, about 6 to 12, about 6 to 10, about 6 to 8, about 8 to 36, about 8 to 34, about 8 to 32, about 8 to 30, about 8 to 28, about 8 to 26, about 8 to 24, about 8 to 22, about 8 to 20, about 8 to 18, about 8 to 16, about 8 to 14, about 8 to 12, about 8 to 10, about 10 to 36, about 10 to 34, about 10 to 32, about 10 to 30, about 10 to 28, about 10 to 26, about 10 to 24, about 10 to 22, about 10 to 20, about 10 to 18, about 10 to 16, about 10 to 14, about 10 to 12, about 12 to 36, about 12 to 34, about 12 to 32, about 12 to 30, about 12 to 28, about 12 to 26, about 12 to 24, about 12 to 22, about 12 to 20, about 12 to 18, about 12 to 16, about 12 to 14, about 14 to 36, about 14 to 34, about 14 to 32, about 14 to 30, about 14 to 28, about 14 to 26, about 14 to 24, about 14 to 22, about 14 to 20, about 14 to 18, about 14 to 16, about 16 to 36, about 16 to 34, about 16 to 32, about 16 to 30, about 16 to 28, about 16 to 26, about 16 to 24, about 16 to 22, about 16 to 20, about 16 to 18, about 18 to 36, about 18 to 34, about 18 to 32, about 18 to 30, about 18 to 28, about 18 to 26, about 18 to 24, about 18 to 22, about 18 to 20, about 20 to 36, about 20 to 34, about 20 to 32, about 20 to 30, about 20 to 28, about 20 to 26, about 20 to 24, about 20 to 22, about 22 to 36, about 22 to 34, about 22 to 32, about 22 to 30, about 22 to 28, about 22 to 26, about 22 to 24, about 24 to 36, about 24 to 34, about 24 to 32, about 24 to 30, about 24 to 28, about 24 to 26, about 26 to 36, about 26 to 34, about 26 to 32, about 26 to 30, about 26 to 28, about 28 to 36, about 28 to 34, about 28 to 32, about 28 to 30, about 30 to 36, about 30 to 34, about 30 to 32, about 32 to 36, about 32 to 34, or about 34 to 36 months.

In some embodiments, the microbial compositions of the present disclosure are shelf stable at refrigeration temperatures (35-40° F.), at room temperature (68-72° F.), between 50-77° F., between −23-35° F., between 70-100° F., or between 101-213° F. for a period of 1 to 36, 1 to 34, 1 to 32, 1 to 30, 1 to 28, 1 to 26, 1 to 24, 1 to 22, 1 to 20, 1 to 18, 1 to 16, 1 to 14, 1 to 12, 1 to 10, 1 to 8, 1 to 6, 1 to 4,1 to 2, 4 to 36, 4 to 34, 4 to 32, 4 to 30, 4 to 28, 4 to 26, 4 to 24, 4 to 22, 4 to 20, 4 to 18, 4 to 16, 4 to 14, 4 to 12, 4 to 10, 4 to 8, 4 to 6, 6 to 36, 6 to 34, 6 to 32, 6 to 30, 6 to 28, 6 to 26, 6 to 24, 6 to 22, 6 to 20, 6 to 18, 6 to 16, 6 to 14, 6 to 12, 6 to 10, 6 to 8, 8 to 36, 8 to 34, 8 to 32, 8 to 30, 8 to 28, 8 to 26, 8 to 24, 8 to 22, 8 to 20, 8 to 18, 8 to 16, 8 to 14, 8 to 12, 8 to 10, 10 to 36, 10 to 34, 10 to 32, 10 to 30, 10 to 28, 10 to 26, 10 to 24, 10 to 22, 10 to 20, 10 to 18, 10 to 16, 10 to 14, 10 to 12, 12 to 36, 12 to 34, 12 to 32, 12 to 30, 12 to 28, 12 to 26, 12 to 24, 12 to 22, 12 to 20, 12 to 18, 12 to 16, 12 to 14, 14 to 36, 14 to 34, 14 to 32, 14 to 30, 14 to 28, 14 to 26, 14 to 24, 14 to 22, 14 to 20, 14 to 18, 14 to 16, 16 to 36, 16 to 34, 16 to 32, 16 to 30, 16 to 28, 16 to 26, 16 to 24, 16 to 22, 16 to 20, 16 to 18, 18 to 36, 18 to 34, 18 to 32, 18 to 30, 18 to 28, 18 to 26, 18 to 24, 18 to 22, 18 to 20, 20 to 36, 20 to 34, 20 to 32, 20 to 30, 20 to 28, 20 to 26, 20 to 24, 20 to 22, 22 to 36, 22 to 34, 22 to 32, 22 to 30, 22 to 28, 22 to 26, 22 to 24, 24 to 36, 24 to 34, 24 to 32, 24 to 30, 24 to 28, 24 to 26, 26 to 36, 26 to 34, 26 to 32, 26 to 30, 26 to 28, 28 to 36, 28 to 34, 28 to 32, 28 to 30, 30 to 36, 30 to 34, 30 to 32, 32 to 36, 32 to 34, or 34 to 36 months.

In some embodiments, the microbial compositions of the present disclosure are shelf stable at refrigeration temperatures (35-40° F.), at room temperature (68-72° F.), between 50-77° F., between −23-35° F., between 70-100° F., or between 101-213° F. for a period of about 1 to 36, about 1 to 34, about 1 to 32, about 1 to 30, about 1 to 28, about 1 to 26, about 1 to 24, about 1 to 22, about 1 to 20, about 1 to 18, about 1 to 16, about 1 to 14, about 1 to 12, about 1 to 10, about 1 to 8, about 1 to 6, about 1 one 4, about 1 to 2, about 4 to 36, about 4 to 34, about 4 to 32, about 4 to 30, about 4 to 28, about 4 to 26, about 4 to 24, about 4 to 22, about 4 to 20, about 4 to 18, about 4 to 16, about 4 to 14, about 4 to 12, about 4 to 10, about 4 to 8, about 4 to 6, about 6 to 36, about 6 to 34, about 6 to 32, about 6 to 30, about 6 to 28, about 6 to 26, about 6 to 24, about 6 to 22, about 6 to 20, about 6 to 18, about 6 to 16, about 6 to 14, about 6 to 12, about 6 to 10, about 6 to 8, about 8 to 36, about 8 to 34, about 8 to 32, about 8 to 30, about 8 to 28, about 8 to 26, about 8 to 24, about 8 to 22, about 8 to 20, about 8 to 18, about 8 to 16, about 8 to 14, about 8 to 12, about 8 to 10, about 10 to 36, about 10 to 34, about 10 to 32, about 10 to 30, about 10 to 28, about 10 to 26, about 10 to 24, about 10 to 22, about 10 to 20, about 10 to 18, about 10 to 16, about 10 to 14, about 10 to 12, about 12 to 36, about 12 to 34, about 12 to 32, about 12 to 30, about 12 to 28, about 12 to 26, about 12 to 24, about 12 to 22, about 12 to 20, about 12 to 18, about 12 to 16, about 12 to 14, about 14 to 36, about 14 to 34, about 14 to 32, about 14 to 30, about 14 to 28, about 14 to 26, about 14 to 24, about 14 to 22, about 14 to 20, about 14 to 18, about 14 to 16, about 16 to 36, about 16 to 34, about 16 to 32, about 16 to 30, about 16 to 28, about 16 to 26, about 16 to 24, about 16 to 22, about 16 to 20, about 16 to 18, about 18 to 36, about 18 to 34, about 18 to 32, about 18 to 30, about 18 to 28, about 18 to 26, about 18 to 24, about 18 to 22, about 18 to 20, about 20 to 36, about 20 to 34, about 20 to 32, about 20 to 30, about 20 to 28, about 20 to 26, about 20 to 24, about 20 to 22, about 22 to 36, about 22 to 34, about 22 to 32, about 22 to 30, about 22 to 28, about 22 to 26, about 22 to 24, about 24 to 36, about 24 to 34, about 24 to 32, about 24 to 30, about 24 to 28, about 24 to 26, about 26 to 36, about 26 to 34, about 26 to 32, about 26 to 30, about 26 to 28, about 28 to 36, about 28 to 34, about 28 to 32, about 28 to 30, about 30 to 36, about 30 to 34, about 30 to 32, about 32 to 36, about 32 to 34, or about 34 to 36 years.

In some embodiments, the microbial compositions of the present disclosure are shelf stable at refrigeration temperatures (35-40° F.), at room temperature (68-72° F.), between 50-77° F., between −23-35° F., between 70-100° F., or between 101-213° F. for a period of 1 to 36, 1 to 34, 1 to 32, 1 to 30, 1 to 28, 1 to 26, 1 to 24, 1 to 22, 1 to 20, 1 to 18, 1 to 16, 1 to 14, 1 to 12, 1 to 10, 1 to 8, 1 to 6, 1 to 4, 1 to 2, 4 to 36, 4 to 34, 4 to 32, 4 to 30, 4 to 28, 4 to 26, 4 to 24, 4 to 22, 4 to 20, 4 to 18, 4 to 16, 4 to 14, 4 to 12, 4 to 10, 4 to 8, 4 to 6, 6 to 36, 6 to 34, 6 to 32, 6 to 30, 6 to 28, 6 to 26, 6 to 24, 6 to 22, 6 to 20, 6 to 18, 6 to 16, 6 to 14, 6 to 12, 6 to 10, 6 to 8, 8 to 36, 8 to 34, 8 to 32, 8 to 30, 8 to 28, 8 to 26, 8 to 24, 8 to 22, 8 to 20, 8 to 18, 8 to 16, 8 to 14, 8 to 12, 8 to 10, 10 to 36, 10 to 34, 10 to 32, 10 to 30, 10 to 28, 10 to 26, 10 to 24, 10 to 22, 10 to 20, 10 to 18, 10 to 16, 10 to 14, 10 to 12, 12 to 36, 12 to 34, 12 to 32, 12 to 30, 12 to 28, 12 to 26, 12 to 24, 12 to 22, 12 to 20, 12 to 18, 12 to 16, 12 to 14, 14 to 36, 14 to 34, 14 to 32, 14 to 30, 14 to 28, 14 to 26, 14 to 24, 14 to 22, 14 to 20, 14 to 18, 14 to 16, 16 to 36, 16 to 34, 16 to 32, 16 to 30, 16 to 28, 16 to 26, 16 to 24, 16 to 22, 16 to 20, 16 to 18, 18 to 36, 18 to 34, 18 to 32, 18 to 30, 18 to 28, 18 to 26, 18 to 24, 18 to 22, 18 to 20, 20 to 36, 20 to 34, 20 to 32, 20 to 30, 20 to 28, 20 to 26, 20 to 24, 20 to 22, 22 to 36, 22 to 34, 22 to 32, 22 to 30, 22 to 28, 22 to 26, 22 to 24, 24 to 36, 24 to 34, 24 to 32, 24 to 30, 24 to 28, 24 to 26, 26 to 36, 26 to 34, 26 to 32, 26 to 30, 26 to 28, 28 to 36, 28 to 34, 28 to 32, 28 to 30, 30 to 36, 30 to 34, 30 to 32, 32 to 36, 32 to 34, or 34 to 36 years.

In some embodiments, the microbial compositions of the present disclosure are shelf stable at any of the disclosed temperatures and/or temperature ranges and spans of time at a relative humidity of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, or 98%.

In some embodiments, the microbial composition of the present disclosure possesses a water activity (aw) of less than 0.750, 0.700, 0.650, 0.600, 0.550, 0.500, 0.475, 0.450, 0.425, 0.400, 0.375, 0.350, 0.325, 0.300, 0.275, 0.250, 0.225, 0.200, 0.190, 0.180, 0.170, 0.160, 0.150, 0.140, 0.130, 0.120, 0.110, 0.100, 0.095, 0.090, 0.085, 0.080, 0.075, 0.070, 0.065, 0.060, 0.055, 0.050, 0.045, 0.040, 0.035, 0.030, 0.025, 0.020, 0.015, 0.010, or 0.005.

In some embodiments, the microbial composition of the present disclosure possesses a water activity (a_{w}) of less than about 0.750, about 0.700, about 0.650, about 0.600, about 0.550, about 0.500, about 0.475, about 0.450, about 0.425, about 0.400, about 0.375, about 0.350, about 0.325, about 0.300, about 0.275, about 0.250, about 0.225, about 0.200, about 0.190, about 0.180, about 0.170, about 0.160, about 0.150, about 0.140, about 0.130, about 0.120, about 0.110, about 0.100, about 0.095, about 0.090, about 0.085, about 0.080, about 0.075, about 0.070, about 0.065, about 0.060, about 0.055, about 0.050, about 0.045, about 0.040, about 0.035, about 0.030, about 0.025, about 0.020, about 0.015, about 0.010, or about 0.005.

The water activity values are determined by the method of Saturated Aqueous Solutions (Multon, “Techniques d'Analyse E De Controle Dans Les Industries Agroalimentaires” APRIA (1981)) or by direct measurement using a viable Robotronic BT hygrometer or other hygrometer or hygroscope.

In some embodiments, the microbial composition comprises at least two different microbes, and wherein the at least two microbes are present in the composition at a ratio of 1:2, 1:3, 1:3, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:40, 1:50, 1:60, 1:100, 1:125, 1:150, 1:175, or 1:200 or the inverse thereof. In some embodiments, the microbial composition comprises at least three different microbes, and wherein the three microbes are present in the composition at a ratio of 1:2:1, 1:1:2, 2:2:1, 1:3:1, 1:1:3, 3:1:1, 3:3:1, 1:5:1, 1:1:5, 5:1:1, 5:5:1, or 1:5:5.

### Encapsulating Compositions

In some embodiments, any one of the microbes, microbial consortia or microbial compositions of the disclosure are encapsulated in an encapsulating composition. An encapsulating composition protects the microbes from external stressors. In some embodiments, external stressors include thermal and physical stressors. In some embodiments, external stressors include chemicals present in the compositions. Encapsulating compositions further create an environment that may be beneficial to the microbes, such as minimizing the oxidative stresses of an aerobic environment on anaerobic microbes. See Kalsta et al. (U.S. Pat. No. 5,104,662A), Ford (U.S. Pat. No. 5,733,568A), and Mosbach and Nilsson (U.S. Pat. No. 4,647,536A) for encapsulation compositions of microbes, and methods of encapsulating microbes.

In one embodiment, any one of the microbes, microbial consortia or microbial compositions of the present disclosure exhibits a thermal tolerance, which is used interchangeably with heat tolerance and heat resistance. In one embodiment, thermal tolerant compositions of the present disclosure are resistant to heat-killing and denaturation of the cell wall components and the intracellular environment.

In one embodiment, any one of the microbes, microbial consortia or microbial compositions of the present disclosure exhibits a pH tolerance, which is used interchangeably with acid tolerance and base tolerance. In one embodiment, pH tolerant compositions of the present disclosure are tolerant of the rapid swings in pH (high to low, low to high, high to neutral, low to neutral, neutral to high, and neutral to low) associated with one or more steps of preparing the composition.

In one embodiment, the encapsulation is a reservoir-type encapsulation. In one embodiment, the encapsulation is a matrix-type encapsulation. In one embodiment, the encapsulation is a coated matrix-type encapsulation. Burgain et al. (2011. J. Food Eng. 104:467-483) discloses numerous encapsulation embodiments and techniques.

In some embodiments, the microbes, microbial consortia or microbial compositions of the present disclosure are encapsulated in one or more of the following: gellan gum, xanthan gum, K-Carrageenan, cellulose acetate phthalate, chitosan, starch, milk fat, whey protein, Ca-alginate, raftilose, raftiline, pectin, saccharide, glucose, maltodextrin, gum arabic, guar, seed flour, alginate, dextrins, dextrans, celluloase, gelatin, gelatin, albumin, casein, gluten, acacia gum, tragacanth, wax, paraffin, stearic acid, monodiglycerides, and diglycerides. In some embodiments, the compositions of the present disclosure are encapsulated by one or more of a polymer, carbohydrate, sugar, plastic, glass, polysaccharide, lipid, wax, oil, fatty acid, or glyceride. In one embodiment, the microbial composition is encapsulated by glucose. In one embodiment, the microbial composition is encapsulated by a glucose-containing composition. In one embodiment, formulations of the microbial composition comprise a glucose encapsulant. In one embodiment, formulations of the microbial composition comprise a glucose-encapsulated composition.

In some embodiments, the encapsulation of the microbes, microbial consortia or microbial compositions of the present disclosure is carried out by an extrusion, emulsification, coating, agglomeration, lyophilization, vitrification, foam drying, preservation by vaporization, vacuum-drying, or spray-drying.

In some embodiments, the encapsulated compositions of the present disclosure are vitrified. In some embodiments, encapsulation involves a process of drying a composition of the present disclosure in the presence of a substance which forms a glassy, amorphous solid state, a process known as vitrification, and in doing so encapsulates the composition. In some embodiments, the vitrified composition is protected from degradative conditions that would typically destroy or degrade microbes. Many common substances have the property of vitrification; that is, they will form a glassy solid state under certain conditions. Among these substances are several sugars, including sucrose and maltose, and other more complex compounds, such as polyvinylpyrrolidone (PVP). As any solution dries down, the molecules in the solution can either crystalize, or they can vitrify. A solute which has an extensive asymmetry may be a superior vitrifier, because of the hindrances to nucleation of crystals during drying. A substance that inhibits the crystallization of another substance may result in the combined substances forming a superior vitrification, such as raffinose in the presence of sucrose. See U.S. Pat. Nos. 5,290,765 and 9,469,835.

In some embodiments, a microbial composition is produced that is encapsulated in a vitrified substance. The vitrified composition may be created by selecting a mixture including cells; combining said mixture with sufficient quantity of one or more vitrifying solutes to protect said mixture during drying and to inhibit destructive reactions; and drying said combination by exposing said combination to a desiccant, or desiccating conditions, at a temperature above that which said combination will freeze and below that at which said vitrifying solutes achieve the vitrified state, at approximately normal atmospheric pressure, until said combination is substantially dry.

In one embodiment, the encapsulating composition comprises microcapsules having a multiplicity of liquid cores encapsulated in a solid shell material. For purposes of the disclosure, a “multiplicity” of cores is defined as two or more.

One category of fusible materials useful as encapsulating shell materials is that of waxes. Representative waxes contemplated for use herein are as follows: animal waxes, such as beeswax, lanolin, shell wax, and Chinese insect wax; vegetable waxes, such as carnauba, candelilla, bayberry, and sugar cane; mineral waxes, such as paraffin, microcrystalline petroleum, ozocerite, ceresin, and montan; synthetic waxes, such as low molecular weight polyolefin (e.g., CARBOWAX), and polyol ether-esters (e.g., sorbitol); Fischer-Tropsch process synthetic waxes; and mixtures thereof. Water-soluble waxes, such as CARBOWAX and sorbitol, are not contemplated herein if the core is aqueous. Still other fusible compounds useful herein are fusible natural resins, such as rosin, balsam, shellac, and mixtures thereof.

In some embodiments, the microbes, microbial consortia or microbial compositions of the present disclosure is embedded in a wax, such as the waxes described in the present disclosure. In some embodiments, the microbes or microbial composition is embedded in wax balls. In some embodiments, the microbes or microbial composition is already encapsulated prior to being embedded in wax balls. In some embodiments, the wax balls are 10 microns, 20 microns, 30 microns, 40 microns, 50 microns, 60 microns, 70 microns, 80 microns, 90 microns, 100 microns, 150 microns, 200 microns, 250 microns, 300 microns, 350 microns, 400 microns, 450 microns, 500 microns, 550 microns, 600 microns, 650 microns, 700 microns, 750 microns, 800 microns, 850 microns, 900 microns, 950 microns, or 1,000 microns in diameter.

In some embodiments, the wax balls are about 10 microbes, about 20 microns, about 30 microns, about 40 microns, about 50 microns, about 60 microns, about 70 microns, about 80 microns, about 90 microns, about 100 microns, about 150 microns, about 200 microns, about 250 microns, about 300 microns, about 350 microns, about 400 microns, about 450 microns, about 500 microns, about 550 microns, about 600 microns, about 650 microns, about 700 microns, about 750 microns, about 800 microns, about 850 microns, about 900 microns, about 950 microns, or about 1,000 microns in diameter.

In some embodiments, the wax balls are between 10-20 microns, 10-30 microns, 10-40 microns, 10-50 microns, 10-60 microns, 10-70 microns, 10-80 microns, 10-90 microns, 10-100 microns, 10-250 microns, 10-500 microns, 10-750 microns, 10-1,000 microns, 20-30 microns, 20-40 microns, 20-50 microns, 20-60 microns, 20-70 microns, 20-80 microns, 20-90 microns, 20-100 microns, 20-250 microns, 20-500 microns, 20-750 microns, 20-1,000 microns, 30-40 microns, 30-50 microns, 30-60 microns, 30-70 microns, 30-80 microns, 30-90 microns, 30-100 microns, 30-250 microns, 30-500 microns, 30-750 microns, 30-1,000 microns, 40-50 microns, 40-60 microns, 40-70 microns, 40-80 microns, 40-90 microns, 40-100 microns, 40-250 microns, 40-500 microns, 40-750 microns, 40-1,000 microns, 50-60 microns, 50-70 microns, 50-80 microns, 50-90 microns, 50-100 microns, 50-250 microns, 50-500 microns, 50-750 microns, 50-1,000 microns, 60-70 microns, 60-80 microns, 60-90 microns, 60-100 microns, 60-250 microns, 60-500 microns, 60-750 microns, 60-1,000 microns, 70-80 microns 70-90 microns, 70-90 microns, 70-100 microns, 70-250 microns, 70-500 microns, 70-750 microns, 70-1,000 microns, 80-90 microns, 80-100 microns, 80-250 microns, 80-500 microns, 80-500 microns, 80-750 microns, 80-1,000 microns, 90-100 microns, 90-250 microns, 90-500 microns, 90-750 microns, 90-1,000 microns, 100-250 microns, 100-500 microns, 100-750 microns, 100-1,000 microns, 250-500 microns, 250-750 microns, 250-1,000 microns, 500-750 microns, 500-1,000 microns, or 750-1,000 microns in diameter.

In some embodiments, the wax balls are between about 10-20 microns, about 10-30 microns, about 10-40 microns, about 10-50 microns, about 10-60 microns, about 10-70 microns, about 10-80 microns, about 10-90 microns, about 10-100 microns, about 10-250 microns, about 10-500 microns, about 10-750 microns, about 10-1,000 microns, about 20-30 microns, about 20-40 microns, about 20-50 microns, about 20-60 microns, about 20-70 microns, about 20-80 microns, about 20-90 microns, about 20-100 microns, about 20-250 microns, about 20-500 microns, about 20-750 microns, about 20-1,000 microns, about 30-40 microns, about 30-50 microns, about 30-60 microns, about 30-70 microns, about 30-80 microns, about 30-90 microns, about 30-100 microns, about 30-250 microns, about 30-500 microns, about 30-750 microns, about 30-1,000 microns, about 40-50 microns, about 40-60 microns, about 40-70 microns, about 40-80 microns, about 40-90 microns, about 40-100 microns, about 40-250 microns, about 40-500 microns, about 40-750 microns, about 40-1,000 microns, about 50-60 microns, about 50-70 microns, about 50-80 microns, about 50-90 microns, about 50-100 microns, about 50-250 microns, about 50-500 microns, about 50-750 microns, about 50-1,000 microns, about 60-70 microns, about 60-80 microns, about 60-90 microns, about 60-100 microns, about 60-250 microns, about 60-500 microns, about 60-750 microns, about 60-1,000 microns, about 70-80 microns about 70-90 microns, about 70-90 microns, about 70-100 microns, about 70-250 microns, about 70-500 microns, about 70-750 microns, about 70-1,000 microns, about 80-90 microns, about 80-100 microns, about 80-250 microns, about 80-500 microns, about 80-500 microns, about 80-750 microns, about 80-1,000 microns, about 90-100 microns, about 90-250 microns, about 90-500 microns, about 90-750 microns, about 90-1,000 microns, about 100-250 microns, about 100-500 microns, about 100-750 microns, about 100-1,000 microns, about 250-500 microns, about 250-750 microns, about 250-1,000 microns, about 500-750 microns, about 500-1,000 microns, or about 750-1,000 microns in diameter.

Various adjunct materials are contemplated for incorporation in fusible materials according to the present disclosure. For example, antioxidants, light stabilizers, dyes and lakes, flavors, essential oils, anti-caking agents, fillers, pH stabilizers, sugars (monosaccharides, disaccharides, trisaccharides, and polysaccharides) and the like can be incorporated in the fusible material in amounts which do not diminish its utility for the present disclosure.

The core material contemplated herein constitutes from about 0.1% to about 50%, about 1% to about 35%, or about 5% to about 30% by weight of the microcapsules. In some embodiments, the core material contemplated herein constitutes no more than about 30% by weight of the microcapsules. In some embodiments, the core material contemplated herein constitutes about 5% by weight of the microcapsules. The core material is contemplated as either a liquid or solid at contemplated storage temperatures of the microcapsules.

The cores may include other additives well-known in the agricultural art, including Other potentially useful supplemental core materials will be apparent to those of ordinary skill in the art. Emulsifying agents may be employed to assist in the formation of stable emulsions. Representative emulsifying agents include glyceryl monostearate, polysorbate esters, ethoxylated mono- and diglycerides, and mixtures thereof.

For ease of processing, and particularly to enable the successful formation of a reasonably stable emulsion, the viscosities of the core material and the shell material should be similar at the temperature at which the emulsion is formed. In particular, the ratio of the viscosity of the shell to the viscosity of the core, expressed in centipoise or comparable units, and both measured at the temperature of the emulsion, should be from about 22:1 to about 1:1, desirably from about 8:1 to about 1:1, and preferably from about 3:1 to about 1:1. A ratio of 1:1 would be ideal, but a viscosity ratio within the recited ranges is useful.

Encapsulating compositions are not limited to microcapsule compositions as disclosed above. In some embodiments encapsulating compositions encapsulate the microbial compositions in an adhesive polymer that can be natural or synthetic without toxic effect. In some embodiments, the encapsulating composition may be a matrix selected from sugar matrix, gelatin matrix, polymer matrix, silica matrix, starch matrix, foam matrix, glass/glassy matrix etc. See Pirzio et al. (U.S. Pat. No. 7,488,503). In some embodiments, the encapsulating composition may be selected from polyvinyl acetates; polyvinyl acetate copolymers; ethylene vinyl acetate (EVA) copolymers; polyvinyl alcohols; polyvinyl alcohol copolymers; celluloses, including ethylcelluloses, methylcelluloses, hydroxymethylcelluloses, hydroxypropylcelluloses and carboxymethylcellulose; polyvinylpyrolidones; polysaccharides, including starch, modified starch, dextrins, maltodextrins, alginate and chitosans; monosaccharides; fats; fatty acids, including oils; proteins, including gelatin and zeins; gum arabics; shellacs; vinylidene chloride and vinylidene chloride copolymers; calcium lignosulfonates; acrylic copolymers; polyvinylacrylates; polyethylene oxide; acrylamide polymers and copolymers; polyhydroxyethyl acrylate, methylacrylamide monomers; and polychloroprene.

In some embodiments, the encapsulating compositions comprise at least one layer of encapsulation. In some embodiments, the encapsulating compositions comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 layers of encapsulation/encapsulants.

In some embodiments, the encapsulating compositions comprise at least two layers of encapsulation. In some embodiments, each layer of encapsulation confers a different characteristic to the composition. In some embodiments, no two consecutive layers confer the same characteristic. In some embodiments, at least one layer of the at least two layers of encapsulation confers thermostability, shelf stability, ultraviolet resistance, moisture resistance, hydrophobicity, hydrophilicity, lipophobicity, lipophilicity, pH stability, acid resistance, and base resistance.

In some embodiments, the encapsulating compositions comprise two layers of encapsulation; the first layer confers thermostability and/or shelf stability, and the second layer provides pH resistance. In some embodiments, the encapsulating layers confer a timed release of the microbial composition held in the center of the encapsulating layers. In some embodiments, the greater the number of layers confers a greater amount of time before the microbial composition is exposed, post administration.

In some embodiments, the encapsulating shell of the present disclosure can be up to 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 510 μm, 520 μm, 530 μm, 540 μm, 550 μm, 560 μm, 570 μm, 580 μm, 590 μm, 600 μm, 610 μm, 620 μm, 630 μm, 640 μm, 650 μm, 660 μm, 670 μm, 680 μm, 690 μm, 700 μm, 710 μm, 720 μm, 730 μm, 740 μm, 750 μm, 760 μm, 770 μm, 780 μm, 790 μm, 800 μm, 810 μm, 820 μm, 830 μm, 840 μm, 850 μm, 860 μm, 870 μm, 880 μm, 890 μm, 900 μm, 910 μm, 920 μm, 930 μm, 940 μm, 950 μm, 960 μm, 970 μm, 980 μm, 990 μm, 1000 μm, 1010 μm, 1020 μm, 1030 μm, 1040 μm, 1050 μm, 1060 μm, 1070 μm, 1080 μm, 1090 μm, 1100 μm, 1110 μm, 1120 μm, 1130 μm, 1140 μm, 1150 μm, 1160 μm, 1170 μm, 1180 μm, 1190 μm, 1200 μm, 1210 μm, 1220 μm, 1230 μm, 1240 μm, 1250 μm, 1260 μm, 1270 μm, 1280 μm, 1290 μm, 1300 μm, 1310 μm, 1320 μm, 1330 μm, 1340 μm, 1350 μm, 1360 μm, 1370 μm, 1380 μm, 1390 μm, 1400 μm, 1410 μm, 1420 μm, 1430 μm, 1440 μm, 1450 μm, 1460 μm, 1470 μm, 1480 μm, 1490 μm, 1500 μm, 1510 μm, 1520 μm, 1530 μm, 1540 μm, 1550 μm, 1560 μm, 1570 μm, 1580 μm, 1590 μm, 1600 μm, 1610 μm, 1620 μm, 1630 μm, 1640 μm, 1650 μm, 1660 μm, 1670 μm, 1680 μm, 1690 μm, 1700 μm, 1710 μm, 1720 μm, 1730 μm, 1740 μm, 1750 μm, 1760 μm, 1770 μm, 1780 μm, 1790 μm, 1800 μm, 1810 μm, 1820 μm, 1830 μm, 1840 μm, 1850 μm, 1860 μm, 1870 μm, 1880 μm, 1890 μm, 1900 μm, 1910 μm, 1920 μm, 1930 μm, 1940 μm, 1950 μm, 1960 μm, 1970 μm, 1980 μm, 1990 μm, 2000 μm, 2010 μm, 2020 μm, 2030 μm, 2040 μm, 2050 μm, 2060 μm, 2070 μm, 2080 μm, 2090 μm, 2100 μm, 2110 μm, 2120 μm, 2130 μm, 2140 μm, 2150 μm, 2160 μm, 2170 μm, 2180 μm, 2190 μm, 2200 μm, 2210 μm, 2220 μm, 2230 μm, 2240 μm, 2250 μm, 2260 μm, 2270 μm, 2280 μm, 2290 μm, 2300 μm, 2310 μm, 2320 μm, 2330 μm, 2340 μm, 2350 μm, 2360 μm, 2370 μm, 2380 μm, 2390 μm, 2400 μm, 2410 μm, 2420 μm, 2430 μm, 2440 μm, 2450 μm, 2460 μm, 2470 μm, 2480 μm, 2490 μm, 2500 μm, 2510 μm, 2520 μm, 2530 μm, 2540 μm, 2550 μm, 2560 μm, 2570 μm, 2580 μm, 2590 μm, 2600 μm, 2610 μm, 2620 μm, 2630 μm, 2640 μm, 2650 μm, 2660 μm, 2670 μm, 2680 μm, 2690 μm, 2700 μm, 2710 μm, 2720 μm, 2730 μm, 2740 μm, 2750 μm, 2760 μm, 2770 μm, 2780 μm, 2790 μm, 2800 μm, 2810 μm, 2820 μm, 2830 μm, 2840 μm, 2850 μm, 2860 μm, 2870 μm, 2880 μm, 2890 μm, 2900 μm, 2910 μm, 2920 μm, 2930 μm, 2940 μm, 2950 μm, 2960 μm, 2970 μm, 2980 μm, 2990 μm, or 3000 μm thick.

In some embodiments, the encapsulation composition of the present disclosure possesses a water activity (a_{w}) of less than 0.750, 0.700, 0.650, 0.600, 0.550, 0.500, 0.475, 0.450, 0.425, 0.400, 0.375, 0.350, 0.325, 0.300, 0.275, 0.250, 0.225, 0.200, 0.190, 0.180, 0.170, 0.160, 0.150, 0.140, 0.130, 0.120, 0.110, 0.100, 0.095, 0.090, 0.085, 0.080, 0.075, 0.070, 0.065, 0.060, 0.055, 0.050, 0.045, 0.040, 0.035, 0.030, 0.025, 0.020, 0.015, 0.010, or 0.005.

In some embodiments, the encapsulation composition of the present disclosure possesses a water activity (a_{w}) of less than about 0.750, about 0.700, about 0.650, about 0.600, about 0.550, about 0.500, about 0.475, about 0.450, about 0.425, about 0.400, about 0.375, about 0.350, about 0.325, about 0.300, about 0.275, about 0.250, about 0.225, about 0.200, about 0.190, about 0.180, about 0.170, about 0.160, about 0.150, about 0.140, about 0.130, about 0.120, about 0.110, about 0.100, about 0.095, about 0.090, about 0.085, about 0.080, about 0.075, about 0.070, about 0.065, about 0.060, about 0.055, about 0.050, about 0.045, about 0.040, about 0.035, about 0.030, about 0.025, about 0.020, about 0.015, about 0.010, or about 0.005.

In one embodiment, the microbe(s) are first dried by spray dry, lyophilization, or foam drying along with excipients that may include one or more sugars, sugar alcohols, disaccharides, trisaccharides, polysaccharides, salts, amino acids, amino acid salts, or polymers.

In some embodiments, the microbes or compositions comprising the microbes are milled to a size of 10 microns, 20 microns, 30 microns, 40 microns, 50 microns, 60 microns, 70 microns, 80 microns, 90 microns, 100 microns, 150 microns, 200 microns, 250 microns, 300 microns, 350 microns, 400 microns, 450 microns, 500 microns, 550 microns, 600 microns, 650 microns, 700 microns, 750 microns, 800 microns, 850 microns, 900 microns, 950 microns, or 1,000 microns in diameter.

In some embodiments, the microbes or compositions comprising the microbes are milled to a size of about 10 microns, about 20 microns, about 30 microns, about 40 microns, about 50 microns, about 60 microns, about 70 microns, about 80 microns, about 90 microns, about 100 microns, about 150 microns, about 200 microns, about 250 microns, about 300 microns, about 350 microns, about 400 microns, about 450 microns, about 500 microns, about 550 microns, about 600 microns, about 650 microns, about 700 microns, about 750 microns, about 800 microns, about 850 microns, about 900 microns, about 950 microns, or about 1,000 microns in diameter.

In some embodiments, the microbes or compositions comprising the microbes are milled to a size of between 10-20 microns, 10-30 microns, 10-40 microns, 10-50 microns, 10-60 microns, 10-70 microns, 10-80 microns, 10-90 microns, 10-100 microns, 10-250 microns, 10-500 microns, 10-750 microns, 10-1,000 microns, 20-30 microns, 20-40 microns, 20-50 microns, 20-60 microns, 20-70 microns, 20-80 microns, 20-90 microns, 20-100 microns, 20-250 microns, 20-500 microns, 20-750 microns, 20-1,000 microns, 30-40 microns, 30-50 microns, 30-60 microns, 30-70 microns, 30-80 microns, 30-90 microns, 30-100 microns, 30-250 microns, 30-500 microns, 30-750 microns, 30-1,000 microns, 40-50 microns, 40-60 microns, 40-70 microns, 40-80 microns, 40-90 microns, 40-100 microns, 40-250 microns, 40-500 microns, 40-750 microns, 40-1,000 microns, 50-60 microns, 50-70 microns, 50-80 microns, 50-90 microns, 50-100 microns, 50-250 microns, 50-500 microns, 50-750 microns, 50-1,000 microns, 60-70 microns, 60-80 microns, 60-90 microns, 60-100 microns, 60-250 microns, 60-500 microns, 60-750 microns, 60-1,000 microns, 70-80 microns 70-90 microns, 70-90 microns, 70-100 microns, 70-250 microns, 70-500 microns, 70-750 microns, 70-1,000 microns, 80-90 microns, 80-100 microns, 80-250 microns, 80-500 microns, 80-500 microns, 80-750 microns, 80-1,000 microns, 90-100 microns, 90-250 microns, 90-500 microns, 90-750 microns, 90-1,000 microns, 100-250 microns, 100-500 microns, 100-750 microns, 100-1,000 microns, 250-500 microns, 250-750 microns, 250-1,000 microns, 500-750 microns, 500-1,000 microns, or 750-1,000 microns in diameter.

In some embodiments, the microbes or compositions comprising the microbes are milled to a size of between about 10-20 microns, about 10-30 microns, about 10-40 microns, about 10-50 microns, about 10-60 microns, about 10-70 microns, about 10-80 microns, about 10-90 microns, about 10-100 microns, about 10-250 microns, about 10-500 microns, about 10-750 microns, about 10-1,000 microns, about 20-30 microns, about 20-40 microns, about 20-50 microns, about 20-60 microns, about 20-70 microns, about 20-80 microns, about 20-90 microns, about 20-100 microns, about 20-250 microns, about 20-500 microns, about 20-750 microns, about 20-1,000 microns, about 30-40 microns, about 30-50 microns, about 30-60 microns, about 30-70 microns, about 30-80 microns, about 30-90 microns, about 30-100 microns, about 30-250 microns, about 30-500 microns, about 30-750 microns, about 30-1,000 microns, about 40-50 microns, about 40-60 microns, about 40-70 microns, about 40-80 microns, about 40-90 microns, about 40-100 microns, about 40-250 microns, about 40-500 microns, about 40-750 microns, about 40-1,000 microns, about 50-60 microns, about 50-70 microns, about 50-80 microns, about 50-90 microns, about 50-100 microns, about 50-250 microns, about 50-500 microns, about 50-750 microns, about 50-1,000 microns, about 60-70 microns, about 60-80 microns, about 60-90 microns, about 60-100 microns, about 60-250 microns, about 60-500 microns, about 60-750 microns, about 60-1,000 microns, about 70-80 microns about 70-90 microns, about 70-90 microns, about 70-100 microns, about 70-250 microns, about 70-500 microns, about 70-750 microns, about 70-1,000 microns, about 80-90 microns, about 80-100 microns, about 80-250 microns, about 80-500 microns, about 80-500 microns, about 80-750 microns, about 80-1,000 microns, about 90-100 microns, about 90-250 microns, about 90-500 microns, about 90-750 microns, about 90-1,000 microns, about 100-250 microns, about 100-500 microns, about 100-750 microns, about 100-1,000 microns, about 250-500 microns, about 250-750 microns, about 250-1,000 microns, about 500-750 microns, about 500-1,000 microns, or about 750-1,000 microns in diameter.

In some embodiments, the microbes or compositions comprising the microbes are combined with a wax, fat, oil, fatty acid, or fatty alcohol, and spray congealed into beads of about 10 microns, about 20 microns, about 30 microns, about 40 microns, about 50 microns, about 60 microns, about 70 microns, about 80 microns, about 90 microns, about 100 microns, about 150 microns, about 200 microns, about 250 microns, about 300 microns, about 350 microns, about 400 microns, about 450 microns, about 500 microns, about 550 microns, about 600 microns, about 650 microns, about 700 microns, about 750 microns, about 800 microns, about 850 microns, about 900 microns, about 950 microns, or about 1,000 microns in diameter.

In some embodiments, the microbes or compositions comprising the microbes are combined with a wax, fat, oil, fatty acid, or fatty alcohol, and spray congealed into beads of between 10-20 microns, 10-30 microns, 10-40 microns, 10-50 microns, 10-60 microns, 10-70 microns, 10-80 microns, 10-90 microns, 10-100 microns, 10-250 microns, 10-500 microns, 10-750 microns, 10-1,000 microns, 20-30 microns, 20-40 microns, 20-50 microns, 20-60 microns, 20-70 microns, 20-80 microns, 20-90 microns, 20-100 microns, 20-250 microns, 20-500 microns, 20-750 microns, 20-1,000 microns, 30-40 microns, 30-50 microns, 30-60 microns, 30-70 microns, 30-80 microns, 30-90 microns, 30-100 microns, 30-250 microns, 30-500 microns, 30-750 microns, 30-1,000 microns, 40-50 microns, 40-60 microns, 40-70 microns, 40-80 microns, 40-90 microns, 40-100 microns, 40-250 microns, 40-500 microns, 40-750 microns, 40-1,000 microns, 50-60 microns, 50-70 microns, 50-80 microns, 50-90 microns, 50-100 microns, 50-250 microns, 50-500 microns, 50-750 microns, 50-1,000 microns, 60-70 microns, 60-80 microns, 60-90 microns, 60-100 microns, 60-250 microns, 60-500 microns, 60-750 microns, 60-1,000 microns, 70-80 microns 70-90 microns, 70-90 microns, 70-100 microns, 70-250 microns, 70-500 microns, 70-750 microns, 70-1,000 microns, 80-90 microns, 80-100 microns, 80-250 microns, 80-500 microns, 80-500 microns, 80-750 microns, 80-1,000 microns, 90-100 microns, 90-250 microns, 90-500 microns, 90-750 microns, 90-1,000 microns, 100-250 microns, 100-500 microns, 100-750 microns, 100-1,000 microns, 250-500 microns, 250-750 microns, 250-1,000 microns, 500-750 microns, 500-1,000 microns, or 750-1,000 microns in diameter.

In some embodiments, the microbes or compositions comprising the microbes are combined with a wax, fat, oil, fatty acid, or fatty alcohol, and spray congealed into beads of between about 10-20 microns, about 10-30 microns, about 10-40 microns, about 10-50 microns, about 10-60 microns, about 10-70 microns, about 10-80 microns, about 10-90 microns, about 10-100 microns, about 10-250 microns, about 10-500 microns, about 10-750 microns, about 10-1,000 microns, about 20-30 microns, about 20-40 microns, about 20-50 microns, about 20-60 microns, about 20-70 microns, about 20-80 microns, about 20-90 microns, about 20-100 microns, about 20-250 microns, about 20-500 microns, about 20-750 microns, about 20-1,000 microns, about 30-40 microns, about 30-50 microns, about 30-60 microns, about 30-70 microns, about 30-80 microns, about 30-90 microns, about 30-100 microns, about 30-250 microns, about 30-500 microns, about 30-750 microns, about 30-1,000 microns, about 40-50 microns, about 40-60 microns, about 40-70 microns, about 40-80 microns, about 40-90 microns, about 40-100 microns, about 40-250 microns, about 40-500 microns, about 40-750 microns, about 40-1,000 microns, about 50-60 microns, about 50-70 microns, about 50-80 microns, about 50-90 microns, about 50-100 microns, about 50-250 microns, about 50-500 microns, about 50-750 microns, about 50-1,000 microns, about 60-70 microns, about 60-80 microns, about 60-90 microns, about 60-100 microns, about 60-250 microns, about 60-500 microns, about 60-750 microns, about 60-1,000 microns, about 70-80 microns about 70-90 microns, about 70-90 microns, about 70-100 microns, about 70-250 microns, about 70-500 microns, about 70-750 microns, about 70-1,000 microns, about 80-90 microns, about 80-100 microns, about 80-250 microns, about 80-500 microns, about 80-500 microns, about 80-750 microns, about 80-1,000 microns, about 90-100 microns, about 90-250 microns, about 90-500 microns, about 90-750 microns, about 90-1,000 microns, about 100-250 microns, about 100-500 microns, about 100-750 microns, about 100-1,000 microns, about 250-500 microns, about 250-750 microns, about 250-1,000 microns, about 500-750 microns, about 500-1,000 microns, or about 750-1,000 microns in diameter.

In some embodiments, the microbes or compositions comprising the microbes are combined with a wax, fat, oil, fatty acid, or fatty alcohol as well as a water-soluble polymer, salt, polysaccharide, sugar, polypeptide, protein, or sugar alcohol and spray congealed into beads, the size of which are described herein. In some embodiments, the water-soluble polymer, salt, polysaccharide, sugar, or sugar alcohol serves as a disintegrant. In some embodiments, the disintegrant forms pores once the beads are dispersed in the soil.

In some embodiments, the composition of the water-soluble polymer, salt, polysaccharide, sugar, polypeptide, protein, or sugar alcohol is modified such that the disintegrant dissolves within 1, 5, 10, 15, 20, 25, 30, 35, 40, 45, 50, 55, 60 minutes of being administered. In some embodiments, the composition of the water-soluble polymer, salt, polysaccharide, sugar, polypeptide, protein, or sugar alcohol is modified such that the disintegrant dissolves within about 1, about 5, about 10, about 15, about 20, about 25, about 30, about 35, about 40, about 45, about 50, about 55, or about 60 minutes of being administered.

In some embodiments, the composition of the water-soluble polymer, salt, polysaccharide, sugar, polypeptide, protein, or sugar alcohol is modified such that the disintegrant dissolves within 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 hours of being administered. In some embodiments, the composition of the water-soluble polymer, salt, polysaccharide, sugar, polypeptide, protein, or sugar alcohol is modified such that the disintegrant dissolves within about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, or about 12 hours of being administered.

In some embodiments, the composition of the water-soluble polymer, salt, polysaccharide, sugar, polypeptide, protein, or sugar alcohol is modified such that the disintegrant dissolves at a temperature of at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50° C. In some embodiments, the composition of the water-soluble polymer, salt, polysaccharide, sugar, polypeptide, protein, or sugar alcohol is modified such that the disintegrant dissolves at a temperature of at least about 10, least about 11, least about 12, least about 13, least about 14, least about 15, least about 16, least about 17, least about 18, least about 19, least about 20, least about 21, least about 22, least about 23, least about 24, least about 25, least about 26, least about 27, least about 28, least about 29, least about 30, least about 31, least about 32, least about 33, least about 34, about 35, about 36, about 37, about 38, about 39, about 40, about 41, about 42, about 43, about 44, least about 45, least about 46, least about 47, least about 48, least about 49, or least about 50° C.

In some embodiments, the composition of the water-soluble polymer, salt, polysaccharide, sugar, polypeptide, protein, or sugar alcohol is modified such that the disintegrant dissolves at a pH of at least 3.8, 3.9, 4. 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 or 10.0. In some embodiments, the composition of the water-soluble polymer, salt, polysaccharide, sugar, polypeptide, protein, or sugar alcohol is modified such that the disintegrant dissolves at a pH of at least about 3.8, least about 3.9, least about 4. least about 4.1, least about 4.2, least about 4.3, least about 4.4, least about 4.5, least about 4.6, least about 4.7, least about 4.8, least about 4.9, least about 5.0, least about 5.1, least about 5.2, least about 5.3, least about 5.4, least about 5.5, least about 5.6, least about 5.7, least about 5.8, least about 5.9, least about 6.0, least about 6.2, least about 6.3, least about 6.4, least about 6.5, least about 6.6, least about 6.7, least about 6.8, least about 6.9, least about 7.0, least about 7.1, least about 7.2, least about 7.3, least about 7.4, least about 7.5, least about 7.6, least about 7.7, least about 7.8, least about 7.9, least about 8.0, least about 8.1, least about 8.2, least about 8.3, least about 8.4, least about 8.5, least about 8.6, least about 8.7, least about 8.8, least about 8.9, least about 9.0, least about 9.1, least about 9.2, least about 9.3, least about 9.4, least about 9.5, least about 9.6, least about 9.7, least about 9.8, least about 9.9, or least about 10.0.

In some embodiments, the microbes or compositions comprising the microbes are coated with a polymer, a polysaccharide, sugar, sugar alcohol, gel, wax, fat, fatty alcohol, or fatty acid

In some embodiments, the microbes or compositions comprising the microbes are coated with a polymer, a polysaccharide, sugar, sugar alcohol, gel, wax, fat, fatty alcohol, or fatty acid.

In some embodiments, the coating of the microbes or compositions comprising the microbes is modified such that the coating dissolves within 1, 5, 10, 15, 20, 25, 30, 35, 40, 45, 50, 55, 60 minutes of being administered. In some embodiments, the coating of the microbes or compositions comprising the microbes is modified such that the coating dissolves within about 1, about 5, about 10, about 15, about 20, about 25, about 30, about 35, about 40, about 45, about 50, about 55, or about 60 minutes of being administered.

In some embodiments, the coating of the microbes or compositions comprising the microbes is modified such that the coating dissolves within 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 hours of being administered. In some embodiments, the coating of the microbes or compositions comprising the microbes is modified such that the coating dissolves within about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, or about 12 hours of being administered.

In some embodiments, the coating of the microbes or compositions comprising the microbes is modified such that the coating dissolves at a temperature of at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50° C. In some embodiments, the coating of the microbes or compositions comprising the microbes is modified such that the coating dissolves at a temperature of at least about 10, least about 11, least about 12, least about 13, least about 14, least about 15, least about 16, least about 17, least about 18, least about 19, least about 20, least about 21, least about 22, least about 23, least about 24, least about 25, least about 26, least about 27, least about 28, least about 29, least about 30, least about 31, least about 32, least about 33, least about 34, about 35, about 36, about 37, about 38, about 39, about 40, about 41, about 42, about 43, about 44, least about 45, least about 46, least about 47, least about 48, least about 49, or least about 50° C.

In some embodiments, the coating of the microbes or compositions comprising the microbes is modified such that the coating dissolves at a pH of at least 3.8, 3.9, 4. 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 or 10.0. In some embodiments, the coating of the microbes or compositions comprising the microbes is modified such that the coating dissolves at a pH of at least about 3.8, least about 3.9, least about 4. least about 4.1, least about 4.2, least about 4.3, least about 4.4, least about 4.5, least about 4.6, least about 4.7, least about 4.8, least about 4.9, least about 5.0, least about 5.1, least about 5.2, least about 5.3, least about 5.4, least about 5.5, least about 5.6, least about 5.7, least about 5.8, least about 5.9, least about 6.0, least about 6.2, least about 6.3, least about 6.4, least about 6.5, least about 6.6, least about 6.7, least about 6.8, least about 6.9, least about 7.0, least about 7.1, least about 7.2, least about 7.3, least about 7.4, least about 7.5, least about 7.6, least about 7.7, least about 7.8, least about 7.9, least about 8.0, least about 8.1, least about 8.2, least about 8.3, least about 8.4, least about 8.5, least about 8.6, least about 8.7, least about 8.8, least about 8.9, least about 9.0, least about 9.1, least about 9.2, least about 9.3, least about 9.4, least about 9.5, least about 9.6, least about 9.7, least about 9.8, least about 9.9, or least about 10.0.

### Agricultural Applications of Microbial Compositions

The microbial compositions disclosed herein may be in the form of a dry powder, a slurry of powder and water, a granular material, or a flowable seed treatment. The compositions comprising microbe populations disclosed herein may be coated on a surface of a seed, and may be in liquid form.

The composition can be fabricated in bioreactors such as continuous stirred tank reactors, batch reactors, and on the farm. In some examples, compositions can be stored in a container, such as a jug or in mini bulk. In some examples, compositions may be stored within an object selected from the group consisting of a bottle, jar, ampule, package, vessel, bag, box, bin, envelope, carton, container, silo, shipping container, truck bed, and/or case.

In some examples, one or more compositions may be coated onto a seed. In some examples, one or more compositions may be coated onto a seedling. In some examples, one or more compositions may be coated onto a surface of a seed. In some examples, one or more compositions may be coated as a layer above a surface of a seed. In some examples, a composition that is coated onto a seed may be in liquid form, in dry product form, in foam form, in a form of a slurry of powder and water, or in a flowable seed treatment. In some examples, one or more compositions may be applied to a seed and/or seedling by spraying, immersing, coating, encapsulating, and/or dusting the seed and/or seedling with the one or more compositions. In some examples, multiple bacteria or bacterial populations can be coated onto a seed and/or a seedling of the plant. In some examples, at least two, at least three, at least four, at least five, at least six, at least seven, at least eight, at least nine, at least ten, or more than ten bacteria of a bacterial combination can be selected from any one of the microbes disclosed herein.

Examples of compositions may include seed coatings for commercially important agricultural crops, for example, sorghum, canola, tomato, strawberry, barley, rice, maize, and wheat. Examples of compositions can also include seed coatings for corn, soybean, canola, sorghum, potato, rice, vegetables, cereals, and oilseeds. Seeds as provided herein can be genetically modified organisms (GMO), non-GMO, organic, or conventional. In some examples, compositions may be sprayed on the plant aerial parts, or applied to the roots by inserting into furrows in which the plant seeds are planted, watering to the soil, or dipping the roots in a suspension of the composition. In some examples, compositions may be dehydrated in a suitable manner that maintains cell viability and the ability to artificially inoculate and colonize host plants. The bacterial species may be present in compositions at a concentration of between 10₈ to 10₁₀ CFU/ml. In some examples, compositions may be supplemented with trace metal ions, such as molybdenum ions, iron ions, manganese ions, or combinations of these ions. The concentration of ions in examples of compositions as described herein may between about 0.1 mM and about 50 mM. Some examples of compositions may also be formulated with a carrier, such as beta-glucan, carboxylmethyl cellulose (CMC), bacterial extracellular polymeric substance (EPS), sugar, animal milk, or other suitable carriers. In some examples, peat or planting materials can be used as a carrier, or biopolymers in which a composition is entrapped in the biopolymer can be used as a carrier. The compositions comprising the bacterial populations described herein can improve plant traits, such as promoting plant growth, maintaining high chlorophyll content in leaves, increasing fruit or seed numbers, and increasing fruit or seed unit weight.

The compositions comprising the bacterial populations described herein may be coated on to the surface of a seed. As such, compositions comprising a seed coated with one or more bacteria described herein are also contemplated. The seed coating can be formed by mixing the bacterial population with a porous, chemically inert granular carrier. Alternatively, the compositions may be inserted directly into the furrows into which the seed is planted or sprayed onto the plant leaves or applied by dipping the roots into a suspension of the composition. An effective amount of the composition can be used to populate the sub-soil region adjacent to the roots of the plant with viable bacterial growth, or populate the leaves of the plant with viable bacterial growth. In general, an effective amount is an amount sufficient to result in plants with improved traits (e.g. a desired level of nitrogen fixation).

In some embodiments, the microbes, microbial consortia or microbial compositions of the present disclosure may be formulated using an agriculturally acceptable carrier. The formulation useful for these embodiments may include at least one member selected from the group consisting of a tackifier, a microbial stabilizer, a fungicide, an antibacterial agent, a preservative, a stabilizer, a surfactant, an anti-complex agent, an herbicide, a nematicide, an insecticide, a plant growth regulator, a fertilizer, a rodenticide, a dessicant, a bactericide, a nutrient, a hormone, or any combination thereof. In some examples, compositions may be shelf-stable. For example, any of the compositions described herein can include an agriculturally acceptable carrier (e.g., one or more of a fertilizer such as a nonnaturally occurring fertilizer, an adhesion agent such as a non-naturally occurring adhesion agent, and a pesticide such as a non-naturally occurring pesticide). A non-naturally occurring adhesion agent can be, for example, a polymer, copolymer, or synthetic wax. For example, any of the coated seeds, seedlings, or plants described herein can contain such an agriculturally acceptable carrier in the seed coating. In any of the compositions or methods described herein, an agriculturally acceptable carrier can be or can include a non-naturally occurring compound (e.g., a non-naturally occurring fertilizer, a non-naturally occurring adhesion agent such as a polymer, copolymer, or synthetic wax, or a non-naturally occurring pesticide). Non-limiting examples of agriculturally acceptable carriers are described below. Additional examples of agriculturally acceptable carriers are known in the art.

In some cases, the microbes, microbial consortia or microbial compositions of the present disclosure may be mixed with an agriculturally acceptable carrier. The carrier can be a solid carrier or liquid carrier, and in various forms including microspheres, powders, emulsions and the like. The carrier may be any one or more of a number of carriers that confer a variety of properties, such as increased stability, wettability, or dispersability. Wetting agents such as natural or synthetic surfactants, which can be nonionic or ionic surfactants, or a combination thereof can be included in the composition. Water-in-oil emulsions can also be used to formulate a composition that includes the isolated bacteria (see, for example, U.S. Pat. No. 7,485,451). Suitable formulations that may be prepared include wettable powders, granules, gels, agar strips or pellets, thickeners, and the like, microencapsulated particles, and the like, liquids such as aqueous flowables, aqueous suspensions, water-in-oil emulsions, etc. The formulation may include grain or legume products, for example, ground grain or beans, broth or flour derived from grain or beans, starch, sugar, or oil.

In some embodiments, the agricultural carrier may be soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the bacteria, such as barley, rice, or other biological materials such as seed, plant parts, sugar cane bagasse, hulls or stalks from grain processing, ground plant material or wood from building site refuse, sawdust or small fibers from recycling of paper, fabric, or wood.

For example, a fertilizer can be used to help promote the growth or provide nutrients to a seed, seedling, or plant. Non-limiting examples of fertilizers include nitrogen, phosphorous, potassium, calcium, sulfur, magnesium, boron, chloride, manganese, iron, zinc, copper, molybdenum, and selenium (or a salt thereof). Additional examples of fertilizers include one or more amino acids, salts, carbohydrates, vitamins, glucose, NaCl, yeast extract, NH₄H₂PO₄, (NH₄)₂SO₄, glycerol, valine, L-leucine, lactic acid, propionic acid, succinic acid, malic acid, citric acid, KH tartrate, xylose, lyxose, and lecithin. In one embodiment, the formulation can include a tackifier or adherent (referred to as an adhesive agent) to help bind other active agents to a substance (e.g., a surface of a seed). Such agents are useful for combining bacteria with carriers that can contain other compounds (e.g., control agents that are not biologic), to yield a coating composition. Such compositions help create coatings around the plant or seed to maintain contact between the microbe and other agents with the plant or plant part. In one embodiment, adhesives are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers.

In some embodiments, the adhesives can be, e.g. a wax such as carnauba wax, beeswax, Chinese wax, shellac wax, spermaceti wax, candelilla wax, castor wax, ouricury wax, and rice bran wax, a polysaccharide (e.g., starch, dextrins, maltodextrins, alginate, and chitosans), a fat, oil, a protein (e.g., gelatin and zeins), gum arables, and shellacs. Adhesive agents can be nonnaturally occurring compounds, e.g., polymers, copolymers, and waxes. For example, nonlimiting examples of polymers that can be used as an adhesive agent include: polyvinyl acetates, polyvinyl acetate copolymers, ethylene vinyl acetate (EVA) copolymers, polyvinyl alcohols, polyvinyl alcohol copolymers, celluloses (e.g., ethylcelluloses, methylcelluloses, hydroxymethylcelluloses, hydroxypropylcelluloses, and carboxymethylcelluloses), polyvinylpyrolidones, vinyl chloride, vinylidene chloride copolymers, calcium lignosulfonates, acrylic copolymers, polyvinylacrylates, polyethylene oxide, acylamide polymers and copolymers, polyhydroxyethyl acrylate, methylacrylamide monomers, and polychloroprene.

In some examples, one or more of the adhesion agents, anti-fungal agents, growth regulation agents, and pesticides (e.g., insecticide) are non-naturally occurring compounds (e.g., in any combination). Additional examples of agriculturally acceptable carriers include dispersants (e.g., polyvinylpyrrolidone/vinyl acetate PVPIVA S-630), surfactants, binders, and filler agents. The formulation can also contain a surfactant. Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

In certain cases, the formulation includes a microbial stabilizer. Such an agent can include a desiccant, which can include any compound or mixture of compounds that can be classified as a desiccant regardless of whether the compound or compounds are used in such concentrations that they in fact have a desiccating effect on a liquid inoculant. Such desiccants are ideally compatible with the bacterial population used, and should promote the ability of the microbial population to survive application on the seeds and to survive desiccation. Examples of suitable desiccants include one or more of trehalose, sucrose, glycerol, and Methylene glycol. Other suitable desiccants include, but are not limited to, non-reducing sugars and sugar alcohols (e.g., mannitol or sorbitol). The amount of desiccant introduced into the formulation can range from about 5% to about 50% by weight/volume, for example, between about 10% to about 40%, between about 15% to about 35%, or between about 20% to about 30%. In some cases, it is advantageous for the formulation to contain agents such as a fungicide, an antibacterial agent, an herbicide, a nematicide, an insecticide, a plant growth regulator, a rodenticide, bactericide, or a nutrient. In some examples, agents may include protectants that provide protection against seed surface-borne pathogens. In some examples, protectants may provide some level of control of soil-borne pathogens. In some examples, protectants may be effective predominantly on a seed surface.

### Methods of Improving Soil and Promoting Plant Growth

The disclosure provides methods of improving soil for plant growth comprising applying any one of the microbes, microbial consortia and/or microbial compositions disclosed herein. The disclosure provides methods of promoting plant growth comprising applying any one of the microbes, microbial consortia and/or microbial compositions disclosed herein. In some embodiments, the microbial consortia is applied before planting. In some embodiments, the microbial consortia is applied after plant germination. In some embodiments, the microbial consortia is applied as a seed treatment. In some embodiments, the microbial consortia is applied as a spray. In some embodiments, the microbial consortia is applied as a soil drench.

In some embodiments, the microbial composition is administered in a dose volume comprising a total of, or at least 0.5 ml, 1 ml, 2 ml, 3 ml, 4 ml, 5 ml, 6 ml, 7 ml, 8 ml, 9 ml, 10 ml, 11 ml, 12 ml, 13 ml, 14 ml, 15 ml, 16 ml, 17 ml, 18 ml, 19 ml, 20 ml, 21 ml, 22 ml, 23 ml, 24 ml, 25 ml, 26 ml, 27 ml, 28 ml, 29 ml, 30 ml, 31 ml, 32 ml, 33 ml, 34 ml, 35 ml, 36 ml, 37 ml, 38 ml, 39 ml, 40 ml, 41 m, 42 ml, 43 ml, 44 ml, 45 ml, 46 ml, 47 ml, 48 ml, 49 ml, 50 ml, 60 ml, 70 ml, 80 ml, 90 ml, 100 ml, 200 ml, 300 ml, 400 ml, 500 ml, 600 ml, 700 ml, 800 ml, 900 ml, or 1,000 ml.

In some embodiments, the microbial composition is administered in a dose comprising a total of, or at least 10¹⁸, 10¹⁷, 10¹⁶, 10¹⁵, 10¹⁴, 10¹³, 10¹², 10¹¹, 10¹⁰, 10⁹, 10⁸, 10⁷, 10⁶, 10⁵, 10⁴, 10³, or 10² microbial cells. In some embodiments, these microbial cells are quantified by colony forming units (CFUs).

In some embodiments, the dose of the microbial composition is administered such that there exists 10² to 10¹², 10³ to 10¹², 10⁴ to 10¹², 10⁵ to 10¹², 10⁶ to 10¹², 10⁷ to 10¹², 10⁸ to 10¹², 10⁹ to 10¹², 10¹⁰ to 10¹², 10¹¹ to 10¹², 10² to 10¹¹, 10³ to 10¹¹, 10⁴ to 10¹¹, 10⁵ to 10¹¹, 10⁶ to 10¹¹, 10⁷ to 10¹¹, 10⁸ to 10¹¹, 10⁹ to 10¹¹, 10¹⁰ to 10¹¹, 10² to 10¹⁰, 10³ to 10¹⁰, 10⁴ to 10¹⁰, 10⁵ to 10¹⁰, 10⁶ to 10¹⁰, 10⁷ to 10¹⁰, 10⁸ to 10¹⁰, 10⁹ to 10¹⁰, 10² to 10⁹, 10³ to 10⁹, 10⁴ to 10⁹, 10⁵ to 10⁹, 10⁶ to 10⁹ , 10⁷ to 10⁹, 10⁸ to 10⁹, 10² to 10⁸, 10³ to 10⁸, 10⁴ to 8¹¹, 10⁵ to 10⁸, 10⁶ to 10⁸, 10⁷ to 10⁸, 10² to 10⁷, 10³ to 10⁷, 10⁴ to 10⁷, 10⁵ to 10⁷, 10⁶ to 10⁷, 10² to 10⁶, 10³ to 10⁶, 10⁴ to 10⁶, 10⁵ to 10⁶, 10² to 10⁵, 10³ to 10⁵, 10⁴ to 10⁵, 10² to 10⁴, 10³ to 10⁴, 10² to 10³, 10¹², 10¹¹, 10¹⁰, 10⁹, 10⁸, 10⁷, 10⁶, 10⁵, 10⁴, 10³, or 10² total microbial cells per gram or milliliter of the composition.

In some embodiments, the administered dose of the microbial composition comprises 10² to 10¹⁸, 10³ to 10¹⁸, 10⁴ to 10¹⁸, 10⁵ to 10¹⁸, 10⁶ to 10¹⁸, 10⁷ to 10¹⁸, 10⁸ to 10¹⁸, 10⁹ to 10¹⁸, 10¹⁰ to 10¹⁸, 10¹¹ to 10¹⁸, 10¹² to 10¹⁸, 10¹³ to 10¹⁸, 10¹⁴ to 10¹⁸, 10¹⁵ to 10¹⁸, 10¹⁶ to 10¹⁸, 10¹⁷ to 10¹⁸, 10² to 10¹², 10³ to 10¹², 10⁴ to 10¹², 10⁵ to 10¹², 10⁶ to 10¹², 10⁷ to 10¹², 10⁸ to 10¹², 10⁹ to 10¹², 10¹⁰ to 10¹², 10¹¹ to 10¹², 10² to 10¹¹, 10³ to 10¹¹, 10⁴ to 10¹¹, 10⁵ to 10¹¹, 10⁶ to 10¹¹, 10⁷ to 10¹¹, 10⁸ to 10¹¹, 10⁹ to 10¹¹, 10¹⁰ to 10¹¹, 10² to 10¹⁰, 10³ to 10¹⁰, 10⁴ to 10¹⁰, 10⁵ to 10¹⁰, 10⁶ to 10¹⁰, 10⁷ to 10¹⁰, 10⁸ to 10¹⁰, 10⁹ to 10¹⁰, 10² to 10⁹, 10³ to 10⁹, 10⁴ to 10⁹, 10⁵ to 10⁹, 10⁶ to 10⁹, 10⁷ to 10⁹, 10⁸ to 10⁹, 10² to 10⁸, 10³ to 10⁸, 10⁴ to 10⁸, 10⁵ to 10⁸, 10⁶ to 10⁸, 10⁷ to 10⁸, 10² to 10⁷, 10³ to 10⁷, 10⁴ to 10⁷, 10⁵ to 10⁷, 10⁶ to 10⁷, 10² to 10⁶, 10³ to 10⁶, 10⁴ to 10⁶, 10⁵ to 10⁶, 10² to 10⁵, 10³ to 10⁵, 10⁴ to 10⁵, 10² to 10⁴, 10³ to 10⁴, 10² to 10³, 10¹⁸, 10¹⁷, 10¹⁶, 10¹⁵, 10¹⁴, 10¹³, 10¹², 10¹¹, 10¹⁰, 10⁹, 10⁸, 10⁷, 10⁶, 10⁵, 10⁴, 10³, or 10² total microbial cells.

In some embodiments, the composition is administered 1 or more times per month. In some embodiments, the composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10,3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8,8 to 10, 8 to 9, 9 to 10, 1, 2, 3, 4, 5, 6, 7, 8, 9, or 10 times per week.

In some embodiments, the microbial composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per month.

In some embodiments, the microbial composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per year.

In some embodiments, the microbial cells can be coated freely onto any number of compositions or they can be formulated in a liquid or solid composition before being coated onto a composition. For example, a solid composition comprising the microorganisms can be prepared by mixing a solid carrier with a suspension of the spores until the solid carriers are impregnated with the spore or cell suspension. This mixture can then be dried to obtain the desired particles.

In some embodiments, it is contemplated that the solid or liquid microbial compositions of the present disclosure further contain functional agents e.g., activated carbon, minerals, vitamins, and other agents capable of improving the quality of the products or a combination thereof.

In some embodiments, the microbes or microbial compositions of the present disclosure exhibit a synergistic effect, on one or more of the traits described herein, in the presence of one or more of the microbes or microbial compositions coming into contact with one another. The synergistic effect obtained by the taught methods can be quantified, for example, according to Colby's formula (i.e., (E)=X+Y−(X*Y/100)). See Colby, R. S., “Calculating Synergistic and Antagonistic Responses of Herbicide Combinations,” 1967. Weeds. Vol. 15, pp. 20-22, incorporated herein by reference in its entirety. Thus, “synergistic” is intended to reflect an outcome/parameter/effect that has been increased by more than an additive amount.

In some embodiments, the microbes or microbial compositions are administered in a time-release fashion between 1 to 5, 1 to 10, 1 to 15, 1 to 20, 1 to 24, 1 to 25, 1 to 30, 1 to 35, 1 to 40, 1 to 45, 1 to 50, 1 to 55, 1 to 60, 1 to 65, 1 to 70, 1 to 75, 1 to 80, 1 to 85, 1 to 90, 1 to 95, or 1 to 100 hours.

In some embodiments, the microbes or microbial compositions are administered in a time-release fashion between 1 to 2, 1 to 3, 1 to 4, 1 to 5, 1 to 6, 1 to 7, 1 to 8, 1 to 9, 1 to 10, 1 to 11, 1 to 12, 1 to 13, 1 to 14, 1 to 15, 1 to 16, 1 to 17, 1 to 18, 1 to 19, 1 to 20, 1 to 21, 1 to 22, 1 to 23, 1 to 24, 1 to 25, 1 to 26, 1 to 27, 1 to 28, 1 to 29, or 1 to 30 days.

### Soil Shift and Abundance of Microbes

In some embodiments, the soil microbiome comprises a diverse array of microbes with a wide variety of metabolic capabilities. In some embodiments, the present disclosure is drawn to administering any one of the microbial compositions described herein to modulate or shift soil microbiomes.

In some embodiments, the soil microbiome is shifted through the administration of any one of the microbes and/or microbial consortia to one or more layers or regions of the soil. In some embodiments, the microbiome is shifted through the administration of any one of the microbes and/or microbial consortia to the soil. In some embodiments, the soil microbiome shift or modulation includes a decrease or loss of specific microbes that were present prior to the administration of one or more microbes and/or microbial consortia of the present disclosure. In some embodiments, the microbiome shift or modulation includes an increase in microbes that were present prior to the administration of one or more microbes and/or microbial consortia of the present disclosure. In some embodiments, the microbiome shift or modulation includes a gain of one or more microbes that were not present prior to the administration of one or more microbes of the present disclosure. In a further embodiment, the gain of one or more microbes is a microbe that was not specifically included in the administered microbial composition. In some embodiments, the microbiome shift or modulation includes shifts in functional activities or gene expression of one or more microbes and/or microbial consortia in the soil.

In some embodiments, the administration of microbes and/or microbial consortia of the present disclosure results in a sustained modulation of the soil microbiome such that the administered microbes are present in the soil microbiome for a period of at least 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8,8 to 10, 8 to 9, 9 to 10, 1, 2, 3, 4, 5, 6, 7, 8, 9, or 10 days.

In some embodiments, the administration of microbes and/or microbial consortia of the present disclosure results in a sustained modulation of the soil microbiome such that the administered microbes are present in the soil microbiome for a period of at least 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8,8 to 10, 8 to 9, 9 to 10, 1, 2, 3, 4, 5, 6, 7, 8, 9, or 10 weeks.

In some embodiments, the administration of microbes and/or microbial consortia of the present disclosure results in a sustained modulation of the soil microbiome such that the administered microbes are present in the soil microbiome for a period of at least 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8,8 to 10, 8 to 9, 9 to 10, 1, 2, 3, 4, 5, 6, 7, 8, 9, 10, 11, or 12 months.

In some embodiments, administration of one or more microbes and/or microbial consortia results in a shift in the soil microbiome that increases the number and/or type of microbes belonging to one or more of the taxonomic groups disclosed herein by at least 0.5%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 200%, at least 300%, at least 400%, at least 500%, at least 600%, or at least 700%. In some embodiments, administration of one or more microbial composition results in a shift in the soil microbiome that increases the number and/or type of microbes belonging to one or more of the taxonomic groups disclosed herein by at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, or at least about 700%.

In some embodiments, administration of one or more microbes and/or microbial consortia results in a shift in the soil microbiome that decreases the number and/or type of microbes belonging to one or more of the taxonomic groups disclosed herein. In some embodiments, administration of one or more microbial composition results in a shift in the soil microbiome that reduces the number and/or type of microbes belonging to one or more of the taxonomic groups disclosed herein by at least 0.5%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, administration of one or more microbial composition results in a shift in the soil microbiome that decreases the number and/or type of microbes belonging to one or more of the taxonomic groups disclosed herein by at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

In some embodiments, administration of one or more microbes and/or microbial consortia results in a shift in the soil microbiome that decreases the number and/or type of microbes belonging to one or more of the taxonomic groups disclosed herein. In some embodiments, administration of one or more microbial composition results in a shift in the soil microbiome that reduces the number and/or type of microbes belonging to one or more of the taxonomic groups disclosed herein by at least 0.5%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, administration of one or more microbial composition results in a shift in the soil microbiome that decreases the number and/or type of microbes belonging to one or more of the taxonomic groups disclosed herein by at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

In some embodiments, administration of one or more microbes and/or microbial consortia results in a shift in the soil microbiome that increases the number and/or type of microbes belonging to one or more of the taxonomic groups disclosed herein. In some embodiments, administration of one or more microbial composition results in a shift in the soil microbiome that increases the number and/or type of microbes belonging to one or more of the taxonomic groups disclosed herein by at least 0.5%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 200%, at least 300%, at least 400%, at least 500%, at least 600%, or at least 700%. In some embodiments, administration of one or more microbial composition results in a shift in the soil microbiome that increases the number and/or type of microbes belonging to one or more of the taxonomic groups disclosed herein by at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, or at least about 700%.

### Carbon Amendments to Soil

The disclosure provides carbon amendments that impose directional selection for soil microbes using single and mixed nutrient amendments. Without being bound to a particular theory or mechanism of action, it is believed that soil carbon amendments can be used to modify nutrient utilization and to selectively enrich for advantageous soil-borne microbial populations (such as, for example, those with pathogen suppressive activity) among complex and highly-variable naturally-occurring soil microbial communities.

As used herein, the term “amendment” refers broadly to any material added to soil to improve its physical or chemical properties. As used herein, the terms “carbon-based soil amendment” or “carbon amendment” encompass any carbon-based material that, when added to the soil, yields an amended soil having improved physical or chemical properties. Non-limiting examples of carbon-based soil amendments include simple nutrients such as sugars, e.g. fructose, glucose, sucrose, lactose, galactose, dextrose, maltose, raffinose, ribose, ribulose, xylulose, xylose, amylase, arabinose, etc.; and sugar alcohols, e.g. adonitol, sorbitol, mannitol, maltitol, ribitol, galactitol, glucitol, etc., as well as complex substrates, including cellulose and lignin. In some embodiments, the carbon amendment comprises a combination of one or more simple nutrients, sugar alcohols or complex substrates disclosed herein.

Carbon amendments may be used at a concentration in the range of about 10 grams of carbon per meter-squared to about 500 grams of carbon per meter-squared, for example, about 50 grams of carbon per meter-squared, about 100 grams of carbon per meter-squared, about 200 grams of carbon per meter-squared, about 300 of carbon per meter-squared, about 400 of carbon per meter-squared, or about 500 of carbon per meter-squared, including all values and subranges that lie therebetween. In some embodiments, application of a carbon amendment to soil is accomplished by any known method in the art. In some embodiments, the carbon amendment is applied using standard agricultural equipment. In some embodiments, carbon amendments are applied as a liquid, granular, or seed-coating, in-furrow, to the soil, or to the foliage. The frequency of application of the carbon amendment may depend on several factors, including the nature of the soil, the type of carbon-based soil amendment, and the environmental conditions, as well as other factors. In some embodiments, the carbon amendment is applied at a frequency in the range of about several times a day to about once every few years, for example daily, weekly, every two weeks, monthly, or yearly, including all subranges and values that lie therebetween.

The disclosure provides methods of enhancing the antibiotic inhibitory capacity of individual microbes within a population of microbes in soil, said method comprising the steps of: applying a carbon source to said soil. The disclosure further provides methods of enriching the densities of inhibitory microorganisms within a population of microbes in soil, said method comprising the steps of: applying a carbon source to said soil. The disclosure further provides methods of suppressing the growth of pathogens within a soil containing microbes with soil-borne pathogen inhibitory potential, said method comprising the steps of: applying a carbon source to said soil. In some embodiments, the carbon source is selected from the group consisting of: glucose, fructose, lignin, ground rice powder, malic acid, and mixtures thereof. In some embodiments, the carbon source is applied at least 2, 3, 4, 5, 6, 7, 8, 9, 10, 11, or 12 times in a one year period.

The disclosure further provides methods of enhancing the antibiotic inhibitory capacity of individual microbes within a population of microbes in soil, wherein crops grown in said soil suffer from one or more soil-borne pathogens, said method comprising the steps of: a) applying a carbon source to the soil; b) assessing the antibiotic inhibitory capacity of microbes within the microbial population in the soil; and c) repeating steps (a) and (b) one or more times, until the antibiotic inhibitory capacity of microbes within the microbial population reaches a desired level. In some embodiments, the antibiotic inhibitory capacity of the microbes is assessed based on the presence or absence of symptoms exhibited by the crop due to the soil-borne pathogens. In some embodiments, steps (a) and (b) are repeated until the crops cease to exhibit symptoms from the soil-borne pathogens.

The disclosure provides methods of enriching the densities of inhibitory microorganisms within a population of microbes in soil, wherein crops grown in said soil suffer from one or more soil-borne pathogens, said method comprising the steps of: a) applying a carbon source to the soil; b) assessing the densities of inhibitory microorganisms within the soil; and c) repeating steps (a) and (b) one or more times, until the densities of inhibitory microorganisms within the soil reaches a desired level. In some embodiments, the densities of inhibitory microorganisms is assessed based on the presence or absence of symptoms exhibited by the crop due to the soil-borne pathogens. In some embodiments, steps (a) and (b) are repeated until the crops cease to exhibit symptoms from the soil-borne pathogens.

The disclosure provides methods of suppressing the growth of pathogens within a soil, containing microbes with soil-borne pathogen inhibitory potential, said method comprising the steps of: a) applying a carbon source to the soil; b) determining pathogen density in the soil; and c) repeating steps (a) and (b) one or more times, until the pathogen density reaches a desired level. In some embodiments, the densities of inhibitory microorganisms is assessed based on the presence or absence of pathogenic symptoms exhibited by a crop grown on the soil. In some embodiments, steps (a) and (b) are repeated until crops grown on the soil cease to exhibit symptoms from the soil-borne pathogens. In some embodiments, step (b) is conducted 1, 2, 3, 4, 5, 6, 7, 8, 9, 10, 11, or 12 months after step (a).

The disclosure also provides methods of enhancing the colonization of one or more microbial inoculants in the soil, said method comprising the steps of: applying a carbon source to said soil. In some embodiments, the method further comprises applying microbial inoculants to the soil. In some embodiments, the microbial inoculant may be applied before, at the same time as, or after applying the carbon source. In some embodiments, the microbial inoculant is a Streptomyces isolate. In some embodiments, the microbial inoculant is applied to the soil before planting. In some embodiments, the carbon source is selected from the group consisting of: cellulose, glucose, fructose, lignin, ground rice powder, malic acid, and mixtures thereof.

### Carbon Amendments+Microbial Application Combination Treatments

As was alluded to in the section above, carbon amendments to the soil can enhance the antibiotic inhibitory capacity of individual microbes within a population of microbes in soil, and can enrich the densities of inhibitory microorganisms within a population of microbes in soil. The present inventors leveraged this unexpected discovery by combining the microbial compositions and treatments of the present disclosure, with the carbon amendments disclosed above.

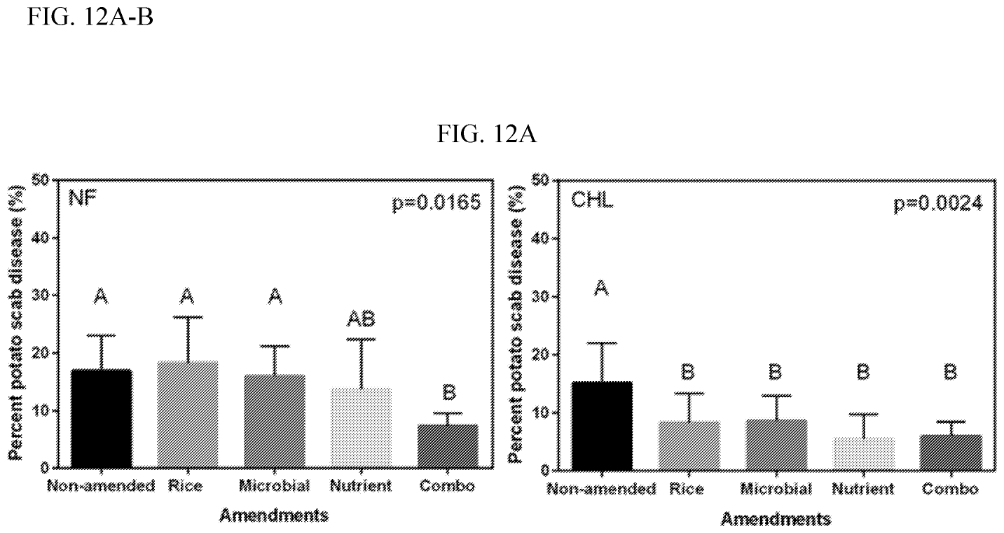

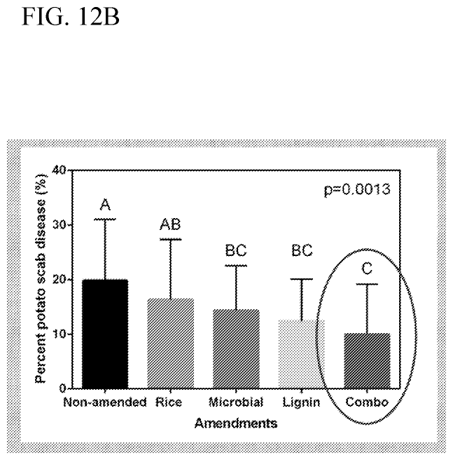

In some embodiments, carbon amendments can be used as an enhancer of microbial treatments of the present disclosure. That is, in some embodiments the present disclosure teaches that the co-administration of a carbon amendment with a microbial isolate or microbial consortia can enhance said microbial isolate or consortia's colonization or effectiveness (e.g., pathogen suppressive activity or plant growth enhancement activity). FIG. 12 A of the present disclosure for example, demonstrates that the co-administration of microbial isolate with carbon amendment (combo treatment) resulted in significant reductions in potato scab disease compared to non-amended soils. Indeed, the combination microbial+carbon amendment treatments also exhibited synergistic effects, providing significantly greater disease reduction than either the microbial treatment alone (microbial in figure), or carbon amendments by itself (nutrient in figure).

In some embodiments, the present disclosure teaches compositions comprising at least one microbial isolate and a carbon amendment. In some embodiments, the composition comprise a microbial consortia and a carbon amendment. In some embodiments the composition comprises a microbial isolate selected from the group consisting of Streptomyces GS1 (Streptomyces lydicus), Streptomyces PS1 (Streptomyces sp. 3211.1) and Brevibacillus PS3 (Brevibacillus laterosporus). In some embodiments, the microbial consortia comprises Streptomyces GS1 (Streptomyces lydicus), Streptomyces PS1 (Streptomyces sp. 3211.1) and Brevibacillus PS3 (Brevibacillus laterosporus).

### Prescriptive Biocontrol of Plant Pathogens

The disclosure provides methods for prescriptive biocontrol of a pathogen, for example, a soil-borne plant pathogen. In some embodiments, the method comprises (a) identifying the soil-borne plant pathogen(s) present in soil or plant tissue from a locus in need of prescriptive biocontrol; and (b) creating a customized soil-borne plant pathogen inhibiting microbial consortia capable of suppressing the growth of the soil-borne plant pathogen identified in step (a). In some embodiments, the step of creating said customized microbial consortia comprises: (i) accessing a soil-borne plant pathogen suppressive microbial library, said library comprising one or more ecological function balancing nodal libraries selected from the group consisting of: a mutual inhibitory activity microbial library, a carbon nutrient utilization complementarity microbial library, an antimicrobial signaling capacity and responsiveness microbial library, and a plant growth promotion ability microbial library; (ii) performing a multi-dimensional ecological function balancing (MEFB) nodal analysis utilizing said one or more nodal libraries; and (iii) selecting at least two microbes from the soil-borne plant pathogen suppressive microbial library based on the MEFB nodal analysis, thereby producing a soil-borne plant pathogen inhibiting microbial consortia, wherein said microbial consortia is capable of suppressing the growth of the soil-borne plant pathogen(s) identified in step (a). Further details of the “selecting at least two microbes” step are provided within this disclosure, including in the “assembling microbial consortia according to SPPIMC” section supra.

The disclosure further provides methods for prescriptive biocontrol of a soil-borne plant pathogen consortia, said method comprising: (a) identifying and/or culturing the soil-borne plant pathogen(s) present in soil or plant tissue from a locus in need of prescriptive biocontrol; and (b) creating a customized soil-borne plant pathogen inhibiting microbial consortia capable of suppressing the growth of the soil-borne plant pathogen identified and/or cultured in step (a). In some embodiments, the step of creating the customized microbial consortia comprises the steps of: accessing a soil-borne plant pathogen suppressive microbial library, utilizing microbes from the library of step i) to create one or more ecological function balancing nodal microbial libraries, selected from the group consisting of: a mutual inhibitory activity microbial library, a carbon nutrient utilization complementarity microbial library, an antimicrobial signaling capacity and responsiveness microbial library, and a plant growth promotion ability microbial library; performing a multi-dimensional ecological function balancing (MEFB) nodal analysis utilizing said one or more nodal microbial libraries; and selecting at least two microbes from the soil-borne plant pathogen suppressive microbial library based on the MEFB nodal analysis, thereby producing a soil-borne plant pathogen inhibiting microbial consortia, wherein said microbial consortia is capable of suppressing the growth of the soil-borne plant pathogen(s) identified in step (a).

In some embodiments, identifying the soil-borne plant pathogen(s) present in soil or plant tissue comprises identification of the pathogen genus based on symptoms of plants grown in said locus in need of prescriptive biocontrol. In some embodiments, the step of creating a mutual inhibitory activity microbial library comprises the steps of: i) assembling a library of test microbial consortia, each test consortia comprising a combination of at least two microbial isolates from the soil-borne plant pathogen suppressive microbial library; ii) screening test microbial consortia of the assembled library for the relative degree of mutual inhibitory activity displayed by each microbial isolate towards every other microbial isolate in the test microbial consortia; and iii) developing an n-dimensional mutual inhibitory activity matrix for test microbial consortia based on the mutual inhibitory activities screened in step (i).

In some embodiments, the step of creating a carbon nutrient utilization complementarity microbial library comprises the step of: i) screening a population of microbial isolates from the soil-borne plant pathogen suppressive microbial library for carbon nutrient utilization by growing said microbial isolates in a plurality of different nutrient media that comprise a distinct single carbon source to create a carbon nutrient utilization profile for each individual microbial isolate in said population.

In some embodiments, the step of creating a antimicrobial signaling capacity and responsiveness microbial library comprises the steps of: i) screening a population of microbial isolates from the soil-borne plant pathogen suppressive microbial library for the ability of each microbial isolate to signal and modulate the production of antimicrobial compounds in other microbial isolates from the population of microbial isolates; and/or ii) screening a population of microbial isolates from the soil-borne plant pathogen suppressive microbial library for the ability of each microbial isolate to be signaled and have their production of antimicrobial compounds modulated by other microbial isolates from the population of microbial isolates, thereby creating an antimicrobial signaling capacity and responsiveness profile for each screened individual microbial isolate.

The disclosure provides methods for prescriptive biocontrol of a soil-borne plant pathogen. In some embodiments, the method comprises identifying the soil-borne plant pathogen(s) present in soil or plant tissue from a locus in need of prescriptive biocontrol. In some embodiments, the method comprises creating a customized soil-borne plant pathogen inhibiting microbial consortia capable of suppressing the growth of the soil-borne plant pathogen identified in step (a). In some embodiments, creating said customized microbial consortia comprises the step of accessing a soil-borne plant pathogen suppressive microbial library. In some embodiments, the library comprises one or more ecological function balancing nodal libraries selected from the group consisting of: a mutual inhibitory activity microbial library, a carbon nutrient utilization complementarity microbial library, an antimicrobial signaling capacity and responsiveness microbial library, a plant growth promotion ability microbial library, and an antimicrobial resistance to clinical antimicrobials library. In some embodiments, creating said customized microbial consortia comprises the step of performing a multi-dimensional ecological function balancing (MEFB) nodal analysis utilizing said one or more nodal libraries. In some embodiments, creating said customized microbial consortia comprises the step of selecting at least two microbes from the soil-borne plant pathogen suppressive microbial library based on the MEFB nodal analysis, thereby producing a soil-borne plant pathogen inhibiting microbial consortia, wherein said microbial consortia is capable of suppressing the growth of the soil-borne plant pathogen(s) identified in step (a).

The disclosure provides methods for prescriptive biocontrol of a soil-borne plant pathogen. In some embodiments, the method comprises identifying and/or culturing the soil-borne plant pathogen(s) present in soil or plant tissue from a locus in need of prescriptive biocontrol. In some embodiments, the methods comprises creating a customized soil-borne plant pathogen inhibiting microbial consortia capable of suppressing the growth of the soil-borne plant pathogen identified and/or cultured in step (a). In some embodiments, creating said customized microbial consortia comprises the step of accessing a soil-borne plant pathogen suppressive microbial library. In some embodiments, creating said customized microbial consortia comprises the step of utilizing microbes from the library of step i) to create one or more ecological function balancing nodal microbial libraries, selected from the group consisting of: a mutual inhibitory activity microbial library, a carbon nutrient utilization complementarity microbial library, an antimicrobial signaling capacity and responsiveness microbial library, a plant growth promotion ability microbial library, and an antimicrobial resistance to clinical antimicrobials library. In some embodiments, creating said customized microbial consortia comprises the step of performing a multi-dimensional ecological function balancing (MEFB) nodal analysis utilizing said one or more nodal microbial libraries. In some embodiments, creating said customized microbial consortia comprises the step of selecting at least two microbes from the soil-borne plant pathogen suppressive microbial library based on the MEFB nodal analysis, thereby producing a soil-borne plant pathogen inhibiting microbial consortia, wherein said microbial consortia is capable of suppressing the growth of the soil-borne plant pathogen(s) identified in step (a).

In some embodiments, identifying the soil-borne plant pathogen(s) present in soil or plant tissue comprises identification of the pathogen genus based on symptoms of plants grown in said locus in need of prescriptive biocontrol. In some embodiments, the step of accessing a soil-borne plant pathogen suppressive microbial library comprises creating the soil-borne plant pathogen suppressive microbial library. In some embodiments, creating the soil-borne plant pathogen suppressive microbial library comprises screening a population of microbial isolates in the presence of the soil-borne plant pathogen identified and/or cultured in step (a), to create a soil-borne plant pathogen suppressive profile for each individual microbial isolate in said population. In some embodiments, the plant pathogen suppressive profile indicates each microbial isolate's ability to suppress the soil-borne plant pathogen identified and/or cultured in step (a).

In some embodiments, the step of creating or accessing a mutual inhibitory activity microbial library comprises the step of assembling a library of test microbial consortia, each test consortia comprising a combination of at least two microbial isolates from the soil-borne plant pathogen suppressive microbial library. In some embodiments, the step of creating or accessing a mutual inhibitory activity microbial library comprises the step of screening test microbial consortia of the assembled library for the relative degree of mutual inhibitory activity displayed by each microbial isolate towards every other microbial isolate within its own test microbial consortia. In some embodiments, the step of creating or accessing a mutual inhibitory activity microbial library comprises developing an n-dimensional mutual inhibitory activity matrix for test microbial consortia based on the mutual inhibitory activities screened in step (i).

In some embodiments, the step of creating or accessing a carbon nutrient utilization complementarity microbial library comprises the step of: i) screening a population of microbial isolates from the soil-borne plant pathogen suppressive microbial library for carbon nutrient utilization by growing said microbial isolates in a plurality of different nutrient media that comprise a distinct single carbon source to create a carbon nutrient utilization profile for each individual microbial isolate in said population.

In some embodiments, the step of creating an antimicrobial signaling capacity and responsiveness microbial library comprises the step of screening a population of microbial isolates from the soil-borne plant pathogen suppressive microbial library for the ability of each microbial isolate to signal and modulate the production of antimicrobial compounds in other microbial isolates from the population of microbial isolates. In some embodiments, the step of creating an antimicrobial signaling capacity and responsiveness microbial library comprises screening a population of microbial isolates from the soil-borne plant pathogen suppressive microbial library for the ability of each microbial isolate to be signaled and have their production of antimicrobial compounds modulated by other microbial isolates from the population of microbial isolates, thereby creating an antimicrobial signaling capacity and responsiveness profile for each screened individual microbial isolate.

In some embodiments, the step of creating an antimicrobial resistance to clinical antimicrobials library comprises the step of screening microbial isolates from the soil-borne plant pathogen suppressive microbial library for resistance to a plurality of antibiotics to create an n-dimensional antibiotic resistance profile.

In some embodiments, the step of creating a plant growth promotion ability microbial library comprises the step of applying microbial isolates from the soil-borne plant pathogen suppressive microbial library to a test plant. In some embodiments, the step of creating a plant growth promotion ability microbial library comprises the step of cultivating the test plant to maturity. In some embodiments, the step of creating a plant growth promotion ability microbial library comprises comparing the growth of the test plant against that of a control plant that did not receive the microbial isolate, wherein differences in the growth between the test plant and the control plant demonstrate a microbial isolate's plant growth promotion ability.

In some embodiments, the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii. In some embodiments, the soil-borne pathogen is selected from the group consisting of: species of Erwinia, Rhizomonas, Streptomyces scabies, Pseudomonas, and Xanthomonas.

### Prescriptive Biocontrol: Soil Carbon Quantity and Diversity

The disclosure provides methods for prescriptive biocontrol of a soil-borne plant pathogen. In some embodiments, the method comprises creating a soil nutrient profile from soil from a locus in need of prescriptive biocontrol. In some embodiments, the method comprises creating a customized carbon amendment for application on the locus of step a), wherein the customized carbon amendment supplements a carbon deficiency in the nutrient soil profile. In some embodiments, the method further comprises the step of c) applying the customized soil carbon amendment to the locus. In some embodiments, the method further comprises the step of repeating steps a)-b) one or more times. In some embodiments, each repetition of steps a)-b) occurs at least 1, 2, 3, 4 5 6, 7, 8, 9, 10, 11, or 12 months after the last carbon amendment application to the soil. In some embodiments, the step of creating a soil nutrient profile comprises the step of providing a soil sample from the locus in need of prescriptive biocontrol. In some embodiments, the step of creating a soil nutrient profile comprises analyzing the carbon nutrient contents of said soil sample.

In some embodiments, the carbon nutrient contents of the soil sample are measure via a chromatographic method. In some embodiments, the carbon nutrient contents of the soil sample are measure via an analysis method selected from the group consisting of: Gas Chromatography, Liquid Chromatography, Mass Spectrometer, wet digestion and dry combustion, aerial spectroscopy, Loss on Ignition, Elemental Analyzer, and Reflectance Spectroscopy.

In some embodiments, the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii. In some embodiments, the soil-borne pathogen is selected from the group consisting of: species of Erwinia, Rhizomonas, Streptomyces scabies, Pseudomonas, and Xanthomonas.

The disclosure provides methods of enhancing the antibiotic inhibitory capacity of individual microbes within a population of microbes in soil, said method comprising the steps of: applying a carbon source to said soil. The disclosure provides methods of enriching the densities of inhibitory microorganisms within a population of microbes in soil, said method comprising the steps of: applying a carbon source to said soil. The disclosure provides methods of suppressing the growth of pathogens within a soil containing microbes with soil-borne pathogen inhibitory potential, said method comprising the steps of: applying a carbon source to said soil. In some embodiments, the carbon source is selected from the group consisting of: glucose, fructose, lignin, ground rice powder, malic acid, and mixtures thereof. In some embodiments, the carbon source is applied at least 2, 3, 4, 5, 6, 7, 8, 9, 10, 11, or 12 times in a one year period.

The disclosure of enhancing the antibiotic inhibitory capacity of individual microbes within a population of microbes in soil, wherein crops grown in said soil suffer from one or more soil-borne pathogens. In some embodiments, the method comprises applying a carbon source to the soil. In some embodiments, the method comprises assessing the antibiotic inhibitory capacity of microbes within the microbial population in the soil. In some embodiments, the method comprises repeating steps (a) and (b) one or more times, until the antibiotic inhibitory capacity of microbes within the microbial population reaches a desired level. In some embodiments, the antibiotic inhibitory capacity of the microbes is assessed based on the presence or absence of symptoms exhibited by the crop due to the soil-borne pathogens. In some embodiments, steps (a) and (b) are repeated until the crops cease to exhibit symptoms from the soil-borne pathogens.

The disclosure provides methods of enriching the densities of inhibitory microorganisms within a population of microbes in soil, wherein crops grown in said soil suffer from one or more soil-borne pathogens. In some embodiments, the method comprises applying a carbon source to the soil. In some embodiments, the method comprises assessing the densities of inhibitory microorganisms within the soil. In some embodiments, the method comprises repeating steps (a) and (b) one or more times, until the densities of inhibitory microorganisms within the soil reaches a desired level.

In some embodiments, the densities of inhibitory microorganisms is assessed based on the presence or absence of symptoms exhibited by the crop due to the soil-borne pathogens. In some embodiments, steps (a) and (b) are repeated until the crops cease to exhibit symptoms from the soil-borne pathogens.

The disclosure provides methods of suppressing the growth of pathogens within a soil, containing microbes with soil-borne pathogen inhibitory potential. In some embodiments, the method comprises the steps of applying a carbon source to the soil. In some embodiments, the method comprises determining pathogen density in the soil. In some embodiments, the method comprises repeating steps (a) and (b) one or more times, until the pathogen density reaches a desired level. In some embodiments, the densities of inhibitory microorganisms is assessed based on the presence or absence of pathogenic symptoms exhibited by a crop grown on the soil. In some embodiments, steps (a) and (b) are repeated until crops grown on the soil cease to exhibit symptoms from the soil-borne pathogens. In some embodiments, step (b) is conducted 1, 2, 3, 4, 5, 6, 7, 8, 9, 10, 11, or 12 months after step (a).

The disclosure provides methods of treating a soil-borne pathogen in soil. In some embodiments, the method comprises applying a combination composition to the soil. In some embodiments, the composition comprises a soil-borne pathogen suppressing microbe a carbon source; thereby reducing the symptoms of the soil-borne pathogen on a crop grown in said soil.

In some embodiments, the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii. In some embodiments, the soil-borne pathogen is selected from the group consisting of: species of Erwinia, Rhizomonas, Streptomyces scabies, Pseudomonas, and Xanthomonas.

In some embodiments, the soil-borne pathogen suppressing microbe is a microbial isolate. In some embodiments, the microbial isolate is selected from the group consisting Streptomyces GS1 (Streptomyces lydicus), Streptomyces PS1 (Streptomyces sp. 3211.1) and Brevibacillus PS3 (Brevibacillus laterosporus). In some embodiments, the soil-borne pathogen suppressing microbe is administered as a microbial consortia. In some embodiments, the microbial consortia comprises Streptomyces GS1 (Streptomyces lydicus), Streptomyces PS1 (Streptomyces sp. 3211.1) and Brevibacillus PS3 (Brevibacillus laterosporus).

The disclosure provides compositions comprising i) a soil-borne pathogen suppressing microbe; and i) a carbon source, wherein said composition is capable of suppressing the growth of a soil-borne pathogen. In some embodiments, the soil-borne pathogen suppressing microbe is a microbial isolate. In some embodiments, the microbial isolate is selected from the group consisting Streptomyces GS1 (Streptomyces lydicus), Streptomyces PS1 (Streptomyces sp. 3211.1) and Brevibacillus PS3 (Brevibacillus laterosporus). In some embodiments, the soil-borne pathogen suppressing microbe is administered as a microbial consortia. In some embodiments, the microbial consortia comprises Streptomyces GS1 (Streptomyces lydicus), Streptomyces PS1 (Streptomyces sp. 3211.1) and Brevibacillus PS3 (Brevibacillus laterosporus). In some embodiments, the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii. In some embodiments, the soil-borne pathogen is selected from the group consisting of: species of Erwinia, Rhizomonas, Streptomyces scabies, Pseudomonas, and Xanthomonas.

### Sequences of the Present Disclosure

A summary of the sequences of the present disclosure, included in the sequence listing, is provided in Table 8, below:

TABLE 8

| SEQ ID | | | |
|---|---|---|---|
| NO. | Description | Organism | Remarks |
| 1 | 16S rRNA of | Streptomyces | 16S rRNA, small subunit |
| | microbial isolate | lydicus | ribosomal RNA |
| | GS1 | | |
| 2 | 16S rRNA of | Streptomyces | 16S rRNA, small subunit |
| | microbial isolate | sp. 3211.1 | ribosomal RNA |
| | PS1 | | |
| 3 | 16S rRNA of | Brevibacillus | Sequence obtained from Sanger |
| | microbial isolate | laterosporus | sequencing of the 16S rRNA |
| | PS3 | | using forward primer |
| 4 | 16s rRNA of | Brevibacillus | Sequence obtained from Sanger |
| | microbial isolate | laterosporus | sequencing of the 16S rRNA |
| | PS3 | | using reverse primer |
| 5 | 16s rRNA of | Brevibacillus | Reverse complement of SEQ |
| | microbial isolate | laterosporus | ID No. 4 |
| | PS3 | | |

It is to be understood that the description above as well as the examples that follow are intended to illustrate, and not limit, the scope of the invention. Other aspects, advantages and modifications within the scope of the invention will be apparent to those skilled in the art to which the invention pertains.

### EXAMPLES

### Example 1

### Soil-Borne Plant Pathogen Inhibiting Microbial Consortia (SPPIMC) Development Platform

The SPPIMC platform enables the identification of soil-borne microbes, which have plant pathogen-inhibiting activity. Furthermore, the platform enables the generation of multi-strain compositions (interchangeably referred to herein as “consortium”) comprising two or more plant pathogen-inhibiting microbes, that have superior properties (for example, capability to inhibit a wider range of plant pathogens and capability to utilize a wider range of nutrients) as compared to the individual microbes. A microbial consortia developed using this platform and its superior properties are described in Example 9.

FIG. 14 illustrates a flowchart of the Soil-borne Plant Pathogen Inhibiting Microbial Consortia (SPPIMC) development platform. The first step of the platform involves creating an enriched microbial library. Secondly, the pathogen suppressive activity of each of the isolates is determined. Subsequently, a multi-dimensional ecological function balancing (MEFB) Nodal Analysis is performed, which comprises analyzing the different characteristics of the isolates, which are important for its application as a pathogen suppressant. The MEFB Nodal Analysis includes nodes such as determining mutual inhibition activities, determining nutrient utilization complementarity, determining antimicrobial signaling/responsiveness capacity, and determining plant growth promotion ability. The MEFB Nodal Analysis can be performed in a serial, parallel, and/or iterative manner. FIG. 15 shows the steps comprising each of these analysis nodes. Each of the “nodes” of the platform are depicted in greater detail in the Examples below.

### Example 2

### Creating a Bacterial Library Exhibiting Antimicrobial Activity Against Plant Pathogens

Over one thousand Streptomyces, Bacillus, Fusarium and Pseudomonas isolates were collected across all continents, save for Asia, from agricultural soils, forest soils, prairie soils, wetland soils, savannah soils, and peat soils.

The bacteria were isolated using standard nutrient medium (including water agar, oatmeal agar, sodium caseinate agar, and others). Habitats were chosen both to capture a wide range of ecological conditions, but also to target settings where: i) inhibitory phenotypes are likely to be enriched (Kinkel, L. L., Schlatter, D. S., Bakker, M. B., and Arenz, B. (2012). Streptomyces competition and coevolution in relation to disease suppression. Research in Microbiology: dx.doi.org/10.1016/j.resmic.2012.07.005); or ii) long-term agricultural management has been imposed.

Microbial isolates collected according to the methods of this Example can be saved into an “Enriched Microbial Library” for use in producing valuable microbial consortia with agricultural uses. In some embodiments, the Enriched Microbial Library also includes information regarding each collected microbial isolate, including information from one or more of the Multi-Dimensional Ecological Function Balancing (MEFB) Nodal Analysis discussed in the Examples below, and illustrated in FIG. 14. Thus, the Enriched Microbial Library can, in some embodiments, comprise not only a group of collected microbial isolates, but also a database comprising information about said microbial isolates, including their pathogen suppressive activity, their mutual inhibition activity, their nutrient utilization complementarity, their antimicrobial signaling/responsiveness capacity, and their plant growth promotion ability.

### Example 3

### Characterization of Isolates of the Bacterial Library for Antimicrobial Activity Against Plant Pathogens (“Determining Pathogen Suppressive Activity”).

As shown in FIG. 14, each of the microbes in the library was subjected to one of the nodes involved with “multi-dimensional ecological function balancing (MEFB) nodal analysis,” i.e. “determine pathogen suppressive activity” as described below.

### Methods:

Fungal pathogen working stock isolates were grown on 0.5× potato dextrose agar (15 ml) for two (Rhizoctonia sp. and Sclerotinia sp.) or five days (Fusarium graminearum and F. oxysporum) on the benchtop (22° C.-25° C.). Fusarium virguliforme cultures were grown on SMDA (Soymilk Dextrose Agar) for approximately 20 days in the dark at (22° C.-25° C.). Pythium sp. were grown on V8 agar for two days on the benchtop (22° C.-25° C.). Phytophthora sp. were grown on V8 agar for two days on the benchtop (22° C.-25° C.). Tested pathogens included the causative agents of scab and Verticillium wilt (V. dahliae or V. albo-atrum). The pathogens also include species of Phytophthora, Pythium, Rhizoctonia, Sclerotinia, and Fusarium virguliforme.

Antagonist Streptomyces isolates were dotted in 5 μl aliquot from a frozen 20% glycerol spore suspension onto plates containing 15 ml of oatmeal agar (OA) (if plates are being tested against Pythium sp. or Phytophthora sp. pathogen isolates, the Streptomyces were plated on V8 agar), evenly spaced around a plate/3 isolates per plate. There was a replicate plate of each dotted antagonist isolate series. Inoculated oatmeal agar plates were then incubated for three days at 28° C. After three days, the Streptomyces isolates were killed by inverting plates over a watchglass containing 4 ml of chloroform for one hour. After one hour, plates were moved to a Class II Type A2 Biological Safety Cabinet (BSC) and the plates were opened to allow the residual chloroform to evaporate (30 min). This process kills or prevents the Streptomyces isolate from producing more antibiotic and allows residual chloroform to evaporate so that the growth of the fungal isolates will not be affected. Antibiotics already produced by Streptomyces will have diffused into the medium prior to killing.

Subsequently, the chloroformed plates were inoculated with the fungal pathogen. An 8-mm plug of medium was removed from the center of the plate containing the now killed antagonist isolates using a cork borer, and it was replaced with an 8 mm plug of medium containing the target fungal pathogen (selected from the growing margin of the fungal pathogen working stock cultures described above), mycelium-side facing up.

For plates inoculated with Rhizoctonia solani and Sclerotinia sclerotiorum, once the fungal plug was added, plates were parafilmed and placed on the benchtop for 3 days to incubate at room temp (22° C.-25° C.). For Fusarium oxysporum, Fusarium graminearum, and Phytophthora sp. (on V8 agar) pathogen assays, the plug was added, and plates were parafilmed and placed on the benchtop to incubate for seven days at room temp. Plates with Fusarium virguliforme were incubated in the dark (in a cardboard box placed on a lab bench) for 21 days (3 weeks) at room temp. The plates containing the Pythium sp. isolate (which are grown on V8 agar) were incubated on the lab bench at room temp for three days.

Fungal isolates grew radially from the center of the plate (where the plug had been placed) to the edge of the petri plate, except for zones in which pathogen-inhibitory antibiotics produced by the Streptomyces were present in the medium. These “clearing zone(s)” or “cleared zone(s)” of no growth were measured and represent a measure of pathogen-inhibitory capacity of the Streptomyces isolate, as described above. The inhibition zone was quantified with two, 90° length measurements from the edge of the killed Streptomyces dotted isolate to the edge of the cleared zone where fungal pathogen isolates had not grown.

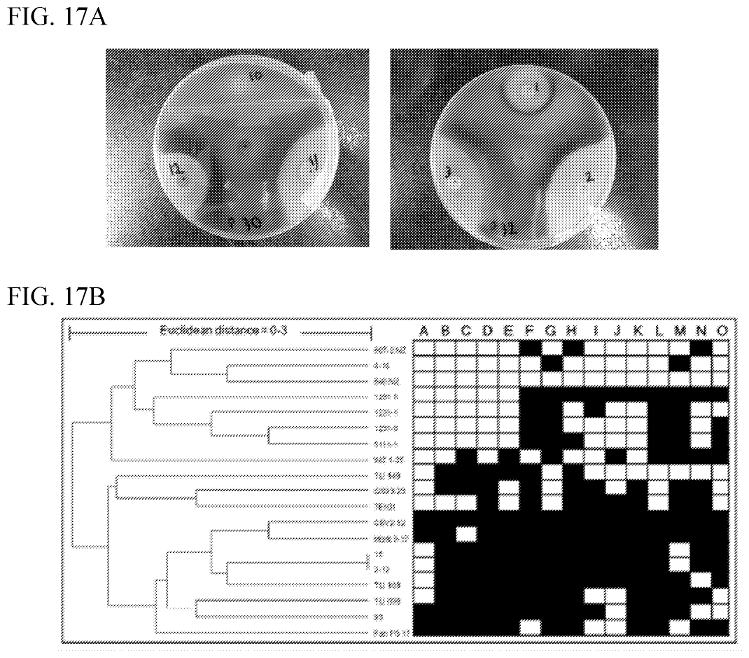

The growth of the pathogens was evaluated in relation to each bacterium, with inhibition evidenced by clear zones of inhibition of pathogen growth surrounding colonies of the bacterial isolates (i.e. cleared zone). Each bacterial isolate of the library is indicated as having antimicrobial activity (+) or not having antimicrobial activity (−) against each soil-borne plant pathogen utilized in characterizing the isolates of the bacterial library. In addition, the degree of pathogen inhibition by each isolate is quantified by measuring the zone of inhibition of that pathogen (in mm, see FIG. 17A for illustration of an inhibition assay).

Based on the results of the pathogen suppressive activity assays, as shown in Table 1, two or more Streptomyces isolates that have complementary abilities to inhibit the different pathogens and pathogenic isolates listed above, may be selected and combined to form a “multi strain inoculant composition” (interchangeably referred to herein as “consortium”).

### Results:

FIG. 17A shows an example of pathogen inhibition assays. The left panel of this figure shows inhibition of plant pathogenic Fusarium by Streptomyces isolates 11 (bottom right of petri dish) and 12 (bottom left of petri dish), but not 10 (top of petri dish). The right panel of this figure shows inhibition of plant pathogenic Fusarium by Streptomyces isolates 1 (top of petri dish), 2 (bottom right of petri dish), and 3 (bottom left of petri dish).

Table 1 shows the zone of inhibition (mm) for each of the plant pathogens tested in this example in the presence of Streptomyces isolates tested herein.

TABLE 1

| | Plant pathogen | | | | | | | | | | | |
|---|---|---|---|---|---|---|---|---|---|---|---|---|
| | | Genus/species | | | | | | | | | | |
| | | | | | | | | | | | Pythium | |
| | | Rhizoctonia | Sclerotinia | Fusarium | | Fusarium | | Phytophthora | | Phytophthora | ultimum var. | F. |
| | | solani | sclerotiorum | oxysporum | | graminearum | | medicaginis | | sojae | sporangiferum | culmorum |
| | | Plant pathoge | | | | | | | | | | |
| | | P6 | P19 | P27 | P29 | P31 | P34 | P43 | P44 | P52 | P58 | P61 |
| Name of | SS6 | 7.5 | 9.43 | 5.2 | 0 | 8.63 | 9.73 | 0 | 0 | 0 | 0 | 11.73 |
| Streptomyces | SS2 | 9.03 | 12.9 | 4.8 | 6.7 | 12.1 | 15.85 | 12.78 | 13.7 | 0 | 16.68 | 11.43 |
| | GS1 | 9.15 | 8.63 | 0 | 0 | 8.63 | 16.28 | 4.53 | 10.45 | 0 | 9.95 | 4.68 |
| | SS3 | 12.68 | 6.85 | 0 | 6.1 | 11.05 | 9.53 | 0 | 0 | 0 | 0 | 14.8 |
| | PS1 | 0 | 0 | 0 | 0 | 3.05 | 0 | 0 | 0 | 0 | 0 | 0 |
| | SS7 | 0 | 12.03 | 0 | 6.35 | 8.53 | 8.88 | 0 | 0 | 10.1 | 0 | 8.05 |
| | PS2 | 11.6 | 16.2 | 0 | 3.9 | 10.6 | 0 | 1.8 | 1.38 | 0 | 0 | 7.85 |
| | SS8 | 7.75 | 15.1 | 0 | 4.73 | 9.33 | 9.18 | 0 | 0 | 8.75 | 0 | 12.55 |
| | PS3 | 12.25 | 11.48 | 0 | 0 | 10.35 | 14.58 | 12.55 | 6.53 | 0 | 3.73 | 5.48 |

FIG. 17B shows an example of complementarity in pathogen inhibition profiles among compiled from a series of pathogen suppressive activity assay conducted for a collection of Streptomyces. Across the top of the grid, A-O represent different isolates of the plant pathogen, Streptomyces scabies (columns). From top-to-bottom along the center of the table are the identities of a collection of pathogen-inhibitory populations. Darkened boxes indicate interactions in which the specific pathogen-inhibitory isolate inhibited the pathogen. The dendrogram on the left-hand side quantifies the relatedness in inhibitory phenotypes among pathogen-inhibitory populations. FIG. 17B shows that the Streptomyces isolates listed in the bottom half of the grid are inhibitory towards a wider range of S. scabies isolates, as compared to the Streptomyces isolates listed in the top half of the grid. The results also suggest that isolates that are differentiated by a larger Euclidean distance differ more in their inhibitory profiles than isolates separated by a smaller Euclidean distance. However, distance alone is inadequate for optimizing inhibitory capacities, as the total difference between isolates is less important than complementarity in their collective inhibitory capacities.

In some embodiments, the present disclosure teaches selecting microorganisms that have complementary abilities to inhibit the different pathogens affecting plant growth. The selected microorganisms with pathogen suppressive activity may be combined to form a “multi strain inoculant composition” (interchangeably referred to herein as “consortium”)

Such a consortium would then be expected to have an ability to inhibit a wider range of different pathogens than any one isolate present in the consortium. For example, while GS1 inhibits P6 isolate of Rhizoctonia solani, SS7 does not. On the other hand, SS7 inhibits P52 isolate of Phytophthora sojae, while GS1 does not. Based on these data, a consortium containing GS1 and SS7 would be expected to inhibit both P6 isolate of Rhizoctonia solani and P52 isolate of Phytophthora sojae. In addition, adopting the other nodes featured in FIG. 14, and as described further below, novel consortiums of microbial isolates with unique capabilities to inhibit a wide range of plant pathogens can be generated.

Results from the pathogen suppressive activity assays were entered into the enriched microbial library for storage and additional analysis as part of the presently disclosed MEFB nodal analysis.

### Example 4

### Characterization of the Microbial Library for Antimicrobial Resistance to Clinical Antimicrobials

Indigenous soil populations produce a wide array of antibiotics. Resistance to these naturally-produced antibiotics is thus an important attribute for successful inoculants. As shown in FIG. 14, each of the microbes in the library was subjected to one of the nodes involved with “multi-dimensional ecological function balancing (MEFB) nodal analysis,” i.e. “determine antimicrobial resistance to clinical antimicrobials” as described below.

### Methods:

Isolates of a previously generated enriched microbial library collected according to the methods of Example 1 were grown on solid medium in the presence of clinical antimicrobial agents such as tetracycline, chloramphenicol, vancomycin, erythromycin, novobiocin, streptomycin, azithromycin, kanamycin, rifampin, or other antibiotics. Plates containing 15 ml of Starch Casein Agar (SCA) were spread plated with 150 μl of the test bacterial isolate. Next, immediately after plating the bacteria, three replicate antibiotic discs (Amoxicillin/Clavulanic Acid-30 Novobiocin-5 μg & 30 μg, Rifampin 5 Vancomycin 5 μg & 30 μg, Tetracycline 30 μg, Kanamycin 30 μg, Erythromycin 15 μg, Streptomycin 10 μg, and Chloramphenicol 30 μg; antibiotics obtained from Becton, Dickinson, and Company) were placed onto each petri dish. Plates were then incubated at 28° C. for 5 days.

After the incubation period, the length of the inhibition zone from the edge of the antibiotic disc to the edge of where the microbial isolate could not grow was measured at 90° angles and recorded to measure microbial susceptibility to the antibiotic on the disc. Each microbial isolate is indicated as being resistant (inhibition zone=0) or susceptible (inhibition zone>1 mm), with the degree of susceptibility indexed by the size of the zone (greater zone size =more susceptible).

### Results:

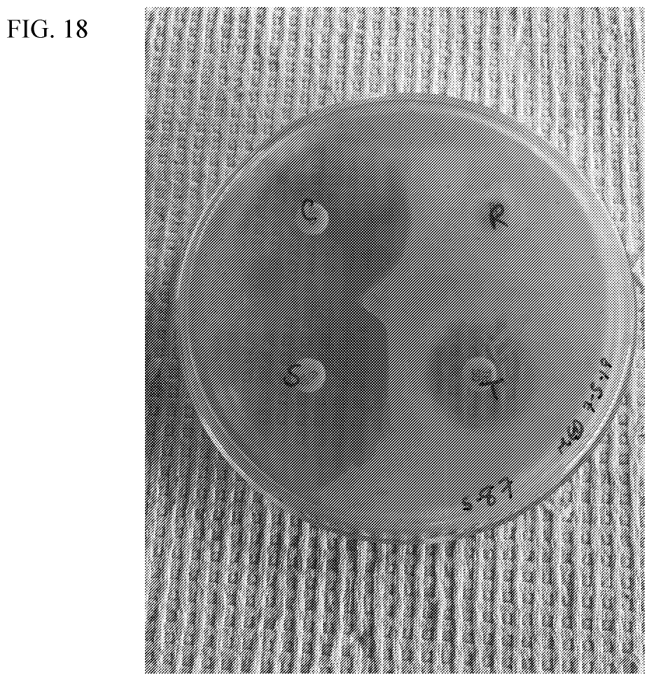

The data show that Streptomyces isolate, S-87, is very strongly inhibited by both chloramphenicol and streptomycin (large clearing zones of inhibition), but less so by tetracycline (smaller zone of inhibition) or rifampin (very small inhibition zone). See FIG. 18

Results from the antimicrobial resistance assays were entered into the enriched microbial library for storage and additional analysis as part of the presently disclosed MEFB nodal analysis.

### Example 5

### Characterization of the Bacterial Library Mutual Inhibition Activity Exhibited Between Bacteria of the Library (“Determine Mutual Inhibition Activity”).

This example illustrates the “determine mutual inhibition activity” node of the “multi-dimensional ecological function balancing (MEFB) nodal analysis,” shown in FIG. 14.

### Methods:

Isolates of a previously generated enriched microbial library collected according to the methods of Example 1 were grown in the presence of other isolates in the microbial library. The growth of the microbial isolates was evaluated for the presence or absence of zones of inhibition at the interphase between the bacterial isolates. The inhibition zone was quantified with two, 90° length measurements from the edge of the dotted isolate to the edge of the cleared zone where the overlaid isolate did not grow.

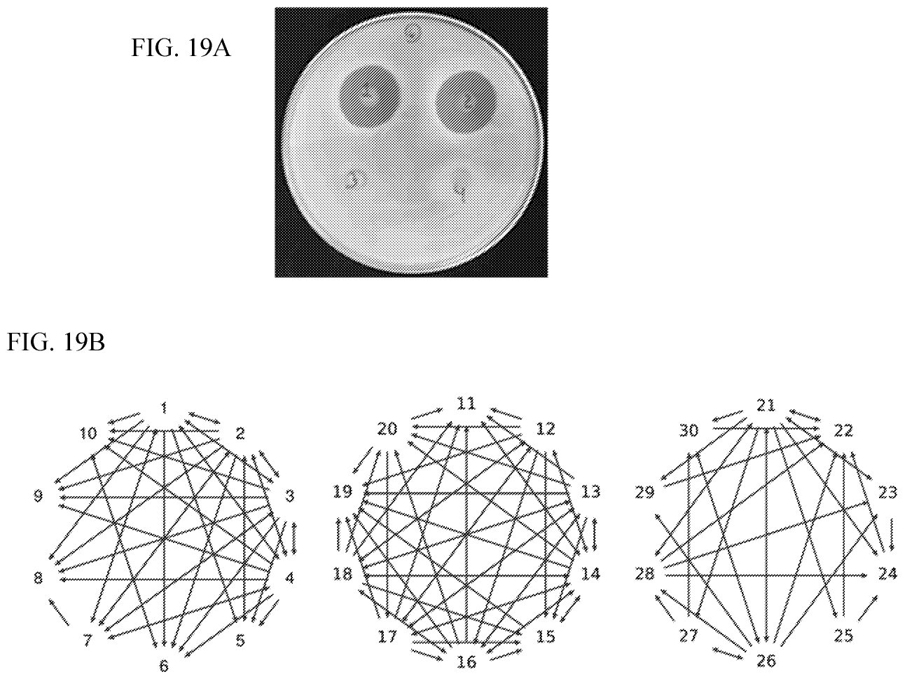

Each interaction was indicated as inhibitory (inhibition zone greater than 1 mm) or non-inhibitory (inhibition zone=0), with the inhibition zone size serving as a quantitative metric of sensitivity (high zone size=high sensitivity). Data from these experimental tests can be visualized as a network of bacterial isolates representing their inhibitory relationships. The resulting networks can be utilized to determine whether combining two or more microbial isolates in a consortia would result in death or coexistence of one or more of the microbial isolates—allowing for optimal bacterial isolate combinations (FIG. 19). See also, Table 2 below.

### Results:

FIG. 19A shows an illustrative pairwise inhibition assay between Streptomyces isolates. In this case, isolates 1-4 were dotted onto the plate and allowed to grow for 3 days. Subsequently, the colonies were killed by inverting plates over a watchglass containing 4 ml of chloroform for one hour. After one hour, plates were moved to a Class II Type A2 Biological Safety Cabinet (BSC) and the plates were opened to allow the residual chloroform to evaporate (30 min). Then, isolate 6 was overlaid onto the plate. The figure shows that Isolates 1 and 2 (top left and top right of petri dish, respectively) inhibit isolate 6, because of the cleared zone surrounding these isolates in which isolate 6 was not able to grow. In contrast, isolates 3 and 4 (bottom left and right of petri dish, respectively) do not inhibit isolate 6, because isolate 6 was able to grow adjacent to the tested isolates, without any cleared zone surrounding the initial dotted isolates. The pairwise inhibition assay described above was repeated with a collection of microbial isolates from the enriched microbial library, the results of which are presented below in Table 2.

Table 2A-2C: Inhibitory interactions among Streptomyces populations coexisting in soil. Numbers indicate the inhibition zone size (mm) of each inhibitory isolate (column) against each of the coexisting isolates (rows). Locations A, B, and C correspond to the first, second, and third interaction networks, respectively, illustrated in FIG. 19B. The networks represent any inhibitory interaction with a zone size greater than 1.0 mm.

TABLE 2A
Location A

| | Inhibitory Isolate | | | | | | | | | |
|---|---|---|---|---|---|---|---|---|---|---|
| | 1 | 2 | 3 | 4 | 5 | 6 | 7 | 8 | 9 | 10 |
| 1 | . | 19.3 | 16.3 | 14.3 | 19.3 | 18.3 | 13.7 | 20.3 | 14.3 | 13.0 |
| 2 | 19.3 | . | 17.3 | 19.7 | 24.0 | 17.3 | 15.7 | 20.3 | 14.3 | 13.3 |
| 3 | 14.0 | 22.3 | . | 20.3 | 23.0 | 17.0 | 14.3 | 19.7 | 13.3 | 13.0 |
| 4 | 12.0 | 16.7 | 17.3 | . | 18.0 | 18.3 | 14.7 | 17.7 | 13.0 | 12.7 |
| 5 | 0.3 | 9.3 | 0.3 | 0.3 | . | 0.0 | 0.0 | 0.0 | 0.0 | 0.0 |
| 6 | 0.0 | 0.0 | 0.0 | 0.0 | 0.0 | . | 0.0 | 0.0 | 0.0 | 7.0 |
| 7 | 20.3 | 21.3 | 2.3 | 4.3 | 0.0 | 0.0 | . | 1.0 | 0.0 | 0.0 |
| 8 | 0.0 | 0.0 | 0.3 | 0.0 | 0.7 | 0.0 | 1.0 | . | 0.0 | 0.0 |
| 9 | 0.0 | 0.0 | 0.0 | 0.0 | 0.0 | 0.0 | 0.0 | 0.0 | . | 0.3 |
| 10 | 0.0 | 0.0 | 0.0 | 0.7 | 0.0 | 4.0 | 0.0 | 0.0 | 0.0 | . |

TABLE 2B
Location B

| | Inhibitory Isolate | | | | | | | | | |
|---|---|---|---|---|---|---|---|---|---|---|
| | 11 | 12 | 13 | 14 | 15 | 16 | 17 | 18 | 19 | 20 |
| 11 | . | 0.0 | 0.0 | 10.5 | 0.0 | 13.5 | 0.0 | 0.0 | 0.0 | 0.0 |
| 12 | 15.5 | . | 0.0 | 9.5 | 13.0 | 24.7 | 16.0 | 8.0 | 25.5 | 22.0 |
| 13 | 20.5 | 0.0 | . | 15.0 | 17.5 | 21.0 | 19.0 | 18.7 | 30.5 | 19.5 |
| 14 | 16.5 | 1.7 | 1.0 | . | 36.0 | 34.0 | 46.0 | 32.0 | 46.0 | 36.0 |
| 15 | 0.0 | 0.0 | 0.0 | 17.5 | . | 0.0 | 0.0 | 0.0 | 1.3 | 0.0 |
| 16 | 17.0 | 0.0 | 2.0 | 16.0 | 24.0 | . | 0.0 | 1.3 | 7.3 | 2.0 |
| 17 | 17.3 | 18.7 | 11.3 | 22.0 | 10.0 | 15.0 | . | 0.0 | 37.0 | 0.0 |
| 18 | 34.0 | 17.7 | 20.0 | 14.5 | 16.3 | 35.0 | 0.0 | . | 37.7 | 1.0 |
| 19 | 0.0 | 0.0 | 0.0 | 0.0 | 0.0 | 0.5 | 0.0 | 0.0 | . | 0.0 |
| 20 | 6.0 | 0.0 | 0.0 | 0.0 | 0.0 | 0.0 | 5.5 | 0.0 | 24.5 | . |

TABLE 2C
Location C

| | Inhibitory Isolate | | | | | | | | | |
|---|---|---|---|---|---|---|---|---|---|---|
| | 21 | 22 | 23 | 24 | 25 | 26 | 27 | 28 | 29 | 30 |
| 21 | . | 15.0 | 14.0 | 16.7 | 19.5 | 18.0 | 17.5 | 13.5 | 16.5 | 18.0 |
| 22 | 5.5 | . | 0.0 | 0.0 | 0.0 | 0.0 | 0.0 | 1.0 | 0.0 | 0.0 |
| 23 | 8.0 | 0.0 | . | 21.7 | 0.0 | 0.0 | 0.0 | 0.0 | 0.0 | 0.0 |
| 24 | 0.0 | 11.0 | 0.0 | . | 0.0 | 0.0 | 0.0 | 0.0 | 0.0 | 0.3 |
| 25 | 0.0 | 9.0 | 0.0 | 18.0 | . | 0.0 | 0.0 | 0.0 | 0.0 | 0.0 |
| 26 | 6.3 | 1.7 | 1.7 | 1.0 | 0.5 | . | 13.5 | 7.0 | 1.7 | 5.0 |
| 27 | 0.0 | 0.0 | 0.0 | 0.0 | 0.0 | 11.5 | . | 8.0 | 0.0 | 21.0 |
| 28 | 16.0 | 7.5 | 0.0 | 18.0 | 0.0 | 0.7 | 0.0 | . | 0.0 | 0.0 |
| 29 | 0.0 | 9.5 | 0.0 | 0.0 | 0.0 | 0.0 | 0.0 | 0.0 | . | 0.0 |
| 30 | 0.0 | 11.5 | 0.0 | 0.0 | 0.0 | 3.3 | 0.0 | 0.0 | 0.0 | . |

The results from these pairwise inhibition assays can be visually represented in the form of a network. FIG. 19B, for example, shows the inhibitory interactions among three different sets of bacterial isolates. Each number represents an individual bacterial isolate. An arrow going from one isolate and pointing to a second isolate indicates that the first isolate inhibits the second isolate. The absence of an arrow indicates no competition. Among these populations, there are mutually-inhibitory isolate pairs (e.g. isolates 16 and 18), non-inhibitory pairs (e.g. 8 and 9), and pairs in which one isolate inhibits the other (e.g. 21 and 30). These data guide selection of isolate pairs, which are most likely to coexist in a consortia without inhibition, for generation of a multi-strain inoculant composition.

Results from the mutual inhibition activity assays were entered into the enriched microbial library for storage and additional analysis as part of the presently disclosed MEFB nodal analysis.

### Example 6

### Characterization of a Microbial Library for Nutrient Utilization Complementarity

Isolates of a previously generated enriched microbial library collected according to the methods of Example 1 were subjected to one of the nodes involved with “multi-dimensional ecological function balancing (MEFB) nodal analysis,” i.e. “determine nutrient utilization complementarity.” When choosing isolates to form a consortium, it is desirable to choose isolates with complementary nutrient preferences or those with the greatest differences in nutrient preferences, in order to minimize resource competitive interactions. This Example describes the generation of nutrient use profiles, and thereby, the identification of nutrient use preferences for each of the bacterial isolates. Sub section under nutrient utilization.

### Methods:

Nutrient use profiles were generated for each of the bacterial isolates as a result of the isolates being grown on 95 different nutrient substrates in Biolog SF-P2 Microplate™ plates (available at the World Wide Web at biolog.com/Certificates%20of%20Analysis%20/%20 Safety%20Data%20 Sheets/ms-1511-1514-sfn2-sfp2-specialty-microplates-2/) for three to seven days. The Biolog SF-P2 plates tested the media listed in Table 3 below.

TABLE 3

| A1 | A2 | A3 | A4 | A5 | A6 |
|---|---|---|---|---|---|
| Water | α-Cyclodextrin | β-Cyclodextrin | Dextrin | Glycogen | Inulin |
| A7 | A8 | A9 | A10 | A11 | A12 |
| Mannan | Tween 40 | Tween 80 | N-Acetyl-D- | N-Acetyl-β-D- | Amygdalin |
| | | | Glucosamine | Mannosamine | |
| B1 | B2 | B3 | B4 | B5 | B6 |
| L-Arabinose | D-Arabitol | Arbulin | D-Cellobiose | D-Fructose | L-Fucose |
| B7 | B8 | B9 | B10 | B11 | B12 |
| D-Galactose | D-Galacturonic | Gentiobiose | D-Gluconic | α-D-Glucose | m-Inositol |
| | Acid | | Acid | | |
| C1 | C2 | C3 | C4 | C5 | C6 |
| α-D-Lactose | Lactulose | Maltose | Maltotriose | D-Mannitol | D-Mannose |
| C7 | C8 | C9 | C10 | C11 | C12 |
| D-Melezitose | D-Melbiose | α-Methyl-D- | β-Methyl-D- | 3-Methyl- | α-Methyl- |
| | | Galactoside | Galactoside | D-Glucose | D-Glucoside |
| D1 | D2 | D3 | D4 | D5 | D6 |
| β-Methyl- | α-Methyl- | Palatinose | D-Psicose | D-Raffinose | L-Rhamnose |
| D-Glucoside | D-Mannoside | | | | |
| D7 | D8 | D9 | D10 | D11 | D12 |
| D-Ribose | Sallcin | Sedoheptulosan | D-Sorbitol | Stachyose | Sucrose |
| E1 | E2 | E3 | E4 | E5 | E6 |
| D-Tagalose | D-Trehalose | Turanose | Xylitol | D-Xylose | Acetic Acid |
| E7 | E8 | E9 | E10 | E11 | E12 |
| α- | β- | γ- | p-Hydroxy- | α-Ketoglutaric | α-Ketovaleric |
| Hydroxybutyric | Hydroxybutyric | Hydroxybutyric | phenylacetic | Acid | Acid |
| Acid | Acid | Acid | Acid | | |
| F1 | F2 | F3 | F4 | F5 | F6 |
| Lactamide | D-Lactic Acid | L-Lactic Acid | D-Malic | L-Malic Acid | Pyruvic Acid |
| | Methyl Ester | | Acid | | Methyl Ester |
| F7 | F6 | F9 | F10 | F11 | F12 |
| Succinic Acid | Propionic Acid | Pyruvic Acid | Succinamic | Succinic Acid | N-Acetyl-L- |
| Mono-Methyl | | | Acid | | Glutamic |
| Ester | | | | | Acid |
| G1 | G2 | G3 | G4 | G5 | G6 |
| L-Alaminamide | D-Alanine | L-Alanine | L-Alanyl- | L-Asparagine | L-Glutamic |
| | | | Glycine | | Acid |
| G7 | G8 | G9 | G10 | G11 | G12 |
| Glycyl-L- | L-Pyroglutamic | L-Serine | Putrescine | 2,3-Butanediol | Glycerol |
| Glutamic | Acid | | | | |
| Acid | | | | | |
| H1 | H2 | H3 | H4 | H5 | H6 |
| Adenosine | 2′-Deoxy | Inosine | Thymidine | Uridine | Adenosine-5′- |
| | Adenosine | | | | Monophospate |
| H7 | H8 | H9 | H10 | H11 | H12 |
| Thymidine-5′- | Uridine-5′- | D-Fructose | α-D-Glucose- | D-Glucose- | D-L-α- |
| Monophospate | Monophospate | 6-Phosphate | 1-Phosphate | 6-Phosphate | Glycerol |
| | | | | | Phosphate |

Growth was determined by measuring optical density of the isolates inoculated in each of the nutrient substrates. The nutrient use assays identified the niche width (number of substrates the isolates grew in) and niche overlap (nutrients that may be shared between all pairwise combinations of isolates).

### Results:

FIG. 20 is a heat map representation of the in nutrient use preferences across 95 nutrients for a collection of microbial isolates, including PS1 which is denoted as strain 3211.1, from the enriched microbial library. The figure summarizes growth on 95 nutrients (represented in columns) with the growth on each nutrient indexed by intensity of the color of that column (darker color=more growth resulting from better nutrient usage). Based on these results, isolates which have complementary nutrient preferences or the greatest differences in nutrient preferences, may be selected to be part of a consortium.

Results from the nutrient utilization complementarity assays were entered into the enriched microbial library for storage and additional analysis as part of the presently disclosed MEFB nodal analysis

### Example 7

### Characterization of the Bacterial Library for Production and Responsiveness to Cellular Signals Resulting in Expression of Antimicrobials (Antimicrobial Signaling/Responsiveness Capacity)

Isolates of a previously generated enriched microbial library collected according to the methods of Example 1 were subjected to one of the nodes involved with “multi-dimensional ecological function balancing (MEFB) nodal analysis,” i.e. “determine antimicrobial signaling/responsiveness capacity.” This Example describes experiments performed to obtain comprehensive information on the capacity of individual isolates to enhance or repress the pathogen inhibitory capacity of each of the other isolates. This information can guide the selection of isolates, which are most likely to maximize the pathogen inhibitory capacity of each other, to be part of a consortium. Among other things, this assay is capable of identifying isolates that can enhance the inhibitory capacity of other isolates, despite lacking inhibitory functions of their own. Thus, in some embodiments, consortia produced as part of the MEFB nodal analysis can include one or more isolates with no individual suppressive capacity.

Methods:

Paired microbial isolates were inoculated 1 cm apart on plates containing 15 ml of the rich medium ISP2, which contains malt extract (10 g/L), yeast extract (4 g/L), dextrose (4 g/L), and agar (20 g/L), with four replicates of each pair per plate. Control plates were inoculated with each isolate individually, with four replicates per plate. Isolates were inoculated as 4 μl drops of spore suspensions containing approximately 5×10⁷ spores. Spore suspensions of Streptomyces isolates were made by collecting spores of each isolate grown on Oatmeal Agar plates on 30% glycerol. Suspensions were filtered through cotton and concentration of spores in each filtrate was quantified by dilution plating.

Plates were incubated at 28° C. for three days, at which time Bacillus or other pathogen overlays were spread onto each plate. Briefly, for example, a 12-hour culture of the Bacillus targets grown in Nutrient Broth (DIFCO, Becton, Dickinson and Co., Franklin Lakes, N.J.) to an OD600=0.800 was diluted 1:10 in Nutrient Broth containing 0.8% agar and added as a 10 ml overlay on the plates. Alternatively, a fungal plug is introduced into the center of the plate as described above (Example 3). After 24 hours at 30° C., inhibition zones on the pathogen lawns were measured. The inhibition zone was quantified with two, measurements from the edge of the dotted isolate to the edge of the cleared zone where the overlaid isolate did not grow.

Two perpendicular 90° length measurements were made per zone from the edge of the Streptomyces isolate test colonies to the end of the inhibition zone, where the overlaid isolate began to grow, away from the paired isolate, and the average was used for statistical analyses. Inhibition zones of paired Streptomyces were compared with zones generated by the Streptomyces isolates grown on ISP2 plates alone (controls). The effect of a paired isolate was evaluated by testing the significance and direction (increase or decrease of inhibition) of differences between the inhibition zones of isolates in the presence and the absence of a paired isolate. Results from these assays can be represented as networks describing the signaling relationship between microbial isolates.

### Results:

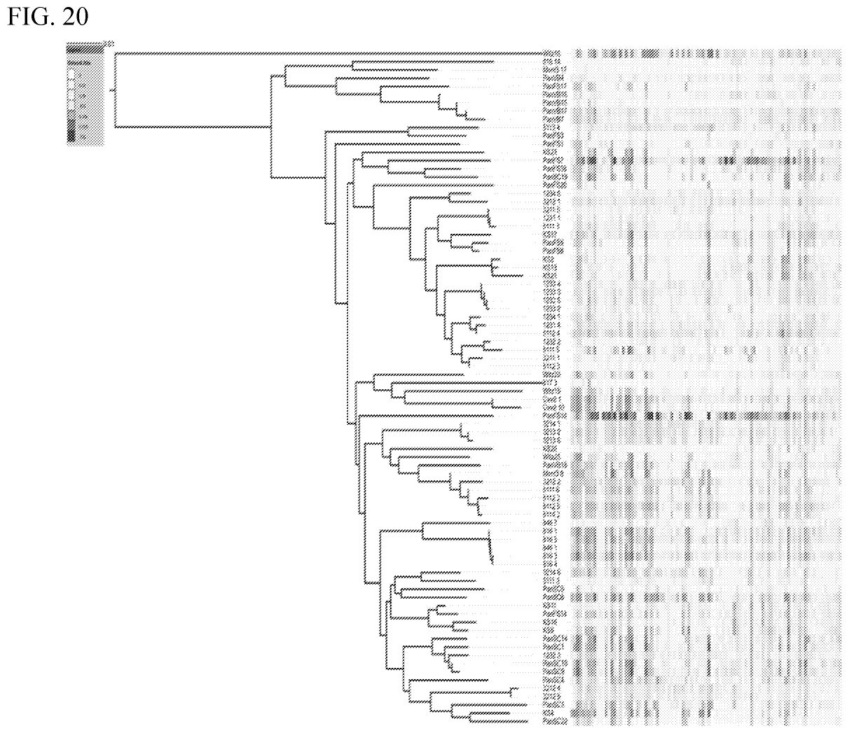

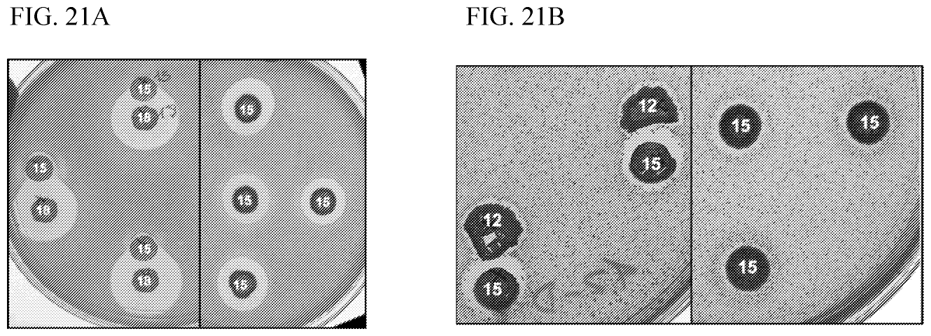

FIG. 21 illustrates an exemplary change in inhibition zones for Streptomyces isolate 15 in the presence vs. absence of another Streptomyces isolate (either isolate 18 or isolate 12). Specifically, FIG. 21A shows that isolate 15 is suppressed in its to inhibit the target overlay in the presence of isolate 18 (paired inhibition on the left, inhibition when alone on the right). In contrast, FIG. 21B shows that isolate 15 is better at inhibiting the target overlay when in the presence of isolate 12 (left-hand side) vs. when alone (right-hand side).

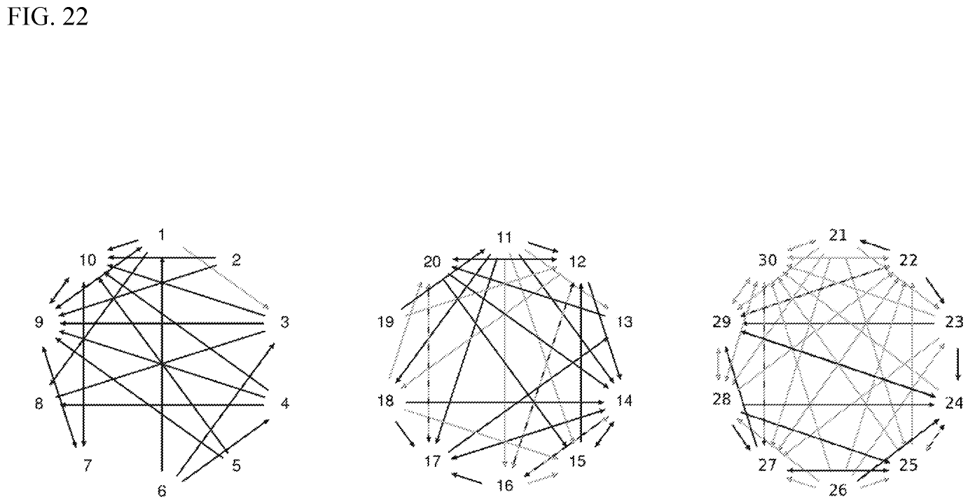

FIG. 22 shows signaling interactions that alter inhibitory phenotypes among 3 different collections of bacteria. Each number represents an individual microbial strain. A green arrow from one isolate to another means that the first isolate suppresses antibiotic inhibition by the second isolate. A blue arrow from one isolate to another indicates that the first isolate enhances antibiotic inhibition by the second isolate. These data provide a platform for selecting isolates for forming new consortiums, and optimizing the pathogen suppressive potential of these consortiums.

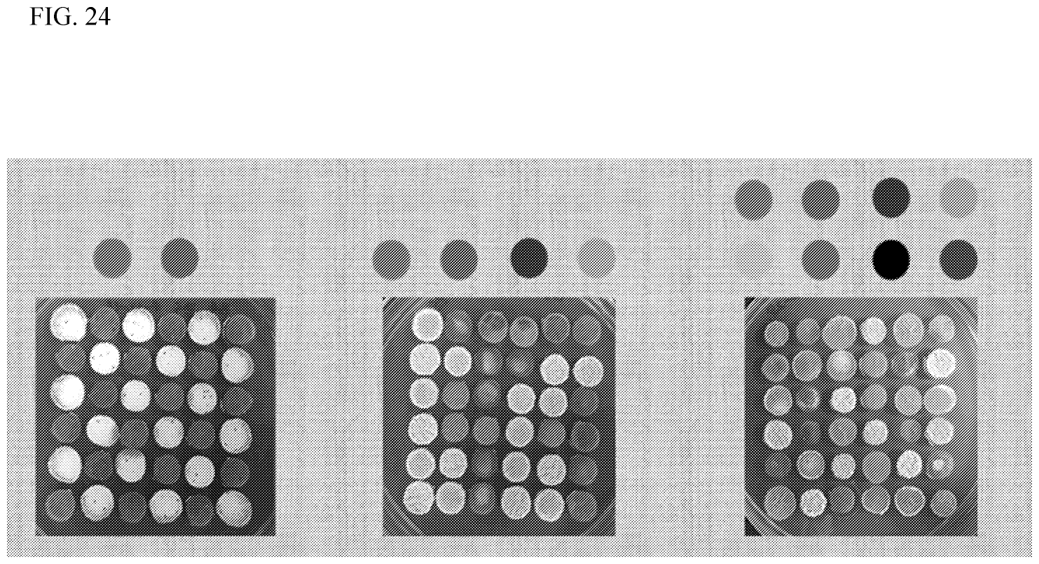

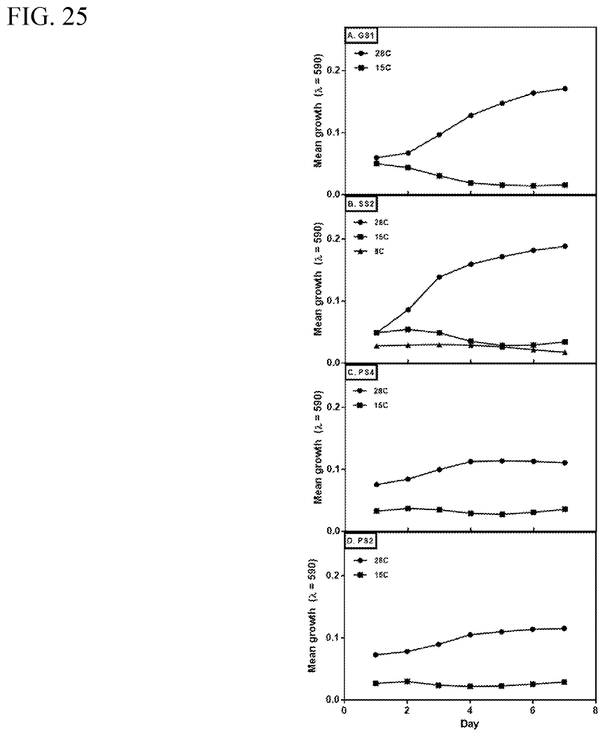

Furthermore, enhanced assays were developed for quantifying microbial signaling interactions. Specifically, bacterial isolates were grown in combinations of 2, 4, 8 (or any number) on ISP2 medium (which contains malt extract (10 g/L), yeast extract (4 g/L), dextrose (4 g/L), and agar (20 g/L)) (see FIG. 24 showing the plating strategy for this experiment). Microbial suspensions were dotted in randomly-determined arrays onto the medium, and spores were harvested from the petri dish using a sterile cotton swab after 72 h incubation. RNA was extracted from the resulting spore mixtures, and sequenced. The resulting transcriptomic data was analyzed for each isolate when grown alone vs. in combination with one or more other isolates. These data were used to quantify the extent to which critical secondary metabolic pathways (including antibiotic biosynthetic pathways as well as genes associated with plant growth promoting compounds or other crop-beneficial traits) were upregulated or exhibit changes in gene expression in the presence of one or more other isolates.

Results from the antimicrobial signaling/responsiveness capacity assays were entered into the enriched microbial library for storage and additional analysis as part of the presently disclosed MEFB nodal analysis.

### Example 8

### Characterization of the Bacterial Library for Plant Growth Promotion Activity and/or Other Beneficial Phenotypes (“Determine Plant Growth Promotion Ability”).

Isolates of a previously generated enriched microbial library collected according to the methods of Example 1 were subjected to one of the nodes involved with “multi-dimensional ecological function balancing (MEFB) nodal analysis,” i.e. “determine plant growth promotion ability.” This Example describes how the bacterial isolates may be tested for their growth promotion activity of different plants, and how that analysis may be used in the formation of consortia containing different combinations of isolates.

### Methods:

Wheat, alfalfa, corn, and soybean plants were evaluated for the growth response to microbial inoculants in growth chamber and greenhouse conditions. For each crop, containers (6.5 cm diameter x 25 cm long) were filled with steamed (sterile) greenhouse soil mix (500 ml) and planted with two (corn, soy, wheat) or three (alfalfa) seeds. Crop varieties were: SOY: AG 0832; CORN: Viking, WHEAT: Prosper, ALFALFA: Saranac. Seeds were inoculated with microbial isolates from the enriched microbial library immediately after planting (2 ml of water agar containing the inoculant per seed, 1×10¹¹ cells inoculant/ml). Control plants were allowed to grow without the microbial inoculants Plants were grown in the greenhouse and harvested at 3 weeks (corn), 4 weeks (wheat), 4.5 weeks (soybean) and 5 weeks (alfalfa). At harvest, the roots were carefully washed, and above- and belowground fresh weights were recorded (g). Plants were subsequently placed in paper bags and maintained in the 95° F. drying oven for 3 days, after which dry weights were also recorded. Plant weights were compared between inoculated and non-inoculated treatments.

### Results:

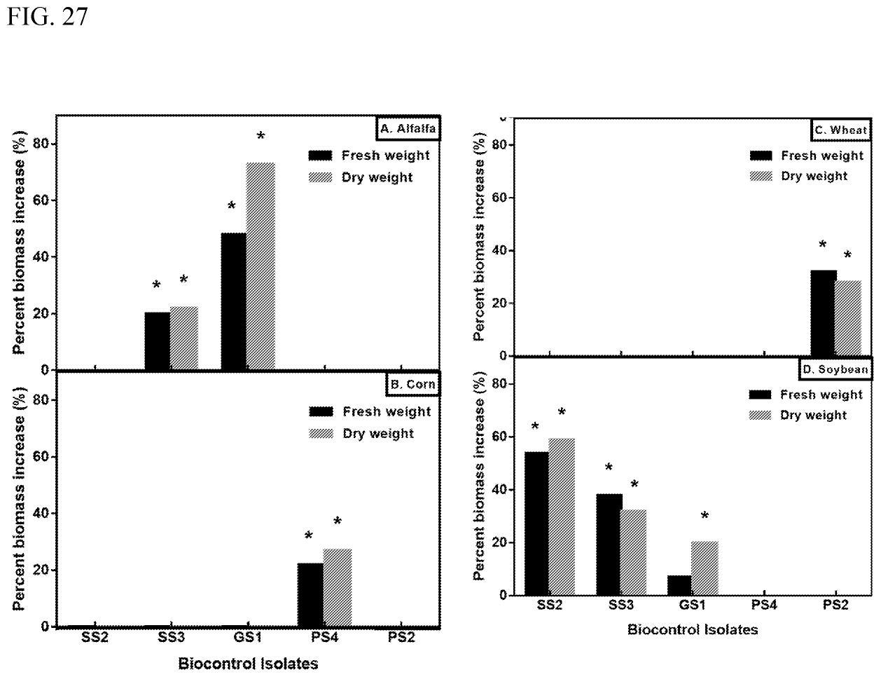

FIG. 27 shows the variation in growth promotion by different microbial inoculants on different plant hosts. Data are presented only for those plant host-microbial inoculant combinations in which statistically significant increases in plant biomass were observed on inoculated vs. non-inoculated plants. These results show that the microbial isolates—SS2, SS3, GS1 (Streptomyces lydicus), PS4, and PS2—have a statistically significant effect on promoting the growth of inoculated plants, as compared to non-inoculated. The results also show that the growth promotion activity of a microbial isolate may be specific to the type of plant. For example, PS4 and PS2 show significant growth promotion of corn and wheat, respectively, while the other isolates do not. As another example, only SS3 and GS1 promote the growth of alfalfa.

The analysis described here can be used to select isolates, which have growth promotion activity towards the specific plant of interest, to generate novel consortiums.

Results from the plant growth promotion ability assays were entered into the enriched microbial library for storage and additional analysis as part of the presently disclosed MEFB nodal analysis

### Example 9

### Characterization of Component Strains of a Multi-strain Inoculant Composition (Complete MEFB Nodal Analysis to Develop Novel Microbial Consortia).

This example illustrates the use of the MEFB nodal analysis to develop a novel microbial consortia. Isolates of a previously generated enriched microbial library collected according to the methods of Example 1 were accessed as illustrated in the first node of FIG. 14, i.e. “access an enriched microbial library.” Data included in the database, produced from each of the MEFB nodes was used to identify a three component microbial consortia capable of protecting potato plants against scab, a disease which greatly reduces the quality of the harvested vegetable and makes them unsuitable for sale. Furthermore, this three-component microbial consortia was also found to protect potato plants against Verticillium and Rhizoctonia, and soybean plants against pathogens such as Pythium, Phytophthora and Fusarium virguliforme. This three-component microbial consortia was devised based on the optimization of the three functions—antagonism, signaling and plant growth promotion—among the three isolates. Further, minimizing inhibition by antimicrobials and niche overlap were also important factors considered while devising this three-component microbial consortia.

One of these isolates, Streptomyces GS1 (Streptomyces lydicus), was collected according to Example 1 from a naturally-occurring disease-suppressive soil. This field was maintained in potato monoculture for more than 30 years, which imposed sustained directional selection on the soil microbial community. This soil was maintained under standard agricultural production practices, including plowing, potato harvest, and annual inputs of nutrients and agricultural chemicals. Microbial isolates from this field have been selected under the long-term, high-nutrient conditions of potato production, and have been shown to be particularly effective in suppressing plant pathogens.

The other isolates in this combination, Streptomyces PS1(Streptomyces sp. 3211.1) and Brevibacillus PS3 (Brevibacillus laterosporus), were also collected according to Example 1, from different locations in a long-term prairie site approximately 150 miles from the agricultural site that is the source of the first isolate. In contrast to the agricultural site, the prairie site was maintained for over 50 years without plowing or soil disturbance, and without agricultural chemicals. Microbial populations in this site have been selected under long-term low-nutrient conditions.

Without wishing to be bound by any one theory, the present inventors believe that within these long-term and low-nutrient communities, it is possible to identify microbes that support plant growth and are especially valuable to plant fitness. The inventors hypothesize that signals that mediate antibiotic production in low-nutrient habitats also reduce the costs of antibiotic activities to indigenous microbial populations. For example, the low-nutrient site described above included the plant growth-promoting isolate Brevibacillus PS3, and the signal-producing isolate Streptomyces PS1, which is especially effective in upregulating antibiotic production in diverse Streptomyces.

Finally, beyond the exemplary multiple strain inoculant mixture described immediately above, one can design alternative inoculant mixtures of microbial isolates that have been characterized with respect to their capacities to suppress diverse plant pathogens, grow on multiple nutrients at varying temperatures, resist antibiotics, signal or respond to signals, and/or promote growth of targeted crop species. This collection and the associated dataset provide a platform for the development of targeted or prescription microbial inoculant mixtures for disease suppression and plant growth promotion across diverse cropping systems, including specific crops or crop cultivars, geographic regions, and disease challenges. This represents a novel approach to and a unique resource for the development of microbial inoculants. The remaining portions of this Example describe the MEFB nodal analysis conducted in order to identify the three-strain consortia described above.

### A. Determination of Pathogen Suppressive Activity of Component Strains of the Multi-Strain Inoculant Composition

As depicted in FIG. 14, the component strains of the multi-strain inoculant composition described above were subjected to step 2 “determine pathogen suppressive activity”, as described below.

### Methods:

Fungal pathogen working stock isolates were grown on 0.5× potato dextrose agar (15 ml) for two (Rhizoctonia sp. and Sclerotinia sp.) or five days (Fusarium graminearum and F. oxysporum) on the benchtop (22° C.-25° C.). Fusarium virguliforme cultures were grown on SMDA (Soymilk Dextrose Agar) for approximately 20 days in the dark at (22° C.-25° C.). Pythium sp. were grown on V8 agar for two days on the benchtop (22° C.-25° C.). Phytophthora sp. were grown on V8 agar for two days on the benchtop (22° C.-25° C.).

Antagonist microbial isolates—GS1, PS1 and PS3—were dotted in 5 μl aliquot from a frozen 20% glycerol spore suspension onto plates containing 15 ml of oatmeal agar (OA) (if plates are being tested against Pythium sp. or Phytophthora sp. pathogen isolates, the Streptomyces were plated on V8 agar), evenly spaced around a plate/3 isolates per plate. There was a replicate plate of each dotted antagonist isolate series. Inoculated oatmeal agar plates were then incubated for three days at 28° C. After three days, the microbial isolates were killed by inverting plates over a watchglass containing 4 ml of chloroform for one hour. After one hour, plates were moved to a Class II Type A2 Biological Safety Cabinet (BSC) and the plates were opened to allow the residual chloroform to evaporate (30 min). This process kills or prevents the microbial isolate from producing more antibiotic and allows residual chloroform to evaporate so that the growth of the fungal isolates will not be affected. Antibiotics already produced by microbial isolates will have diffused into the medium prior to killing.

Subsequently, the chloroformed plates were inoculated with the fungal pathogen. An 8-mm plug of medium was removed from the center of the plate using a cork borer, and it was replaced with an 8 mm plug of medium containing the target fungal pathogen (selected from the growing margin of the working stock culture), mycelium-side facing up.

For plates inoculated with Rhizoctonia solani and Sclerotinia sclerotiorum, once the fungal plug was added, plates were parafilmed and placed on the benchtop for 3 days to incubate at room temp (22° C.-25° C.). For Fusarium oxysporum, Fusarium graminearum, and Phytophthora sp. (on V8 agar) pathogen assays, the plug was added, and plates were parafilmed and placed on the benchtop to incubate for seven days at room temp. Plates with Fusarium virguliforme were incubated in the dark (in a cardboard box placed on a lab bench) for 21 days (3 weeks) at room temp. The plates containing the Pythium sp. isolate (which are grown on V8 agar) were incubated on the lab bench at room temp for three days.

Fungal isolates grew radially from the center of the plate to the edge of the petri plate, except for zones in which pathogen-inhibitory antibiotics produced by the microbial isolates were present in the medium. These ‘clearing zones’, ‘cleared zones’, or ‘inhibitory zone’ of no growth were measured and represent a measure of pathogen-inhibitory capacity of the microbial isolates. The inhibition zone was quantified with two, 90° measurements from the edge of the microbial dotted isolate to the edge of the cleared zone.

### Results:

Table 4 shows complementarity in pathogen suppression among the pathogen-suppressive isolates in the exemplary multiple-strain inoculant mixture. The numbers indicate the intensity of inhibition (mean inhibitory zone size measured in mm) of each pathogen lineage (rows, e.g., P5) by each antagonist (columns, e.g., GS1). Among the full collection of pathogens represented here, every pathogen is strongly-inhibited by at least one antagonist isolate. The combination of the three isolates was thus expected to provide strong pathogen suppression activity.

TABLE 4
Pathogen suppression by component strains of inoculant mixture.

| | Pathogen | | | |
|---|---|---|---|---|
| | target | Microbial isolate | | |
| Pathogen Target | isolate | GS1 | PS1 | PS3 |
| Rhizoctonia solani | P5 | 8.95 | 0 | 3.25 |
| | P6 | 9.15 | 0 | 1.7875 |
| | P12 | 9.78 | 0 | 1.5 |
| Sclerotinia sclerotiorum | P16 | .* | . | 4.516667 |
| | P17 | 16.8 | 12.8 | 5.566667 |
| | P18 | 12.8 | 3.25 | 4.975 |
| | P19 | 8.63 | 0 | 5.8 |
| Fusarium virguhforme | P20 | 0 | 0 | 5.2 |
| | P21 | 1.3 | 0 | 9.5 |
| | P22 | 0 | 0 | 8.975 |
| | P23 | 0 | 3.58 | 6.175 |
| Fusarium oxysporum | P25 | 2.15 | 0 | 3.725 |
| | P26 | 9.38 | 0 | 4.1 |
| | P28 | 0 | 0 | 5.6 |
| | P29 | 0 | 0 | 4.225 |
| Fusarium graminearum | P30 | 14.58 | 0 | +** |
| | P31 | 8.63 | 3.05 | + |
| | P32 | 11.5 | 0 | + |
| | P33 | 13.13 | 0 | + |
| | P34 | 16.28 | 0 | + |
| | P35 | 11.03 | 0 | + |
| Phytophthora medicaginis | P43 | 4.53 | 0 | 5.6 |
| | P44 | 10.45 | 0 | 9.125 |
| Phytophthora sojae | P52 | 0 | 0 | 10.3 |
| Pythium ultimum | P58 | 9.95 | 0 | 6.6 |

*. refers to instances where data is not available
**+ refers to instances where there is some evidence for inhibition immediately over the surface of the dotted strain, but no clear inhibition zone beyond the dotted colony's edge.

### B. Determination of Mutual Inhibition Activity of the Component Strains of the Multi-Strain Inoculant Composition

As shown in FIG. 14, the microbes—GS1, PS1 and PS3—were subjected to one of the nodes involved with “multi-dimensional ecological function balancing (MEFB) nodal analysis,” i.e. “mutual inhibition activity.”

### Methods:

Each of the microbes—GS1, PS1 and PS3—was grown in the presence of every other isolate (i.e., GS1 with PS1; GS1 with PS3; and PS1 with PS3). Briefly, isolates GS1, PS1 and PS3 were dotted onto the center of individual petri dishes and allowed to grow for 3 days. Dotted isolates were then killed by placing petri dishes over a chloroform-filled watch glass in a safety cabinet for one hour. Subsequently, overlays of the other two isolates spread across the entire petri dish surface. In this way, each isolate was tested as a potential inhibitor (dotted isolates) in combination with every isolate as a target (overlaid isolates).

The growth of the microbial isolates was evaluated for the presence or absence of zones of inhibition at the interphase between the bacterial isolates. Each interaction is indicated as inhibitory (inhibition zone greater than 1 mm) or non-inhibitory (inhibition zone=0), with the inhibition zone size serving as a quantitative metric of sensitivity (high zone size=high sensitivity). The inhibition zone was quantified with two, 90° measurements from the edge of the microbial dotted isolate to the edge of the inhibition zone. Results from this analysis show the potential for isolates to coexist without inhibiting one another.

### Results:

Neither isolate PS3 nor GS3 inhibit either of the other members of the combination. PS1 shows mild inhibition of both GS1 and GS3 (zone size <5 mm).

### C. Determination of Nutrient Utilization Complementarity of Component Strains of the Multi-Strain Inoculant Composition

As shown in FIG. 14, the microbes in the multi-strain inoculant composition were subjected to one of the nodes involved with “multi-dimensional ecological function balancing (MEFB) nodal analysis,” i.e. “determine nutrient utilization complementarity.”

Nutrient utilization phenotypes were determined for each microbial isolate on 95 nutrient sources using Biolog SF-P2 Microplate™ plates (available at the World Wide Web at biolog.com/Certificates%20of%20Analysis%20/%20 Safety%20Data%20Sheets/ms-1511-1514-sfn2-sfp2-specialty-microplates-2/) (Schlatter et al., 2013). Briefly, isolates were inoculated into Biolog SF-P2 plates, and growth on each nutrient was monitored over three to seven days using optical density (OD) readings. For every isolate, OD in the control well is subtracted from every experimental well. All nutrients on which the resulting number is greater than 0.005 OD are determined to provide support for growth on that nutrient, and the total number of nutrients with an OD>0.005 for an isolate is determined to be that isolate's Niche Width. Total growth is calculated as the sum of growth (OD) across all nutrients on which an individual isolate can grow, and represents the collective growth potential for an isolate. Finally, preferred nutrients are defined as those on which the greatest ODs are observed. In total, these data provided insight into the diversity of nutrients utilized by each microbial isolate, and their preferred nutrients for growth.

Table 5 shows the nutrient use characteristics and complementarity of the strains in the exemplary inoculant mixture determined using Biolog data collected at 72 h. Niche Width is the number of nutrients on which the isolate grew.

| Isolate | Total Growth (%) | Niche Width | Top 10 Preferred Nutrients |
|---|---|---|---|
| GS1 | 9.18 | 86 | Tween 40 |
| | | | Dextrin |
| | | | D-Trehalose |
| | | | α-D-Glucose |
| | | | L-Asparagine |
| | | | Maltotriose |
| | | | Putrescine |
| | | | D-Gluconic acid |
| | | | L-Glutamic acid |
| | | | L-Alanine |
| PS1 | 4.66 | 81 | Dextrin |
| | | | D-Trehalonse |
| | | | Gentiobiose |
| | | | m-Inositol |
| | | | Amygdalin |
| | | | Inulin |
| | | | α-D-Lactose |
| | | | Sucrose |
| | | | D-Melibiose |
| | | | Lactose |
| PS3 | 7.82 | 72 | L-Malic acid |
| | | | L-Asparagine |
| | | | Gentiobiose |
| | | | L-Alanyl-Glycine |
| | | | D-Malic acid |
| | | | Inulin |
| | | | α-D-Glucose |
| | | | D-Trehalose |
| | | | Maltotriose |
| | | | Lactulose |

Overall, these three isolates all have moderately large niche widths and growth efficiencies, with limited mutual inhibition. They differ substantially in preferred nutrients for growth, and contribute diverse functional capacities to the mixture. Consequently, these isolates are expected to coexist, provide complementary functionalities to suppress plant diseases and enhance plant growth, and optimize plant productivity.

### D. Determination of Antibiotic Resistant Characteristics of Component Strains of the Multi-Strain Inoculant Composition

Isolates vary in their capacities to resist common antibiotics, which can be an important factor in determining soil colonization. Indigenous soil populations produce a wide array of antibiotics. Resistance to these naturally-produced antibiotics is thus an important attribute for successful inoculants.

### Methods

Spore suspensions of the microbial isolates—GS1, PSI and PS3—were made by collecting spores of each isolate grown on Oatmeal Agar plates on 30% glycerol. Suspensions were filtered through cotton and concentration of spores in each filtrate was quantified by dilution plating. Plates containing 15 ml of Starch Casein Agar (SCA) were spread plated with 150 μl of the test bacterial isolate. Next, immediately after plating the bacteria, three replicate antibiotic discs (Amoxicillin/Clavulanic Acid-30 μg, Novobiocin-5 μg & 30 μg, Rifampin 5 μg, Vancomycin 5 μg & 30 μg, Tetracycline 30 μg, Kanamycin 30 μg, Erythromycin 15 μg, Streptomycin 10 μg, and Chloramphenicol 30 μg) were placed onto each petri dish. Plates were then incubated at 28° C. for three days. After the incubation period, the inhibition zone from the edge of the antibiotic disc to the edge of where the bacteria could not grow was measured at 90° angles and recorded to measure bacterial susceptibility to the antibiotic on the disc. Each microbial isolate is indicated as being resistant (R—inhibition zone=0) or susceptible (S—inhibition zone>1 mm), with the degree of susceptibility indexed by the size of the zone (greater zone size=more susceptible).

### Results:

TABLE 6
Antibiotic resistance characteristics of each
component strain in the exemplary inoculant mixture.

| | Tested Microbial Strain | | |
|---|---|---|---|
| Antibiotic | PS1 | GS1 | PS3 |
| Tetracycline | MR | R | MR |
| Streptomycin | MS | S | S |
| Rifampin | MR | R | MR |
| Erythromycin | MR | S | S |
| Amoxicillin Clavulanate | MR | R | MR |
| Chloramphenicol | R | R | MS |
| Vancomycin 5 | MS | MS | MS |
| Vancomycin 30 | MS | MS | S |
| Novobiocin 5 | MR | MS | S |
| Novobiocin 30 | MS | MS | S |
| Penicillin | R | R | * |
| Kanamycin | S | S | S |

R-resistant (0 inhibition zone);
S-susceptible (>10 mm);
MR-moderately resistant (1-5 mm); and
MS-moderately susceptible (6-10 mm).

These data suggest that all 3 isolates possess some resistance to diverse antibiotics that may be present in soil environments.

### E. Determination of Antimicrobial Signaling/Responsiveness Capacity of the Component Strains of the Multi-Strain Inoculant Composition

As shown in FIG. 14, the microbes—GS1, PS1 and PS3—were subjected to one of the nodes involved with “multi-dimensional ecological function balancing (MEFB) nodal analysis,” i.e. “antimicrobial signaling/responsiveness capacity.”

### Methods:

Paired microbial isolates (GS1 and PS1; GS1 and PS3; and PS1 and PS3) were inoculated 1 cm apart on plates containing 15 ml of the rich medium ISP2, which contains malt extract (10 g/L), yeast extract (4 g/L), dextrose (4 g/L), and agar (20 g/L), with four replicates of each pair per plate. Control plates were inoculated with each isolate individually, with four replicates per plate. Isolates were inoculated as 4 μl drops of spore suspensions containing approximately 5×107 spores. Plates were incubated at 28° C. for three days, at which time Bacillus overlays were spread onto each plate. Briefly, a 12-hour culture of the Bacillus targets grown in Nutrient Broth (DIFCO, Becton, Dickinson and Co., Franklin Lakes, N.J.) to an OD600=0.800 was diluted 1:10 in Nutrient Broth containing 0.8% agar and added as a 10 ml overlay on the plates. After 24 hours at 30° C., inhibition zones on the Bacillus lawns were measured.

Two perpendicular measurements were made per zone from the edge of the microbial isolate colony to the end of the inhibition zone, away from the paired isolate, and the average was used for statistical analyses. Inhibition zones of paired microbial isolates were compared with zones generated by the microbial isolates grown on ISP2 plates alone (controls). The effect of a paired isolate was evaluated by testing the significance and direction (increase or decrease of inhibition) of differences between the inhibition zones of isolates in the presence and the absence of a paired isolate.

### Results:

The results showed that isolate PS1 enhance antibiotic production by the two other isolates (i.e., it resulted in increased inhibition zones around GS1 and PS3 compared to their single inoculant controls). Indeed, isolate PS1 was able to enhance antibiotic production by 30% of a random collection of Streptomyces from field soils, making it nearly 50% more effective than other isolates in enhancing antibiotic production. This isolate is effective in increasing antibiotic production by microbes in indigenous microbial populations in soil and/or in inoculant mixtures.

### F—Determination of Plant Growth Promotion Ability of the Component Strains of the Multi-Strain Inoculant Composition

As shown in FIG. 14, the microbes developed via the SPPIME platform, including GS1, PS1 and PS3, were subjected to one of the nodes involved with “multi-dimensional ecological function balancing (MEFB) nodal analysis,” i.e. “plant growth promoting activity.”

i. Cultivation Protocol for Microbial Inoculants Including, Microbial Consortium LLK3-2017 (Comprising GS1, PS1 and PS3).

Streptomyces isolates GS1 and PS1 were cultivated on oatmeal agar. Briefly, 20 g of oats were placed in a 2L Nalgene beaker containing 500 g of deionized (DI) H₂O and autoclaved at 121° C. for a 30 min sterilization period (or a standard liquid 30 cycle). Oatmeal was strained from the broth using a double-layer of cheesecloth. 250 ml of broth was mixed with 250 ml DI H₂O, 7.5 g Bacto Agar (BD, #214010), and 0.5 g Casamino Acids (BD, #223050). The medium was autoclaved for 30 min at 121° C. Streptomyces isolates were spread or streaked onto the medium in petri dishes or slants, and incubated at 28° C. Colonies appeared in 3-5 days, and plates were at full density in 10-14 days.

The Brevibacillus isolate PS3 was grown on plates containing Tryptic Soy Agar (TSA; Sigma, #22091-500 g). Brevibacillus were spread or streaked onto the medium in petri dishes or slants and incubated at 37° C. Colonies appeared in 24 hours, and were at full density in 3-5 days.

The isolates are combined in 1:1:1 density ratios. After the isolates are grown up individually, their inoculum densities are characterized, and then they are mixed at equal densitites immediately before inoculation into soil.

ii. Plant Growth-Promotion Testing: Greenhouse

For each crop (wheat, alfalfa, soybean, corn), a container (6.5 cm dia×25 cm long; approximately 500 cc of soil) was filled with steamed (sterile) greenhouse soil mix and planted with two of each seed (corn, soy, wheat) or three seeds (alfalfa) at a 0.5 inch depth. Crop varieties were: SOY: AG 0832; CORN: Viking, WHEAT: Prosper, ALFALFA: Saranac. Wheat, soybean and corn seeds (but not alfalfa) were surface sterilized before planting. Seeds were not treated with any fungicides or other chemicals. All crops were thinned to one seed per container following emergence. Ten replicate containers were planted for each inoculant and for the non-inoculated control.

Streptomyces cultures GS1 and PS1 were grown for 7-10 days on plates containing oatmeal agar (OA), and collected in 20% glycerol stocks. Stocks were serially diluted and plated on water agar (WA) for quantification. Stocks were then diluted to approximately 1×10¹¹ colony-forming units per ml.

For each inoculant, 2 ml of stock was distributed over the seeds at planting. Seeds and containers were watered when needed. Plants were incubated in the greenhouse and harvested at 3 weeks (corn), 4 weeks (wheat), 4.5 weeks (soybean) and 5 weeks (alfalfa). No additional nitrogen was added. At harvest for each crop, the roots were carefully washed, and above- and belowground fresh weights were recorded (g). Plants were subsequently placed in paper bags and maintained in the 95° F. drying oven for 3 days, after which dry weights were also recorded. Plant weights were compared between inoculated and non-inoculated treatments.

As shown in FIG. 27, the results showed substantial specificity in growth promotion among plant species (crop species) and microbial inoculant strains. For example, when inoculated onto different crop species, isolate GS 1 showed significant enhancements in both fresh and dry weights of alfalfa, but not corn or wheat. GS 1 also produced significant increases in dry weight when inoculated onto soybean.

iii. Plant Growth-Promotion Testing: Phytophthora Sojae Control on Soybean and Phytophthora Medicaginis Control in Alfalfa and Increased Plant Growth with Microbials

Soybean seeds (var. McCall) were surface-sterilized and planted into glass test tubes containing sterile moist vermiculite. Immediately following planting, 2 ml of at 10⁸ CFU/ml Streptomyces inoculant GS1 was inoculated into each test tube. Streptomyces inoculants, including GS1, were from stored 20% glycerol stocks. Test tubes containing plants were maintained at room temp (24-26° C.). Two days after planting, 1 ml zoospore inoculum of the pathogen Phytophthora sojae was added to each tube. Control tubes received only sterile water. Each inoculant-pathogen treatment was replicated 10 times in a completely randomized design. Tubes were flooded with sterile water and kept saturated for 7 days to encourage infection. Soybeans were harvested 21 days after planting. Disease incidence and severity were rated for each plant, as well as plant biomass, were determined for every plant. Briefly, disease severity for both alfalfa and soybean was rated using a 5-class scale: 0, healthy or no apparent discoloration; 1, less than 25% discoloration of the root; 2, 25-50% discoloration of the root; 3, 5075% discoloration o f the root; and 4, more than 75% discoloration of the root OR dead plant. Average disease severity was determined over all replicates for each treatment.

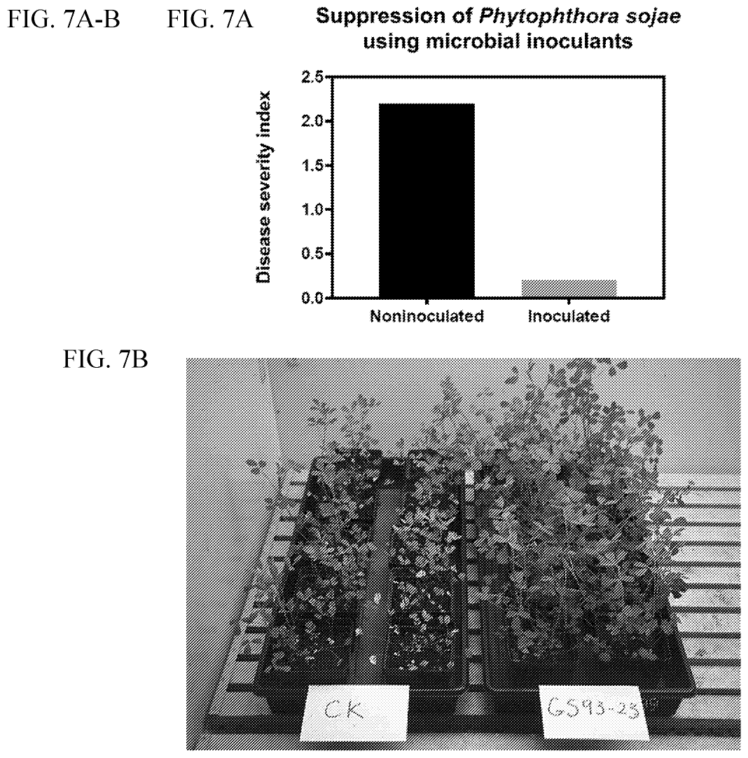

The results show that in growth chamber trials, microbial inoculants suppressed severity of Phytophthora sojae infection and increased percent healthy plants on soybean. See FIG. 7A. In growth chamber trials using soil infested with P. sojae, disease severity was reduced 93% on plants inoculated with strain GS1. Thus, these data show that microbial inoculant reduces Phytophthora infection on soybean. Inoculant included in this figure: GS1.

In a related greenhouse trial, Streptomyces inoculant GS1 also resulted in significant increases in plant biomass and reduced disease on alfalfa grown in field soil naturally-infested with the pathogen Phytophthora medicagini. Inoculum was prepared as described above, and used to inoculate seeds planted in field soil placed in sterile tins. Inoculation occurred immediately after planting. After 28 days, plants were both significantly larger and healthier in inoculated vs. non-inoculated tins (see FIG. 7B). GS1 was thus shown to promote plant growth.

iv. Plant Growth-Promotion: Conclusions

The Multi-Dimensional Ecological Function Balancing (MEFB) nodal analysis of steps i-v above lead to the production of microbial consortia LLK3-2017. Isolate PSI was included in the composition for its ability to enhance antibiotic production by other isolates, and PS3 for its ability to suppress plant pathogens and enhance plant growth (PS3). Isolate PS1 was able to enhance antibiotic production by 30% of a random collection of Streptomyces from field soils, making it nearly 50% more effective than other isolates in enhancing antibiotic production. This isolate is effective in increasing antibiotic production by microbes in indigenous microbial populations in soil and/or in inoculant mixtures. Isolate GS1 was included due to its strong capacity to suppress plant pathogens, and its capacity to enhance plant growth.

### Example 10

### Various Protocols, and Greenhouse and Field Trials Utilizing Microbes Developed via the Platform

### A. Becker, Rosemount, & Saint Paul Potato Field Trials

A Randomized Complete Block Design (RCBD) was used for all field experiments. Numbers of treatments and blocks varied among locations (Becker: 12 treatments, 7 blocks; Rosemount: 12 treatments, 7 blocks; and Saint Paul: 10 treatments, 10 blocks). Microbial inoculants were tested in combinations of 2 or 3 with a non-inoculated control, and a non-inoculated rice control. Microbial inoculants grown on rice (granular carrier) were applied in furrow at planting. Rice (500 g) was inoculated with 50 ml of Bacillus stock culture or 40 ml of Streptomyces stock culture. Bacillus and Streptomyces rice cultures were maintained separately, incubated for 48 h. Approximately 75-80 g of inoculum was added in a 1 sq. ft. area around the tuber in furrow. Microbial combinations were mixed in field; triple combinations had 25 g of rice inoculant mixture per tuber, double combinations had 40 g per inoculant, and the rice control had 75-80 grams per tuber. Potato variety Red Pontiac was used in all locations, and standard field production practices were utilized for management.

Inoculum preparation: Each microbial isolate was grown on plates containing 20 ml of oatmeal agar (OA) for 10-14 days. For each inoculant, spores were harvested from ten petri plates into 40 ml of 20% glycerol, and each tube was serially diluted and plated on to 1% water agar (WA) for quantification. Resulting stocks were stored at −20° C., and then inoculated onto sterile white rice at a density of 10¹² CFU/ml. Inoculated rice was incubated for 10-12 days at 28° C., and shaken to distribute inoculum daily. Rice was dried in the fume hood for 3 days prior to inoculation in-furrow at planting.

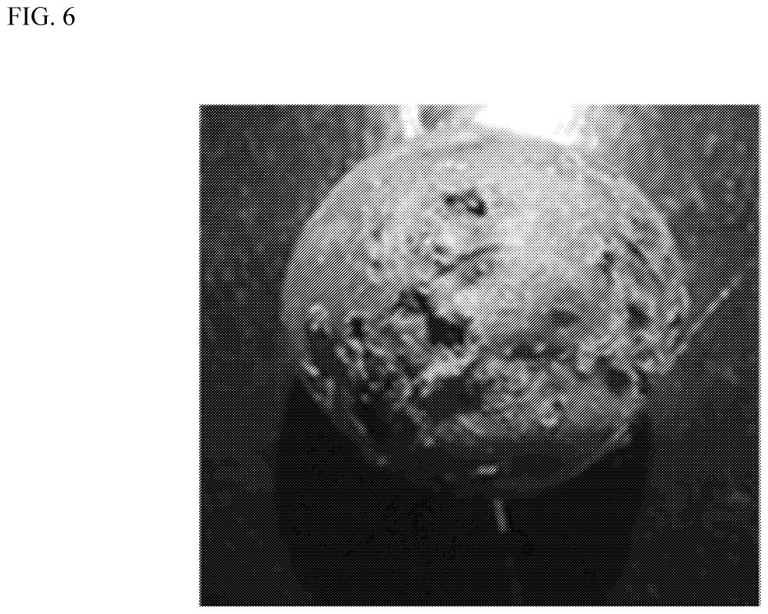

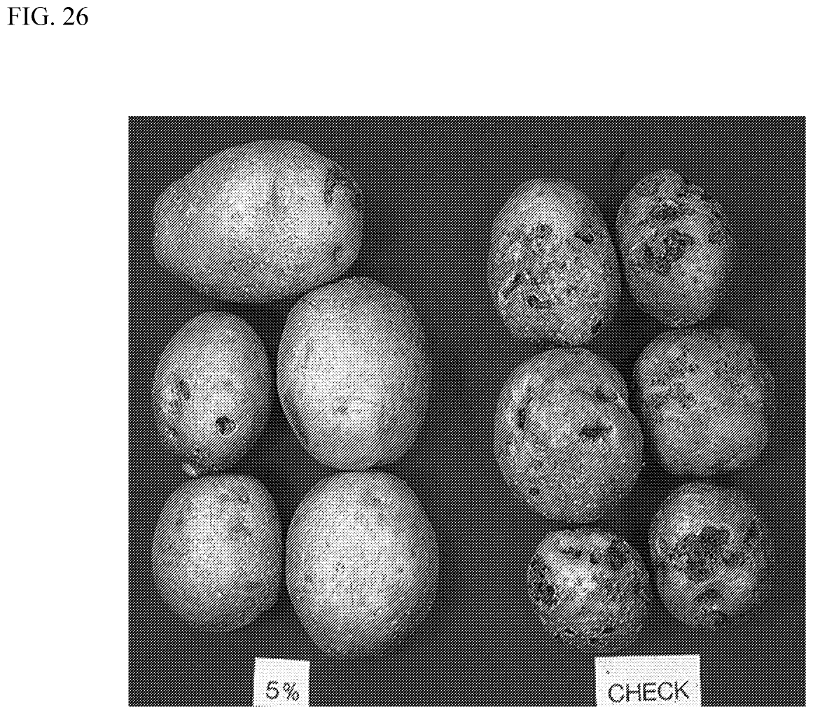

Tubers were harvested, and evaluated for symptoms of potato scab disease. As examples, images of healthy potatoes and potatoes showing symptoms of potato scab disease are depicted in FIG. 6 and FIG. 26. The results on the reduction in scab severity are described below under Section D, below.

### B. Becker Fumigation Field Trials

Plots were fumigated with chloropicrin, Vapam in the fall of 2015. The experiment included a non-fumigated control. In April, 2016, fumigated soil was removed from the plots, mixed thoroughly, and placed into 3 gallon bottomless plastic pots which had been sunk into the ground. Carbon amendments, microbial inoculants, or both were added to each soil; in addition, a non-amended control was maintained in every block. A single Red Pontiac potato tuber was planted into each pot. The experiment was established in a randomized complete block design, with 8 blocks. The field plots were maintained according to standard potato production practices. Tubers were harvested in August, and evaluated for disease (potato scab) and yield measurements.

### C. Field Trials: Becker and Rosemount Inoculants x Carbon Experiments: Potato (2014)

The experiments were established as a randomized complete block design with 9 treatments (3 inoculant treatments x 3 carbon amendments). Potatoes (Red Norland) were treated in-furrow with the carbon and/or microbial inoculants at planting. Microbial inoculants were established on sterile growstones mixed with oatmeal broth. Plants were maintained under standard potato production conditions.

Tubers were harvested in August, and evaluated for disease (potato scab) and yield measurements.

The results on the reduction in scab severity and yield measurements are described below under Section D, below.

### D. Field Trials: Becker and Rosemount Inoculants: Potato (2015)

We planted potatoes into fields that had been grown with soybean, wheat, or potato in 2014. Microbe isolates were inoculated at planting as in 2014, using inoculum that was prepared as described for 2014 (described above in Trial C). Plants were maintained under standard potato production conditions. Tubers were harvested in August, and evaluated for disease (potato scab) and yield measurements.

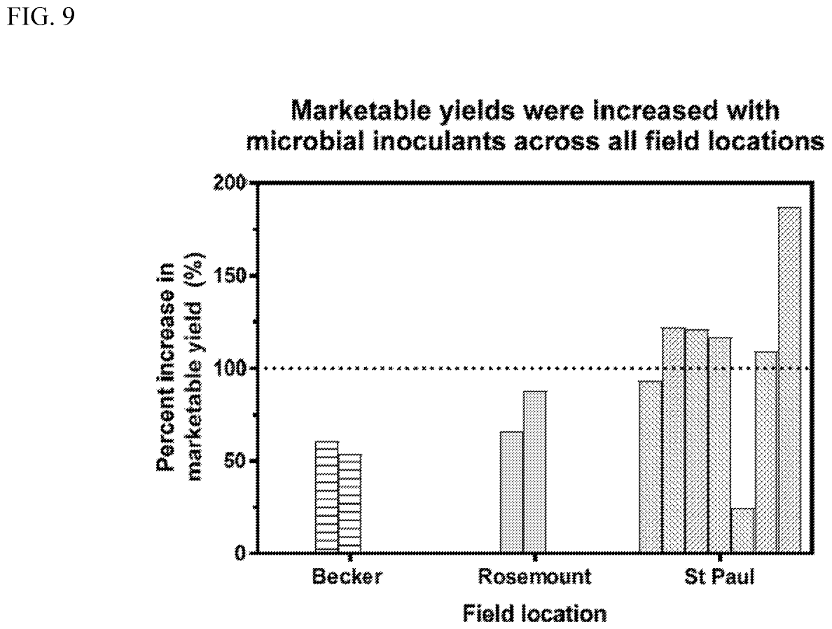

An amalgamation of results pertaining to marketable tuber yields from Sections A and D is shown in FIG. 9. Marketable tuber yields are defined as total yield of tubers with less than 5% scab coverage; percent increases in marketable yield were calculated against the control (noninoculated plots). Marketable tuber yields (total yield of tubers with less than 5% scab) were increased significantly in all 2016 field trials. Increases in marketable yields ranged from around 25% to over 180%. Thus, marketable yields were similarly increased across a wide range of disease pressures and field conditions. See FIG. 9. Inoculants represented in this figure include GS1, SS2, SS3, SS4, SS5, SS13, SS19, PS1, PS3, PS4, PS5.

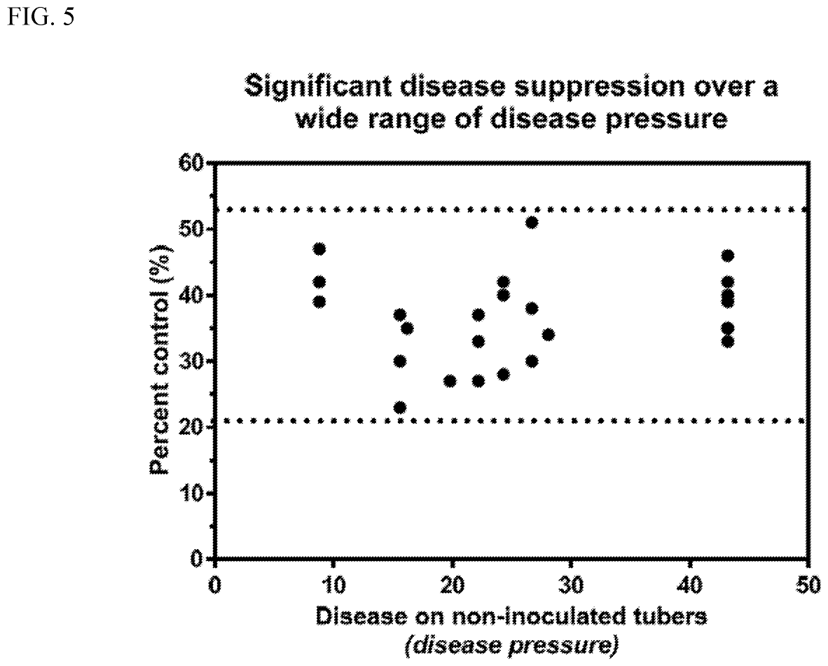

An amalgamation of results pertaining to the reduction in scab severity from Sections A, C and D is shown in FIG. 5. The reduction in scab severity in inoculated plots versus non inoculated plots in the same field trial (or “percent scab control”) was measured. See FIG. 5 and Table 7 below.

TABLE 7
Individual points for FIG. 5

| (EXPERIMENT) | Disease on Non- | Percent |
|---|---|---|
| Isolate(s) | inoculated Tubers | Control |
| (BPP) GS1 | 22.2 | 33 |
| (BPP) PS3 | 22.2 | 27 |
| (BPP) GS1 & PS3 | 22.2 | 37 |
| (BSP) GS1 | 24.3 | 42 |
| (BSP) PS3 | 24.3 | 40 |
| (BSP) GS1 & PS3 | 24.3 | 28 |
| (BWP) GS1 | 26.7 | 38 |
| (BWP) PS3 | 26.7 | 30 |
| (BWP) GS1 & PS3 | 26.7 | 51 |
| (RPP) GS1 | 15.6 | 37 |
| (RPP) PS3 | 15.6 | 23 |
| (RPP) GS1 & PS3 | 15.6 | 30 |
| (RSP) GS1 | 8.8 | 39 |
| (RSP) PS3 | 8.8 | 42 |
| (RSP) GS1 & PS3 | 8.8 | 47 |
| (BMDA) GS1 & PS3 | 19.8 | 27 |
| (BSCR) GS1 | 16.2 | 35 |
| (STPSCR) GS1, SS2, SS3 | 43.2 | 46 |
| (STPSCR) GS1, SS2, PS7 | 43.2 | 35 |
| (STPSCR) GS1, PS3, PS7 | 43.2 | 42 |
| (STPSCR) GS1, SS2, PS1 | 43.2 | 39 |
| (STPSCR) GS1, PS3, PS1 | 43.2 | 33 |
| (STPSCR) PS3, SS2, PS1 | 43.2 | 35 |
| (STPSCR) GS1, SS5, PS5 | 43.2 | 40 |
| (RSCRG1) PS3 | 28.1 | 34 |

Disease suppression was both high-level and consistent across a wide range of disease pressures; i.e. high levels of disease suppression was observed both from low disease pressure (less than 10% severity of scabs on non-inoculated tubers) to very high disease pressure (more than 40% severity of scabs on non-inoculated tubers). Disease pressure is indexed by the amount of disease in the non-inoculated control. High disease pressure could mean that smaller densities of pathogens were present or that environmental conditions were less conducive to infection (e.g. wet conditions reduce scab infection). Disease reduction averaged 36.4% among the 25 cases where disease reduction was statistically significant (range 27-51%). Thus, disease suppression occurred across a wide range of disease pressures and field conditions. Inoculants included in this figure: GS1, GS2, GS3, GS4, GS5, GS7, GS13, GS16, GS17, GS18, GS19, GS20, GS21, PS1, PS3, PS4, PS5.

### E. Field Trials: Becker and Rosemount Soybean 2014

In 2014, microbial and carbon amendments were evaluated in field plots at Becker and Rosemount, Minn. Microbial inoculant treatments (3; GS1, PS3, or non-inoculated control) and carbon amendments (3; non-amended, 2 cellulose doses) were evaluated in a factorial design. Soybean plots were marked out in a large field using GPS to designate a 2 x 6 foot plot for each treatment. Subsequently, the area representing the center 2 rows of soybeans (a 2 ft×6 in wide area centered on the marked row) was inoculated with the specific microbial x carbon-amended treatment (200 g granular inoculum per treatment area). The soybeans were subsequently drill-planted over the treatments. An average of 8-10 soybean plants were treated within each 2 ft treatment area. Soybean was AG 0732. At harvest, 2 plants from each treatment row (4 plants total per plot) were cut at the soil line and collected in paper bags. These were placed in plant drying ovens for 3 days at 95° F., at which time dry weights (biomass) were recorded and seeds per plant were determined for every plant.

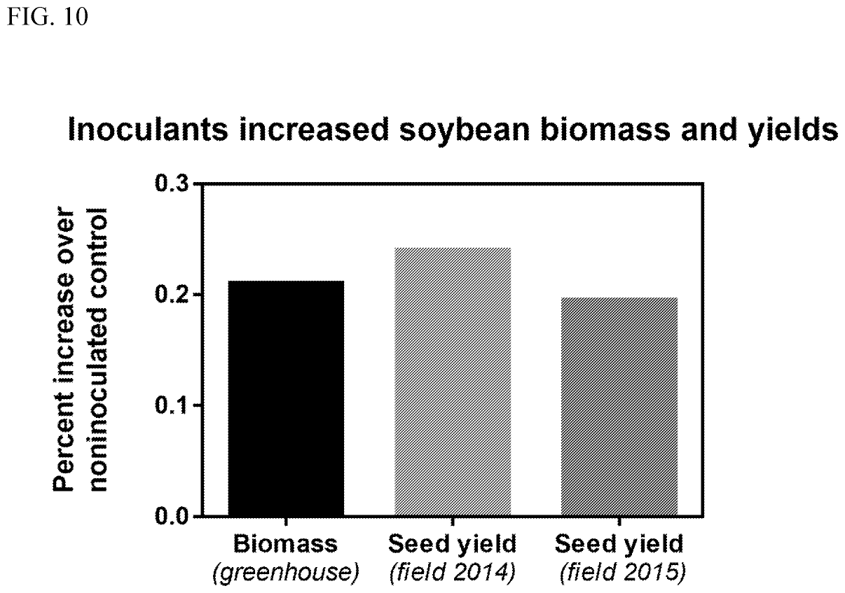

The results pertaining to enhancements in yield are described below under Section G, below, and in FIG. 10.

### F. Field Trials: Becker, Rosemount, Waseca, and Morris Inoculants: Soybean 2014 & 2015

Soybeans were planted at 4 locations (Becker, Rosemount, Waseca, and Morris). We evaluated both inoculants and carbon amendments in 2014, and microbial inoculants alone in 2015. Inoculum was prepared as described above for 2014 and 2015. At midseason and at harvest (September), 10 individual soybean plants along a center row of each plot was harvested. Biomass was determined at both midseason and harvest, and both seed counts and seed weights were determined for every plant at harvest.

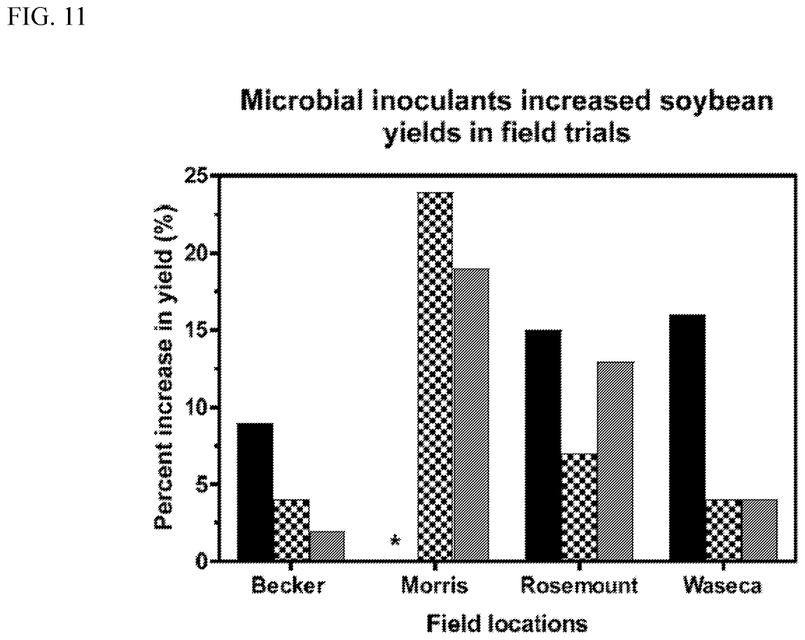

The results pertaining to enhancements in yield are described below under Section G, below, and in FIG. 10. Additionally, the % increase in yield with the use of microbial inoculants in field plots at each of the four locations in Minnesota (Becker, Rosemount, Waseca, and Morris) is shown in FIG. 11. The results show that yields are increased with inoculants across diverse field sites varying widely in soil chemistry, environmental conditions, and disease pressure.

### G. Greenhouse Trials: Soybean in 2016 (FV)

Pathogen inoculum: Fusarium virguliforme isolate (P21) was grown on SMDA (Soymilk Dextrose Agar) for 3 weeks on the benchtop in the dark (in a box) until hyphae and mycelium almost covered the plate. Hyphae and spores were scraped using a disposable inoculation loop. Ten plates were scraped into 40 ml of sterile DIH₂O in a 50 ml Falcon tube. Tubes containing the Fusarium virguliforme slurry were then homogenized and ground for 30 sec stints in the Waring blender.

A “seed hole” was created for soybeans in containers filled with steamed soil. Two ml of the pathogen inoculum slurry was added to each seed hole, then covered with soil again. Soybean seeds (variety AG 0832) were planted immediately adjacent to (but not within) the seed hole, and 2 ml of biological control inoculum were added as a soybean seed treatment. Seed were covered with soil, and watered as needed. Each microbial inoculant was evaluated on 10 replicates. Soybeans were grown for 6 weeks in the greenhouse, and soybean biomass and disease severity (quantified as the length of disease lesion along the taproot; cm) was determined for every plant.

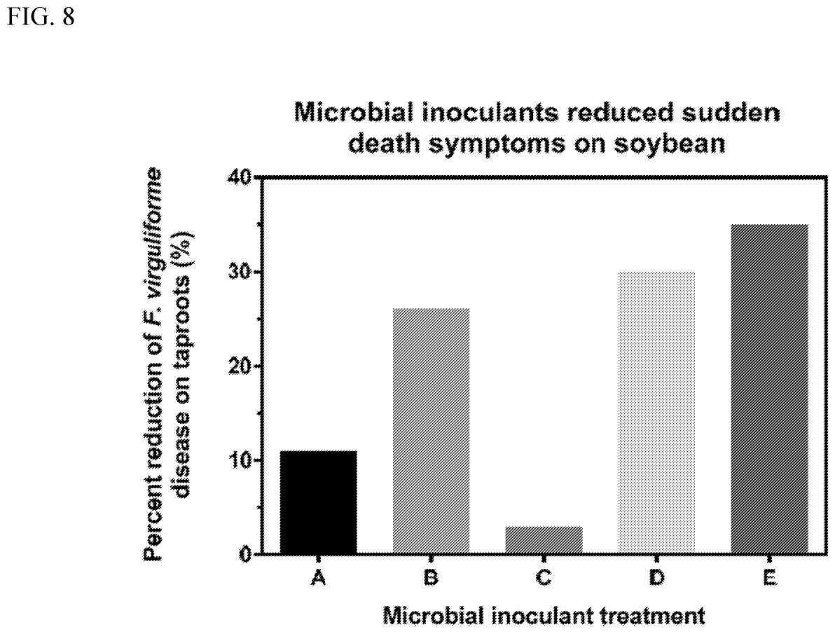

The results showed that in greenhouse trials, microbial inoculants reduced severity of disease symptoms caused by Fusarium virguliforme, though reductions were not statistically significant. In greenhouse trials in which the pathogen was inoculated into soil prior to planting, 5 of 7 inoculant treatments showed reductions in disease (lesion length). See FIG. 8. Reductions ranged from 3-35% (average 21%). Thus, microbial inoculants significantly reduced Fusarium infection on soybean. Inoculants represented in this figure include GS1, SS2, SS3, SS6, PS3, PS5.

Amalgamation of results pertaining to yield measurements from trials E, F and G are shown in FIG. 10. The results showed that in greenhouse trials, microbial inoculants produced significant enhancements in soybean biomass (dry weight at 6 weeks). Biomass increased on average 20% on inoculated vs. non-inoculated plants. In field studies in 2014, yields were increased with microbial inoculants in 3 of 4 field trials at two locations, though differences were statistically significant in only one of these trials (24% seed yield increase over non-inoculated plots. Thus, microbial inoculants significantly increased soybean yields. See FIG. 10. In field trials at 4 locations in Minnesota in 2015, soybean yields were increased with microbial inoculants in 11 of 12 cases. Average yield increase across locations was 13% (22% at Morris, 8% at Becker, 12% at Rosemount, and 8% at Waseca). Thus, microbial inoculants significantly increased soybean yields. Inoculants included GS1, SS2, SS3, SS6, SS7, SS8, PS2, PS3, PS5.

### Example 11

### Soil Carbon Amendments Alter Nutrient Use and Pathogen-Suppressive Potential of Soil Streptomyces

The input of organic matter is commonly used for increasing microbial activity and biomass in agricultural soils. Soil carbon amendments alter the specific resource availability in agricultural soil and influence the composition and function of the soil microbial community. The addition of carbon to soil can lead to enrichment of microbial populations that are beneficial for sustainable agricultural production. Furthermore, the impacts of soil carbon amendments on microbial metabolic capacity and species interactions are not well understood.

The objective of this study was to evaluate the effects of soil carbon amendments on nutrient use profiles and antibiotic inhibitory phenotypes of Streptomyces populations from agricultural soils.

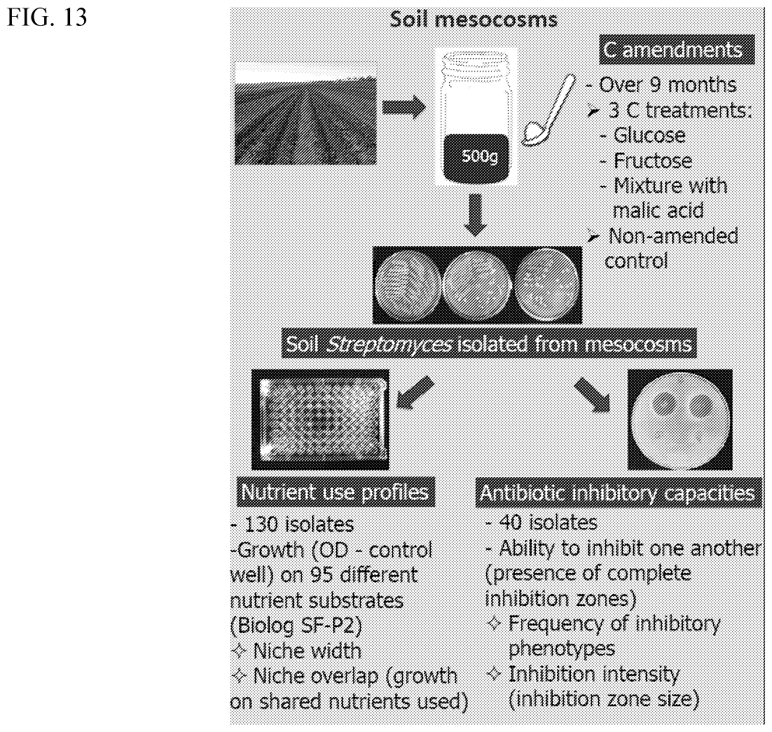

Agricultural soils were collected, and deposited into soil mesocosms (500 g of soil per mesocosm). There were four mesocosms: (1) non-amended control, (2) glucose, (3) fructose, and (4) mixture of glucose and fructose with malic acid. The soil of mesocosms (2), (3), and (4) was amended every other week for the first two months, and subsequently monthly over the course of nine months with their respective amendment of glucose, fructose, or the mixture of glucose and fructose with malic acid. FIG. 13 shows a schematic of the protocol for determining the effects of soil carbon amendment on Streptomyces soil isolates relative to non-amended control soil described here. At the conclusion of the nine months, Streptomyces isolates were isolated from each of the mesocosms. A total of around 40 isolates (38-44) were collected for each carbon treatment, with isolates coming from at least 6 replicate mesocosms for each treatment. The Streptomyces isolates were assayed for nutrient use profiles and antibiotic inhibitory capacities.

Nutrient use profiles were generated for 130 isolates as a result of the isolates being grown on 95 different nutrient substrates in Biolog SF-P2 Microplate™ for three to seven days. Growth was determined by measuring optical density (OD) of the isolates inoculated in each of the nutrient substrates, and subtracting the OD observed in the control (no-nutrient) well from the OD observed on that substrate to yield the growth measurement. The nutrient use assays identified the niche width (number of substrates the isolates grew on), while niche overlap for every isolate pair was determined based upon growth on shared nutrients using the equation:

Niche overlap (Y against X)=Σ₁₋₉₅(min [Xᵢ, Yᵢ]/Xᵢ)/(niche width [isolate X]), wherein X and Y each denote a different isolate.

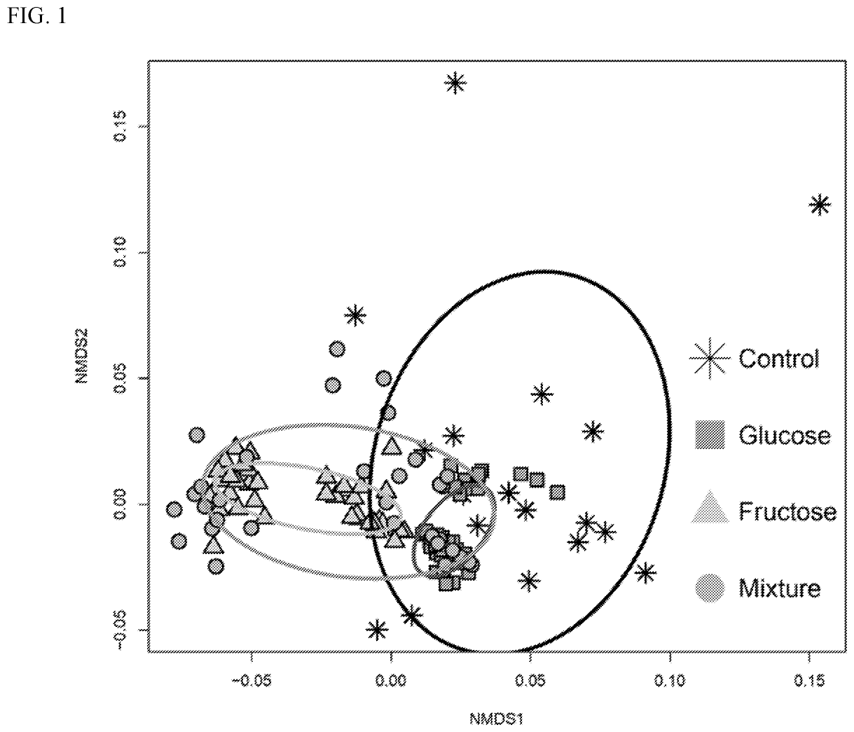

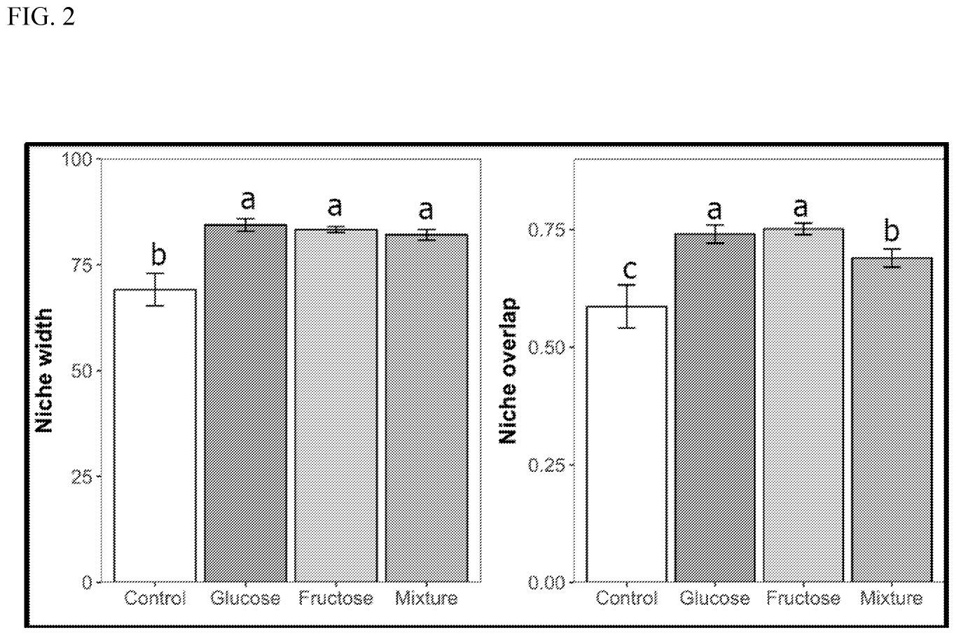

The nutrient use profiles of the Streptomyces isolates varied among isolates from the carbon amended soils and the non-amended soils (ADONIS, p<0.005; Anderson, M. J. 2001. A new method for non-parametric multivariate analysis of variance. Austral Ecology, 26: 32-46). The mesocosms amended with simple substrates resulted in Streptomyces populations with more uniform nutrient use profiles (FIG. 1). Streptomyces isolates from carbon-amended soils exhibited a greater niche width (FIG. 2, left panel). In other words, the % of nutrients that could be utilized by the Streptomyces isolates was higher when they were isolated from carbon-amended soil, as compared to non-amended soil. The isolates further exhibited a greater niche overlap among isolates from carbon-amended soils than from non-amended soils (FIG. 2, right panel). That is, the Streptomyces isolates isolated from carbon-amended soil had higher percentage of nutrient use shared between all possible pairwise combinations of isolates, as compared to Streptomyces isolates isolated from non-amended soil.

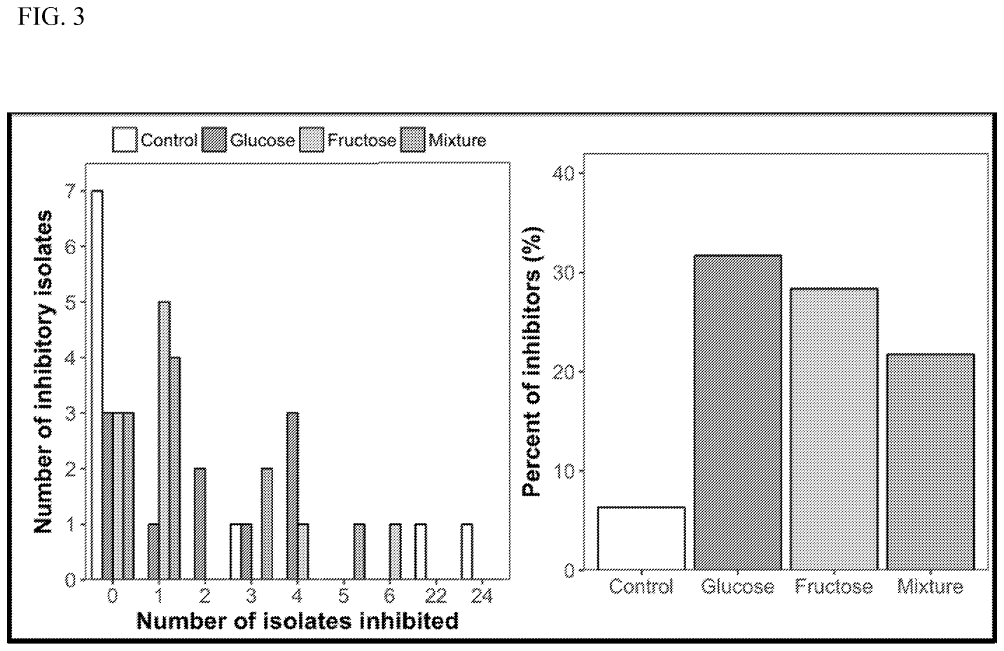

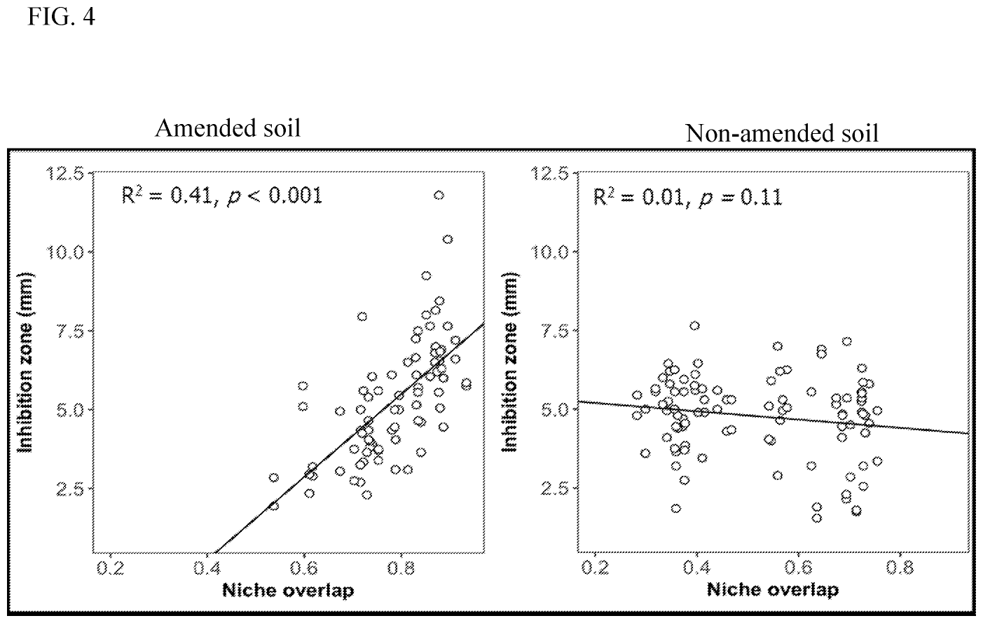

Antibiotic inhibitory capacity profiles were generated for 40 isolates. The isolates were randomly selected from the pool of isolates collected for each treatment, constrained to represent isolates from at least 3 different replicate mesocosms for each treatment. The data was generated by determining the ability of the isolates to inhibit one another—plating the isolates together and identifying the presence of complete inhibition zones on the plates. These assays identified the frequency of inhibitory phenotypes and the intensity of the inhibition (inhibition zone size). The soil carbon amendments increased the frequency of the Streptomyces inhibitory phenotypes, especially those that were predominantly inhibitory against Streptomyces from non-amended soils (FIG. 3). The inhibition intensity and niche overlap were positively correlated between inhibitory and susceptible Streptomyces isolates from carbon-amended soils and non-amended soils, respectively (FIG. 4). For isolates from amended soil, the results show that there is a positive correlation between niche overlap and inhibition zone (FIG. 4, left panel), whereas no such correlation is observed in isolates from non-amended soil (FIG. 4, right panel).

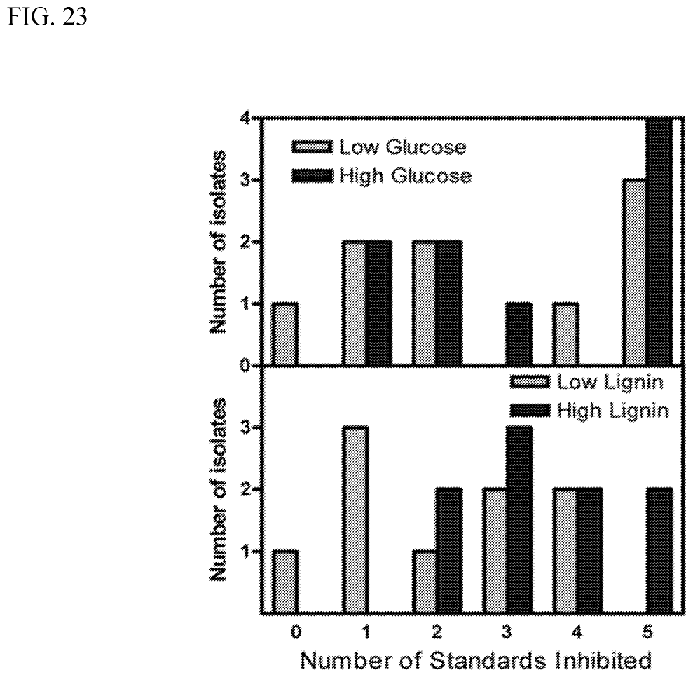

Finally, in a related study using the same methods as described above, Streptomyces populations were more inhibitory against a set of 5 plant pathogen targets when they were ‘fed’ high doses vs. low doses of the same carbon (either glucose or lignin) over the 9 month period (FIG. 23).

Our results show that shifts in the metabolic capacities of Streptomyces in response to carbon amendments could lead to potential intensification of competition for preferred resources. The carbon amendments selectively enriched the Streptomyces strains with broad niche widths and antagonistic capacities. The carbon amendments intensified selection for Streptomyces phenotypes that were inhibitory against the strongest resource competitors. Taken as a whole, the results described here show that the addition of carbon amendments to the soil acted as a selective force that can lead to the emergence of ecologically distinct populations with distinct life history strategies and functional characteristics. Furthermore, the addition of carbon amendments to the soil could facilitate the establishment of pathogen-suppressive soils in agricultural systems.

### Example 12

### Microbial Inoculants and Soil Carbon Amendments Suppress Diseases and Increase Yields in the Field

The present data is from field-based experiment relating to the use of nutrients to alter: i) disease suppression; or ii) soil colonization by inoculants; or iii) both. The data supports the conclusions from the aforementioned mesocosm studies.

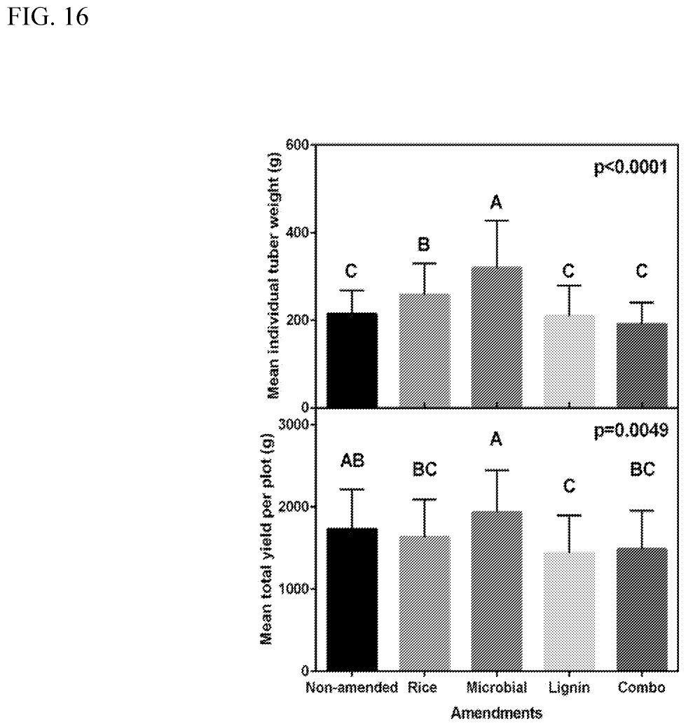

Disease suppression was compared with microbial inoculants and/or carbon amendments (lignin) in chloropicrin-fumigated vs. non-fumigated soils from a field naturally-infested with the potato scab pathogen. Briefly, plots were fumigated with chloropicrin or left as non-fumigated controls in the fall preceding the experiment. In the following spring (April), fumigated or non-fumigated soil was added to 3 gallon pots and removed to an adjacent, non-fumigated field. Treatments to each pot included: non-treated control; rice only control; microbial inoculant on rice; lignin only; or lignin plus the microbial inoculant. For lignin-treated pots, the lignin was mixed thoroughly with the soil prior to placement in pots (see below). Streptomyces and Bacillus growth as described previously, and were inoculated as a combination into soil on a rice carrier; the rice treatment was included to confirm that the nutrients added by the rice carrier were not the source of disease suppression or yield benefits. Soil was transferred to bottomless pots that were buried to the soil line to facilitate drainage. Microbial inoculants or the rice carrier were placed adjacent to the seed piece (in-furrow) at planting (see below). Red Pontiac (small red) potato tubers were planted into each pot (1 tuber per pot). Pots were maintained according to standard potato practice for MN. After harvest in August, all tubers were collected from each pot and evaluated for disease (potato scab) and yield. The experimental design was a randomized complete block design, and each treatment was replicated 8 times. Disease reductions were greatest with the lignin +inoculant treatments in both fumigated and non-fumigated soil; but the relative benefits of carbon addition were greater in non-fumigated than in fumigated soil (FIG. 12A and FIG. 12B). In contrast, yield enhancements were greatest with the microbial inoculants alone (FIG. 16).

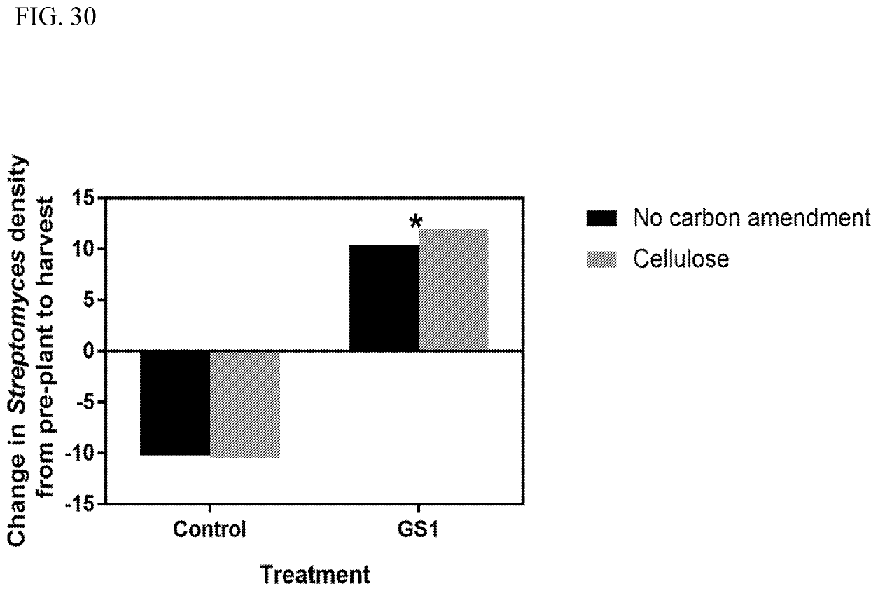

In a related experiment, the effects of carbon amendments on the colonization of inoculants was determined in field plots. Field plots were inoculated with Streptomyces, with or without carbon amendment (cellulose) at planting. Streptomyces inoculant was prepared by growing spores on oatmeal agar mixed with growstone carrier. The resulting granular product was inoculated into field plots. Specifically, potato variety Red Norland was planted into open furrows with the carbon and microbial inoculant treatments added into the furrow at planting. Streptomyces densities (numbers of Streptomyces per gram of soil) were evaluated prior to planting and at harvest for all treatments, providing a means of characterizing the dynamics of Streptomyces in inoculated and non-inoculated plots, and in cellulose amended and non-amended plots. All plots started with no significant differences in Streptomyces densities. Densities in non-inoculated soils decreased over the growing season. In contrast, densities of Streptomyces increased in inoculated plots, and the increases in density were significantly greater with cellulose amendments than without (FIG. 30).

### Example 13

### Multi-Strain Inoculants Provide Better Disease Suppression Compared to Single Strains

This example highlights the value of inoculant combinations as opposed to single-strain inoculants in disease suppression. In this experiment, individual antagonistic isolates were grown on sterilized vermiculite mixed with oatmeal broth (4000 cc vermiculite +1 liter oatmeal broth in a sterile turkey roasting pan sealed with aluminum foil). Inoculum trays were incubated for 9 weeks at room temperature and shaken daily to ensure even distribution.

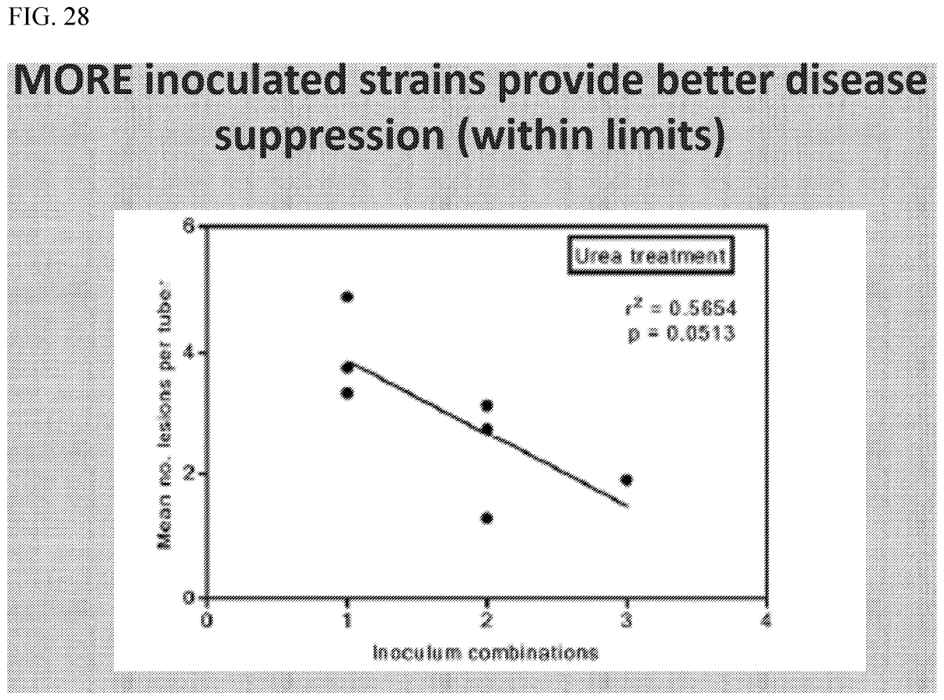

Inoculant was mixed with field soil at a total dose of 9×10⁸ cells per liter of soil. For inoculants that included multiple Streptomyces isolates, inoculum was mixed immediately before planting, and mixed thoroughly with field soil. Potatoes (var. Red Pontiac, a scab-susceptible variety) were planted into the resulting soil with or without added urea (1.38 per pot, equivalent to 110 pounds of nitrogen per acre), with each treatment replicated 8 times (8, 8-liter pots). Potatoes were grown for 16 weeks in the greenhouse. All potatoes were harvested from every pot, and percent tuber area covered with scab lesions, the number of type 3, 4, and 5 lesions (3=periderm broken; 4=pit; and 5=deep pit), and tuber weight was recorded for every tuber. As shown in FIG. 28, the disease intensity (mean number of lesions per tuber, y-axis) was seen to decrease with increasing numbers of Streptomyces isolates in the inoculant (inoculum combinations, x-axis).

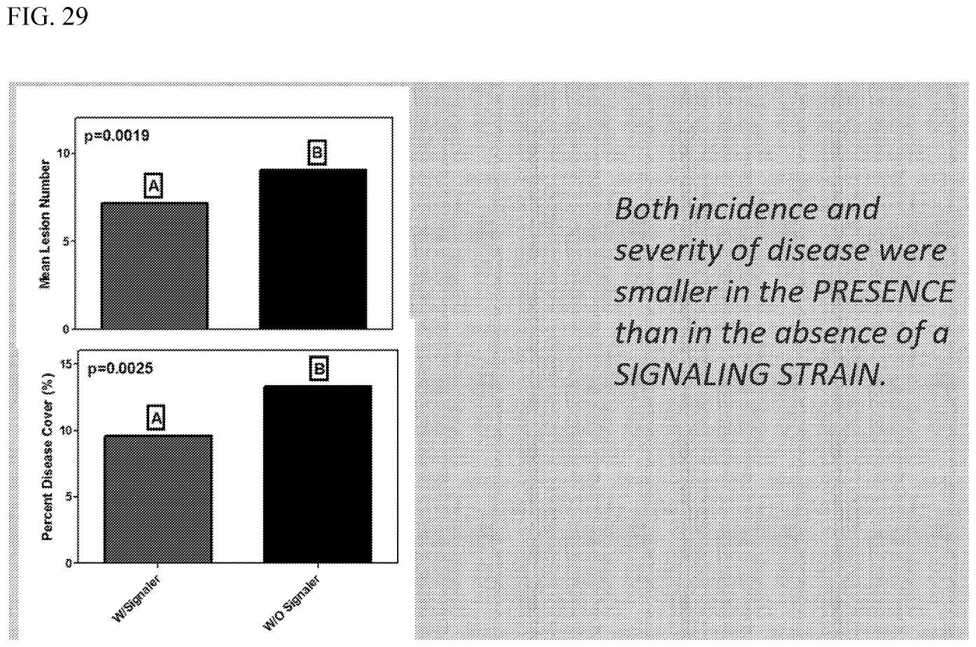

FIG. 29 depicts the benefits of incorporation of a signaling inoculant into the mixture for enhancing disease suppression. Specifically, potato scab disease intensities (either mean lesion number—top panel, or percent surface infected—bottom panel) are significantly smaller when a signaler is inoculated with a collection of antagonists vs. when there no added signaler. Each bar represents the mean disease intensity over 5 different inoculants mixtures PLUS the signaler (Isolate 1231.5), or with no added signaler.

Methods for inoculum preparation, inoculation, and disease assessment duplicate what is described for FIG. 28, except that this experiment was performed in the field. Specifically, inoculum was mixed with field soil and the inoculated soil was subsequently placed into 8-liter pots whose bottom had been removed. Pots with soil were sunk into field plots, each planted in late April with an individual potato (var. Red Pontiac), and maintained under standard management conditions over the growing season. Potatoes were harvested in early September, and disease was assessed as described above

### Example 14

### Prescriptive Soil Amendment Example (Prophetic)

A soil sample isolated from a grower's field will be received and analyzed using the SPPIMC development platform disclosed herein, in order to determine the kind of amendments that could improve soil productivity. First, the types of plant pathogens that infect the soil will be identified. Identification of the plant pathogens inhabiting the soil may be done by analyzing the soil, comprising one or more of the following: isolating pathogens from the soil sample, culturing the pathogens, sequencing whole or parts of the genome of the pathogens (for example, sequencing the 16A rRNA region of the genome), observing the morphology of the pathogen, and observing the appearance of the pathogen culture in liquid media and/or pathogen colonies on solid media. The disease phenotype of the plants grown in the soil will also be examined to help identify the types of pathogens. The microbes in an enriched microbial library, described in Example 2, will be accessed, and screened for activity to suppress the pathogens identified in the soil sample to generate a soil-borne plant pathogen suppressive profile for each microbe, as described in Example 3. Subsequently, the ability of the microbes in the library to inhibit at least one other isolate will be evaluated to give rise to the mutual inhibitory activity microbial library, as described in Example 5; the ability of the microbes in the library to utilize different types of nutrients will be evaluated to give rise to the carbon nutrient utilization profile, as described in Example 6; the ability of the microbes in the library to signal or be signaled by at least one other microbial isolate will be evaluated to give rise to antimicrobial signaling capacity and responsiveness profile, as described in Example 7; and the ability of each of the microbes to promote plant growth will be evaluated, as described in Example 8. In addition, the ability of each of the microbes to resist antibiotics will be evaluated to generate an antibiotic resistance profile, as described in Example 4, and the ability of each of the microbes to grow at different temperatures will be tested to generate a temperature sensitivity profile, as described in Example 15.

Based on the data generated from the experiments above, a library of microbial consortia comprising two or more microbes will be generated and further, screened for all the nodes described above. Microbial consortia in which the individual microbes have complementary pathogen suppressive activity, complementary nutrient utilization activity, less/no mutual inhibition, complementary resistance to antibiotics and an ability to promote plant growth will be selected. Further, microbial consortia, in which at least one microbe signals the production of antimicrobial compounds from other microbes in the consortia will be selected. Microbial consortia possessing these features will be selected for further study in greenhouse and field trials. Finally, the selected microbial consortia will be added as amendments to the field from which the soil samples were collected, and these amendments will be expected to lead to an improvement in plant productivity and suppression of plant disease.

### Example 15

### Characterization of the Microbial Library for Temperature Sensitivity (Prophetic)

As shown in FIG. 14, each of the microbes in the library will be subjected to one of the nodes involved with “multi-dimensional ecological function balancing (MEFB) nodal analysis,” i.e. “determine temperature sensitivity” as follows. Isolates of a previously generated enriched microbial library collected according to the methods of Example 1 will be grown on solid and/or liquid media at different temperatures (for example, 8° C., 15° C., 20° C., 25° C., or 30° C.). The effect of temperature on the growth of the microbe will be assessed. Growth of the microbe may be measured by any one of the methods disclosed herein, for example, by measuring the optical density of the microbial culture after a certain period of growth. Based on the data generated from the experiments above, a library of microbial consortia comprising two or more microbes will be generated and further, screened for the ability to grow at the different temperatures tested above.

The temperature sensitivity of the microbial consortia and individual microbes will be evaluated in view of the temperature of the location where the microbial consortia are intended for use as amendments to soil. Thus, if the soil to be amended with the microbial consortia is located in a tropical climate, then microbial consortia and individual microbes, which have the ability to survive and thrive at higher temperatures (such as 35° C.) will be selected. Likewise, if the soil to be amended with the microbial consortia is located in a more temperature climate, then microbial consortia and individual microbes, which have the ability to survive and thrive at lower temperatures such as 25° C. will be selected.

### Example 16

### Prescription Biocontrol: Soil Carbon Quantity and Diversity are Critical Determinants for Optimizing Inoculant Mixture Niche Differentiation/Overlap Characteristics

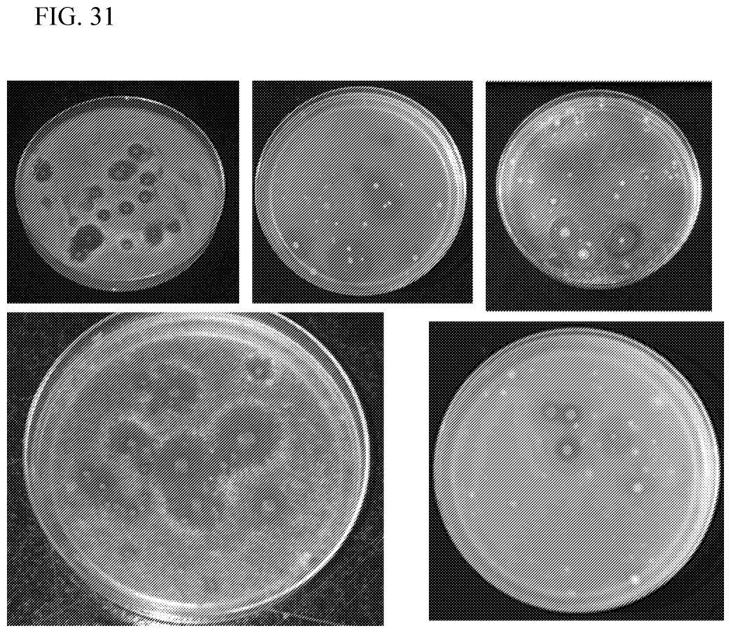

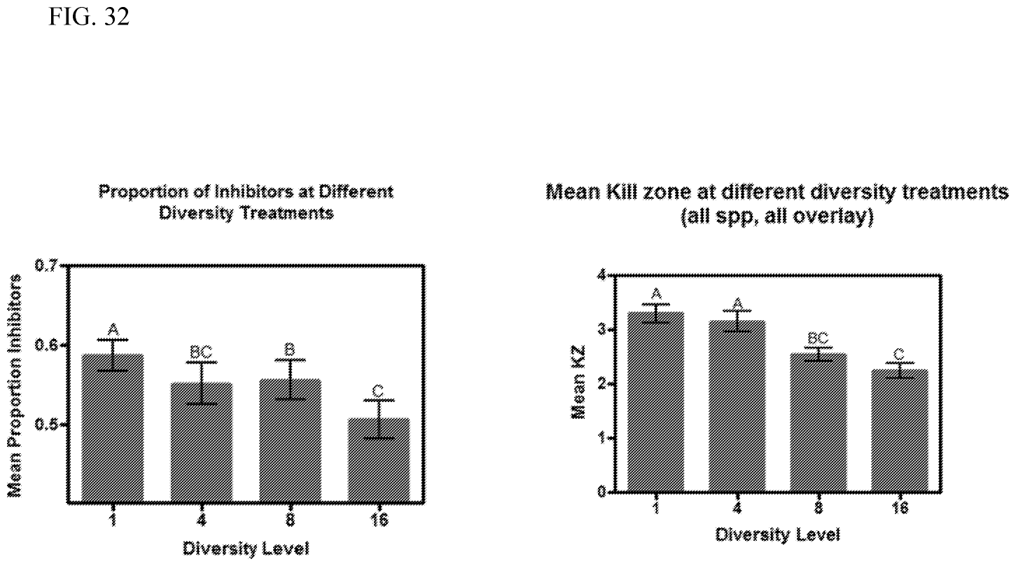

Soil microbiomes were characterized in long-term experimental prairie plots that were planted with one (monoculture), 4, 8, or 16 plant species. Samples were collected in the 17 years after the plots were established. The pathogen-inhibitory capacities of the indigenous soil Streptomyces were determined for every soil using a modified Herr's assay (as described in Wiggins and Kinkel, Phytopatholo. 2005 Feb;95(2):178-85, which is incorporated herein by reference in its entirety). Briefly, soil samples are processed in sterile water for 1 h on a reciprocal shaker (175 cycles/min; 4 C). The resulting suspensions are plated onto water agar, and immediately overlaid with an additional 5 ml water agar. After 5 days, total Streptomyces are quantified on each plate, and an additional layer of agar medium seeded with a plant pathogen (Streptomyces scabies, Verticillium dahliae, Fusarium graminearum, Rhizoctonia solani, or Fusarium oxysporum) is overlaid onto the plate. Following growth of the pathogen, the total number of inhibitory Streptomyces, and the inhibition zone (kill zone) for each Streptomyces is determined. The results show that soils with plants growing in monoculture support significantly greater proportions of inhibitory Streptomyces, and Streptomyces that are better at killing plant pathogens (greater mean kill zones). See FIGS. 31 and 32.

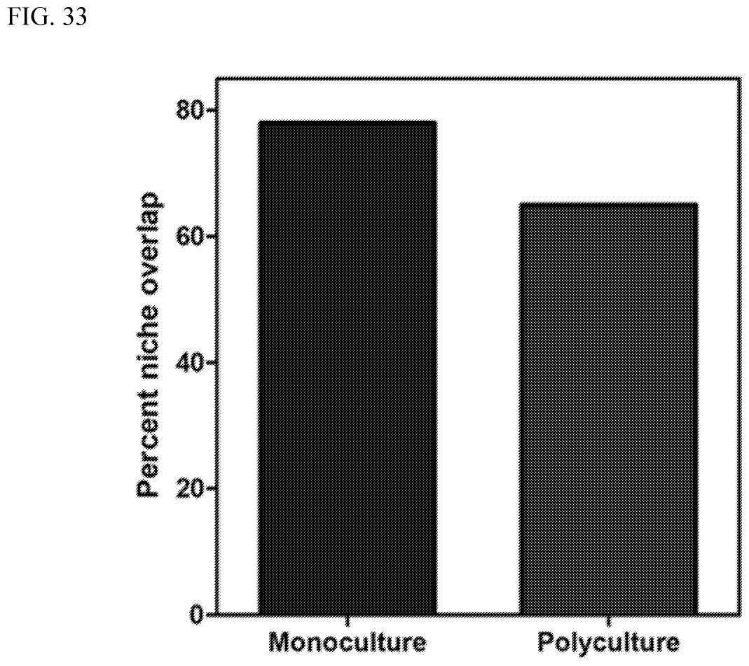

Subsequent research showed that the decreases in inhibitory phenotypes were correlated with increased niche differentiation (significantly smaller niche overlap) among Streptomyces in high-diversity (16 plant species) vs. low-diversity plant communities. Specifically, a random collection of Streptomyces were collected from soils associated with plants growing in monoculture vs. 16-species polyculture, and the resource utilization for every isolate was determined based upon growth on the Biology SF-P2 plates. Niche overlap was determined for all possible pairwise isolate combinations within each plant diversity treatment (FIG. 33). These data support the principle that niche differentiation is a successful strategy for minimizing conflict among Streptomyces in soil, but that the success of this strategy depends upon the specific characteristics of the soil environment.

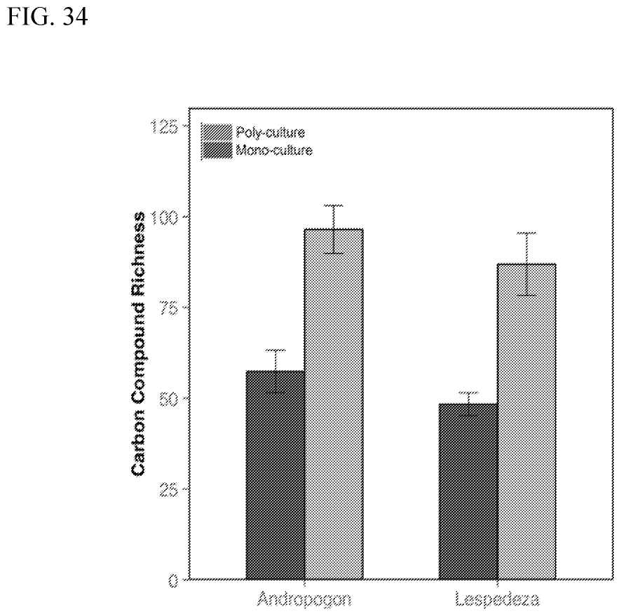

Further research focused in on the soil characteristics that are associated with polyculture vs. monoculture, and specifically the soil characteristics likely to influence the success of a niche differentiation strategy in mediating coexistence. Both total soil carbon and the diversity of soil carbon (number of carbon peaks) are significantly greater in polyculture than in monoculture plots (FIG. 34). This highlights the significant role of soil carbon characteristics in determining optimal strategies for designing microbial inoculant mixtures for different soils. Specifically, these data are consistent with the use of niche-differentiated nutrient specialists for high soil carbon, high carbon diversity soils. In contrast, in low-nutrient soils with low carbon diversity, use of nutrient generalists (which have correspondingly greater niche overlap/less niche differentiation) are recommended for inoculant success.

### Budapest Treaty on the International Recognition of the Deposit of Microorganisms for the Purpose of Patent Procedures

Microorganisms described in this Application were deposited with the American Type Culture Collection (ATCC®), located at 10801 University Blvd., Manassas, Va. 20110, USA. The deposits were made under the terms of the Budapest Treaty on the International Recognition of the Deposit of Microorganisms for the Purposes of Patent Procedure. The ATCC accession numbers for the aforementioned Budapest Treaty deposits are provided herein. A representative sample of microbial consortia LLK3-2017 has been deposited on Jul. 18, 2017, under ATCC accession number PTA-124320. The deposit was tested on Aug. 15, 2017, and found to be viable.

Additional microorganisms described in this Application were deposited with the National Center for Agricultural Utilization Research Agricultural Research Service at the U.S. Department of Agriculture, located at 1815 North University Street, Peoria, Ill. 61604 U.S.A.

The deposits were made under the terms of the Budapest Treaty on the International Recognition of the Deposit of Microorganisms for the Purposes of Patent Procedure. The NRRL accession numbers for the aforementioned Budapest Treaty deposits are provided herein. A representative sample of microbial isolate GS1 has been deposited on Jul. 11, 2019, under accession number NRRL B-67821. A representative sample of microbial isolate PS1 has been deposited on Jul. 11, 2019, under accession number NRRL B-67820. A representative sample of microbial isolate PS3 has been deposited on Jul. 11, 2019, under accession numberNRRL B-67819. All three deposits at the NRRL were confirmed to be viable on Jul. 16, 2019.

Provided herein, are deposits of the following designation: LLK3-2017 (PTA-124320), GS1 (NRRL B-67821), PS1 (NRRL B-67820), and PS3 (B-67819), which are further described herein.

### Incorporation by Reference

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### Embodiments

1. A method for creating a soil-borne plant pathogen inhibiting microbial consortia, comprising:
   a) accessing or creating a soil-borne plant pathogen suppressive microbial library;
   b) utilizing microbes from the library of step a) to access or create one or more ecological function balancing nodal microbial libraries, selected from the group consisting of: a mutual inhibitory activity microbial library, a carbon nutrient utilization complementarity microbial library, an antimicrobial signaling capacity and responsiveness microbial library, a plant growth promotion ability microbial library, an antimicrobial resistance to clinical antimicrobials library, and optionally a temperature sensitivity library;
   c) performing a multi-dimensional ecological function balancing (MEFB) nodal analysis utilizing said one or more nodal microbial libraries; and
   d) selecting at least two microbes from the soil-borne plant pathogen suppressive microbial library based on the MEFB nodal analysis, thereby producing a soil-borne plant pathogen inhibiting microbial consortia having a targeted ecological function in at least one dimension.
2. A method for creating a soil-borne plant pathogen inhibiting microbial consortia, comprising:
   a) accessing or creating a soil-borne plant pathogen suppressive microbial library;
   b) utilizing microbes from the library of step a) to access or create one or more ecological function balancing nodal microbial libraries, selected from the group consisting of: a mutual inhibitory activity microbial library, a carbon nutrient utilization complementarity microbial library, an antimicrobial signaling capacity and responsiveness microbial library, a plant growth promotion ability microbial library, an antimicrobial resistance to clinical antimicrobials library, and optionally a temperature sensitivity library;
   c) performing a multi-dimensional ecological function balancing (MEFB) nodal analysis utilizing said one or more nodal microbial libraries;
   d) assembling a library of microbial consortia, each microbial consortia comprising at least two microbes from the soil-borne plant pathogen suppressive microbial library, selected based on the MEFB nodal analysis;
   e) screening microbial consortia from the library of microbial consortia in the presence of a plurality of soil-borne plant pathogens to produce a soil-borne plant pathogen suppressive profile for each screened microbial consortia;
   f) optionally ranking microbial consortia from the library of screened microbial consortia based upon at least one dimension of the soil-borne plant pathogen suppressive profile of each microbial consortia; and
   g) selecting a soil-borne plant pathogen inhibiting microbial consortia having the desired soil-borne plant pathogen suppressive profile from the library.
3. The method of embodiment 2, comprising: repeating steps a) through e) one or more times.
4. The method of embodiment 2, comprising: repeating steps a) through f) one or more times.
5. The method of embodiment 2, comprising: repeating steps b) through e) one or more times.
6. The method of embodiment 2, comprising: repeating steps b) through f) one or more times.
7. The method of embodiment 2, comprising: repeating steps d) through e) one or more times.
8. The method of embodiment 2, comprising: repeating steps d) through f) one or more times.
9. The method of any one of embodiments 1-8, wherein the step of creating a soil-borne plant pathogen suppressive microbial library comprises creating the soil-borne plant pathogen suppressive microbial library, comprising:
   i) screening a population of microbial isolates in the presence of the soil-borne plant pathogen identified and/or cultured in step (a), to create a soil-borne plant pathogen suppressive profile for each individual microbial isolate in said population,
      wherein said plant pathogen suppressive profile indicates each microbial isolate's ability to suppress the soil-borne plant pathogen identified and/or cultured in step (a).
10. The method of any one of embodiments 1-9, wherein the step of creating a mutual inhibitory activity microbial library comprises the steps of:
   i) assembling a library of test microbial consortia, each test consortia comprising a combination of at least two microbial isolates from the soil-borne plant pathogen suppressive microbial library;
   ii) screening test microbial consortia of the assembled library for the relative degree of mutual inhibitory activity displayed by each microbial isolate towards every other microbial isolate within its own test microbial consortia; and
   iii) developing an n-dimensional mutual inhibitory activity matrix for test microbial consortia based on the mutual inhibitory activities screened in step (i).
11. The method of any one of embodiments 1-10, wherein the step of creating a carbon nutrient utilization complementarity microbial library comprises the step of: i) screening a population of microbial isolates from the soil-borne plant pathogen suppressive microbial library for carbon nutrient utilization by growing said microbial isolates in a plurality of different nutrient media that each comprise a distinct single carbon source to create a carbon nutrient utilization profile for each individual microbial isolate in said population.
12. The method of any one of embodiments 1-11, wherein the step of creating an antimicrobial signaling capacity and responsiveness microbial library comprises the steps of:
   i) screening a population of microbial isolates from the soil-borne plant pathogen suppressive microbial library for the ability of each microbial isolate to signal and modulate the production of antimicrobial compounds in other microbial isolates from the population of microbial isolates; and/or
   ii) screening a population of microbial isolates from the soil-borne plant pathogen suppressive microbial library for the ability of each microbial isolate to be signaled and have their production of antimicrobial compounds modulated by other microbial isolates from the population of microbial isolates;
   thereby creating an antimicrobial signaling capacity and responsiveness profile for each screened individual microbial isolate.
13. The method of any one of embodiments 1-12, wherein the step of creating an antimicrobial resistance to clinical antimicrobials library comprises the steps of:
   i) screening microbial isolates from the soil-borne plant pathogen suppressive microbial library for resistance to a plurality of antibiotics to create an n-dimensional antibiotic resistance profile.
14. The method of any one of embodiments 1-13, wherein the step of creating a plant growth promotion ability microbial library comprises the steps of:
   i) applying microbial isolates from the soil-borne plant pathogen suppressive microbial library to a test plant,
   ii) cultivating the test plant to maturity, and
   iii) comparing the growth of the test plant against that of a control plant that did not receive the microbial isolate;
      wherein differences in the growth between the test plant and the control plant demonstrate a microbial isolate's plant growth promotion ability.
15. The method of any one of embodiments 1-14, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.
16. The method of any one of embodiments 1-14, wherein the soil-borne pathogen is selected from the group consisting of: species of Erwinia, Rhizomonas, Streptomyces scabies, Pseudomonas, and Xanthomonas.
17. A method for creating a soil-borne plant pathogen inhibiting microbial consortia having a targeted and complementary soil-borne plant pathogen suppressive profile, comprising:
   a) screening a population of microbial isolates in the presence of a plurality of soil-borne plant pathogens, including a target soil-borne pathogen, to create a soil-borne plant pathogen suppressive profile for each individual microbial isolate in said population;
   b) assembling a library of microbial consortia, each microbial consortia comprising a combination of microbial isolates from those screened in step a),
      i. wherein each microbial consortia in said library has a predicted soil-borne plant pathogen suppressive profile that is distinct from any individual microbial isolate soil-borne plant pathogen suppressive profile from step a) in at least one dimension of the soil-borne plant pathogen suppressive profile;
      ii. wherein at least one microbial isolate in each of the assembled microbial consortia suppresses the growth of the target soil-borne pathogen;
   c) optionally ranking microbial consortia from the library of microbial consortia based upon at least one dimension of the predicted soil-borne plant pathogen suppressive profile of each microbial consortia; and
   d) selecting a soil-borne plant pathogen inhibiting microbial consortia from the library of microbial consortia, said selected consortia having the desired targeted and complementary soil-borne plant pathogen suppressive profile.
18. A method for creating a soil-borne plant pathogen inhibiting microbial consortia having a targeted and complementary soil-borne plant pathogen suppressive profile, comprising:
   a) screening a population of microbial isolates in the presence of a plurality of soil-borne plant pathogens, including a target soil-borne pathogen, to create a soil-borne plant pathogen suppressive profile for each individual microbial isolate in said population;
   b) assembling a library of microbial consortia, each microbial consortia comprising a combination of microbial isolates from those screened in step a),
      i. wherein each microbial consortia in said library has a predicted soil-borne plant pathogen suppressive profile that is distinct from any individual microbial isolate soil-borne plant pathogen suppressive profile from step a) in at least one dimension of the soil-borne plant pathogen suppressive profile;
      ii. wherein at least one microbial isolate in each of the assembled microbial consortia suppresses the growth of the target soil-borne pathogen;
   c) screening microbial consortia from the library of microbial consortia in the presence of a plurality of soil-borne plant pathogens, including the target soil-borne pathogen, to produce a soil-borne plant pathogen suppressive profile for each screened microbial consortia;
   d) optionally ranking microbial consortia from the library of screened microbial consortia based upon at least one dimension of the soil-borne plant pathogen suppressive profile of each microbial consortia; and
   e) selecting a soil-borne plant pathogen inhibiting microbial consortia having the desired targeted and complementary soil-borne plant pathogen suppressive profile from the library.
19. The method of embodiment 18, comprising: repeating steps a) through d) one or more times.
20. The method of embodiment 18, comprising: repeating steps a) through c) one or more times.
21. The method of embodiment 18, comprising: repeating steps b) through d) one or more times.
22. The method of embodiment 18, comprising: repeating steps b) through c) one or more times.
23. The method of any one of embodiments 17-22, wherein each microbial consortia in said library assembled in step b) has a soil-borne plant pathogen suppressive profile that is distinct from any individual microbial isolate soil-borne plant pathogen suppressive profile from step a) in at least one dimension selected from the group consisting of:
   i. strength of suppressive activity against any one or more member of the plurality of soil-borne plant pathogens,
   ii. specificity against any one or more member of the plurality of soil-borne plant pathogens, and
   iii. breadth of activity against any one or more member of the plurality of soil-borne plant pathogens.
24. The method of any one of embodiments 17-23, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.
25. The method of any one of embodiments 17-23, wherein the soil-borne pathogen is selected from the group consisting of: species of Erwinia, Rhizomonas, Streptomyces scabies, Pseudomonas, and Xanthomonas.
26. A method for creating a soil-borne plant pathogen inhibiting microbial consortia having a designed level of mutual inhibitory activity, comprising:
   a) assembling a library of microbial consortia, each consortia comprising a combination of at least two microbial isolates;
   b) screening microbial consortia of the assembled library for relative degree of mutual inhibitory activity displayed by each microbial isolate towards every other microbial isolate within its microbial consortia;
   c) developing an n-dimensional mutual inhibitory activity matrix for microbial consortia based on the mutual inhibitory activities screened in step (b); and
   d) selecting a soil-borne plant pathogen inhibiting microbial consortia having the designed level of mutual inhibitory activity from the library based upon the n-dimensional mutual inhibitory activity matrix.
27. The method of embodiment 26, wherein the screening of the microbial consortia for the relative degree of mutual inhibitory activity is conducted in pairs, such that each microbial isolate in the consortia is individually tested with one other microbial isolate in the microbial consortia.
28. The method of embodiment 26, wherein the screening of the microbial consortia for the relative degree of mutual inhibitory activity is conducted in groups of three or more, such that a first microbial isolate is screened for mutual inhibitory activity toward another microbial isolate, when said first microbial isolate is grown adjacent or in contact with a third microbial isolate; wherein the first, second and third microbial isolates are all part of the screened microbial consortia.
29. The method of embodiment 26, wherein the screening of the microbial consortia for the relative degree of mutual inhibitory activity comprises testing the relative degree of inhibitory activity against each microbial isolate in the consortia, caused by the combination of all other remaining microbial isolates in the microbial consortia.
30. The method of any one of embodiments 26-29, comprising the step of screening a population of microbial isolates in the presence of a plurality of soil-borne plant pathogens to create a soil-borne plant pathogen suppressive profile for each individual microbial isolate in said population, wherein the microbial consortia of step (a) comprise soil-borne plant pathogen suppressive profile.
31. A method for creating a soil-borne plant pathogen inhibiting microbial consortia having a designed level of mutual inhibitory activity, comprising:
   a) screening a population of microbial isolates for relative degree of mutual inhibitory activity displayed by each microbial isolate towards at least one other individual microbial isolate in the screened population, to create an n-dimensional mutual inhibitory activity matrix based on the mutual inhibitory activities for the screened population;
   b) assembling a library of microbial consortia, each microbial consortia comprising a combination of microbial isolates from those screened in step a),
      i. wherein the microbial isolates of each microbial consortia in said library are expected to be able to grow together based on the n-dimensional mutual inhibitory activity matrix of step a); and
   c) selecting a soil-borne plant pathogen inhibiting microbial consortia having the level of mutual inhibitory activity from the library of step b).
32. A method for creating a soil-borne plant pathogen inhibiting microbial consortia having a designed level of mutual inhibitory activity, comprising:
   a) screening a population of microbial isolates for relative degree of mutual inhibitory activity displayed by each microbial isolate towards at least one other individual microbial isolate in the screened population, to create an n-dimensional mutual inhibitory activity matrix based on the mutual inhibitory activities for the screened population;
   b) assembling a library of microbial consortia, each microbial consortia comprising a combination of microbial isolates from those screened in step a),
      ii. wherein the microbial isolates of each microbial consortia in said library are expected to be able to grow together based on the n-dimensional mutual inhibitory activity matrix of step a)
   c) screening consortia from the library of microbial consortia by growing said consortia in a growth medium and monitoring the continued presence of each microbial isolate within each consortia
   d) selecting a soil-borne plant pathogen inhibiting microbial consortia having the level of mutual inhibitory activity from the library of step b).
33. The method of embodiment 32, comprising: repeating steps a) through c) one or more times.
34. The method of embodiment 32, comprising: repeating steps b) through c) one or more times.
35. The method of any one of embodiments 32-34, comprising the step of screening a population of microbial isolates in the presence of a plurality of soil-borne plant pathogens to create a soil-borne plant pathogen suppressive profile for each individual microbial isolate in said population, wherein the population of microbial isolates of step (a) comprise soil-borne plant pathogen suppressive profile.

36. The method of any one of embodiments embodiment 26-35, wherein the n-dimensional mutual inhibitory activity matrix comprises a dimension selected from the group consisting of:
i. strength of inhibitory activity against one or more member microbial isolates,
ii. specificity against one or more member of a plurality of microbial isolates, and
iii. breadth of activity against any one or more member of the plurality of microbial isolates.
37. The method of any one of embodiments 26-36, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.
38. The method of any one of embodiments 26-36, wherein the soil-borne pathogen is selected from the group consisting of: species of Erwinia, Rhizomonas, Streptomyces scabies, Pseudomonas, and Xanthomonas.
39. A method for creating a soil-borne plant pathogen inhibiting microbial consortia having an optimal and designed level of carbon nutrient utilization complementarity, comprising:
   a) screening a population of microbial isolates for carbon nutrient utilization by growing said microbial isolates in a plurality of different nutrient media that comprise a distinct single carbon source to create a carbon nutrient utilization profile for each individual microbial isolate in said population;
   b) assembling a library of microbial consortia, each microbial consortia comprising a combination of microbial isolates from those screened in step a),
      i. wherein each microbial consortia in said library has a predicted carbon nutrient utilization profile that is distinct from any individual microbial isolate carbon nutrient utilization profile from step a) in at least one dimension of the carbon nutrient utilization profile;
   c) optionally ranking microbial consortia from the library of microbial consortia based upon at least one dimension of the carbon nutrient utilization profile of each microbial consortia in said library; and
   d) selecting a soil-borne plant pathogen inhibiting microbial consortia having an optimal and designed level of carbon nutrient utilization complementarity from the library.
40. The method of embodiment 39, comprising the step of screening a population of microbial isolates in the presence of a plurality of soil-borne plant pathogens to create a soil-borne plant pathogen suppressive profile for each individual microbial isolate in said population, wherein the population of microbial isolates of step (a) comprise soil-borne plant pathogen suppressive profile.
41. The method of any one of embodiments 39 and 40, comprising: repeating steps a) through c) one or more times.
42. The method of any one of embodiments 39 and 40, comprising: repeating steps a) through b) one or more times.
43. The method of any one of embodiments 39 and 40, comprising: repeating steps b) through c) one or more times.
44. The method of any one of embodiments 39 and 40, comprising: repeating step b) one or more times.
45. The method of any one of embodiments 39-44, wherein each microbial consortia in said library assembled in step b) has a carbon nutrient utilization profile that is distinct from any individual microbial isolate carbon nutrient utilization profile from step a) in at least one dimension selected from the group consisting of:
   i. binary ability to grow in any one distinct single carbon source found in said plurality of different nutrient media,
   ii. strength of ability to grow in any one distinct single carbon source found in said plurality of different nutrient media,
   iii. binary ability to grow in at least two distinct single carbon sources found in said plurality of different nutrient media, and
   iv. strength of ability to grow in at least two distinct single carbon sources found in said plurality of different nutrient media.
46. A method for creating a soil-borne plant pathogen inhibiting microbial consortia having an optimal and designed level of carbon nutrient utilization complementarity, comprising:
   a) screening a population of microbial isolates for carbon nutrient utilization by growing said microbial isolates in a plurality of different nutrient media that comprise a distinct single carbon source to create a carbon nutrient utilization profile for each individual microbial isolate in said population;
   b) assembling a library of microbial consortia, each microbial consortia comprising a combination of microbial isolates from those screened in step a),
      i. wherein each microbial consortia in said library has a predicted carbon nutrient utilization profile that is distinct from any individual microbial isolate carbon nutrient utilization profile from step a) in at least one dimension of the carbon nutrient utilization profile;
   c) optionally screening consortia from the library of microbial consortia by growing said consortia in a growth medium and monitoring the continued presence of each microbial isolate within each consortia; and
   d) selecting a soil-borne plant pathogen inhibiting microbial consortia having an optimal and designed level of carbon nutrient utilization complementarity from the library.
47. The method of embodiment 46, wherein the population of microbial isolates screened in step a) comprise a soil-borne plant pathogen suppressive profile.
48. A method for creating a soil-borne plant pathogen inhibiting microbial consortia having an optimal and designed level of carbon nutrient utilization complementarity, comprising:
   a) accessing a carbon nutrient utilization complementarity microbial library;
   b) assembling a library of microbial consortia, each microbial consortia comprising a combination of microbial isolates from the carbon nutrient utilization complementarity microbial library,
      i. wherein each microbial consortia in said library has a predicted carbon nutrient utilization profile that is distinct from any individual microbial isolate carbon nutrient utilization profile from step a) in at least one dimension of the carbon nutrient utilization profile;
   c) optionally screening consortia from the library of microbial consortia by growing said consortia in a growth medium and monitoring the continued presence of each microbial isolate within each consortia; and
   d) selecting a soil-borne plant pathogen inhibiting microbial consortia having an optimal and designed level of carbon nutrient utilization complementarity from the library.
49. The method of embodiment 48, wherein the microbial isolates assembled in step b) comprise a soil-borne plant pathogen suppressive profile.
50. The method of any one of embodiments 46-49, wherein the growth medium of step (c) is medium from the locus where the microbial consortia will be applied, or is medium mimicking a carbon nutrient profile of the locus where the microbial consortia will be applied.
51. The method of any one of embodiments 46-50, comprising: repeating steps a) through c) one or more times.
52. The method of any one of embodiments 46-50, comprising: repeating steps b) through c) one or more times.
53. A method for creating a soil-borne plant pathogen inhibiting microbial consortia having an optimal and designed level of carbon nutrient utilization complementarity, comprising:
   a) assembling a library of microbial consortia, each microbial consortia comprising a combination of microbial isolates comprising a carbon nutrient utilization profile,
      ii. wherein each microbial consortia in said library has a carbon nutrient utilization profile that is distinct from any individual microbial isolate carbon nutrient utilization profile from step a) in at least one dimension of the carbon nutrient utilization profile;
   b) optionally screening consortia from the library of microbial consortia by growing said consortia in a growth medium and monitoring the continued presence of each microbial isolate within each consortia; and
   c) selecting a soil-borne plant pathogen inhibiting microbial consortia having an optimal and designed level of carbon nutrient utilization complementarity from the library.
54. The method of embodiment 53, wherein the growth medium of step (c) is medium from the locus where the microbial consortia will be applied, or is medium mimicking a carbon nutrient profile of the locus where the microbial consortia will be applied.
55. The method of any one of embodiments 53-54, wherein the microbial isolates assembled in step a) comprise a soil-borne plant pathogen suppressive profile.
56. The method of any one of embodiments 53-55, comprising: repeating steps a) through b) one or more times.
57. The method of any one of embodiments 53-56, wherein each assembled microbial consortia has a carbon nutrient utilization profile that is distinct from any individual microbial isolate carbon nutrient utilization profile from step a) in at least one dimension selected from the group consisting of:
   i. binary ability to grow in any one distinct single carbon source found in said plurality of different nutrient media,
   ii. strength of ability to grow in any one distinct single carbon source found in said plurality of different nutrient media,
   iii. binary ability to grow in at least two distinct single carbon sources found in said plurality of different nutrient media, and
   iv. strength of ability to grow in at least two distinct single carbon sources found in said plurality of different nutrient media.
58. The method of any one of embodiments 39-57, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.
59. The method of any one of embodiments 39-57, wherein the soil-borne pathogen is selected from the group consisting of: species of Erwinia, Rhizomonas, Streptomyces scabies, Pseudomonas, and Xanthomonas.
60. A method for creating a soil-borne plant pathogen inhibiting microbial consortia having an optimal and designed level of antibiotic resistance, said method comprising the following steps:
   a) creating an n-dimensional antibiotic resistance profile for each individual microbial isolate of a microbial population;
   b) assembling a library of microbial consortia, each consortia comprising a plurality of microbial isolates from those screened in step a);
   c) optionally ranking microbial consortia from the library of microbial consortia based upon at least one dimension of the antibiotic resistance profile of each microbial consortia in said library; and
   d) selecting a soil-borne plant pathogen inhibiting microbial consortia having an optimal and designed level of antibiotic resistance from the library.
61. A method for creating a soil-borne plant pathogen inhibiting microbial consortia having an optimal and designed level of antibiotic resistance comprising the following steps:
   a) screening a population of microbial isolates for resistance to a plurality of antibiotics to create an n-dimensional antibiotic resistance profile (or, alternatively, accessing a previously created antibiotic resistance profile);
   b) assembling a library of microbial consortia, each microbial consortia comprising a combination of microbial isolates from step a), wherein each microbial consortia in said library is expected to share the antibiotic resistance profile of the individual microbial isolates within the consortia;
   c) screening consortia from the library of microbial consortia by growing said microbial consortia in a growth medium comprising an antibiotic that all of the microbial isolates in the microbial consortia are individually resistant to, and monitoring the continued presence of each microbial isolate within each consortia; and
   d) selecting a soil-borne plant pathogen inhibiting microbial consortia having the optimal and designed level of antibiotic resistance.
62. The method of any one of embodiments 60 and 61, wherein the microbial isolates assembled in step b) comprise a soil-borne plant pathogen suppressive profile.
63. The method of any one of embodiments 61 and 62, comprising: repeating steps a)-c) one or more times.
64. The method of any one of embodiments 61 and 62, comprising: repeating steps a)-d) one or more times.
65. The method of any one of embodiments 61 and 62, comprising: repeating steps b)-c) one or more times.
66. The method of any one of embodiments 61 and 62, comprising: repeating steps b)-d) one or more times.
67. The method of any one of embodiments 60-66, wherein the n-dimensional antibiotic resistance profile comprises at least one dimension selected from the group consisting of:
   i. binary ability to grow in the presence of an antibiotic,
   ii. the concentration of antibiotic under which the microbial isolate is still capable of growing; and
   iii. range of antibiotics against which resistance is shown.
68. The method of any one of embodiments 60-67, wherein the antibiotic is selected from the group consisting of tetracycline, chloramphenicol, vancomycin, erythromycin, novobiocin, streptomycin, azithromycin, kanamycin, and rifampin.
69. The method of any one of embodiments 60-68, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.
70. The method of any one of embodiments 60-68, wherein the soil-borne pathogen is selected from the group consisting of: species of Erwinia, Rhizomonas, Streptomyces scabies, Pseudomonas, and Xanthomonas.
71. A method for creating a soil-borne plant pathogen inhibiting microbial consortia having an optimal and designed level of antimicrobial signaling capacity and responsiveness, comprising:
   a) creating an antimicrobial signaling capacity and responsiveness profile for each individual microbial isolate of a microbial population, comprising:
      i. screening the population of microbial isolates for the ability of each microbial isolate to signal and modulate the production of antimicrobial compounds in other microbial isolates from the population of microbial isolates; and/or
      ii. screening a population of microbial isolates for the ability of each microbial isolate to be signaled and have their production of antimicrobial compounds modulated by other microbial isolates from the population of microbial isolates;
   b) assembling a library of microbial consortia, each consortia comprising a plurality of microbial isolates from those screened in step a),
      i. wherein each microbial consortia in said library has an antimicrobial signaling capacity and responsiveness profile that is distinct from any individual microbial isolate antimicrobial signaling capacity and responsiveness profile from step a) in at least one dimension of the antimicrobial signaling capacity and responsiveness profile;
   c) optionally ranking microbial consortia from the library of microbial consortia based upon at least one dimension of the antimicrobial signaling capacity and responsiveness profile of each microbial consortia in said library; and
   d) selecting a soil-borne plant pathogen inhibiting microbial consortia having an optimal and designed level of antimicrobial signaling capacity and responsiveness from the library.
72. A method for creating a soil-borne plant pathogen inhibiting microbial consortia having an optimal and designed level of antimicrobial signaling capacity and responsiveness, comprising:
   a) creating an antimicrobial signaling capacity and responsiveness profile for each individual microbial isolate of a microbial population, comprising:
      i. screening the population of microbial isolates for the ability of each microbial isolate to signal and modulate the production of antimicrobial compounds in other microbial isolates from the population of microbial isolates; and/or
      ii. screening a population of microbial isolates for the ability of each microbial isolate to be signaled and have their production of antimicrobial compounds modulated by other microbial isolates from the population of microbial isolates;
   b) assembling a library of microbial consortia, each consortia comprising a plurality of microbial isolates from those screened in step a),
      iii. wherein at least one microbial isolate in the microbial consortia exhibits the ability to signal and modulate the production of antimicrobial compounds in another microbial isolate in the consortia;
   c) optionally screening microbial consortia from the library of microbial consortia in the presence of a soil-borne pathogen targeted by the antimicrobial compound(s) produced as a consequence of the antimicrobial signaling capacity or responsiveness of at least one microbial isolate in the microbial consortia from step (a); and
   d) selecting a soil-borne plant pathogen inhibiting microbial consortia having an optimal and designed level of antimicrobial signaling capacity and responsiveness from the library.
73. A method for creating a soil-borne plant pathogen inhibiting microbial consortia having an optimal and designed level of antimicrobial signaling capacity and responsiveness, comprising:
   a) accessing previously gathered antimicrobial signaling capacity and responsiveness profiles for individual microbial isolate of a microbial population;
   b) assembling a library of microbial consortia, each consortia comprising a plurality of microbial isolates from those of step a),
      i. wherein at least one microbial isolate in the microbial consortia exhibits the ability to signal and modulate the production of antimicrobial compounds in another microbial isolate in the consortia;
   c) optionally screening microbial consortia from the library of microbial consortia in the presence of a soil-borne pathogen targeted by the antimicrobial compound(s) produced as a consequence of the antimicrobial signaling capacity or responsiveness of at least one microbial isolate in the microbial consortia from step (a); and
   d) selecting a soil-borne plant pathogen inhibiting microbial consortia having an optimal and designed level of antimicrobial signaling capacity and responsiveness from the library.
74. The method of any one of embodiments 71-73, wherein the microbial isolates assembled in step b) comprise a soil-borne plant pathogen suppressive profile.
75. The method of embodiment 71, comprising: repeating steps a) through c) one or more times.
76. The method of embodiment 71, comprising: repeating steps b) through c) one or more times.
77. The method of any one of embodiments 72-74, comprising: repeating steps a) through c) one or more times.
78. The method of any one of embodiments 72-74, comprising: repeating steps b) through c) one or more times.
79. The method of any one of embodiments 71-78, wherein the antimicrobial signaling capacity and responsiveness profile comprises at least one dimension selected from the group consisting of:
   i. binary ability to signal and modulate the production of antimicrobial compounds in other microbial isolates, and
   ii. strength of ability to signal and modulate the production of antimicrobial compounds in other microbial isolates.
80. The method of any one of embodiments 71-78, wherein the antimicrobial signaling capacity and responsiveness profile comprises at least one dimension selected from the group consisting of:
   i. binary ability to be signaled and have their production of antimicrobial compounds modulated by other microbial isolates, and
   ii. strength of ability to be signaled and have their production of antimicrobial compounds modulated by other microbial isolates.
81. The method of any one of embodiments 72-80, wherein the screening of a population of microbial isolates in step a) comprises: utilizing genomic information, transcriptomic information, and/or growth culture information.
82. A method for creating a soil-borne plant pathogen inhibiting microbial consortia having an optimal and designed level of antimicrobial signaling capacity and responsiveness, comprising:
   a) assembling a library of microbial consortia, each consortia comprising a plurality of microbial isolates wherein at least one of said microbial isolates exhibits antimicrobial signaling capacity towards at least one other microbial isolate in the microbial consortia;
   b) screening microbial consortia from the library of microbial consortia in the presence of a soil-borne pathogen targeted by the antimicrobial compound(s) produced as a consequence of the antimicrobial signaling capacity or responsiveness of at least one microbial isolate in the microbial consortia;
   c) optionally ranking microbial consortia from the library of screened microbial consortia based upon at least one dimension of the antimicrobial signaling capacity and responsiveness profile of each screened microbial consortia; and
   d) selecting a soil-borne plant pathogen inhibiting microbial consortia having an optimal and designed level of antimicrobial signaling capacity and responsiveness from the library.
83. The method of embodiment 82, wherein the microbial isolates assembled in step b) comprise a soil-borne plant pathogen suppressive profile.
84. The method of embodiment 82, comprising: repeating steps a) through c) one or more times.
85. The method of embodiment 82, comprising: repeating steps a) through b) one or more times.
86. A method for screening and evaluating a population of microbial isolates for their plant growth ability, said method comprising the steps of
   a) applying microbial isolates from the population to a test plant,
   b) cultivating the test plant to maturity, and
   c) comparing the growth of the test plant against that of a control plant that did not receive the microbial isolate;
      wherein differences in the growth between the test plant and the control plant demonstrate a microbial isolate's plant growth promotion ability.
87. A method for creating a soil-borne plant pathogen inhibiting microbial consortia having an optimal and designed level of plant growth promoting ability, comprising the following steps:
   (a) creating plant growth promoting ability profile for each individual microbial isolate of a microbial population by screening and evaluating each microbial isolate's ability to promote growth of one or more plants;
   (b) assembling a library of microbial consortia, each consortia comprising a plurality of microbial isolates from those screened in step a);
   (c) optionally ranking microbial consortia from the library of microbial consortia based upon at least one dimension of the plant growth promoting ability of each microbial consortia in said library; and
   (d) selecting a soil-borne plant pathogen inhibiting microbial consortia having an optimal and designed level of plant growth promoting ability from the library.
88. A method for creating a soil-borne plant pathogen inhibiting microbial consortia having an optimal and designed level of plant growth promoting ability, comprising the following steps:
   (a) creating plant growth promoting ability profile for each individual microbial isolate of a microbial population by screening and evaluating each microbial isolate's ability to promote growth of one or more plants;
   (b) assembling a library of microbial consortia, each consortium comprising a plurality of microbial isolates from those screened in step a);
   (c) screening microbial consortia from the library of microbial consortia by:
      i) applying microbial consortia from the library to a test plant,
      ii) cultivating the test plant to maturity, and
      iii) comparing the growth of the test plant against that of a control plant that did not receive the microbial consortia; thereby producing a plant growth promoting ability profile for each screened microbial consortia;
   (d) optionally ranking microbial consortia from the library of screened microbial consortia based upon at least one dimension of the plant growth promoting ability profile of each screened microbial consortia; and
   (e) selecting a soil-borne plant pathogen inhibiting microbial consortia having an optimal and designed level of plant growth promoting ability from the library.
89. The method of embodiment 88, comprising: repeating steps a) through c) one or more times.
90. The method of embodiment 88, comprising: repeating steps a) through d) one or more times.
91. The method of embodiment 88, comprising: repeating steps b) through c) one or more times.
92. The method of embodiment 88, comprising: repeating steps b) through d) one or more times.
93. The method of any one of embodiments 87-92, wherein the microbial isolates assembled in step b) comprise a soil-borne plant pathogen suppressive profile.
94. The method of any one of embodiments 87-93, wherein plant growth promoting ability profile for each screened microbial consortia comprises at least one dimension selected from the group consisting of:
   i. binary ability to promote growth of a particular plant;
   ii. the degree of growth promotion for the particular plant; and
   iii. the mechanism by which the consortia promotes the growth of the particular plant.
95. The method of any one of embodiments 87-94, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.
96. The method of any one of embodiments 87-94, wherein the soil-borne pathogen is selected from the group consisting of: species of Erwinia, Rhizomonas, Streptomyces scabies, Pseudomonas, and Xanthomonas.
97. A plant pathogen inhibiting microbial consortia, comprising:
   a) a first microbial species that provides pathogen suppression; and
   b) a second microbial species that has the ability to signal and modulate the production of antimicrobial compounds in the first microbial species.
98. A plant pathogen inhibiting microbial consortia, comprising:
   a) Brevibacillus laterosporus;
   b) Streptomyces lydicus; and
   c) Streptomyces sp. 3211.1
99. A plant pathogen inhibiting microbial consortia, comprising:
   a) a Brevibacillus sp. comprising a 16S nucleic acid sequence sharing at least 97% sequence identity to SEQ ID NO: 3;
   b) a Streptomyces sp. comprising a 16S nucleic acid sequence sharing at least 97% sequence identity to SEQ ID NO: 2; and
   c) a Streptomyces sp. comprising a 16S nucleic acid sequence sharing at least 97% sequence identity to SEQ ID NO: 1.
100. A plant pathogen inhibiting microbial consortia, comprising:
   a) a Brevibacillus having deposit accession number NRRL B-67819, or a strain having all of the identifying characteristics of Brevibacillus NRRL B-67819, or a mutant thereof;
   b) a Streptomyces having deposit accession numberNRRL B-67820, or a strain having all of the identifying characteristics of Streptomyces NRRL B-67820, or a mutant thereof; and
   c) a Streptomyces having deposit accession number NRRL B-67821, or a strain having all of the identifying characteristics of Streptomyces NRRL B-67821, or a mutant thereof.
101. A plant pathogen inhibiting microbial consortia, comprising: a microbial consortia having deposit accession number PTA-124320, or an consortia of strains having all of the identifying characteristics of PTA-124320, or mutants thereof.
102. A plant pathogen inhibiting microbial consortia, comprising: a microbial consortia wherein a representative sample of cells have been deposited under accession number PTA-124320, or an consortia of strains having all of the identifying characteristics of PTA-124320, or mutants thereof.
103. A method of improving soil for plant growth, comprising applying the microbial consortia of any one of embodiments 97-102 to the soil.
104 The method of embodiment 103, wherein the microbial consortia is applied before planting.
105. The method of embodiment 103, wherein the microbial consortia is applied after plant germination.
106. The method of embodiment 103, wherein the microbial consortia is applied as a seed treatment.
107. The method of embodiment 103, wherein the microbial consortia is applied as a spray.
108. The method of embodiment 103, wherein the microbial consortia is applied as a soil drench.
109. A method of improving soil for plant growth, comprising applying a microbe to the soil; wherein the microbe is a Brevibacillus having deposit accession number NRRL B-67819, or a strain having all of the identifying characteristics of Brevibacillus NRRL B-67819, or a mutant thereof.
110. A method of improving soil for plant growth, comprising applying a microbe to the soil; wherein the microbe is a Streptomyces having deposit accession number NRRL B-67820, or a strain having all of the identifying characteristics of Streptomyces NRRL B-67820, or a mutant thereof.
111. A method of improving soil for plant growth, comprising applying a microbe to the soil; wherein the microbe is a Streptomyces having deposit accession number NRRL B-67821, or a strain having all of the identifying characteristics of Streptomyces NRRL B-67821, or a mutant thereof.
112. The method of any one of embodiments 109-111, wherein the microbial consortia is applied before planting.
113. The method of any one of embodiments 109-111, wherein the microbial consortia is applied after plant germination.
114. The method of any one of embodiments 109-111, wherein the microbial consortia is applied as a seed treatment.
115. The method of any one of embodiments 109-111, wherein the microbial consortia is applied as a spray.
116. The method of any one of embodiments 109-111, wherein the microbial consortia is applied as a soil drench.
117. A method for prescriptive biocontrol of a soil-borne plant pathogen, said method comprising:
   a) Identifying the soil-borne plant pathogen(s) present in soil or plant tissue from a locus in need of prescriptive biocontrol;
   b) creating a customized soil-borne plant pathogen inhibiting microbial consortia capable of suppressing the growth of the soil-borne plant pathogen identified in step (a), wherein creating said customized microbial consortia comprises the steps of:
      i. accessing a soil-borne plant pathogen suppressive microbial library, said library comprising one or more ecological function balancing nodal libraries selected from the group consisting of: a mutual inhibitory activity microbial library, a carbon nutrient utilization complementarity microbial library, an antimicrobial signaling capacity and responsiveness microbial library, a plant growth promotion ability microbial library, and an antimicrobial resistance to clinical antimicrobials library;
      ii. performing a multi-dimensional ecological function balancing (MEFB) nodal analysis utilizing said one or more nodal libraries; and
      iii. selecting at least two microbes from the soil-borne plant pathogen suppressive microbial library based on the MEFB nodal analysis, thereby producing a soil-borne plant pathogen inhibiting microbial consortia, wherein said microbial consortia is capable of suppressing the growth of the soil-borne plant pathogen(s) identified in step (a).
118. A method for prescriptive biocontrol of a soil-borne plant pathogen, said method comprising:
   c) identifying and/or culturing the soil-borne plant pathogen(s) present in soil or plant tissue from a locus in need of prescriptive biocontrol;
   d) creating a customized soil-borne plant pathogen inhibiting microbial consortia capable of suppressing the growth of the soil-borne plant pathogen identified and/or cultured in step (a), wherein creating said customized microbial consortia comprises the steps of:
      i. accessing a soil-borne plant pathogen suppressive microbial library,
      ii. utilizing microbes from the library of step i) to create one or more ecological function balancing nodal microbial libraries, selected from the group consisting of: a mutual inhibitory activity microbial library, a carbon nutrient utilization complementarity microbial library, an antimicrobial signaling capacity and responsiveness microbial library, a plant growth promotion ability microbial library, an antimicrobial resistance to clinical antimicrobials library, and optionally a temperature sensitivity library;
      iii. performing a multi-dimensional ecological function balancing (MEFB) nodal analysis utilizing said one or more nodal microbial libraries; and
      iv. selecting at least two microbes from the soil-borne plant pathogen suppressive microbial library based on the MEFB nodal analysis, thereby producing a soil-borne plant pathogen inhibiting microbial consortia, wherein said microbial consortia is capable of suppressing the growth of the soil-borne plant pathogen(s) identified in step (a).
119. The method of embodiment 117 or 118, wherein identifying the soil-borne plant pathogen(s) present in soil or plant tissue comprises identification of the pathogen genus based on symptoms of plants grown in said locus in need of prescriptive biocontrol.
120. The method of any one of embodiments 117-119, wherein the step of accessing a soil-borne plant pathogen suppressive microbial library comprises creating the soil-borne plant pathogen suppressive microbial library, comprising:
   i) screening a population of microbial isolates in the presence of the soil-borne plant pathogen identified and/or cultured in step (a), to create a soil-borne plant pathogen suppressive profile for each individual microbial isolate in said population,
      wherein said plant pathogen suppressive profile indicates each microbial isolate's ability to suppress the soil-borne plant pathogen identified and/or cultured in step (a).
121. The method of any one of embodiments 117-120, wherein the step of creating or accessing a mutual inhibitory activity microbial library comprises the steps of:
   i) assembling a library of test microbial consortia, each test consortia comprising a combination of at least two microbial isolates from the soil-borne plant pathogen suppressive microbial library;
   ii) screening test microbial consortia of the assembled library for the relative degree of mutual inhibitory activity displayed by each microbial isolate towards every other microbial isolate within its own test microbial consortia; and
   iii) developing an n-dimensional mutual inhibitory activity matrix for test microbial consortia based on the mutual inhibitory activities screened in step (i).
122. The method of any one of embodiments 117-121, wherein the step of creating or accessing a carbon nutrient utilization complementarity microbial library comprises the step of: i) screening a population of microbial isolates from the soil-borne plant pathogen suppressive microbial library for carbon nutrient utilization by growing said microbial isolates in a plurality of different nutrient media that comprise a distinct single carbon source to create a carbon nutrient utilization profile for each individual microbial isolate in said population.
123. The method of any one of embodiments 117-122, wherein the step of creating an antimicrobial signaling capacity and responsiveness microbial library comprises the steps of:
   i) screening a population of microbial isolates from the soil-borne plant pathogen suppressive microbial library for the ability of each microbial isolate to signal and modulate the production of antimicrobial compounds in other microbial isolates from the population of microbial isolates; and/or
   ii) screening a population of microbial isolates from the soil-borne plant pathogen suppressive microbial library for the ability of each microbial isolate to be signaled and have their production of antimicrobial compounds modulated by other microbial isolates from the population of microbial isolates;
   thereby creating an antimicrobial signaling capacity and responsiveness profile for each screened individual microbial isolate.
124. The method of any one of embodiments 117-123, wherein the step of creating an antimicrobial resistance to clinical antimicrobials library comprises the steps of:
   i) screening microbial isolates from the soil-borne plant pathogen suppressive microbial library for resistance to a plurality of antibiotics to create an n-dimensional antibiotic resistance profile.
125. The method of any one of embodiments 117-124, wherein the step of creating a plant growth promotion ability microbial library comprises the steps of:
   i) applying microbial isolates from the soil-borne plant pathogen suppressive microbial library to a test plant,
   ii) cultivating the test plant to maturity, and
   iii) comparing the growth of the test plant against that of a control plant that did not receive the microbial isolate;
      wherein differences in the growth between the test plant and the control plant demonstrate a microbial isolate's plant growth promotion ability.
126. The composition of any one of embodiments 117-125, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.
127. The composition of any one of embodiments 117-125, wherein the soil-borne pathogen is selected from the group consisting of: species of Erwinia, Rhizomonas, Streptomyces scabies, Pseudomonas, and Xanthomonas.
128. A method for prescriptive biocontrol of a soil-borne plant pathogen, said method comprising:
   a) creating a soil nutrient profile from soil from a locus in need of prescriptive biocontrol;
   b) creating a customized carbon amendment for application on the locus of step a), wherein the customized carbon amendment supplements a carbon deficiency in the nutrient soil profile.
129. The method of embodiment 128, further comprising the step of c) applying the customized soil carbon amendment to the locus.
130. The method of embodiment 129, further comprising the steps of repeating steps a)-b) one or more times.
131. The method of embodiment 129, wherein each repetition of steps a)-b) occurs at least 1, 2, 3, 4 5 6, 7, 8, 9, 10, 11, or 12 months after the last carbon amendment application to the soil.
132. The method of any one of embodiments 128-131, wherein the step of creating a soil nutrient profile comprises the steps of:
   i) providing a soil sample from the locus in need of prescriptive biocontrol; and
   ii) analyzing the carbon nutrient contents of said soil sample.
133. The method of embodiment 132, wherein the carbon nutrient contents of the soil sample are measure via a chromatographic method.
134. The method of embodiment 132, wherein the carbon nutrient contents of the soil sample are measure via an analysis method selected from the group consisting of: Gas Chromatography, Liquid Chromatography, Mass Spectrometer, wet digestion and dry combustion, aerial spectroscopy, Loss on Ignition, Elemental Analyzer, and Reflectance Spectroscopy.
135. The composition of any one of embodiments 128-134, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.
136. The composition of any one of embodiments 128-134, wherein the soil-borne pathogen is selected from the group consisting of: species of Erwinia, Rhizomonas, Streptomyces scabies, Pseudomonas, and Xanthomonas.
137. A method of enhancing the antibiotic inhibitory capacity of individual microbes within a population of microbes in soil, said method comprising the steps of: applying a carbon source to said soil.
138. A method of enriching the densities of inhibitory microorganisms within a population of microbes in soil, said method comprising the steps of: applying a carbon source to said soil.
139. A method of suppressing the growth of pathogens within a soil containing microbes with soil-borne pathogen inhibitory potential, said method comprising the steps of: applying a carbon source to said soil.
140. The method of any one of embodiments 137-139, wherein the carbon source is selected from the group consisting of: glucose, fructose, lignin, ground rice powder, malic acid, and mixtures thereof.
141. The method of any one of embodiments 137-140, wherein the carbon source is applied at least 2, 3, 4, 5, 6, 7, 8, 9, 10, 11, or 12 times in a one year period.
142. A method of enhancing the antibiotic inhibitory capacity of individual microbes within a population of microbes in soil, wherein crops grown in said soil suffer from one or more soil-borne pathogens, said method comprising the steps of:
   a) applying a carbon source to the soil;
   b) assessing the antibiotic inhibitory capacity of microbes within the microbial population in the soil; and
   c) repeating steps (a) and (b) one or more times, until the antibiotic inhibitory capacity of microbes within the microbial population reaches a desired level.
143. The method of embodiment 142, wherein the antibiotic inhibitory capacity of the microbes is assessed based on the presence or absence of symptoms exhibited by the crop due to the soil-borne pathogens.
144. The method of embodiment 142, wherein steps (a) and (b) are repeated until the crops cease to exhibit symptoms from the soil-borne pathogens.
145. A method of enriching the densities of inhibitory microorganisms within a population of microbes in soil, wherein crops grown in said soil suffer from one or more soil-borne pathogens, said method comprising the steps of:
   a) applying a carbon source to the soil;
   b) assessing the densities of inhibitory microorganisms within the soil; and
   c) repeating steps (a) and (b) one or more times, until the densities of inhibitory microorganisms within the soil reaches a desired level.
146. The method of embodiment 145, wherein the densities of inhibitory microorganisms is assessed based on the presence or absence of symptoms exhibited by the crop due to the soil-borne pathogens.
147. The method of embodiment 145, wherein steps (a) and (b) are repeated until the crops cease to exhibit symptoms from the soil-borne pathogens.
148. A method of suppressing the growth of pathogens within a soil, containing microbes with soil-borne pathogen inhibitory potential, said method comprising the steps of:
   a) applying a carbon source to the soil;
   b) determining pathogen density in the soil; and
   c) repeating steps (a) and (b) one or more times, until the pathogen density reaches a desired level.
149. The method of embodiment 148, wherein the densities of inhibitory microorganisms is assessed based on the presence or absence of pathogenic symptoms exhibited by a crop grown on the soil.
150. The method of embodiment 148, wherein steps (a) and (b) are repeated until crops grown on the soil cease to exhibit symptoms from the soil-borne pathogens.
151. The method of any one of embodiments 142-150, wherein step (b) is conducted 1, 2, 3, 4, 5, 6, 7, 8, 9, 10, 11, or 12 months after step (a).
152. A method of treating a soil-borne pathogen in soil, said method comprising the steps of:
   a) applying a combination composition to the soil, said composition comprising
      i) a soil-borne pathogen suppressing microbe; and
      i) a carbon source;
   thereby reducing the symptoms of the soil-borne pathogen on a crop grown in said soil.
153. The method of embodiment 152, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.
154. The method of embodiment 152, wherein the soil-borne pathogen is selected from the group consisting of: species of Erwinia, Rhizomonas, Streptomyces scabies, Pseudomonas, and Xanthomonas.

0155. The method of any one of embodiments 152-154, wherein the soil-borne pathogen suppressing microbe is a microbial isolate.
156. The method of embodiment 155, wherein the microbial isolate is selected from the group consisting Streptomyces GS1 (Streptomyces lydicus), Streptomyces PS1 (Streptomyces sp. 3211.1) and Brevibacillus PS3 (Brevibacillus laterosporus).
157. The method of any one of embodiments 152-154, wherein the soil-borne pathogen suppressing microbe is administered as a microbial consortia.
158. The method of embodiment 157, wherein the microbial consortia comprises Streptomyces GS1 (Streptomyces lydicus), Streptomyces PS1 (Streptomyces sp. 3211.1) and Brevibacillus PS3 (Brevibacillus laterosporus).
159. A composition comprising i) a soil-borne pathogen suppressing microbe; and i) a carbon source, wherein said composition is capable of suppressing the growth of a soil-borne pathogen.
160. The composition of embodiment 159, wherein the soil-borne pathogen suppressing microbe is a microbial isolate.
161. The composition of embodiment 160, wherein the microbial isolate is selected from the group consisting Streptomyces GS1 (Streptomyces lydicus), Streptomyces PS1 (Streptomyces sp. 3211.1) and Brevibacillus PS3 (Brevibacillus laterosporus).
162. The composition of embodiment 159, wherein the soil-borne pathogen suppressing microbe is administered as a microbial consortia.
163. The composition of embodiment 162, wherein the microbial consortia comprises Streptomyces GS1 (Streptomyces lydicus), Streptomyces PS1 (Streptomyces sp. 3211.1) and Brevibacillus PS3 (Brevibacillus laterosporus).
164. The composition of any one of embodiments 159-163, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.
165. The composition of any one of embodiments 159-163, wherein the soil-borne pathogen is selected from the group consisting of: species of Erwinia, Rhizomonas, Streptomyces scabies, Pseudomonas, and Xanthomonas.
166. A method of improving soil for plant growth, comprising applying a microbe to the soil; wherein the microbe is a Brevibacillus sp. comprising a 16S nucleic acid sequence sharing at least 97% sequence identity to SEQ ID NO: 3, 4, or 5.
167. A method of improving soil for plant growth, comprising applying a microbe to the soil; wherein the microbe is a Streptomyces sp. comprising a 16S nucleic acid sequence sharing at least 97% sequence identity to SEQ ID NO: 2
168. A method of improving soil for plant growth, comprising applying a microbe to the soil; wherein the microbe is a Streptomyces sp. comprising a 16S nucleic acid sequence sharing at least 97% sequence identity to SEQ ID NO: 1.
169. The method of any one of embodiments 166-168, wherein the microbial consortia is applied before planting.
170. The method of any one of embodiments 166-168, wherein the microbial consortia is applied after plant germination.
171. The method of any one of embodiments 166-168, wherein the microbial consortia is applied as a seed treatment.
172. The method of any one of embodiments 166-168, wherein the microbial consortia is applied as a spray.
173. The method of any one of embodiments 166-168, wherein the microbial consortia is applied as a soil drench.

1. A method for creating a soil-borne plant pathogen inhibiting microbial consortia, comprising:
a) accessing or creating a soil-borne plant pathogen suppressive microbial library;
b) utilizing microbes from the library of step a) to access or create one or more ecological function balancing nodal microbial libraries, selected from the group consisting of: a mutual inhibitory activity microbial library, a carbon nutrient utilization complementarity microbial library, an antimicrobial signaling capacity and responsiveness microbial library, a plant growth promotion ability microbial library, and an antimicrobial resistance to clinical antimicrobials library;
c) performing a multi-dimensional ecological function balancing (MEFB) nodal analysis utilizing said one or more nodal microbial libraries; and
d) selecting at least two microbes from the soil-borne plant pathogen suppressive microbial library based on the MEFB nodal analysis, thereby producing a soil-borne plant pathogen inhibiting microbial consortia having a targeted ecological function in at least one dimension.

2. A method for creating a soil-borne plant pathogen inhibiting microbial consortia, comprising:
a) accessing or creating a soil-borne plant pathogen suppressive microbial library;
b) utilizing microbes from the library of step a) to access or create one or more ecological function balancing nodal microbial libraries, selected from the group consisting of: a mutual inhibitory activity microbial library, a carbon nutrient utilization complementarity microbial library, an antimicrobial signaling capacity and responsiveness microbial library, a plant growth promotion ability microbial library, and an antimicrobial resistance to clinical antimicrobials library;
c) performing a multi-dimensional ecological function balancing (MEFB) nodal analysis utilizing said one or more nodal microbial libraries;
d) assembling a library of microbial consortia, each microbial consortia comprising at least two microbes from the soil-borne plant pathogen suppressive microbial library, selected based on the MEFB nodal analysis;
e) screening microbial consortia from the library of microbial consortia in the presence of a plurality of soil-borne plant pathogens to produce a soil-borne plant pathogen suppressive profile for each screened microbial consortia;
f) optionally ranking microbial consortia from the library of screened microbial consortia based upon at least one dimension of the soil-borne plant pathogen suppressive profile of each microbial consortia; and
g) selecting a soil-borne plant pathogen inhibiting microbial consortia having the desired soil-borne plant pathogen suppressive profile from the library.

3. The method of claim 2, comprising: repeating steps a) through e) one or more times.

4. The method of claim 2, comprising: repeating steps a) through f) one or more times.

5. The method of claim 2, comprising: repeating steps b) through e) one or more times.

6. The method of claim 2, comprising: repeating steps b) through f) one or more times.

7. The method of claim 2, comprising: repeating steps d) through e) one or more times.

8. The method of claim 2, comprising: repeating steps d) through f) one or more times.

9. The method of claim 1, wherein the step of creating a soil-borne plant pathogen suppressive microbial library comprises creating the soil-borne plant pathogen suppressive microbial library, comprising:
i) screening a population of microbial isolates in the presence of the soil-borne plant pathogen identified and/or cultured in step (a), to create a soil-borne plant pathogen suppressive profile for each individual microbial isolate in said population,
wherein said plant pathogen suppressive profile indicates each microbial isolate's ability to suppress the soil-borne plant pathogen identified and/or cultured in step (a).

10. The method of claim 1, wherein the step of creating a mutual inhibitory activity microbial library comprises the steps of:
i) assembling a library of test microbial consortia, each test consortia comprising a combination of at least two microbial isolates from the soil-borne plant pathogen suppressive microbial library;
ii) screening test microbial consortia of the assembled library for the relative degree of mutual inhibitory activity displayed by each microbial isolate towards every other microbial isolate within its own test microbial consortia; and
iii) developing an n-dimensional mutual inhibitory activity matrix for test microbial consortia based on the mutual inhibitory activities screened in step (i).

11. The method of claim 1, wherein the step of creating a carbon nutrient utilization complementarity microbial library comprises the step of: i) screening a population of microbial isolates from the soil-borne plant pathogen suppressive microbial library for carbon nutrient utilization by growing said microbial isolates in a plurality of different nutrient media that each comprise a distinct single carbon source to create a carbon nutrient utilization profile for each individual microbial isolate in said population.

12. The method of claim 1, wherein the step of creating an antimicrobial signaling capacity and responsiveness microbial library comprises the steps of:
i) screening a population of microbial isolates from the soil-borne plant pathogen suppressive microbial library for the ability of each microbial isolate to signal and modulate the production of antimicrobial compounds in other microbial isolates from the population of microbial isolates; and/or
screening a population of microbial isolates from the soil-borne plant pathogen suppressive microbial library for the ability of each microbial isolate to be signaled and have their production of antimicrobial compounds modulated by other microbial isolates from the population of microbial isolates;
thereby creating an antimicrobial signaling capacity and responsiveness profile for each screened individual microbial isolate.

13. The method of claim 1, wherein the step of creating an antimicrobial resistance to clinical antimicrobials library comprises the steps of:
i) screening microbial isolates from the soil-borne plant pathogen suppressive microbial library for resistance to a plurality of antibiotics to create an n-dimensional antibiotic resistance profile.

14. The method of claim 1, wherein the step of creating a plant growth promotion ability microbial library comprises the steps of:
i) applying microbial isolates from the soil-borne plant pathogen suppressive microbial library to a test plant,
ii) cultivating the test plant to maturity, and
iii) comparing the growth of the test plant against that of a control plant that did not receive the microbial isolate;
wherein differences in the growth between the test plant and the control plant demonstrate a microbial isolate's plant growth promotion ability.

15. The method of claim 1, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.

16. The method of claim 1, wherein the soil-borne pathogen is selected from the group consisting of: species of Erwinia, Rhizomonas, Streptomyces scabies, Pseudomonas, and Xanthomonas.

17. A method for creating a soil-borne plant pathogen inhibiting microbial consortia having a targeted and complementary soil-borne plant pathogen suppressive profile, comprising:
a) screening a population of microbial isolates in the presence of a plurality of soil-borne plant pathogens, including a target soil-borne pathogen, to create a soil-borne plant pathogen suppressive profile for each individual microbial isolate in said population;
b) assembling a library of microbial consortia, each microbial consortia comprising a combination of microbial isolates from those screened in step a),
i. wherein each microbial consortia in said library has a predicted soil-borne plant pathogen suppressive profile that is distinct from any individual microbial isolate soil-borne plant pathogen suppressive profile from step a) in at least one dimension of the soil-borne plant pathogen suppressive profile;
ii. wherein at least one microbial isolate in each of the assembled microbial consortia suppresses the growth of the target soil-borne pathogen;
c) optionally ranking microbial consortia from the library of microbial consortia based upon at least one dimension of the predicted soil-borne plant pathogen suppressive profile of each microbial consortia; and
d) selecting a soil-borne plant pathogen inhibiting microbial consortia from the library of microbial consortia, said selected consortia having the desired targeted and complementary soil-borne plant pathogen suppressive profile.

18. A method for creating a soil-borne plant pathogen inhibiting microbial consortia having a targeted and complementary soil-borne plant pathogen suppressive profile, comprising:
a) screening a population of microbial isolates in the presence of a plurality of soil-borne plant pathogens, including a target soil-borne pathogen, to create a soil-borne plant pathogen suppressive profile for each individual microbial isolate in said population;
b) assembling a library of microbial consortia, each microbial consortia comprising a combination of microbial isolates from those screened in step a),
i. wherein each microbial consortia in said library has a predicted soil-borne plant pathogen suppressive profile that is distinct from any individual microbial isolate soil-borne plant pathogen suppressive profile from step a) in at least one dimension of the soil-borne plant pathogen suppressive profile;
ii. wherein at least one microbial isolate in each of the assembled microbial consortia suppresses the growth of the target soil-borne pathogen;
c) screening microbial consortia from the library of microbial consortia in the presence of a plurality of soil-borne plant pathogens, including the target soil-borne pathogen, to produce a soil-borne plant pathogen suppressive profile for each screened microbial consortia;
d) optionally ranking microbial consortia from the library of screened microbial consortia based upon at least one dimension of the soil-borne plant pathogen suppressive profile of each microbial consortia; and
e) selecting a soil-borne plant pathogen inhibiting microbial consortia having the desired targeted and complementary soil-borne plant pathogen suppressive profile from the library.

19. The method of claim 18, comprising: repeating steps a) through d) one or more times.

20. The method of claim 18, comprising: repeating steps a) through c) one or more times.

21. The method of claim 18, comprising: repeating steps b) through d) one or more times.

22. The method of claim 18, comprising: repeating steps b) through c) one or more times.

23. The method of claim 17, wherein each microbial consortia in said library assembled in step b) has a soil-borne plant pathogen suppressive profile that is distinct from any individual microbial isolate soil-borne plant pathogen suppressive profile from step a) in at least one dimension selected from the group consisting of:
i. strength of suppressive activity against any one or more member of the plurality of soil-borne plant pathogens,
ii. specificity against any one or more member of the plurality of soil-borne plant pathogens, and
iii. breadth of activity against any one or more member of the plurality of soil-borne plant pathogens.

24. The method of claim 17, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.

25. The method of claim 17, wherein the soil-borne pathogen is selected from the group consisting of: species of Erwinia, Rhizomonas, Streptomyces scabies, Pseudomonas, and Xanthomonas.

26. A method for creating a soil-borne plant pathogen inhibiting microbial consortia having a designed level of mutual inhibitory activity, comprising:
a) assembling a library of microbial consortia, each consortia comprising a combination of at least two microbial isolates;
b) screening microbial consortia of the assembled library for relative degree of mutual inhibitory activity displayed by each microbial isolate towards every other microbial isolate within its microbial consortia;
c) developing an n-dimensional mutual inhibitory activity matrix for microbial consortia based on the mutual inhibitory activities screened in step (b); and
d) selecting a soil-borne plant pathogen inhibiting microbial consortia having the designed level of mutual inhibitory activity from the library based upon the n-dimensional mutual inhibitory activity matrix.

27. The method of claim 26, wherein the screening of the microbial consortia for the relative degree of mutual inhibitory activity is conducted in pairs, such that each microbial isolate in the consortia is individually tested with one other microbial isolate in the microbial consortia.

28. The method of claim 26, wherein the screening of the microbial consortia for the relative degree of mutual inhibitory activity is conducted in groups of three or more, such that a first microbial isolate is screened for mutual inhibitory activity toward another microbial isolate, when said first microbial isolate is grown adjacent or in contact with a third microbial isolate; wherein the first, second and third microbial isolates are all part of the screened microbial consortia.

29. The method of claim 26, wherein the screening of the microbial consortia for the relative degree of mutual inhibitory activity comprises testing the relative degree of inhibitory activity against each microbial isolate in the consortia, caused by the combination of all other remaining microbial isolates in the microbial consortia.

30. The method of claim 26, comprising the step of screening a population of microbial isolates in the presence of a plurality of soil-borne plant pathogens to create a soil-borne plant pathogen suppressive profile for each individual microbial isolate in said population, wherein the microbial consortia of step (a) comprise soil-borne plant pathogen suppressive profile.

31. A method for creating a soil-borne plant pathogen inhibiting microbial consortia having a designed level of mutual inhibitory activity, comprising:
a) screening a population of microbial isolates for relative degree of mutual inhibitory activity displayed by each microbial isolate towards at least one other individual microbial isolate in the screened population, to create an n-dimensional mutual inhibitory activity matrix based on the mutual inhibitory activities for the screened population;
b) assembling a library of microbial consortia, each microbial consortia comprising a combination of microbial isolates from those screened in step a),
i. wherein the microbial isolates of each microbial consortia in said library are expected to be able to grow together based on the n-dimensional mutual inhibitory activity matrix of step a); and
c) selecting a soil-borne plant pathogen inhibiting microbial consortia having the level of mutual inhibitory activity from the library of step b).

32. A method for creating a soil-borne plant pathogen inhibiting microbial consortia having a designed level of mutual inhibitory activity, comprising:
a) screening a population of microbial isolates for relative degree of mutual inhibitory activity displayed by each microbial isolate towards at least one other individual microbial isolate in the screened population, to create an n-dimensional mutual inhibitory activity matrix based on the mutual inhibitory activities for the screened population;
b) assembling a library of microbial consortia, each microbial consortia comprising a combination of microbial isolates from those screened in step a),
i. wherein the microbial isolates of each microbial consortia in said library are expected to be able to grow together based on the n-dimensional mutual inhibitory activity matrix of step a)
c) screening consortia from the library of microbial consortia by growing said consortia in a growth medium and monitoring the continued presence of each microbial isolate within each consortia
d) selecting a soil-borne plant pathogen inhibiting microbial consortia having the level of mutual inhibitory activity from the library of step b).

33. The method of claim 32, comprising: repeating steps a) through c) one or more times.

34. The method of claim 32, comprising: repeating steps b) through c) one or more times.

35. The method of claim 32, comprising the step of screening a population of microbial isolates in the presence of a plurality of soil-borne plant pathogens to create a soil-borne plant pathogen suppressive profile for each individual microbial isolate in said population, wherein the population of microbial isolates of step (a) comprise soil-borne plant pathogen suppressive profile.

36. The method of claim 26, wherein the n-dimensional mutual inhibitory activity matrix comprises a dimension selected from the group consisting of:
i. strength of inhibitory activity against one or more member microbial isolates,
ii. specificity against one or more member of a plurality of microbial isolates, and
iii. breadth of activity against any one or more member of the plurality of microbial isolates.

37. The method of claim 26, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.

38. The method of claim 26, wherein the soil-borne pathogen is selected from the group consisting of: species of Erwinia, Rhizomonas, Streptomyces scabies, Pseudomonas, and Xanthomonas.

39. A method for creating a soil-borne plant pathogen inhibiting microbial consortia having an optimal and designed level of carbon nutrient utilization complementarity, comprising:
a) screening a population of microbial isolates for carbon nutrient utilization by growing said microbial isolates in a plurality of different nutrient media that comprise a distinct single carbon source to create a carbon nutrient utilization profile for each individual microbial isolate in said population;
b) assembling a library of microbial consortia, each microbial consortia comprising a combination of microbial isolates from those screened in step a),
i. wherein each microbial consortia in said library has a predicted carbon nutrient utilization profile that is distinct from any individual microbial isolate carbon nutrient utilization profile from step a) in at least one dimension of the carbon nutrient utilization profile;
c) optionally ranking microbial consortia from the library of microbial consortia based upon at least one dimension of the carbon nutrient utilization profile of each microbial consortia in said library; and
d) selecting a soil-borne plant pathogen inhibiting microbial consortia having an optimal and designed level of carbon nutrient utilization complementarity from the library.

40. The method of claim 39, comprising the step of screening a population of microbial isolates in the presence of a plurality of soil-borne plant pathogens to create a soil-borne plant pathogen suppressive profile for each individual microbial isolate in said population, wherein the population of microbial isolates of step (a) comprise soil-borne plant pathogen suppressive profile.

41. The method of claim 39, comprising: repeating steps a) through c) one or more times.

42. The method of claim 39, comprising: repeating steps a) through b) one or more times.

43. The method of claim 39, comprising: repeating steps b) through c) one or more times.

44. The method of claim 39, comprising: repeating step b) one or more times.

45. The method of claim 39, wherein each microbial consortia in said library assembled in step b) has a carbon nutrient utilization profile that is distinct from any individual microbial isolate carbon nutrient utilization profile from step a) in at least one dimension selected from the group consisting of:
i. binary ability to grow in any one distinct single carbon source found in said plurality of different nutrient media,
ii. strength of ability to grow in any one distinct single carbon source found in said plurality of different nutrient media,
iii. binary ability to grow in at least two distinct single carbon sources found in said plurality of different nutrient media, and
iv. strength of ability to grow in at least two distinct single carbon sources found in said plurality of different nutrient media.

46. A method for creating a soil-borne plant pathogen inhibiting microbial consortia having an optimal and designed level of carbon nutrient utilization complementarity, comprising:
a) screening a population of microbial isolates for carbon nutrient utilization by growing said microbial isolates in a plurality of different nutrient media that comprise a distinct single carbon source to create a carbon nutrient utilization profile for each individual microbial isolate in said population;
b) assembling a library of microbial consortia, each microbial consortia comprising a combination of microbial isolates from those screened in step a),
i. wherein each microbial consortia in said library has a predicted carbon nutrient utilization profile that is distinct from any individual microbial isolate carbon nutrient utilization profile from step a) in at least one dimension of the carbon nutrient utilization profile;
c) optionally screening consortia from the library of microbial consortia by growing said consortia in a growth medium and monitoring the continued presence of each microbial isolate within each consortia; and
d) selecting a soil-borne plant pathogen inhibiting microbial consortia having an optimal and designed level of carbon nutrient utilization complementarity from the library.

47. The method of claim 46, wherein the population of microbial isolates screened in step a) comprise a soil-borne plant pathogen suppressive profile.

48. A method for creating a soil-borne plant pathogen inhibiting microbial consortia having an optimal and designed level of carbon nutrient utilization complementarity, comprising:
a) accessing a carbon nutrient utilization complementarity microbial library;
b) assembling a library of microbial consortia, each microbial consortia comprising a combination of microbial isolates from the carbon nutrient utilization complementarity microbial library,
i. wherein each microbial consortia in said library has a predicted carbon nutrient utilization profile that is distinct from any individual microbial isolate carbon nutrient utilization profile from step a) in at least one dimension of the carbon nutrient utilization profile;
c) optionally screening consortia from the library of microbial consortia by growing said consortia in a growth medium and monitoring the continued presence of each microbial isolate within each consortia; and
d) selecting a soil-borne plant pathogen inhibiting microbial consortia having an optimal and designed level of carbon nutrient utilization complementarity from the library.

49. The method of claim 48, wherein the microbial isolates assembled in step b) comprise a soil-borne plant pathogen suppressive profile.

50. The method of claim 46, wherein the growth medium of step (c) is medium from the locus where the microbial consortia will be applied, or is medium mimicking a carbon nutrient profile of the locus where the microbial consortia will be applied.

51. The method of claim 46, comprising: repeating steps a) through c) one or more times.

52. The method of claim 46, comprising: repeating steps b) through c) one or more times.

53. A method for creating a soil-borne plant pathogen inhibiting microbial consortia having an optimal and designed level of carbon nutrient utilization complementarity, comprising:
a) assembling a library of microbial consortia, each microbial consortia comprising a combination of microbial isolates comprising a carbon nutrient utilization profile,
i. wherein each microbial consortia in said library has a carbon nutrient utilization profile that is distinct from any individual microbial isolate carbon nutrient utilization profile from step a) in at least one dimension of the carbon nutrient utilization profile;
b) optionally screening consortia from the library of microbial consortia by growing said consortia in a growth medium and monitoring the continued presence of each microbial isolate within each consortia; and
c) selecting a soil-borne plant pathogen inhibiting microbial consortia having an optimal and designed level of carbon nutrient utilization complementarity from the library.

54. The method of claim 53, wherein the growth medium of step (c) is medium from the locus where the microbial consortia will be applied, or is medium mimicking a carbon nutrient profile of the locus where the microbial consortia will be applied.

55. The method of claim 53, wherein the microbial isolates assembled in step a) comprise a soil-borne plant pathogen suppressive profile.

56. The method of claim 53, comprising: repeating steps a) through b) one or more times.

57. The method of claim 56, wherein each assembled microbial consortia has a carbon nutrient utilization profile that is distinct from any individual microbial isolate carbon nutrient utilization profile from step a) in at least one dimension selected from the group consisting of:
i. binary ability to grow in any one distinct single carbon source found in said plurality of different nutrient media,
ii. strength of ability to grow in any one distinct single carbon source found in said plurality of different nutrient media,
iii. binary ability to grow in at least two distinct single carbon sources found in said plurality of different nutrient media, and
iv. strength of ability to grow in at least two distinct single carbon sources found in said plurality of different nutrient media.

58. The method of claim 39, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.

59. The method of claim 39, wherein the soil-borne pathogen is selected from the group consisting of: species of Erwinia, Rhizomonas, Streptomyces scabies, Pseudomonas, and Xanthomonas.

60. A method for creating a soil-borne plant pathogen inhibiting microbial consortia having an optimal and designed level of antibiotic resistance, said method comprising the following steps:
a) creating an n-dimensional antibiotic resistance profile for each individual microbial isolate of a microbial population;
b) assembling a library of microbial consortia, each consortia comprising a plurality of microbial isolates from those screened in step a);
c) optionally ranking microbial consortia from the library of microbial consortia based upon at least one dimension of the antibiotic resistance profile of each microbial consortia in said library; and
d) selecting a soil-borne plant pathogen inhibiting microbial consortia having an optimal and designed level of antibiotic resistance from the library.

61. A method for creating a soil-borne plant pathogen inhibiting microbial consortia having an optimal and designed level of antibiotic resistance comprising the following steps:
a) screening a population of microbial isolates for resistance to a plurality of antibiotics to create an n-dimensional antibiotic resistance profile (or, alternatively, accessing a previously created antibiotic resistance profile);
b) assembling a library of microbial consortia, each microbial consortia comprising a combination of microbial isolates from step a), wherein each microbial consortia in said library is expected to share the antibiotic resistance profile of the individual microbial isolates within the consortia;
c) screening consortia from the library of microbial consortia by growing said microbial consortia in a growth medium comprising an antibiotic that all of the microbial isolates in the microbial consortia are individually resistant to, and monitoring the continued presence of each microbial isolate within each consortia; and
d) selecting a soil-borne plant pathogen inhibiting microbial consortia having the optimal and designed level of antibiotic resistance.

62. The method of claim 60, wherein the microbial isolates assembled in step b) comprise a soil-borne plant pathogen suppressive profile.

63. The method of claim 61, comprising: repeating steps a)-c) one or more times.

64. The method of claim 61, comprising: repeating steps a)-d) one or more times.

65. The method of claim 61, comprising: repeating steps b)-c) one or more times.

66. The method of claim 61, comprising: repeating steps b)-d) one or more times.

67. The method of claim 60, wherein the n-dimensional antibiotic resistance profile comprises at least one dimension selected from the group consisting of:
i. binary ability to grow in the presence of an antibiotic,
ii. the concentration of antibiotic under which the microbial isolate is still capable of growing; and
iii. range of antibiotics against which resistance is shown.

68. The method of claim 60, wherein the antibiotic is selected from the group consisting of tetracycline, chloramphenicol, vancomycin, erythromycin, novobiocin, streptomycin, azithromycin, kanamycin, and rifampin.

69. The method of claim 60, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.

70. The method of claim 60, wherein the soil-borne pathogen is selected from the group consisting of: species of Erwinia, Rhizomonas, Streptomyces scabies, Pseudomonas, and Xanthomonas.

71. A method for creating a soil-borne plant pathogen inhibiting microbial consortia having an optimal and designed level of antimicrobial signaling capacity and responsiveness, comprising:
a) creating an antimicrobial signaling capacity and responsiveness profile for each individual microbial isolate of a microbial population, comprising:
i. screening the population of microbial isolates for the ability of each microbial isolate to signal and modulate the production of antimicrobial compounds in other microbial isolates from the population of microbial isolates; and/or
ii. screening a population of microbial isolates for the ability of each microbial isolate to be signaled and have their production of antimicrobial compounds modulated by other microbial isolates from the population of microbial isolates;
b) assembling a library of microbial consortia, each consortia comprising a plurality of microbial isolates from those screened in step a),
iii. wherein each microbial consortia in said library has an antimicrobial signaling capacity and responsiveness profile that is distinct from any individual microbial isolate antimicrobial signaling capacity and responsiveness profile from step a) in at least one dimension of the antimicrobial signaling capacity and responsiveness profile;
c) optionally ranking microbial consortia from the library of microbial consortia based upon at least one dimension of the antimicrobial signaling capacity and responsiveness profile of each microbial consortia in said library; and
d) selecting a soil-borne plant pathogen inhibiting microbial consortia having an optimal and designed level of antimicrobial signaling capacity and responsiveness from the library.

72. A method for creating a soil-borne plant pathogen inhibiting microbial consortia having an optimal and designed level of antimicrobial signaling capacity and responsiveness, comprising:
a) creating an antimicrobial signaling capacity and responsiveness profile for each individual microbial isolate of a microbial population, comprising:
i. screening the population of microbial isolates for the ability of each microbial isolate to signal and modulate the production of antimicrobial compounds in other microbial isolates from the population of microbial isolates; and/or
ii. screening a population of microbial isolates for the ability of each microbial isolate to be signaled and have their production of antimicrobial compounds modulated by other microbial isolates from the population of microbial isolates;
b) assembling a library of microbial consortia, each consortia comprising a plurality of microbial isolates from those screened in step a),
i. wherein at least one microbial isolate in the microbial consortia exhibits the ability to signal and modulate the production of antimicrobial compounds in another microbial isolate in the consortia;
c) optionally screening microbial consortia from the library of microbial consortia in the presence of a soil-borne pathogen targeted by the antimicrobial compound(s) produced as a consequence of the antimicrobial signaling capacity or responsiveness of at least one microbial isolate in the microbial consortia from step (a); and
d) selecting a soil-borne plant pathogen inhibiting microbial consortia having an optimal and designed level of antimicrobial signaling capacity and responsiveness from the library.

73. A method for creating a soil-borne plant pathogen inhibiting microbial consortia having an optimal and designed level of antimicrobial signaling capacity and responsiveness, comprising:
a) accessing previously gathered antimicrobial signaling capacity and responsiveness profiles for individual microbial isolate of a microbial population;
b) assembling a library of microbial consortia, each consortia comprising a plurality of microbial isolates from those of step a),
i. wherein at least one microbial isolate in the microbial consortia exhibits the ability to signal and modulate the production of antimicrobial compounds in another microbial isolate in the consortia;
c) optionally screening microbial consortia from the library of microbial consortia in the presence of a soil-borne pathogen targeted by the antimicrobial compound(s) produced as a consequence of the antimicrobial signaling capacity or responsiveness of at least one microbial isolate in the microbial consortia from step (a); and
d) selecting a soil-borne plant pathogen inhibiting microbial consortia having an optimal and designed level of antimicrobial signaling capacity and responsiveness from the library.

74. The method of claim 71, wherein the microbial isolates assembled in step b) comprise a soil-borne plant pathogen suppressive profile.

75. The method of claim 71, comprising: repeating steps a) through c) one or more times.

76. The method of claim 71, comprising: repeating steps b) through c) one or more times.

77. The method of claim 72, comprising: repeating steps a) through c) one or more times.

78. The method of claim 72, comprising: repeating steps b) through c) one or more times.

79. The method of claim 71, wherein the antimicrobial signaling capacity and responsiveness profile comprises at least one dimension selected from the group consisting of:
i. binary ability to signal and modulate the production of antimicrobial compounds in other microbial isolates, and
ii. strength of ability to signal and modulate the production of antimicrobial compounds in other microbial isolates.

80. The method of claim 71, wherein the antimicrobial signaling capacity and responsiveness profile comprises at least one dimension selected from the group consisting of:
i. binary ability to be signaled and have their production of antimicrobial compounds modulated by other microbial isolates, and
ii. strength of ability to be signaled and have their production of antimicrobial compounds modulated by other microbial isolates.

81. The method of claim 72, wherein the screening of a population of microbial isolates in step a) comprises: utilizing genomic information, transcriptomic information, and/or growth culture information.

82. A method for creating a soil-borne plant pathogen inhibiting microbial consortia having an optimal and designed level of antimicrobial signaling capacity and responsiveness, comprising:
a) assembling a library of microbial consortia, each consortia comprising a plurality of microbial isolates wherein at least one of said microbial isolates exhibits antimicrobial signaling capacity towards at least one other microbial isolate in the microbial consortia;
b) screening microbial consortia from the library of microbial consortia in the presence of a soil-borne pathogen targeted by the antimicrobial compound(s) produced as a consequence of the antimicrobial signaling capacity or responsiveness of at least one microbial isolate in the microbial consortia;
c) optionally ranking microbial consortia from the library of screened microbial consortia based upon at least one dimension of the antimicrobial signaling capacity and responsiveness profile of each screened microbial consortia; and
d) selecting a soil-borne plant pathogen inhibiting microbial consortia having an optimal and designed level of antimicrobial signaling capacity and responsiveness from the library.

83. The method of claim 82, wherein the microbial isolates assembled in step b) comprise a soil-borne plant pathogen suppressive profile.

84. The method of claim 82, comprising: repeating steps a) through c) one or more times.

85. The method of claim 82, comprising: repeating steps a) through b) one or more times.

86. A method for screening and evaluating a population of microbial isolates for their plant growth ability, said method comprising the steps of
a) applying microbial isolates from the population to a test plant,
b) cultivating the test plant to maturity, and
c) comparing the growth of the test plant against that of a control plant that did not receive the microbial isolate;
wherein differences in the growth between the test plant and the control plant demonstrate a microbial isolate's plant growth promotion ability.

87. A method for creating a soil-borne plant pathogen inhibiting microbial consortia having an optimal and designed level of plant growth promoting ability, comprising the following steps:
(a) creating plant growth promoting ability profile for each individual microbial isolate of a microbial population by screening and evaluating each microbial isolate's ability to promote growth of one or more plants;
(b) assembling a library of microbial consortia, each consortia comprising a plurality of microbial isolates from those screened in step a);
(c) optionally ranking microbial consortia from the library of microbial consortia based upon at least one dimension of the plant growth promoting ability of each microbial consortia in said library; and
(d) selecting a soil-borne plant pathogen inhibiting microbial consortia having an optimal and designed level of plant growth promoting ability from the library.

88. A method for creating a soil-borne plant pathogen inhibiting microbial consortia having an optimal and designed level of plant growth promoting ability, comprising the following steps:
(a) creating plant growth promoting ability profile for each individual microbial isolate of a microbial population by screening and evaluating each microbial isolate's ability to promote growth of one or more plants;
(b) assembling a library of microbial consortia, each consortium comprising a plurality of microbial isolates from those screened in step a);
(c) screening microbial consortia from the library of microbial consortia by:
i) applying microbial consortia from the library to a test plant,
ii) cultivating the test plant to maturity, and
iii) comparing the growth of the test plant against that of a control plant that did not receive the microbial consortia; thereby producing a plant growth promoting ability profile for each screened microbial consortia;
(d) optionally ranking microbial consortia from the library of screened microbial consortia based upon at least one dimension of the plant growth promoting ability profile of each screened microbial consortia; and
(e) selecting a soil-borne plant pathogen inhibiting microbial consortia having an optimal and designed level of plant growth promoting ability from the library.

89. The method of claim 88, comprising: repeating steps a) through c) one or more times.

90. The method of claim 88, comprising: repeating steps a) through d) one or more times.

91. The method of claim 88, comprising: repeating steps b) through c) one or more times.

92. The method of claim 88, comprising: repeating steps b) through d) one or more times.

93. The method of claim 87, wherein the microbial isolates assembled in step b) comprise a soil-borne plant pathogen suppressive profile.

94. The method of claim 87, wherein plant growth promoting ability profile for each screened microbial consortia comprises at least one dimension selected from the group consisting of:
i. binary ability to promote growth of a particular plant;
ii. the degree of growth promotion for the particular plant; and
iii. the mechanism by which the consortia promotes the growth of the particular plant.

95. The method of claim 87, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.

96. The method of claim 87, wherein the soil-borne pathogen is selected from the group consisting of: species of Erwinia, Rhizomonas, Streptomyces scabies, Pseudomonas, and Xanthomonas.

97. A plant pathogen inhibiting microbial consortia, comprising:
a) a first microbial species that provides pathogen suppression; and
b) a second microbial species that has the ability to signal and modulate the production of antimicrobial compounds in the first microbial species.

98. A plant pathogen inhibiting microbial consortia, comprising:
a) Brevibacillus laterosporus;
b) Streptomyces lydicus; and
c) Streptomyces sp. 3211.1

99. A plant pathogen inhibiting microbial consortia, comprising:
a) a Brevibacillus sp. comprising a 16S nucleic acid sequence sharing at least 97% sequence identity to SEQ ID NO: 3;
b) a Streptomyces sp. comprising a 16S nucleic acid sequence sharing at least 97% sequence identity to SEQ ID NO: 2; and
c) a Streptomyces sp. comprising a 16S nucleic acid sequence sharing at least 97% sequence identity to SEQ ID NO: 1.

100. A plant pathogen inhibiting microbial consortia, comprising:
a) a Brevibacillus having deposit accession number NRRL B-67819, or a strain having all of the identifying characteristics of Brevibacillus NRRL B-67819, or a mutant thereof;
b) a Streptomyces having deposit accession numberNRRL B-67820, or a strain having all of the identifying characteristics of Streptomyces NRRL B-67820, or a mutant thereof; and
c) a Streptomyces having deposit accession number NRRL B-67821, or a strain having all of the identifying characteristics of Streptomyces NRRL B-67821, or a mutant thereof.

101. A plant pathogen inhibiting microbial consortia, comprising: a microbial consortia having deposit accession number PTA-124320, or an consortia of strains having all of the identifying characteristics of PTA-124320, or mutants thereof.

102. A plant pathogen inhibiting microbial consortia, comprising: a microbial consortia wherein a representative sample of cells have been deposited under accession number PTA-124320, or an consortia of strains having all of the identifying characteristics of PTA-124320, or mutants thereof.

103. A method of improving soil for plant growth, comprising applying the microbial consortia of claim 97 to the soil.

104. The method of claim 103, wherein the microbial consortia is applied before planting.

105. The method of claim 103, wherein the microbial consortia is applied after plant germination.

106. The method of claim 103, wherein the microbial consortia is applied as a seed treatment.

107. The method of claim 103, wherein the microbial consortia is applied as a spray.

108. The method of claim 103, wherein the microbial consortia is applied as a soil drench.

109. A method of improving soil for plant growth, comprising applying a microbe to the soil; wherein the microbe is a Brevibacillus having deposit accession number NRRL B-67819, or a strain having all of the identifying characteristics of Brevibacillus NRRL B-67819, or a mutant thereof.

110. A method of improving soil for plant growth, comprising applying a microbe to the soil; wherein the microbe is a Streptomyces having deposit accession number NRRL B-67820, or a strain having all of the identifying characteristics of Streptomyces NRRL B-67820, or a mutant thereof.

111. A method of improving soil for plant growth, comprising applying a microbe to the soil;
wherein the microbe is a Streptomyces having deposit accession number NRRL B-67821, or a strain having all of the identifying characteristics of Streptomyces NRRL B-67821, or a mutant thereof.

112. The method of claim 109, wherein the microbial consortia is applied before planting.

113. The method of claim 109, wherein the microbial consortia is applied after plant germination.

114. The method of claim 109, wherein the microbial consortia is applied as a seed treatment.

115. The method of claim 109, wherein the microbial consortia is applied as a spray.

116. The method of claim 109, wherein the microbial consortia is applied as a soil drench.

117. A method for prescriptive biocontrol of a soil-borne plant pathogen, said method comprising:
a) Identifying the soil-borne plant pathogen(s) present in soil or plant tissue from a locus in need of prescriptive biocontrol;
b) creating a customized soil-borne plant pathogen inhibiting microbial consortia capable of suppressing the growth of the soil-borne plant pathogen identified in step (a), wherein creating said customized microbial consortia comprises the steps of:
i. accessing a soil-borne plant pathogen suppressive microbial library, said library comprising one or more ecological function balancing nodal libraries selected from the group consisting of: a mutual inhibitory activity microbial library, a carbon nutrient utilization complementarity microbial library, an antimicrobial signaling capacity and responsiveness microbial library, a plant growth promotion ability microbial library, and an antimicrobial resistance to clinical antimicrobials library;
ii. performing a multi-dimensional ecological function balancing (MEFB) nodal analysis utilizing said one or more nodal libraries; and
iii. selecting at least two microbes from the soil-borne plant pathogen suppressive microbial library based on the MEFB nodal analysis, thereby producing a soil-borne plant pathogen inhibiting microbial consortia, wherein said microbial consortia is capable of suppressing the growth of the soil-borne plant pathogen(s) identified in step (a).

118. A method for prescriptive biocontrol of a soil-borne plant pathogen, said method comprising:
a) identifying and/or culturing the soil-borne plant pathogen(s) present in soil or plant tissue from a locus in need of prescriptive biocontrol;
b) creating a customized soil-borne plant pathogen inhibiting microbial consortia capable of suppressing the growth of the soil-borne plant pathogen identified and/or cultured in step (a), wherein creating said customized microbial consortia comprises the steps of:
i. accessing a soil-borne plant pathogen suppressive microbial library,
ii. utilizing microbes from the library of step i) to create one or more ecological function balancing nodal microbial libraries, selected from the group consisting of: a mutual inhibitory activity microbial library, a carbon nutrient utilization complementarity microbial library, an antimicrobial signaling capacity and responsiveness microbial library, a plant growth promotion ability microbial library, and an antimicrobial resistance to clinical antimicrobials library;
iii. performing a multi-dimensional ecological function balancing (MEFB) nodal analysis utilizing said one or more nodal microbial libraries; and
iv. selecting at least two microbes from the soil-borne plant pathogen suppressive microbial library based on the MEFB nodal analysis, thereby producing a soil-borne plant pathogen inhibiting microbial consortia, wherein said microbial consortia is capable of suppressing the growth of the soil-borne plant pathogen(s) identified in step (a).

119. The method of claim 117, wherein identifying the soil-borne plant pathogen(s) present in soil or plant tissue comprises identification of the pathogen genus based on symptoms of plants grown in said locus in need of prescriptive biocontrol.

120. The method of claim 117, wherein the step of accessing a soil-borne plant pathogen suppressive microbial library comprises creating the soil-borne plant pathogen suppressive microbial library, comprising:
i) screening a population of microbial isolates in the presence of the soil-borne plant pathogen identified and/or cultured in step (a), to create a soil-borne plant pathogen suppressive profile for each individual microbial isolate in said population,
wherein said plant pathogen suppressive profile indicates each microbial isolate's ability to suppress the soil-borne plant pathogen identified and/or cultured in step (a).

121. The method of claim 117, wherein the step of creating or accessing a mutual inhibitory activity microbial library comprises the steps of:
i) assembling a library of test microbial consortia, each test consortia comprising a combination of at least two microbial isolates from the soil-borne plant pathogen suppressive microbial library;
ii) screening test microbial consortia of the assembled library for the relative degree of mutual inhibitory activity displayed by each microbial isolate towards every other microbial isolate within its own test microbial consortia; and
iii) developing an n-dimensional mutual inhibitory activity matrix for test microbial consortia based on the mutual inhibitory activities screened in step (i).

122. The method of claim 117, wherein the step of creating or accessing a carbon nutrient utilization complementarity microbial library comprises the step of: i) screening a population of microbial isolates from the soil-borne plant pathogen suppressive microbial library for carbon nutrient utilization by growing said microbial isolates in a plurality of different nutrient media that comprise a distinct single carbon source to create a carbon nutrient utilization profile for each individual microbial isolate in said population.

123. The method of claim 117, wherein the step of creating an antimicrobial signaling capacity and responsiveness microbial library comprises the steps of:
i) screening a population of microbial isolates from the soil-borne plant pathogen suppressive microbial library for the ability of each microbial isolate to signal and modulate the production of antimicrobial compounds in other microbial isolates from the population of microbial isolates; and/or
ii) screening a population of microbial isolates from the soil-borne plant pathogen suppressive microbial library for the ability of each microbial isolate to be signaled and have their production of antimicrobial compounds modulated by other microbial isolates from the population of microbial isolates;
thereby creating an antimicrobial signaling capacity and responsiveness profile for each screened individual microbial isolate.

124. The method of claim 117, wherein the step of creating an antimicrobial resistance to clinical antimicrobials library comprises the steps of:
i) screening microbial isolates from the soil-borne plant pathogen suppressive microbial library for resistance to a plurality of antibiotics to create an n-dimensional antibiotic resistance profile.

125. The method of claim 117, wherein the step of creating a plant growth promotion ability microbial library comprises the steps of:
i) applying microbial isolates from the soil-borne plant pathogen suppressive microbial library to a test plant,
ii) cultivating the test plant to maturity, and
iii) comparing the growth of the test plant against that of a control plant that did not receive the microbial isolate;
wherein differences in the growth between the test plant and the control plant demonstrate a microbial isolate's plant growth promotion ability.

126. The composition of claim 117, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.

127. The composition of claim 117, wherein the soil-borne pathogen is selected from the group consisting of: species of Erwinia, Rhizomonas, Streptomyces scabies, Pseudomonas, and Xanthomonas.

128. A method for prescriptive biocontrol of a soil-borne plant pathogen, said method comprising:
a) creating a soil nutrient profile from soil from a locus in need of prescriptive biocontrol;
b) creating a customized carbon amendment for application on the locus of step a), wherein the customized carbon amendment supplements a carbon deficiency in the nutrient soil profile.

129. The method of claim 128, further comprising the step of c) applying the customized soil carbon amendment to the locus.

130. The method of claim 129, further comprising the steps of repeating steps a)-b) one or more times.

131. The method of claim 129, wherein each repetition of steps a)-b) occurs at least 1, 2, 3, 4 5 6, 7, 8, 9, 10, 11, or 12 months after the last carbon amendment application to the soil.

132. The method of claim 128, wherein the step of creating a soil nutrient profile comprises the steps of:
i) providing a soil sample from the locus in need of prescriptive biocontrol; and
ii) analyzing the carbon nutrient contents of said soil sample.

133. The method of claim 132, wherein the carbon nutrient contents of the soil sample are measure via a chromatographic method.

134. The method of claim 132, wherein the carbon nutrient contents of the soil sample are measure via an analysis method selected from the group consisting of: Gas Chromatography, Liquid Chromatography, Mass Spectrometer, wet digestion and dry combustion, aerial spectroscopy, Loss on Ignition, Elemental Analyzer, and Reflectance Spectroscopy.

135. The composition of any one of claims 128-134, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.

136. The composition of claim 128, wherein the soil-borne pathogen is selected from the group consisting of: species of Erwinia, Rhizomonas, Streptomyces scabies, Pseudomonas, and Xanthomonas.

137. A method of enhancing the antibiotic inhibitory capacity of individual microbes within a population of microbes in soil, said method comprising the steps of: applying a carbon source to said soil.

138. A method of enriching the densities of inhibitory microorganisms within a population of microbes in soil, said method comprising the steps of: applying a carbon source to said soil.

139. A method of suppressing the growth of pathogens within a soil containing microbes with soil-borne pathogen inhibitory potential, said method comprising the steps of: applying a carbon source to said soil.

140. The method of claim 137, wherein the carbon source is selected from the group consisting of: glucose, fructose, lignin, ground rice powder, malic acid, and mixtures thereof.

141. The method of claim 137, wherein the carbon source is applied at least 2, 3, 4, 5, 6, 7, 8, 9, 10, 11, or 12 times in a one year period.

142. A method of enhancing the antibiotic inhibitory capacity of individual microbes within a population of microbes in soil, wherein crops grown in said soil suffer from one or more soil-borne pathogens, said method comprising the steps of:
a) applying a carbon source to the soil;
b) assessing the antibiotic inhibitory capacity of microbes within the microbial population in the soil; and
c) repeating steps (a) and (b) one or more times, until the antibiotic inhibitory capacity of microbes within the microbial population reaches a desired level.

143. The method of claim 142, wherein the antibiotic inhibitory capacity of the microbes is assessed based on the presence or absence of symptoms exhibited by the crop due to the soil-borne pathogens.

144. The method of claim 142, wherein steps (a) and (b) are repeated until the crops cease to exhibit symptoms from the soil-borne pathogens.

145. A method of enriching the densities of inhibitory microorganisms within a population of microbes in soil, wherein crops grown in said soil suffer from one or more soil-borne pathogens, said method comprising the steps of:
a) applying a carbon source to the soil;
b) assessing the densities of inhibitory microorganisms within the soil; and
c) repeating steps (a) and (b) one or more times, until the densities of inhibitory microorganisms within the soil reaches a desired level.

146. The method of claim 145, wherein the densities of inhibitory microorganisms is assessed based on the presence or absence of symptoms exhibited by the crop due to the soil-borne pathogens.

147. The method of claim 145, wherein steps (a) and (b) are repeated until the crops cease to exhibit symptoms from the soil-borne pathogens.

148. A method of suppressing the growth of pathogens within a soil, containing microbes with soil-borne pathogen inhibitory potential, said method comprising the steps of:
a) applying a carbon source to the soil;
b) determining pathogen density in the soil; and
c) repeating steps (a) and (b) one or more times, until the pathogen density reaches a desired level.

149. The method of claim 148, wherein the densities of inhibitory microorganisms is assessed based on the presence or absence of pathogenic symptoms exhibited by a crop grown on the soil.

150. The method of claim 148, wherein steps (a) and (b) are repeated until crops grown on the soil cease to exhibit symptoms from the soil-borne pathogens.

151. The method of claim 142, wherein step (b) is conducted 1, 2, 3, 4, 5, 6, 7, 8, 9, 10, 11, or 12 months after step (a).

152. A method of treating a soil-borne pathogen in soil, said method comprising the steps of:
a) applying a combination composition to the soil, said composition comprising
i) a soil-borne pathogen suppressing microbe; and
ii) a carbon source;
thereby reducing the symptoms of the soil-borne pathogen on a crop grown in said soil.

153. The method of claim 152, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.

154. The method of claim 152, wherein the soil-borne pathogen is selected from the group consisting of: species of Erwinia, Rhizomonas, Streptomyces scabies, Pseudomonas, and Xanthomonas.

155. The method of claim 152, wherein the soil-borne pathogen suppressing microbe is a microbial isolate.

156. The method of claim 155, wherein the microbial isolate is selected from the group consisting Streptomyces GS1 (Streptomyces lydicus), Streptomyces PS1 (Streptomyces sp. 3211.1) and Brevibacillus PS3 (Brevibacillus laterosporus).

157. The method of claim 152, wherein the soil-borne pathogen suppressing microbe is administered as a microbial consortia.

158. The method of claim 157, wherein the microbial consortia comprises Streptomyces GS1 (Streptomyces lydicus), Streptomyces PS1 (Streptomyces sp. 3211.1) and Brevibacillus PS3 (Brevibacillus laterosporus).

159. A composition comprising i) a soil-borne pathogen suppressing microbe; and i) a carbon source, wherein said composition is capable of suppressing the growth of a soil-borne pathogen.

160. The composition of claim 159, wherein the soil-borne pathogen suppressing microbe is a microbial isolate.

161. The composition of claim 160, wherein the microbial isolate is selected from the group consisting Streptomyces GS1 (Streptomyces lydicus), Streptomyces PS1 (Streptomyces sp. 3211.1) and Brevibacillus PS3 (Brevibacillus laterosporus).

162. The composition of claim 159, wherein the soil-borne pathogen suppressing microbe is administered as a microbial consortia.

163. The composition of claim 162, wherein the microbial consortia comprises Streptomyces GS1 (Streptomyces lydicus), Streptomyces PS1 (Streptomyces sp. 3211.1) and Brevibacillus PS3 (Brevibacillus laterosporus).

164. The composition of claim 159, wherein the soil-borne pathogen is selected from the group consisting of: species of Colletotrichum, Fusarium, Verticillium, Phytophthora, Cercospora, Rhizoctonia, Septoria, Pythium, or Stagnospora. In some embodiments, target soil-born plant pathogens include fungi and fungi-like organisms, including members of Plasmodiophoromyces, Zygomycetes, Oomycetes, Ascomycetes, and Basidiomycetes. In some embodiments, fungi and fungi-like soil-borne plant pathogens include species of Aphanomyces, Bremia, Phytophthora, Pythium, Monosporascus, Sclerotinia, Rusarium Rhizoctonia, Verticillium, Plasmodiophora brassicae, Spongospora subterranean, Macrophomina phaseolina, Monosporascus cannonballus, Pythium aphanidermatum, and Sclerotium rolfsii.

165. The composition of claim 159, wherein the soil-borne pathogen is selected from the group consisting of: species of Erwinia, Rhizomonas, Streptomyces scabies, Pseudomonas, and Xanthomonas.
